(19) United States
(12) Patent Application Publication
(10) Pub. No.: US 2025/0032603 A1
(43) Pub. Date: Jan. 30, 2025

(54) EXPRESSION OF THE SPIKE S GLYCOPROTEIN OF SARS-COV-2 FROM AVIAN PARAMYXOVIRUS TYPE 3 (APMV3)

(71) Applicant: The U.S.A., as represented by the Secretary, Department of Health and Human Services, Bethesda, MD (US)

(72) Inventors: Ursula Buchholz, Silver Spring, MD (US); Peter L. Collins, Kensington, MD (US); Cyril Le Nouen, Bethesda, MD (US); Hong-Su Park, Bethesda, MD (US); Shirin Munir, Potomac, MD (US); Cindy Luongo, Bethesda, MD (US)

(73) Assignee: The U.S.A., as represented by the Secretary, Department of Health and Human Services, Bethesda, MD (US)

(21) Appl. No.: 18/710,343

(22) PCT Filed: Nov. 17, 2022
(86) PCT No.: PCT/US2022/080015

§ 371 Date: May 15, 2024

Related U.S. Application Data
(60) Provisional application No. 63/280,884, filed on Nov. 18, 2021.

(51) Int. Cl. A61K 39/215; A61K 39/00; A61P 31/14; C07K 14/005; C12N 15/86

(52) U.S. Cl. CPC A61K 39/215; A61P 31/14; C07K 14/005; C12N 15/86; A61K 2039/5256; A61K 2039/541; C12N 2760/18043; C12N 2770/20022; C12N 2770/20034; C12N 2770/20051

(57) ABSTRACT

Coronavirus spike protein, for example, SARS-CoV-2 spike (S) protein, expressed by an avian paramyxovirus type 3 (APMV3) as a vaccine vector for prevention and treatment against infection, such as SARS-CoV-2.

### CROSS REFERENCE TO RELATED APPLICATIONS

This application claims the benefit of U.S. Provisional Application 63/280,884 filed on Nov. 18, 2022. The entire contents of this application are incorporated herein by reference in its entirety.

### FIELD OF THE DISCLOSURE

Immunogenic compositions are provided in the prevention and treatment of coronavirus disease. In particular, the immunogenic compositions can be administered via the intranasal or oral routes, e.g., a spray, mist, or aerosol.

### BACKGROUND

Severe acute respiratory syndrome coronavirus 2 (SARS-CoV-2) is a positive-sense, single-stranded RNA virus that is closely related to SARS-CoV-1 and Middle East respiratory syndrome coronavirus (MERS-CoV), all of which belong to the genus Betacoronavirus of the family Coronaviridae. SARS-CoV-2 emerged in 2019 as the causative agent of coronavirus disease 2019 (COVID-19) and has created a pandemic and global crisis in public health. To efficiently control SARS-CoV-2 worldwide, vaccines and therapeutics are urgently needed.

### SUMMARY

The present disclosure provides for prevention, treatment, reduction of the incidence of and/or severity of clinical symptoms associated with a coronavirus infection, e.g. SARS-CoV-2 infection.

In certain embodiments, a vector comprises a paramyxovirus comprising one or more polynucleotides encoding one or more coronavirus proteins comprising a spike (S) protein, a membrane (M) protein, an envelope (E) protein or a nucleocapsid (N) protein or fragments thereof. In certain aspects, the coronavirus is a severe acute respiratory syndrome coronavirus (SARS-CoV-2). SARS-CoV-2 has a complete genome as shown by NC 045512.2, in which the sequence of nucleic acids 21563 to 25384 is a coding sequence for Spike protein (S). In certain aspects, the coronavirus protein is a SARS-CoV-2 spike (S) protein or fragments thereof. In certain aspects, the paramyxovirus is an avian paramyxovirus type 3 virus (APMV3). In certain aspects, the SARS-CoV-2 S protein or fragments thereof, comprises one or more amino acid substitutions. In certain aspects, the SARS-CoV-2 S protein or fragments thereof, comprises two or more amino acid substitutions. In certain aspects, the SARS-CoV-2 S protein or fragments thereof, comprises six or more amino acid substitutions. In certain aspects, the SARS-CoV-2 S protein or fragments thereof, comprises one or more proline substitutions. In certain aspects, the SARS-CoV-2 S protein or fragments thereof, comprises one or more proline substitutions at amino acid positions 817, 892, 899, 942, 986 and 987. In certain aspects, the SARS-CoV-2 S protein or fragments thereof, comprises six proline substitutions at amino acid positions 817, 892, 899, 942, 986 and 987. In certain aspects, the SARS-CoV-2 S protein further comprises one or more amino acid substitutions comprising L18F, T20N, P26S, D138Y, R190S, K417N, N439K, N440K, L452R, S477G, S477N, E484K, E484Q, N501Y, D614G, H655Y, P681H, or T1027I. In certain aspects, the SARS-CoV-2 S protein further comprises one or more deletions of amino acid H69, V70, Y144, L242, A243, or L244. In certain aspects, the SARS-CoV-2 S protein further comprises one or more amino acid substitutions comprising T19R, V70F, T95I, G142D, E156-, F157-, R158G, A222V, W258L, K417N, L452R, T478K, D614G, P681R, or D950N.

In certain aspects, the S polynucleotide is codon optimized as compared to a wild-type polynucleotide encoding the one or more S protein or fragments thereof protein and comprises nucleotide adapters for insertion into the APMV3 full-length cDNA. In certain aspects, the S polynucleotide is inserted between the APMV3 P and M genes. In certain aspects, the S open reading frame (ORF) is transcribed by the APMV 3 polymerase complex as a distinct mRNA. In certain aspects, the SARS-CoV-2 S protein or fragments thereof, further comprise amino acid substitutions to a polybasic furin cleavage motif. In certain aspects, the polybasic furin cleavage motif comprising an RRAR amino acid sequence is substituted with a GSAS amino acid sequence. In certain aspects, the vector is formulated for administration intranasally, orally, intramuscularly, intraperitoneally or intravenously. In certain aspects, the vector is formulated as a spray, mist or aerosol.

In certain embodiments, a method of producing a coronavirus vaccine comprises inserting a nucleic acid sequence encoding a severe acute respiratory syndrome coronavirus (SARS-CoV-2) spike (S) protein or fragments thereof, into an avian paramyxovirus type 3 virus (APMV3); contacting or transfecting a mammalian cell culture stably expressing T7 RNA polymerase with isolated polynucleotide molecules that include a nucleic acid sequence encoding the genome or antigenome of the APMV3 virus comprising a polynucleotide encoding the SARS-CoV-2 S protein or fragments thereof, together with polynucleotides encoding the N, P, and L proteins of APMV3, harvesting and culturing the APMV3 virus in Vero cells; injecting the APMV3 virus harvested from the Vero cells into an allantoic cavity of embryonated chicken eggs; harvesting and isolating the APMV3 comprising the nucleic acid encoding the SARS-CoV-2 S protein or fragments thereof, and producing the coronavirus vaccine. In certain aspects, the SARS-CoV-2 S protein or fragments thereof, comprises one or more amino acid substitutions. In certain aspects, the SARS-CoV-2 S protein or fragments thereof, comprises two or more amino acid substitutions. In certain aspects, the SARS-CoV-2 S protein or fragments thereof, comprises six or more amino acid substitutions. In certain aspects, the SARS-CoV-2 S protein or fragments thereof, comprises one or more proline substitutions. In certain aspects, the SARS-CoV-2 S protein or fragments thereof, comprises one or more proline substitutions at amino acid positions 817, 892, 899, 942, 986 and 987. In certain aspects, the SARS-CoV-2 S protein or fragments thereof, comprises six proline substitutions at amino acid positions 817, 892, 899, 942, 986 and 987. In certain aspects, the SARS-CoV-2 S protein further comprises one or more amino acid substitutions comprising L18F, T20N, P26S, D138Y, R190S, K417N, N439K, N440K, L452R, S477G, S477N, E484K, E484Q, N501Y, D614G, H655Y, P681H, or T1027I. In certain aspects, the SARS-CoV-2 S protein further comprises one or more deletions of amino acid H69, V70, Y144, L242, A243, or L244. In certain aspects, the SARS-CoV-2 S protein further comprises one or more amino acid substitutions comprising T19R, V70F, T95I, G142D, E156-, F157-, R158G, A222V, W258L, K417N, L452R, T478K, D614G, P681R, or D950N.

In certain aspects, the S polynucleotide is codon optimized as compared to a wild-type polynucleotide encoding the one or more S protein or fragments thereof and comprises nucleotide adapters for insertion into the APMV3 full-length cDNA. In certain aspects, the SARS-CoV-2 S polynucleotide, is inserted between the APMV3 P and M genes. In certain aspects, the S open reading frame (ORF) is transcribed by the APMV 3 polymerase complex as a distinct mRNA. In certain aspects, the SARS-CoV-2 S protein or fragments thereof, further comprise substitutions to inactivate or mutate a polybasic furin cleavage motif. In certain aspects, the polybasic furin cleavage motif comprising an RRAR amino acid sequence is substituted with a GSAS amino acid sequence.

In certain embodiments, a vaccine comprises a paramyxovirus recombinant vector comprising one or more polynucleotides encoding a coronavirus protein comprising a spike (S) protein, a membrane (M) protein, an envelope (E) protein, a nucleocapsid (N) protein and combinations thereof, or fragments thereof, formulated as a spray, mist, or aerosol. In certain aspects, the coronavirus is a severe acute respiratory syndrome coronavirus (SARS-CoV-2). In certain aspects, the coronavirus peptide is a SARS-CoV-2 spike (S) protein or fragments thereof. In certain aspects, the SARS-CoV-2 S protein or fragments thereof, comprises six proline substitutions at amino acid positions 817, 892, 899, 942, 986 and 987. In certain aspects, the SARS-CoV-2 S protein further comprises one or more amino acid substitutions comprising L18F, T20N, P26S, D138Y, R190S, K417N, N439K, N440K, L452R, S477G, S477N, E484K, E484Q, N501Y, D614G, H655Y, P681H, or T1027I. In certain aspects, the SARS-CoV-2 S protein further comprises one or more deletions of amino acid H69, V70, Y144, L242, A243, or L244. In certain aspects, the SARS-CoV-2 S protein further comprises one or more amino acid substitutions comprising T19R, V70F, T95I, G142D, E156-, F157-, R158G, A222V, W258L, K417N, L452R, T478K, D614G, P681R, or D950N. In certain aspects, the paramyxovirus recombinant vector is an avian paramyxovirus type 3 virus (APMV3).

In certain aspects, a vaccine comprises an RNA oligonucleotide encoding a coronavirus peptide. In certain embodiments, the RNA oligonucleotide encodes a SARS-CoV-2 spike (S) protein or fragments thereof. In certain aspects, the RNA oligonucleotide encodes an SARS-CoV-2 S protein or fragments thereof, comprising six proline substitutions at amino acid positions 817, 892, 899, 942, 986 and 987. In certain aspects the RNA oligonucleotide encodes an SARS-CoV-2 S protein which further comprises one or more amino acid substitutions comprising L18F, T20N, P26S, D138Y, R190S, K417N, N439K, N440K, L452R, S477G, S477N, E484K, E484Q, N501Y, D614G, H655Y, P681H, or T1027I. In certain aspects, the RNA oligonucleotide encodes an SARS-CoV-2 S protein comprising one or more deletions of amino acid H69, V70, Y144, L242, A243, or L244. In certain aspects, the RNA oligonucleotide encodes an SARS-CoV-2 S protein comprising one or more amino acid substitutions comprising T19R, V70F, T95I, G142D, E156-, F157-, R158G, A222V, W258L, K417N, L452R, T478K, D614G, P681R, or D950N. In certain embodiments, the RNA is administered to a subject. In certain embodiments, the RNA is included in a delivery vehicle and administered to a subject.

In certain embodiments, the vaccine is administered to induce an immune response in a subject. In certain embodiments, one or more doses are administered at various intervals as determined by a medical practitioner or governmental guidelines. In certain embodiments, one dose is sufficient to induce a protective immune response. In certain embodiments, the vaccine is administered as secondary booster vaccine following priming by mRNA vaccines, peptide vaccines or other injectable or intranasal COVID-19 vaccines, or as a booster dose following natural infection with SARS-CoV-2.

In certain embodiments, a pharmaceutical composition comprising a paramyxovirus recombinant vector comprising one or more polynucleotides encoding a coronavirus protein comprising a spike (S) protein, a membrane (M) protein, an envelope (E) protein, a nucleocapsid (N) protein and combinations thereof, or fragments thereof. In certain aspects, the coronavirus is a severe acute respiratory syndrome coronavirus (SARS-CoV-2). In certain aspects, the coronavirus peptide is a SARS-CoV-2 spike (S) protein or fragments thereof. In certain aspects, the SARS-CoV-2 S protein or fragments thereof, comprises six proline substitutions at amino acid positions 817, 892, 899, 942, 986 and 987. In certain aspects, the SARS-CoV-2 S protein further comprises one or more amino acid substitutions comprising L18F, T20N, P26S, D138Y, R190S, K417N, N439K, N440K, L452R, S477G, S477N, E484K, E484Q, N501Y, D614G, H655Y, P681H, or T1027I. In certain aspects, the SARS-CoV-2 S protein further comprises one or more deletions of amino acid H69, V70, Y144, L242, A243, or L244. In certain aspects, the SARS-CoV-2 S protein further comprises one or more amino acid substitutions comprising T19R, V70F, T95I, G142D, E156-, F157-, R158G, A222V, W258L, K417N, L452R, T478K, D614G, P681R, or D950N.

In certain aspects, the paramyxovirus is an avian paramyxovirus type 3 virus (APMV3). In certain aspects, the pharmaceutical composition further comprises an adjuvant. In certain aspects, the pharmaceutical composition further comprises a secondary therapeutic agent. In certain aspects, the secondary therapeutic agents comprise: a decongestant, an antihistamine, a pain reliever, a fever reducer, a cough suppressant, a cytokine, an antibiotic, an anti-viral agent and combinations thereof.

In certain embodiments, the SARS-CoV-2 S protein further comprises one or more amino acid substitutions comprising L18F, T20N, P26S, D138Y, R190S, K417N, N439K, N440K, L452R, S477G, S477N, E484K, E484Q, N501Y, D614G, H655Y, P681H, or T1027I.

In certain embodiments, the SARS-CoV-2 S protein further comprises one or more deletions of amino acid H69, V70, Y144, L242, A243, or L244.

In certain embodiments, the SARS-CoV-2 S protein further comprises one or more amino acid substitutions comprising T19R, V70F, T95I, G142D, E156-, F157-, R158G, A222V, W258L, K417N, L452R, T478K, D614G, P681R or D950N.

In certain embodiments, a method of preventing and treating a subject at risk of contracting a severe acute respiratory syndrome coronavirus (SARS-CoV-2), comprises administering to the subject a vector(s), a vaccine(s) or the pharmaceutical compositions embodied herein. In certain aspects, administration of the vector or vaccine comprises subcutaneous (sc) delivery, intramuscular (im) delivery, intradermal delivery, transdermal delivery, mucosal delivery, intravaginal delivery, intrarectal delivery, intraperitoneal delivery, inhalation delivery, aerosol delivery and combinations thereof. In certain aspects, the pharmaceutical agent(s), vector(s) or vaccine(s) is administered via inhalation delivery, aerosol delivery or the combination thereof. In certain aspects, the method further comprises administering an adjuvant. In certain aspects, the method further comprises administering a secondary therapeutic agent. In certain aspects, the secondary therapeutic agents comprise a decongestant, an antihistamine, a pain reliever, a fever reducer, a cough suppressant, a cytokine, an antibiotic, an anti-viral agent and combinations thereof.

In certain embodiments, the vector comprises a viral or a bacterial vector. In various embodiments, the viral vector is selected from the group comprising adenovirus, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus or cucumber mosaic virus. In various embodiments, the virus is an oncolytic virus. In various embodiments the virus is a chimeric virus, a synthetic virus, a mosaic virus or a pseudotyped virus.

In certain embodiments, a method is provided for preventing or treating a corona virus infection in a subject in need thereof by aerosolizing the pharmaceutical compositions described herein in a nasal passageway of the subject. In one embodiment, the subject is a human subject. In certain embodiments, the virus is SARS CoV 2.

In still yet another embodiment, a packaged device is provided that includes the pharmaceutical composition described herein optionally together with instructions for use. In one embodiment, the device is selected from the group consisting of aerosol dispenser, pneumatically pressurized device, multi-dose metered dose spray pump, inhaler, pump sprayer, and nebulizer.

Other aspects are discussed infra.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “active agent”, refers to any molecule that is used for the prevention or treatment of a virus infection, e.g., a respiratory virus infection, and include agents which alleviate any symptom, such as runny nose, blocked nose, sore throat, sneezing, chilliness, headache, muscle ache, cough, etc., associated with the virus. Examples of active agents include the pharmaceutical agent, vaccine or vector embodied herein. The “active agent” can be a compound that is directly or indirectly effective in specifically interfering with at least one viral action, such as for example, virus penetration of eukaryotic cells, virus replication in eukaryotic cells, virus assembly, virus release from infected eukaryotic cells, or that is effective in inhibiting a virus titer increase or in reducing a virus titer level in a eukaryotic or mammalian host system. It also refers to a compound that prevents from or reduces the likelihood of getting a viral infection.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes. A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

As used herein, an “adjuvant” refers to a substance that enhances the body's immune response to an antigen or a vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalene or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers comprise nonionic surfactants, in particular esters of sorbitan, of mannide (e.g., anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC™ brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In a preferred embodiment the adjuvant is at a concentration of about 0.01 to about 50%, at a concentration of about 2% to 30%, at a concentration of about 5% to about 25%, at a concentration of about 7% to about 22%, and at a concentration of about 10% to about 20% by volume of the final product. Examples of suitable adjuvants are described in U.S. Patent Application Publication No. US2004/0213817 A1. “Adjuvanted” refers to a composition that incorporates or is combined with an adjuvant.

As used herein, “antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Decongestant activity” as used herein shall relate to any activity of the novel preparations referred to herein that leads to a partial or full deblocking of a temporarily blocked, stuffy nose, irrespective of the cause of such temporary blocking of the nose such as, for example, an allergic or viral impairment.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylenediaminetetraacetic acid, among others.

The term “effective dose” or “effective dosage” is defined as an amount sufficient to achieve or at least partially achieve the desired effect.

An “epitope” is an antigenic determinant that is immunologically active in the sense that once administered to the host, it is able to evoke an immune response of the humoral (B cells) and/or cellular type (T cells). These are particular chemical groups or peptide sequences on a molecule that are antigenic. An antibody specifically binds a particular antigenic epitope on a polypeptide. In the animal, most antigens will present several or even many antigenic determinants simultaneously. Such a polypeptide may also be qualified as an immunogenic polypeptide and the epitope may be identified as described further.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

An “immunogenic or immunological composition” refers to a composition of matter that comprises at least one antigen, which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or γδ T cells, directed specifically to an antigen or antigens included in the composition or vaccine of interest. The host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or virus titer in the tissues or body fluids or excretions of the infected host compared to a healthy control. The reduction in symptoms is statistically significant when compared to a control.

The term “immunogenic fragment” as used herein refers to a polypeptide or a fragment of a polypeptide, or a nucleotide sequence encoding the same which comprises an allele-specific motif, an epitope or other sequence such that the polypeptide or the fragment will bind an MHC molecule and induce a cytotoxic T lymphocyte (“CTL”) response, and/or a B cell response (for example, antibody production), and/or T-helper lymphocyte response, and/or a delayed type hypersensitivity (DTH) response against the antigen from which the immunogenic polypeptide or the immunogenic fragment is derived. A DTH response is an immune reaction in which T cell-dependent macrophage activation and inflammation cause tissue injury. A DTH reaction to the subcutaneous injection of antigen is often used as an assay for cell-mediated immunity.

An “immune response,” as used herein, refers to a biological response within a vertebrate against foreign agents, e.g., virus, or abnormal cells, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell. Accordingly, an immune response can comprise a humoral immune response (e.g., mediated by B-cells), cellular immune response (e.g., mediated by T cells), or both humoral and cellular immune responses. In some aspects, an immune response is an “inhibitory” immune response. An “inhibitory” immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A “stimulatory” immune response is an immune response that results in the generation of effectors cells (e.g., cytotoxic T lymphocytes) that can destroy and clear a target antigen (e.g., tumor antigen or viruses).

As used herein, the term “cellular immune response” can be used interchangeably with the term “cell-mediated immune response” and refers to an immune response that does not predominantly involve antibodies. Instead, a cellular immune response involves the activation of different immune cells (e.g., phagocytes and antigen-specific cytotoxic T-lymphocytes) that produce various effector molecules (e.g., cytokines, perforin, granzymes) upon activation (e.g., via antigen stimulation). As used herein, the term “humoral immune response” refers to an immune response predominantly mediated by macromolecules found in extracellular fluids, such as secreted antibodies, complement proteins, and certain antimicrobial peptides. The term “antibody-mediated immune response” refers to an aspect of a humoral immune response that is mediated by antibodies.

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4⁺ T cells or CD8⁺ T cells). In some aspects, an immune cell that can be targeted with a targeting moiety (e.g., anti-CD3) comprises a naive CD4⁺ T cell. In some aspects, an immune cell comprises a memory CD4⁺ T cell. In some aspects, an immune cell comprises an effector CD4+T cell. In some aspects, an immune cell comprises a naive CD8⁺ T cell. In some aspects, an immune cell comprises a memory CD8⁺ T cell. In some aspects, an immune cell comprises an effector CD8⁺ T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used in this specification and the appended claims, the term “or” is generally employed in its sense including “and/or” unless the content clearly dictates otherwise.

The term “patient” includes human and other mammalian subjects that receive either prophylactic or therapeutic treatment.

As used herein, “a pharmaceutically acceptable carrier” or “pharmaceutical carrier” includes any and all excipients, solvents, growth media, dispersion media, coatings, adjuvants, stabilizing agents, diluents, preservatives, inactivating agents, antimicrobial, antibacterial and antifungal agents, isotonic agents, adsorption delaying agents, and the like. Such ingredients include those that are safe and appropriate for use in veterinary applications. Pharmaceutically acceptable carriers are typically non-toxic, inert, solid or liquid carriers.

The term “prophylactic treatment” as used herein, refers to any intervention using the compositions embodied herein, that is administered to an individual in need thereof or having an increased risk of acquiring a respiratory tract infection, wherein the intervention is carried out prior to the onset of a viral infection, e.g. SARS-CoV-2, and typically has in effect that either no viral infection occurs or no clinically relevant symptoms of a viral infection occur in a healthy individual upon subsequent exposure to an amount of infectious viral agent that would otherwise, i.e. in the absence of such a prophylactic treatment, be sufficient to cause a viral infection.

A “susceptible” host as used herein refers to a cell or an animal that can be infected by a coronavirus, e.g., SARS-CoV-2. When introduced to a susceptible animal, an attenuated SARS-CoV-2 may also induce an immunological response against the SARS-CoV-2 or its antigen, and thereby render the animal immunity against SARS-CoV-2 infection.

As used herein, the term “T cell” or “T-cell” refers to a type of lymphocyte that matures in the thymus. T cells play an important role in cell-mediated immunity and are distinguished from other lymphocytes, such as B cells, by the presence of a T-cell receptor on the cell surface. T-cells include all types of immune cells expressing CD3, including T-helper cells (CD4⁺ cells), cytotoxic T-cells (CD8⁺ cells), natural killer T-cells, T-regulatory cells (Treg), and gamma-delta T cells. A “naïve” T cell refers to a mature T cell that remains immunologically undifferentiated (i.e., not activated). Following positive and negative selection in the thymus, T cells emerge as either CD4⁺ or CD8⁺ naïve T cells. In their naive state, T cells express L-selectin (CD62L⁺), IL-7 receptor-α (IL-7R-α), and CD132, but they do not express CD25, CD44, CD69, or CD45RO. As used herein, “immature” can also refer to a T cell which exhibits a phenotype characteristic of either a naïve T cell or an immature T cell, such as a TSCM cell or a TCM cell. For example, an immature T cell can express one or more of L-selectin (CD62L⁺), IL-7Rα, CD132, CCR7, CD45RA, CD45RO, CD27, CD28, CD95, CXCR3, and LFA-1. Naive or immature T cells can be contrasted with terminal differentiated effector T cells, such as T_{EM} cells and T_{EFF} cells. Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCMX} central memory T (TCM effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

As used herein, the term “effector” T cells or “T_{EFF}” cells refers to a T cell that can mediate the removal of a pathogen or cell without requiring further differentiation. Thus, effector T cells are distinguished from naive T cells and memory T cells, and these cells often have to differentiate and proliferate before becoming effector cells.

As used herein, the term “memory” T cells refer to a subset of T cells that have previously encountered and responded to their cognate antigen. In some aspects, the term is synonymous with “antigen-experienced” T cells. In some aspects, memory T cells can be effector memory T cells or central memory T cells. In some aspects, the memory T cells are tissue-resident memory T cells. As used herein, the term “tissue-resident memory T cells” or “TRM cells” refers to a lineage of T cells that occupies tissues (e.g., skin, lung, gastrointestinal tract) without recirculating. TRM cells are transcriptionally, phenotypically and functionally distinct from central memory and effector memory T cells which recirculate between blood, the T cell zones of secondary lymphoid organs, lymph and nonlymphoid tissues. One of the roles of TRM cells is to provide immune protection against infection in extra lymphoid tissues.

The term “therapeutically effective dose” is defined as an amount sufficient to cure or at least partially arrest the disease, e.g., COVID-19 and its complications in a patient already suffering from the disease.

The term “therapy” or “therapeutic treatment” as used herein relates to the administration of the compositions embodied herein, e.g. the vaccine or vector embodied herein, in order to achieve a reduction in severity and/or frequency of symptoms, elimination of symptoms and/or underlying cause, prevention of the occurrence of symptoms and/or their underlying cause, and/or improvement or remediation of damage directly caused by or indirectly associated, e.g. through secondary infection, with the viral infection.

“Treatment”, or “treating” as used herein, is defined as the application or administration of a therapeutic agent or combination of therapeutic agents (e.g., the vaccine or vector embodied herein; optionally, a secondary therapeutic agent, e.g. anti-viral agent preventing agent) to a patient, or application or administration of the active agent to a patient, who has a virus infection, e.g. SARS-CoV-2 with the purpose to cure, heal, alleviate, relieve, alter, remedy, ameliorate, improve or affect the infection, or symptoms thereof. The term “treatment” or “treating” is also used herein in the context of administering agents prophylactically. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof, or (4) relieving the disease, i.e., causing regression of the state, disorder or condition or at least one of its clinical or subclinical symptoms. The benefit to an individual to be treated is either statistically significant or at least perceptible to the patient or to the physician.

As used herein, “a unit dose” as used herein represents the therapeutically effective amount of active ingredients, e.g., the vaccine or vector embodied herein, that is administered to prevent or treat the viral infection. In cases where the compositions are delivered, by for example, via the nostril, each nostril will receive a unit dose.

The term “vaccine,” as used herein, refers to a composition that includes an antigen. Vaccine may also include a biological preparation that improves immunity to a particular disease. A vaccine may typically contain an agent, referred to as an antigen, that resembles a disease-causing organism, and the agent may often be made from mRNA, vectors expressing the immunogen, peptides, infected cells etc. The antigen may stimulate the body's immune system to recognize the agent as foreign, destroy it, and “remember” it, so that the immune system can more easily recognize and destroy any of these microorganisms that it later encounters. Similarly, the modified toxin preparations, combined with vaccines against other pathogens, could “boost” the immune responses to the pathogen of interest, by acting themselves as vaccine adjuvants. Adjuvants can be classified according to their physiochemical properties or mechanisms of action. The two major classes of adjuvants include compounds that directly act on the immune system such as bacterial toxins that stimulate immune responses, and molecules able to facilitate the presentation of antigens in a controlled manner and behaving as a carrier.

Vaccine formulations will contain a “therapeutically effective amount” of the active ingredient, that is, an amount capable of eliciting an induction of an immunoprotective response in a subject to which the composition is administered. In the treatment and prevention of SARS-CoV-2, for example, a “therapeutically effective amount” would be an amount that enhances resistance of the vaccinated subject to new infection and/or reduces the clinical severity of the disease. Such protection will be demonstrated by either a reduction or lack of symptoms normally displayed by a subject infected with SARS-CoV-2, a quicker recovery time and/or a lowered count of virus particles. Vaccines can be administered prior to infection, as a preventative measure against SARS-CoV-2. Alternatively, vaccines can be administered after the subject already has contracted a disease. Vaccines given after exposure to SARS-CoV-2 may be able to attenuate the disease, triggering a superior immune response than the natural infection itself.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence. The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

Genbank and NCBI submissions indicated by accession number cited herein are incorporated herein by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

FIGS. 1A-1G are a series of graphs, schematics and plaque immunostaining images demonstrating the generation and replication of APMV3 expressing a genetically-stabilized prefusion form of SARS-CoV-2 S protein (APMV3/S-6P), and stability of S expression. FIG. 1A: Diagram of the APMV3 genome and the SARS-CoV-2 S gene and encoded protein. A copy of the full-length SARS-CoV-2 S ORF (aa 1-1,273) from the first available SARS-CoV-2 genome sequence (Genbank NC 045512.2) was codon-optimized for human expression, flanked by an adapter sequence, and inserted between the APMV3 P and M genes under the control of a set of APMV gene-start and gene-end transcription signals for expression as a separate mRNA and with a “Kozak” sequence for optimal initiation of translation (see Materials and Methods). Two additional versions were generated, expressing two different prefusion-stabilized versions of the S protein (APMV3/S-2P and APMV3/S-6P, see Materials and Methods and Results). Both prefusion-stabilized versions of the S ORF include RRAR-to-GSAS changes that ablate the S1/S2 cleavage site. The presence of two (APMV3/S-2P) or six (APMV3/S-6P) stabilizing proline substitutions is indicated by red lines marked by arrows. FIG. 1B: Virus titers of APMV3 and derivatives in the allantoic fluid of embryonated chicken eggs on day 2 pi determined by plaque assay on Vero cells as described in Materials and Methods. Error bars indicate the standard deviation (SD) of the mean from two (APMV3, APMV3/S and APMV3/S-2P) or three (APMV3/S-6P) independent virus stocks. FIG. 1C: Stability of expression of the different versions of SARS-CoV-S protein. Vero cells were inoculated with ten-fold serial dilutions of egg-grown stocks of APMV3 and derivatives, incubated under methylcellulose overlay, fixed on day 4 pi, and incubated with chicken anti-APMV3 antibodies and a human anti-SARS-CoV-2 S monoclonal antibody CR3022, followed by incubation with infrared fluorescent antibody-labeled secondary antibodies. Infrared images were obtained, and APMV3 and SARS-CoV-2 S immunostainings were pseudocolored red and green, respectively. Plaques expressing both APMV3 and SARS-CoV-2 S antigens appear yellow. Chicken IgY and human IgG1 antibodies were used as negative controls. FIG. 1D: Percentage of S-positive plaques out of total plaques in immunoplaque assays of egg-grown stocks of APMV3/S, APMV3/S-2P, and APMV3/S-6P. Error bars indicate SD of the means from two (APMV3, APMV3/S and APMV3/S-2P) or three (APMV3/S-6P) independent virus stocks. **=p<0.0001.

FIGS. 1E and 1F: Multi-cycle growth kinetics of APMV3 and APMV3/S-6P in Vero (FIG. 1E) and A549 cells (FIG. 1F). Replicate sub-confluent monolayers were infected in quadruplicate with each virus using an MOI of 0.01 PFU/cell. After 2 h incubation, Vero cells were washed once and replenished with Opti-MEM containing 1% L-glutamine and 2% TrypLE Select. A549 cells were washed and replenished with F-12K containing 1% L-glutamine and 0.5% TrypLE Select. Every 8 h from 8 to 80 hpi, cells from duplicate wells per virus and time point were harvested by scraping into media, subjected to short centrifugation to collect the clarified supernatant that was aliquoted and snap-frozen followed by storage at −80° C. Virus titers were determined in duplicate by immunoplaque assay on Vero cells at 37° C. as described in Materials and Methods. The third replica wells from the growth curves were used to evaluate the expression of SARS-CoV-2 S and APMV3 N proteins by Western blotting, and the fourth wells were used to analyze the expression of SARS-CoV-2 S protein by flow cytometry (see FIG. 2A and FIGS. 7A-7E). FIG. 1G: Percentage of S-positive plaques out of total plaques generated by APMV3/S-6P in Vero and A549 cells during the multicycle growth kinetics described in E and F. Error bars indicate SD of the mean.

FIGS. 2A-2C are a series of Western blots and images from an electron microscope demonstrating the expression of virus proteins and incorporation of the SARS-CoV-2 S protein into the vector particles. FIG. 2A: Viral protein expression in Vero cells. From the experiment described in FIGS. 1E and 1F, one additional replicate well of Vero cells per virus, which had been infected in duplicate with an MOI of 0.01 PFU/cell of the indicated virus, was harvested at 48 h, and cell lysates were prepared and analyzed by SDS-PAGE under reducing and denaturing conditions. The APMV3 N and SARS-CoV-2 S protein bands were visualized by Western blotting using primary rabbit anti-APMV3 serum and goat anti-SARS-CoV-2 S serum, followed by incubation with IRDye-conjugated secondary antibodies and infrared imaging (Materials and Methods). A mouse anti-β-actin antibody was included to provide a loading control. FIGS. 2B, 2C: Incorporation of the SARS-CoV-2 S protein into the vector particles. FIG. 2B: Preparations of APMV3 and APMV3/S-6P were sucrose-purified, and three μg of protein from each preparation were analyzed per lane by SDS-PAGE under reducing and denaturing condition. One set of lanes from the gel was subjected to silver staining (left panel). In parallel, a duplicate set of lanes from the same gel was analyzed by Western blotting to detect the presence of SARS-CoV-2 S along with APMV3 N (right panel). Images are representative of three independent experiments. The identities of the bands annotated in the silver-stained gel were confirmed by proteomics analysis (see Results). Ladder, molecular size marker. FIG. 2C: Incorporation of SARS-CoV-2 S in APMV3/S-6P virus particles was also evaluated by immunogold staining and electron microscopy. APMV3/S-6P and APMV3 virus preparations were incubated with goat anti-SARS-CoV-2 S serum, followed by sucrose purification. Then, sucrose-purified viruses were fixed and probed with gold-labeled secondary antibody before analysis by electron microscopy. A representative image of three pleomorphic APMV3 particles is shown on the top panel. Representative images of APMV3/S-6P are shown in the bottom panels (one and three virus particles in the left and right panels for APMV3/S-6P, respectively). Gold particles are indicated by red arrows. Scale bars indicate 200 nm.

FIGS. 3A-3E are a series of graphs, immunohistochemistry (IHC) images, and a schematic demonstrating the replication of APMV3 and APMV3/S-6P in hamsters (Experiment #1). FIG. 3A: Timeline of immunization and sampling for Experiment #1. NTs, nasal turbinates; IHC, immunohistochemistry, IN, intranasal. Hamsters (n=45 per group) were immunized intranasally with 6 log₁₀ PFU of APMV3/S-6P or APMV3. On days 3, 5, and 7, six animals per group were sacrificed for virus titration (see FIGS. 3B-3D), and another two animals per group per day were sacrificed for IHC analysis (see FIG. 3E). Sera were collected from the remaining 21 animals per group on day 26 (prebleeds had been taken on day −11) for analysis (see FIGS. 8A-8D). (FIGS. 3B, 3C) Virus titers in nasal turbinates (NT, FIG. 3B) and lungs (FIG. 3C) were determined by immunoplaque assay as described in Materials and Methods. The limit of detection, indicated by a dotted line, was 50 PFU per g of tissue. The numbers of animals with virus detectable at or above the limit of detection are indicated above the x axis. The means with SD are shown. *=p≤0.05. FIG. 3D: Stability of S expression on day 3 post-immunization as determined by dual-staining immunoplaque assay. The means and SD of the percentages of S-positive plaques are shown. FIG. 3E: IHC analysis. Serial sections were immunostained for APMV3 and SARS-CoV-2 S antigen using hyperimmune antisera against APMV3 virions and secreted S-2P protein, respectively. Representative images from day 5 are shown. Areas with bronchial epithelial cells positive for APMV3 and SARS-CoV-2 S are marked by red and blue arrowheads, respectively (10× magnification; a 100 μm size bar is shown in the bottom right corners).

FIGS. 4A-4G is a schematic and a series of plots demonstrating the immunogenicity of APMV3 and APMV3/S-6P in hamsters (Experiment #2). FIG. 4A: Timeline of immunization, sampling, and challenge for Experiment #2. Groups of hamsters were immunized intranasally with 6 log₁₀ PFU of APMV3/S-6P or APMV3. Sera were collected on days −1 and 27 pi. Analysis of the sera are shown in FIGS. 4B-4G; see FIG. 5 for the results of the challenge. FIGS. 4B-4D: The 50% SARS-CoV-2 neutralizing titers (ND₅₀) were determined on Vero E6 cells against isolates WA1/2020 (lineage A) (FIG. 4B), USA/CA_CDC_5574/2020 (lineage B.1.1.7) (C), and USA/MD-H1P01542/2021 (lineage B.1.351) (FIG. 4D). Titers are expressed in log₁₀. The limit of detection, indicated by a dotted line, is 0.8 log₁₀. FIGS. 4E and 4F: End-point ELISA titers expressed in log₁₀ for serum IgG (FIG. 4E) and IgA (FIG. 4F) to a secreted prefusion-stabilized form (aa 1-1208) of the S protein (left panels) or to a fragment of the S protein (aa 328-531) containing SARS-CoV-2 receptor-binding domain (RBD) (right panels). The limit of detection is 2 and 3 log₁₀ for IgG and IgA, respectively (dotted lines).

FIG. 4G: 60% plaque reduction neutralization titers (PRNT₆₀) of the sera collected on days −1 and 27 post-immunization against APMV3. The limit of detection was 1 log₁₀ (dotted line). In each graph, the means and SD are shown. **=p≤0.01, ****=p≤0.0001.

FIGS. 5A-5F are a series of graphs and a heat map demonstrating the protective efficacy of APMV3/S-6P against a SARS-CoV-2 challenge (from Experiment #2). As part of Experiment #2 (see FIG. 4A for the timeline), hamsters (n=10 per group) that had been immunized intranasally with 6 log₁₀ PFU per animal of APMV3/S-6P or APMV3 were challenged intranasally on day 30 with 4.5 log₁₀ TCID₅₀ per animal of the SARS-CoV-2 USA-WA1/2020. FIG. 5A: Body weights were measured from day −1 to day 5 post-challenge and expressed as % body weight relative to the day 0 time point (n=10 hamsters per group from day −1 to day 3 and n=5 hamsters per group from day 4 to day 5). The means and SD are shown. ****=p≤0.0001. FIGS. 5B-5F: Five hamsters per group were euthanized on days 3 and 5 post-challenge, and NTs and lungs were harvested, homogenized, and aliquots were snap-frozen until further processing. (FIGS. 5B-5C) Total RNA was extracted from lung homogenates and subjected to RT-qPCR to evaluate the expression of selected genes related to inflammatory/antiviral responses. qPCR data were analyzed using the ΔΔCt method, normalized to β-actin, and expressed as fold change over the average expression levels of three mock-immunized, mock-challenged hamsters. The three most up-regulated genes (IFNλ, CXCL10 and Mx2) are shown in FIG. 5B while the relative expression levels of the remaining 16 genes on day 3 after challenge are shown in FIG. 5C as a heat map (n=5 per group). **=p<0.01, ***=p<0.001 and ****=p<0.0001. The means and SD are shown in FIG. 5B. FIG. 5D: RT-qPCR quantification of the SARS-CoV-2 genomic N (gN), genomic E (gE) and subgenomic E (sgE) RNAs in lung homogenates of immunized hamsters on days 3 and 5 following challenge with SARS-CoV-2. The RNAs were quantified in triplicate by TaqMan assays at the indicated time points and expressed as log₁₀ copies per g of tissue (n=5 per group per day, ***=p<0.001 and ****=p<0.0001). Standard curves were generated using serial dilutions of plasmids containing the targeted sequence. The mean and SD deviation are shown. (FIGS. 5E, 5F) Quantification, by limiting dilution on Vero E6 cells, of infectious SARS-CoV-2 in NTs (FIG. 5E) and lung homogenates (FIG. 5F) prepared from previously immunized hamsters harvested on the indicated days post challenge (n=5 per group, ****=p<0.0001). The means and SD deviations are shown.

FIG. 6 is a graph demonstrating the effect of exogenous trypsin and harvesting method on APMV3 and APMV3/S-6P yields in Vero cells. Sub-confluent wells of Vero or A549 cells in 6-well plates were inoculated in quadruplicate with APMV3 or APMV3/S-6P at an MOI of 1 PFU/cell. After 2 h incubation, Vero cells were washed once with Opti-MEM and replenished with Opti-MEM containing 1% L-glutamine with (+) or without (−) 2% TrypLE Select, as indicated (two wells per condition). To determine the titers of virus released from the infected cells (“Media”), 1 ml of the tissue culture media was harvested at 24 hpi from duplicate wells per virus per condition, clarified by brief centrifugation (3 min at 1500 rpm), and snap frozen. To determine the titers of released plus cell-associated virus (“Media with cells”), the monolayers (duplicate wells per virus per condition) were scraped, and supernatants were harvested from the remaining replicas by scraping cells into the remaining 1 mL of media, and the suspensions were gently vortexed for 1 min and clarified by brief (3 min at 1500 rpm), after which the clarified supernatants were aliquoted and snap-frozen. Virus titers were determined later in duplicate by immunoplaque assay on Vero cells at 37° C. as described above. Means with SD from duplicate wells are shown. ****=p<0.0001.

FIGS. 7A-7D are a series of plots demonstrating the expression of virus proteins in vitro and incorporation of the SARS-CoV-2 S protein into the vector particles. Viral protein expression in Vero (FIGS. 7A, 7B, 7C) and A549 (FIG. 7D, 7E) cells. From the time course experiment described in FIGS. 1E and 1F, Vero and A549 cells that had been infected in quadruplicate with an MOI of 0.01 PFU/cell of the indicated virus were harvested at 8 h intervals from 8 to 80 hpi. Two wells per time point were harvested for analysis of cell-associated viral proteins by Western blotting (one well) and flow cytometry (one well, Vero cells only). (FIGS. 7A, 7B, 7D, 7E). Western blotting time course. The APMV3 N (FIGS. 7A, 7D) and SARS-CoV-2 S (FIGS. 7B, 7E) protein bands were visualized by Western blotting using a primary rabbit anti-APMV3 serum and a primary goat anti-SARS-CoV-2 S serum, respectively, and labeled with infrared secondary antibodies (Materials and Methods). (FIGS. 7A, 7B) The level of expression of each protein band was normalized to ϵ-actin of the same sample and expressed as fluorescence intensity (FI). (FIG. 7C) Flow cytometry time course. The percentage of live single SARS-CoV-2 S-expressing cells in APMV3/S-6P-infected Vero cells was determined by flow cytometry using a PE-conjugated CR3022 anti-SARS-CoV-2 S monoclonal antibody. (FIGS. 7F, 7G) In an additional set of three independent experiments, Vero (FIG. 7F) or A549 (FIG. 7G) cells were infected with the indicated virus using an MOI of 1 or 10 PFU/cell. Twenty-four hpi, cells were harvested and stained using a PE-conjugated CR3022 anti-SARS-CoV-2 S monoclonal antibody, as described above. Cells were analyzed by flow cytometry and the % of live single cells expressing SARS-CoV-2 S was determined. The mean and SD are shown.

FIGS. 8A-8D are a series of plots demonstrating the immunogenicity of APMV3 and APMV3/S-6P in hamsters (from Experiment #1). From Experiment #1 (as shown in the timeline in FIG. 3A) sera were collected on day 26 pi from 21 hamsters per group that had been inoculated on day 0 with 6 log₁₀ PFU of APMV3/S-6P or APMV3. The sera were analyzed as follows: (FIG. 8A) ND₅₀ titers expressed in log₁₀ against SARS-CoV-2 USA-WA1/2020, evaluated on Vero E6 cells. The limit of detection, indicated by a dotted line, is 0.75 log₁₀. (FIGS. 8B and 8C) End-point ELISA titers expressed in log₁₀ of serum IgG to the full-length S (FIG. 8B) and the RBD of S (FIG. 8C). The limit of detection, indicated by a dotted line, is 2 log₁₀. (FIG. 8D) Titers of APMV3 neutralizing antibodies in sera from day −11 and 26, expressed in log₁₀ 60% plaque reduction neutralization titer (PRNT₆₀). The limit of detection is indicated by a dotted line (1 log₁₀). In each graph, the mean and SD are shown. ****=p≤0.0001.

### DETAILED DESCRIPTION

The disclosure relates to intranasal vaccines to prevent infection and transmission of severe acute respiratory syndrome coronavirus 2 (SARS-CoV-2). Briefly, avian paramyxovirus type 3 (APMV3) was evaluated as a vaccine vector to express the spike (S) protein stabilized in prefusion conformation by six proline substitutions (APMV3/S-6P). In hamsters, a single intranasal dose of APMV3/S-6P induced a strong serum neutralizing antibody response to the homologous SARS-CoV-2 isolate WA1, and a strong serum IgG and IgA response to the S protein and its receptor-binding domain, and a strong serum neutralizing antibody response to the SARS-CoV-2 isolate WA1/2020 (lineage A). Serum antibodies of APMV3/S-6P-immunized hamsters were also effective in neutralizing isolates SARS-CoV-2 variants of concern of lineages B.1.1.7 and B.1.351. Immunized hamsters challenged with SARS-CoV-2 WA1/2020 did not exhibit weight loss and lung inflammation and SARS-CoV-2 replication in the upper and lower respiratory tract was low or undetectable. Thus, a single intranasal dose of APMV3/S-6P in hamsters was highly protective against SARS-CoV-2 challenge, suggesting that APMV3/S-6P is suitable for clinical development.

### Severe Acute Respiratory Syndrome Coronavirus 2

Severe acute respiratory syndrome coronavirus 2 (SARS-CoV-2; NCBI Reference Sequence: NC_045512.2; Wu F, Zhao S, et al., A new coronavirus associated with human respiratory disease in China. Nature. 2020 March; 579(7798):265-269. doi: 10.1038/s41586-020-2008-3. Epub 2020 Feb. 3. Erratum in: Nature. 2020 April; 580(7803):E7. PMID: 32015508; PMCID: PMC7094943) is an enveloped, positive-sense, non-segmented, single-stranded RNA virus that causes coronavirus disease 2019 (COVID-19) and created a pandemic and global public health crisis. Vaccines based on mRNA and on replication-incompetent adenoviruses have been developed and used under emergency authorization, have been very effective in reducing morbidity and mortality due to COVID-19. However, most of the world has not yet had access to effective COVID vaccines, and there is a continuing, urgent need for vaccines and therapeutics. In addition, there is an increasing recognition of breakthrough infections in vaccinated individuals, especially involving the upper respiratory. The structural proteins of SARS-CoV-2 include the envelope protein (E), spike or surface glycoprotein (S), membrane protein (M) and the nucleocapsid protein (N). The spike glycoprotein is found on the outside of the virus particle and gives coronavirus viruses their crown-like appearance. This glycoprotein mediates attachment of the virus particle and entry into the host cell. S protein is an important target for vaccine development, antibody therapies and diagnostic antigen-based tests.

Accordingly, in certain embodiments, a pharmaceutical composition comprises an avian paramyxovirus type 3 (APMV3) comprising one or more polynucleotides encoding one or more coronavirus proteins comprising a spike (S) protein, a membrane (M) protein, an envelope (E) protein or a nucleocapsid (N) protein or fragments thereof.

The SARS-CoV-2 S protein is highly conserved among all human coronaviruses (HCoVs) and is involved in receptor recognition, viral attachment, and entry into host cells. With a size of 180-200 kDa, the S protein consists of an extracellular N-terminus, a transmembrane (TM) domain anchored in the viral membrane, and a short intracellular C-terminal segment. S normally exists in a metastable, prefusion conformation; once the virus interacts with the host cell, extensive structural rearrangement of the S protein occurs, allowing the virus to fuse with the host cell membrane. The spikes are coated with polysaccharide molecules to camouflage them, evading surveillance of the host immune system during entry (Huang, Y., Yang, C., Xu, Xf. et al. Structural and functional properties of SARS-CoV-2 spike protein: potential antivirus drug development for COVID-19. Acta Pharmacol Sin 41, 1141-1149 (2020). DOI: 10.1038/s41401-020-0485-4).

The total length of SARS-CoV-2 S is 1273 aa and consists of a signal peptide (amino acids 1-13) located at the N-terminus, the S1 subunit (14-685 residues), and the S2 subunit (686-1273 residues); the last two regions are responsible for receptor binding and membrane fusion, respectively. In the S1 subunit, there is an N-terminal domain (14-305 residues) and a receptor-binding domain (RBD, 319-541 residues); the fusion peptide (FP) (788-806 residues), beptapeptide repeat sequence 1 (HR1) (912-984 residues), HR2 (1163-1213 residues), TM domain (1213-1237 residues), and cytoplasm domain (1237-1273 residues) comprise the S2 subunit (Xia S, Zhu Y, Liu M, Lan Q, Xu W, Wu Y, et al. Fusion mechanism of 2019-nCoV and fusion inhibitors targeting HR1 domain in spike protein. Cell Mol Immunol. 2020; 17:765-7). S protein trimers visually form a characteristic bulbous, crown-like halo surrounding the viral particle. Based on the structure of coronavirus S protein monomers, the S1 and S2 subunits form the bulbous head and stalk region (Tang T, Bidon M, Jaimes J A, Whittaker G R, Daniel S. Coronavirus membrane fusion mechanism offers a potential target for antiviral development. Antivir Res. 2020; 178:104792). The structure of the SARS-CoV-2 trimeric S protein has been determined by cryo-electron microscopy at the atomic level, revealing different conformations of the S RBD domain in opened and closed states and its corresponding functions (Wrapp 1), et al. Cryo-EM structure of the 2019-nCoV spike in the prefusion conformation. Science. 2020; 367:1260-3; Walls A C, et al. Structure, function, and antigenicity of the SARS-CoV-2 spike glycoprotein. Cell. 2020; 181:281-92 e286).

In certain embodiments, the coronavirus is a severe acute respiratory syndrome coronavirus (SARS-CoV-2). In certain embodiments, the coronavirus protein is a SARS-CoV-2 spike (S) protein or fragments thereof. In certain embodiments, the SARS-CoV-2 S protein, or fragments thereof, comprises one or more amino acid substitutions. In certain embodiments, the SARS-CoV-2 S protein, or fragments thereof, comprises two or more amino acid substitutions. In certain embodiments, the SARS-CoV-2 S protein, or fragments thereof, comprises six or more amino acid substitutions. In certain embodiments, the SARS-CoV-2 S protein, or fragments thereof, comprises one or more proline substitutions. In certain embodiments, the SARS-CoV-2 S protein or fragments thereof, comprises one or more proline substitutions at amino acid positions 817, 892, 899, 942, 986 and 987. In certain embodiments, the S protein comprises one or more pseudoproline analogs comprising (2S,3R)-3-phenylpyrrolidine-2-carboxylic acid, (2S,3S)-3-hydroxypyrrolidine-2-carboxylic acid, (2S,4S)-4-phenyl-pyrrolidine-2-carboxylic acid, (2S, 4R)-4-benzyl-pyrrolidine-2-carboxylic acid, (2S,3aS,7aS)-Octahydro-1H-indole-2-carboxylic acid, (2S,3R)-3-phenylpyrrolidine-2-carboxylic acid, (2S,4R)-(−)-4-t-butoxypyrrolidine-2-carboxylic acid, (2S,4S)-(−)-4-hydroxypyrrolidine-2-carboxylic acid, trans-4-Fluoro-L-proline, cysteine-derived thiazolidine, serine-derived oxazolidine, or threonine-derived oxazolidine; trifluoromethylated pseudoprolines; proline analog or homolog having a constrained conformation; trifluoromethylated azetidine 2-carboxylic acid; trifluoromethylated homoserine; oxetanyl-containing peptidomimetic; N-aminoimidazolidin-2-one analog; and nonchiral pipecolic acid analogs. Exemplary singly- or doubly substituted substituted proline analogs include 5,5′-dimethylproline, 2,4-methano-o-proline, or 2,5-ethano-β-proline.

In certain embodiments, the polynucleotide encoding the S protein has at least a 50% sequence identity to the wild-type the full-length SARS-CoV-2 S sequence (NCBI reference sequence NC_045512.2), at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the wild-type the full-length SARS-CoV-2 S sequence.

In certain embodiments, the S protein has at least a 50% amino acid sequence identity to the wild-type the full-length SARS-CoV-2 S amino acid sequence (NCBI reference sequence NC_045512.2), at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the wild-type the full-length SARS-CoV-2 S amino acid sequence.

### Codon Optimization

In some embodiments, the polynucleotides provided herein encoding one or more viral proteins include codon-optimized sequences. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of A, G, C, U nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285).

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method and others. The CAI method chooses a most frequently used synonymous codon for an entire protein coding sequence. As an example, the most frequently used codon for each amino acid can be deduced from 74,218 protein-coding genes from a human genome. Any polynucleotide, nucleic acid sequence, or codon sequence provided herein can be codon optimized.

In some embodiments, the nucleotide sequence of any region of the RNA or DNA sequence embodied herein may be codon optimized. In certain embodiments, the primary cDNA template may include reducing the occurrence or frequency of appearance of certain nucleotides in the template strand. For example, the occurrence of a nucleotide in a template may be increased or reduced to a level above or below 25% of said nucleotides in the template. In further examples, the occurrence of a nucleotide in a template may be increased or reduced to a level above or below 20% of said nucleotides in the template. In some examples, the occurrence of a nucleotide in a template may be increased or reduced to a level above or below 16% of said nucleotides in the template. The occurrence of a nucleotide in a template may be increased or reduced to a level above or below 15% and may be increased or reduced to a level above or below 12% of said nucleotides in the template.

In certain embodiments, the polynucleotides of the disclosure can comprise one or more chemically modified nucleotides. Examples of nucleic acid monomers include non-natural, modified, and chemically modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

Examples of modified or chemically modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴, N⁴-dialkylcytidines.

Examples of modified or chemically modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴, N⁴-dimethylcytidine.

Examples of modified or chemically modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “SMeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′-O-dimethyl-adenosine, N⁶,N⁶,2′-O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically modified nucleotides include N¹-alkylguanosines, N²-alkylguanosines, thienoguanosines, 7-deazaguanosines, 8-oxoguanosines, 8-bromoguanosines, O⁶-alkylguanosines, xanthosines, inosines, and N¹-alkylinosines.

Examples of modified or chemically modified nucleotides include N¹-methylguanosine, N²-methylguanosine, thienoguanosine, 7-deazaguanosine, 8-oxoguanosine, 8-bromoguanosine, O⁶-methylguanosine, xanthosine, inosine, and N′-methylinosine.

Examples of nucleic acid monomers include modified and chemically modified nucleotides, including any such nucleotides known in the art.

Examples of modified and chemically modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically modified nucleotide monomers include 3′-end stabilized nucleotides, 3′-glyceryl nucleotides, 3′-inverted abasic nucleotides, and 3′-inverted thymidine.

Examples of modified and chemically modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically modified nucleotide monomers include 2′,4′-constrained 2′-O-methoxyethyl (cMOE) and 2′-O-Ethyl (cEt) modified DNAs.

Examples of modified and chemically modified nucleotide monomers include 2′-amino nucleotides, 2′-O-amino nucleotides, 2′-C-allyl nucleotides, and 2′-O-allyl nucleotides.

Examples of modified and chemically modified nucleotide monomers include N6-methyladenosine nucleotides.

Examples of modified and chemically modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically modified nucleotide monomers include 2′-O-aminopropyl substituted nucleotides.

Examples of modified and chemically modified nucleotide monomers include replacing the 2′—OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically modified nucleotide monomers may be found in nature.

### Formulations

The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired, with other active agents, e.g., other analgesic agents.

In certain embodiments, the vectors, vaccines and compositions embodied herein are formulated to comprise an osmolyte, comprising sodium chloride, mannitol, sorbitol, glycerin or any other well known in the art. This formulation can also include sorbic acid, potassium sorbate, parabens, benzalkonium chloride, benzoic acid, sodium benzoate, phenoxyethanol, phenyl ethanol or other suitable preservatives known in the art with or without the addition of chelating agents, such as citric acids, sodium citrate or sodium salts of edetic acid may be added to preserve it, allowing a non-sterile nasal formulation in bottles fitted with standard nasal pumps. In certain embodiments, the formulation is packaged into a nasal spray to be delivered to the nose as one puff of 10 μL-200 μL into each nostril. The solution may contain suitable buffers, such citric acid/sodium citrate, disodium phosphate, dihydrogen sodium phosphate, acetic acid/sodium acetate or other buffers known in the art. The nasal formulation may be sterilized by filtration) and filled aseptically into suitable bottles, snapping appropriate preservative free nasal pumps onto them. The nasal nebulization is particularly useful in treating patients in early stages of a respiratory disease caused by SARS CoV 2, such as pandemic COVID 19. Lung nebulization is helpful for patients and may be practiced by using the same composition in a nebulizer.

The vectors, vaccines and compositions embodied herein, can include a pharmaceutically acceptable carrier, e.g., one or more solvents, dispersion media, coatings, antimicrobial agents, isotonic and absorption delaying agents, and the like, compatible with administration to a mammal, such as a human. Any carrier compatible with the excipient(s) and therapeutic agent(s) is suitable for use. Supplementary active compounds may also be incorporated into the compositions.

In certain embodiments, the pharmaceutical compositions are specifically adapted for administration to the nasal cavity. The pharmaceutical compositions may be applied before or after the outbreak of a respiratory viral infection in a human individual. Even if applied after the outbreak of a viral infection the compositions still prevent or at least ameliorate late complications of respiratory viral infections. Such complications are known in the art and include—but are not limited to—complications in connection with secondary infections by bacteria, and deterioration of pre-existing diseases such as allergy or COPD.

For administration by inhalation, the compositions described herein can conveniently be delivered in the form of an aerosol (e.g., through liquid nebulization, dry powder dispersion or meter-dose administration The aerosol can be delivered from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

By non-limiting example water-based liquid formulations can include non-encapsulating water-soluble excipients. Simple formulations can also include organic-based liquid formulations for nebulization or meter-dose inhaler. By non-limiting example organic-based liquid formulations can include non-encapsulating organic soluble excipients.

Simple formulations can also include dry powder formulations for administration with a dry powder inhaler. By non-limiting example dry powder formulations can include water soluble or organic soluble non-encapsulating excipients with or without a blending agent such as lactose.

Formulations can include water-based liquid formulations for nebulization such as lipids, liposomes, cyclodextrins, microencapsulations, and emulsions.

Formulations can also include organic-based liquid formulations for nebulization or meter-dose inhale such as lipids, microencapsulations, and reverse-phase water-based emulsions.

Formulations can also include low-solubility, water-based liquid formulations for nebulization such as lipid nanosuspensions.

Formulations can also include dry powder formulations for administration using a dry powder inhaler. A non-limiting example, complex dry powder formulations can include the compositions embodied herein as a co-crystal/co-precipitate/spray dried complex or mixture with low-water soluble excipients/salts in dry powder form with or without a blending agent such as lactose.

In certain embodiments, intranasal formulations referred to herein may have a pH value within a range of from 3.5 to 8.0, usually within a range of from about 4.0 to about 8.0. They may comprise one or more nasally compatible pH adjusting agents or buffer systems that prevent pH drift during storage. Such pH adjusting agents include, but are not limited to, boric acid, sodium borate, potassium citrate, citric acid, sodium bicarbonate, and various inorganic phosphate buffers such as Na₂HPO₄, NaH₂PO₄, KH₂PO₄, and mixtures thereof. The minimal ionic strengths introduced by any such pH-adjusting agents do not interfere with the essence of the invention. To prevent precipitation of calcium with phosphate ions from the buffer system, EDTA may be added up to a concentration of 2 mg/ml. In addition, flavors such as eucalyptus, campher, menthol, peppermint or similar, by way of oils or extracts, may be added to the product at concentrations known in the art.

Also, the intranasal formulations referred to herein may comprise one or more intranasally compatible surfactants. The surfactant facilitates the spread of the formulation across the surface of the nasal mucosa and may be non-ionic or anionic. Exemplary non-ionic surfactants may be selected from the group comprising tyloxapol, polyoxyethylene sorbitan esters, polyethoxylated castor oils, poloxamers, polyoxyethylene/polyoxypropylene surfactants, polyoxymethylene stearate, polyoxymethylene propylene glycol stearate, hydroxyalkylphosphonate, lauric or palmitic acid esters and ethers, triethanol amine oleate, or from a combination of the foregoing agents. Still further suitable surfactants may be known to those skilled in the art. The surfactants may typically be present at concentrations of from 0.02% (w/v) to 0.1% (w/v) of the composition.

In various embodiments, the present intranasal preparation may contain one or more preservatives to inhibit microbial growth and to prolong shelf life. Exemplary preservatives include, but are not limited to, disodium edetate (EDTA) and potassium sorbate. The preservative amount is typically less than about 0.02% (w/v) of the total composition, EDTA may be added up to 2 mg/ml.

In addition to the ingredients mentioned above, it is contemplated that a variety of additional or alternative ingredients may be present in the pharmaceutical compositions of the present disclosure, which additional or alternative ingredients include antioxidants such as vitamin E or its commercially available derivatives such as tocopherol polyethylene glycol 1000 succinate (TPGS), ascorbic acid, or sodium metabisulfite.

The pharmaceutical compositions herein are typically provided in sterile form for administration to the nasal cavity and are preferably adjusted for self-administration by the individual in need thereof. In one embodiment, the preparation is a particle-free nasal spray. Other suitable formulations include intranasally acceptable swabs, as well as ointments and gels that can be applied to the nose, optionally as sprays or aerosols.

Pharmaceutical compositions described above can be delivered via different methodologies including sprays, irrigation systems (e.g., netipot), syringes or others. The composition may be provided in a dosage form that is suitable for a nasal aerosol or inhalation administration route. An exemplary method of administration of the composition can include spraying vaporized or nebulized disseminated microparticles under an active dynamic pressure.

Suitable aerosol dispensers for use will be apparent to those skilled in the art and may vary from simple devices analogous to perfume dispensers to pressurized spray cans and even complex apparatus such as might be used in hospitals. Whichever device is used it is generally preferable that it comprises some kind of dosimeter to control the amount of solution administered in one go. One device, which corresponds to a dispenser with a nozzle, effectively incorporates such a dosimeter without any specialized adaptation being necessary, the limit stop of the depressible spray head fixing the maximum single amount of solution dispensable at once. Specially developed spray devices may be made with a hand-held device comprising a reservoir of the composition.

Suitable means for dispersing the spray, preferably in aerosol form, are provided. Examples include pneumatically pressurized devices and devices employing pressurized gas forced across the opening of a tube leading into the reservoir to create an aerosol, and press-button type devices wherein the button, when pressed, creates pressure on the surface of the liquid in the reservoir, forcing it up through a tube and through a fine nozzle to disperse the solution into an aerosol spray. Other examples include aerosol dispensers, inhalers, pump sprayers, nebulizers (such as positive pressure nebulizers), and the like. In some embodiments, the device used is pre-filled with a composition described herein.

For aqueous and other non-pressurized liquid systems, a variety of nebulizers (including small volume nebulizers) can be used to aerosolize the formulations. Compressor-driven nebulizers can utilize jet technology and can use compressed air to generate the liquid aerosol. Such devices are commercially available from, for example, Healthdyne Technologies, Inc.; Invacare, Inc.; Mountain Medical Equipment, Inc.; Pan Respiratory, Inc.; Mada Medical, Inc.; Puritan-Bennet; Schuco, Inc., DeVilbiss Health Care, Inc.; and Hospitak, Inc. Ultrasonic nebulizers generally rely on mechanical energy in the form of vibration of a piezoelectric crystal to generate respirable liquid droplets and are commercially available from, for example, Omron Heathcare, Inc. and DeVilbiss Health Care, Inc. Vibrating mesh nebulizers rely upon either piezoelectric or mechanical pulses to respirable liquid droplets generate Commercial examples of nebulizers that RESPIRGARD II™, AERONEB™, AERONEB™ Pro, and AERONEB™ Go produced by Aerogen; AERX™ and AERX ESSENCE™ produced by Aradigm; PORTA-NEB™, FREEWAY FREEDOM™, Sidestream, Ventstream and I-neb produced by Respironics, Inc.; and PARI LC-PLUS™, PARI LC-STAR™, and e-Flow7m produced by PARI, GmbH. By further non-limiting example, U.S. Pat. No. 6,196,219, is hereby incorporated by reference in its entirety.

In some embodiments, the solution can be formed prior to use of the nebulizer by a patient. In other embodiments, the drug can be stored in the nebulizer in solid form. In this case, the solution can be mixed upon activation of the nebulizer, such as described in U.S. Pat. No. 6,427,682 and PCT Publication No. WO 03/035030, both of which are hereby incorporated by reference in their entirety. In these nebulizers, the drug, optionally combined with excipients to form a solid composition, can be stored in a separate compartment from a liquid solvent.

One embodiment would include a multi-dose metered dose spray pump allowing for spraying of a fixed volume of solution. Alternatively, gas driven (e.g., nitrogen) devices, such as systems that hold the compositions separate from the propellant in aluminum or plastic (or any other type of) bottle. These devices deliver solution at variable diffusion flows and angles when combined with different actuators. Preferred diffusion flows could deliver 0.5-10 ml solution per spraying second at angles of 0-60°.

The compositions described above can be administered as per physician's instructions and depending on the condition. In certain embodiments, a mode of (nasal) administration comprises 1-5 sprays per nostril. In certain embodiments, a mode of (nasal) administration comprises 1 spray per nostril. In certain embodiments, a second dose of the composition can be administered from about six months after the first dose. A routine diagnostic test will reveal whether a second or third dose is required.

In certain embodiments, administration of the compositions of this disclosure may also be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. powders and sustained release capsules and may be prepared by any methods well known in the art of pharmacy.

Liquid suspensions may be prepared using conventional methods to achieve suspension the composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Methods of Treatment

The present disclosure provides a method of treating or preventing coronavirus infections, e.g., severe acute respiratory syndrome coronavirus 2 (SARS-CoV-2) infection. In some embodiments, the method comprises administering to a subject in need thereof, an effective amount of a composition comprising a vector expressing at least one coronavirus proteins comprising a spike (S) protein, a membrane (M) protein, an envelope (E) protein or a nucleocapsid (N) protein or fragments thereof.

The therapeutic methods described herein (that include prophylactic treatment) in general comprise administration of a therapeutically effective amount of the compositions herein to a subject (e.g., animal, human) in need thereof, including a mammal, particularly a human. Such treatment will be suitably administered to subjects, particularly humans, suffering from, having, susceptible to, or at risk for infection by respiratory tract viruses or symptoms thereof. Determination of those subjects “at risk” can be made by any objective or subjective determination by a diagnostic test or opinion of a subject or health care provider. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Dosage, toxicity and therapeutic efficacy of the present compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

Data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a composition (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the disclosure can include a single treatment or a series of treatments.

In some embodiments, the method comprises genetically modifying a cell to express at least one coronavirus proteins comprising a spike (S) protein, a membrane (M) protein, an envelope (E) protein or a nucleocapsid (N) protein or fragments thereof.

In some embodiments, for viral vector-mediated delivery, a dose comprises at least 1×10⁵ particles to about 1×10¹⁵ particles. In some embodiments the delivery is via an avian paramyxovirus type 3 virus (APMV3), such as a single dose containing at least 1×10⁵ particles (also referred to as particle units, pu) of APMV3 vector. In some embodiments, the dose is at least about 1×10⁶ particles (for example, about 1×10⁶-1×10¹² particles), at least about 1×10⁷ particles, at least about 1×10⁸ particles (e.g., about 1×10⁸-1×10¹¹ particles or about 1×10⁸-1×10¹² particles), at least about 1×10⁰ particles (e.g., about 1×10⁹-1×10¹⁰ particles or about 1×10⁹-1×10¹² particles), or at least about 1×10¹⁰ particles (e.g., about 1×10-1×10¹² particles) of the APMV3 vector. Alternatively, the dose comprises no more than about 1×10¹⁴ particles, no more than about 1×10¹³ particles, no more than about 1×10¹² particles, no more than about 1×10¹¹ particles, and no more than about 1×10¹⁰ particles (e.g., no more than about 1×10⁹ particles). Thus, in some embodiments, the dose contains a single dose of APMV3 vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹ pu, about 4×10¹ pu, about 1×10¹² pu, about 2×10¹² pu, or about 4×10¹² pu of APMV3 vector. In some embodiments, the adenovirus is delivered via multiple doses. Other effective dosages can be readily established by one of ordinary skill in the art through routine trials establishing dose response curves (see, for example, U.S. Pat. No. 8,404,658).

### Combination Therapies

The compositions can be administered in conjunction with (e.g., before, simultaneously or following) one or more therapies. For example, in certain embodiments, the method comprises administration of a composition of the disclosure in conjunction with an additional anti-viral therapy and the like.

In certain embodiments, the compositions embodied herein are administered to a patient in combination with one or more other anti-viral agents or therapeutics. The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

### Delivery Vehicles

Lipid Formulations LNPs: Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida M Pharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTRO2 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

Liposomes: Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

Cationic Liposomes: Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications.

LipidNanoparticles: In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine™ reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation. In some aspects, nucleic acid molecules provided herein, and lipids or lipid formulations provided herein form a lipid nanoparticle (LNP).

In other aspects, nucleic acid molecules provided herein are incorporated into a lipid formulation (i.e., a lipid-based delivery vehicle).

In the context of the present disclosure, a lipid-based delivery vehicle typically serves to transport a desired RNA to a target cell or tissue. The lipid-based delivery vehicle can be any suitable lipid-based delivery vehicle known in the art. In some aspects, the lipid-based delivery vehicle is a liposome, a cationic liposome, or a lipid nanoparticle containing a self-replicating RNA of the disclosure. In some aspects, the lipid-based delivery vehicle comprises a nanoparticle or a bilayer of lipid molecules and a self-replicating RNA of the disclosure. In some aspects, the lipid bilayer further comprises a neutral lipid or a polymer. In some aspects, the lipid formulation comprises a liquid medium. In some aspects, the formulation further encapsulates a nucleic acid. In some aspects, the lipid formulation further comprises a nucleic acid and a neutral lipid or a polymer. In some aspects, the lipid formulation encapsulates the nucleic acid.

In certain embodiments, lipid formulations comprise one or more self-replicating RNA molecules encapsulated within the lipid formulation. In some aspects, the lipid formulation comprises liposomes. In some aspects, the lipid formulation comprises cationic liposomes. In some aspects, the lipid formulation comprises lipid nanoparticles.

In some aspects, the RNA is fully encapsulated within the lipid portion of the lipid formulation such that the RNA in the lipid formulation is resistant in aqueous solution to nuclease degradation. In other aspects, the lipid formulations described herein are substantially non-toxic to animals such as humans and other mammals.

The disclosure is further described in detail by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### EXAMPLES

### Example 1 A Single Intranasal Immunization with Avian Paramyxovirus Type 3 Expressing the Prefusion-Stabilized Form of the SARS-CoV-2 Spike Protein Protects Hamsters Against SARS-CoV-2 Challenge

The SARS-CoV-2 envelope contains the spike (S) protein, that through its receptor-binding domain (RBD), binds to the host cellular receptor, angiotensin-converting enzyme 2 (ACE2) (Hoffmann et al., 2020; Zhou et al., 2020). The S protein also mediates virus-cell membrane fusion required for viral entry. It is the primary target for neutralizing antibodies and also a target for T cell responses (Braun et al., 2020; Ju et al., 2020). Newly synthesized S protein is present in a so-called prefusion conformation that has been shown to be a more immunogenic form (Lu et al., 2021; Sanchez-Felipe et al., 2021). The stability of the prefusion conformation of the coronavirus S protein can be increased by genetic engineering, resulting in improved immunogenicity, as exemplified by SARS-CoV-1 and MERS-CoV (Pallesen et al., 2017). The first generation of SARS-CoV-2 S protein stabilized in its prefusion form contained two proline substitutions at amino acids (aa) 986 and 987 (S-2P) (Wrapp et al., 2020). A second generation of prefusion-stabilized S protein contained four additional proline substitutions at aa positions 817, 892, 899, and 942 [S HexaPro or S-6P, (Hsieh et al., 2020)]. The S-6P version exhibited increased expression and stability compared to S-2P when expressed in mammalian cells while retaining its antigenicity (Hsieh et al., 2020; Wrapp et al., 2020). The SARS-CoV-2 vaccine platforms that have been authorized, received emergency authorization, or those in development, are based mostly on the S-2P form.

While the COVID-19 vaccines presently in human use have been remarkably effective in reducing COVID-19 morbidity and mortality, there is increasing recognition of breakthrough infections (Bergwerk et al., 2021; Brown et al., 2021) as well as general concerns about the longevity of protection. Breakthrough infections have been associated with reduced titers of serum SARS-CoV-2-neutralizing antibodies (Bergwerk et al., 2021), which are both a protective effector and a marker for immunity in general. Breakthrough infections do not necessarily involve emerging variants with increased infectivity; infections with older variants have also been observed. However, although severe cases do occur, breakthrough infections usually are much less severe than those in virus-naive individuals and appear to primarily involve the upper respiratory tract. Nonetheless, there frequently is shedding of high titers of virus, which would promote spread. In addition, asymptomatic breakthrough infections—and shedding—likely occur that are not identified and facilitate spread. Current COVID-19 vaccines are administered parenterally and thus do not directly stimulate respiratory tract immunity. Therefore, it is important to evaluate additional vaccine approaches, in particular those involving direct immunization of the respiratory tract. In the present study, we developed a replication-competent, attenuated, intranasal, vectored SARS-CoV-2 vaccine candidate based on avian paramyxovirus (APMV) serotype 3.

APMVs are classified in the Order Mononegavirales, Family Paramyxoviridae, and are non-segmented, negative-sense, single-stranded RNA viruses that replicate in the cytoplasm and acquire a lipid envelope by budding through the plasma membrane. APMVs can be isolated from domestic and wild birds, with 21 serotypes identified to date (Rima et al., 2019). APMV type 1 (APMV1), better known as Newcastle disease virus (NDV), is the most common and best characterized of the APMVs and exemplifies their general properties. NDV infection of humans is rare and is largely restricted to bird handlers, is highly restricted due to host incompatibility, and causes mild or no illness. NDV is antigenically distinct from human pathogens (DiNapoli et al., 2007). Thus, humans generally lack pre-existing immunity to NDV that might otherwise interfere with vector infection and immunogenicity (Hu et al., 2020). In an experimental setting, NDV and other APMVs can infect rodents and non-human primates by the respiratory route and cause a low level of replication that is restricted to the respiratory tract (Bukreyev et al., 2005; DiNapoli et al., 2010b). Parenthetically, NDV also has been used in humans as an oncolytic or cancer immunotherapy agent, usually in high doses given parenterally, and generally has been well-tolerated (Freeman et al., 2006; Pecora et al., 2002). Recombinant NDV also has been used as an experimental vaccine vector to express viral antigens from pathogens including human parainfluenza virus type 3, respiratory syncytial virus, highly pathogenic influenza virus, SARS-CoV-1 and SARS-CoV-2 (Bukreyev et al., 2005; DiNapoli et al., 2007; DiNapoli et al., 2010a; Martinez-Sobrido et al., 2006; Sun et al., 2020). Some vectors were based on strains with low pathogenicity in chickens (lentogenic), and these were quite restricted in replication and immunogenicity in non-avian hosts, including non-human primates (DiNapoli et al., 2009). Vectors based on strains with greater pathogenicity in chickens (velogenic and mesogenic) are less restricted and more immunogenic, but their status as United States Department of Agriculture (USDA) Select Agents limits their usefulness.

The inventors have previously evaluated the replication and immunogenicity of several APMV types in rodents (Samuel et al., 2011; Yoshida et al., 2019), and identified APMV3 (strain Parakeet/Netherlands/449/75) as a promising alternative to NDV. In hamsters, APMV3 was infectious by the intranasal route, and replication was restricted to the respiratory tract (Samuel et al., 2011). This APMV3 strain does not appear to be a significant natural pathogen of poultry (Alexander, 2000), and in experimental infections was only mildly pathogenic in poultry (Kumar et al., 2010). This strain also has been characterized as low-pathogenicity based on standard assays in embryonated chicken eggs and 1-day-old and 2-week old chicks and turkeys (Kumar et al., 2010). A reverse genetics system was previously developed and the P/M intergenic region was identified as an optimal insertion site for additional genes (Yoshida et al., 2019; Yoshida and Samal, 2017). APMV3 has been evaluated in animal models as an experimental vaccine vector expressing the Ebola virus glycoprotein GP (Yoshida et al., 2019) and the hemagglutinin HA protein of highly pathogenic avian influenza virus H5N1 (Shirvani et al., 2020). In the present study, we used APMV3 to express. In the present study, we used APMV3 to express the SARS-CoV-2 S protein, stabilized in its prefusion form, and evaluated the immunogenicity and protective efficacy of a single intranasal dose against SARS-CoV2 challenge in hamsters.

### Materials and Methods

Cells. Human lung epithelial A549 (ATCC CCL-185) cells were cultured in F-12K (ATCC) with 10% fetal bovine serum (FBS, GE Healthcare). Baby hamster kidney cells expressing T7 RNA polymerase (BSR T7/5) were grown in Glasgow minimum essential medium (MEM) (Thermo Fisher Scientific) with 10% FBS, 1% L-glutamine (Thermo Fisher Scientific), and 2% MEM Amino Acids (Thermo Fisher Scientific). African green monkey kidney Vero (ATCC CCL-81) and Vero E6 (ATCC CRL-1586) cells were cultured in Opti-MEM I+GlutaMAX (Thermo Fisher Scientific) supplemented with 5% FBS and 1% L-glutamine. Vero E6 cells were suitable for propagating and titrating SARS-related coronaviruses due to high ACE2 expression (Chu et al., 2020; Ren et al., 2006). Vero E6 cells stably expressing human TMPRSS2 that further improve SARS-CoV-2 replication (Matsuyama et al., 2020) were generated using the Sleeping Beauty transposase system (Liu X et al., under review). All experiments and assays in cell culture were performed at 37° C.

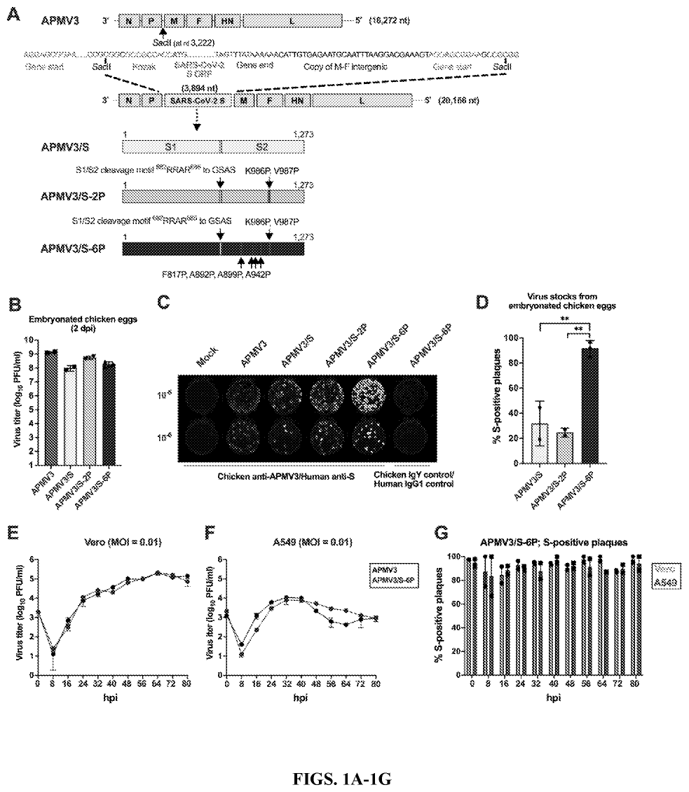

Generation of APMV3 expressing SARS-CoV-2 S. The cDNA clone encoding the antigenome of APMV3 (Parakeet/Netherlands/449/75 (Alexander, 2000)) and N, P, and L support plasmids were described previously (Shirvani et al., 2020; Yoshida and Samal, 2017). The ORF encoding the full-length wild type SARS-CoV-2 S derived from the first available sequence (NCBI reference sequence NC_045512.2) was codon-optimized for human usage and DNAs were synthesized (BioBasic) and three versions were designed: WT S, S-2P, and S-6P (see Results and FIG. 1). The WT S, S-2P, and S-6P cDNAs were designed to be flanked on the 3′-ends by a SacII restriction site followed by a Kozak sequence, and on the 5′-ends by a copy of the P gene end sequence, followed by a copy of the M-F intergenic sequence, an APMV3 gene start (which is identical for each APMV3 ORF) and a second SacII site. The cDNA lengths were designed to maintain the “rule of six” (Kolakofsky et al., 1998). The codon optimized WT version of S was synthesized commercially (BioBasic), and stabilizing mutations were introduced by site-directed mutagenesis following the manufacturer recommendations (Agilent). Each version of S was inserted into the unique SacII site in the 3′ noncoding region of the APMV3 M gene (at nt 3,222; FIG. 1A). The encoded viruses APMV3/S, APMV3/S-2P, and APMV3/S-6P were rescued by reverse genetics on BSR T7/5 cells (Buchholz et al., 1999) and passaged once or twice on Vero cells to generate passage 2 (P2) or passage 3 (P3) virus stocks. The parental empty APMV3 vector was also rescued and passaged once on Vero cells.

Two independent stocks of APMV3, APMV3/S and APMV3/S-2P and three independent stocks of APMV3/S-6P were generated by infecting 11-day-old specific pathogen-free, embryonated chicken eggs (Charles River Laboratories). Specifically, 200-2,000 PFUs of the P2 or P3 Vero-grown stocks were inoculated into the allantoic cavities of 5 to 30 eggs per stock. Allantoic fluids were harvested on day 2 p.i. and samples from individual eggs were screened for hemagglutination by standard hemagglutination (HA) assay using a 0.5% suspension of chicken red blood cells (Lampire). For each independent stock, allantoic fluids with HA titers equal to or higher than 1:512 for WT APMV3 and equal to or higher than 1:64 for the S-expressing APMV3s were pooled and clarified by centrifugation at 2,000 rpm for 10 min at 4° C. Supernatants were aliquoted, snap-frozen on dry ice, and stored at −80° C. until further use.

APMV3 titration by immunoplaque assay on Vero cells and dual-staining immunoplaque assay. Sub-confluent Vero cells in 24-well plates were infected in duplicate with ten-fold serially diluted viruses. After 2 h incubation at 37° C., cells were washed with Opti-MEM (Thermo Fisher Scientific) and overlaid with 1 ml per well of 0.8% methylcellulose dissolved in Opti-MEM containing 2% TrypLE Select (Thermo Fisher Scientific). On day 4 pi, cells were fixed using ice-cold 80% methanol. For dual-staining assays to detect expression of APMV3 and SARS-CoV-2 antigens, wells were incubated for 2 hour at RT with a combination of a primary chicken anti-APMV3 serum (1:2,000) and human anti-SARS-CoV-2 S RBD antibody (CR3022, 1:2,000) (or, as a negative control, was replaced with a combination of normal chicken IgY control (R&D Systems) and human IgG1 isotype control (BioLegend)). After extensive washing with PBS, wells were incubated for 1 hour at RT with 1:2,000 diluted infrared dye (IRDye)-conjugated 680LT donkey anti-chicken IgY and IRDye 800CW goat anti-human IgG secondary antibodies (LI-COR). After washing with PBS, plates were scanned with an Odyssey CLx imager (LI-COR).

Amplification and sequencing of SARS-CoV-2 virus stocks. The SARS-CoV-2 USA-WA1/2020 isolate (lineage A; GenBank MN985325; GISAID: EPI_ISL_404895; obtained from Dr. Natalie Thornburg et al., Centers of Disease Control and Prevention (CDC)) was passaged twice on Vero E6 cells. The amino acid sequence of the S protein of WA1/2020 (GenBank MN985325) is identical to that of the first available SARS-CoV-2 sequence that we used to design APMV3/S (GenBank MN908947). The USA/CA_CDC_5574/2020 isolate (lineage B.1.1.7; GISAID: EPI_ISL_751801; obtained from CDC) and the USA/MD-HP01542/2021 isolate (lineage B.1.351; GISAID: EPI_ISL_890360) were passaged on TMPRSS2-expressing Vero E6 cells. The SARS-CoV-2 stocks were titrated in Vero E6 cells by determination of the 50% tissue culture infectious dose (TCID₅₀) as previously described (Subbarao et al., 2004). (Subbarao et al., 2004). The complete genome sequences of the SARS-CoV-2 USA-WA1/2020 challenge virus and the variants B.1.1.7 and B.1.351 were determined by Illumina deep-sequencing and confirmed that the S gene sequences were identical to the expected consensus sequences. All experiments with SARS-CoV-2 were performed in BSL3 containment laboratories approved by the U.S. Department of Agriculture (USDA) and CDC.

Antibodies and antigens. Rabbit antiserum against sucrose-purified APMV3 were produced using a previously-described subcutaneous chamber method (Clemons et al., 1992). Goat anti-S hyperimmune serum N25-154 was generated using a secreted form of S-2P protein that was expressed in Expi293 cells and purified as described previously (Liu et al., under review). cDNA plasmids encoding the light and heavy chains of the anti-S-RBD human monoclonal IgG antibody CR3022 were generously provided by Drs. Peter Kwong and Baoshan Zhang (Vaccine Research Center, National Institute of Allergy and Infectious Diseases (NIAID), National Institutes of Health (NIH)). Expi293 suspension cells grown at 37° C. with 10% CO₂ in a shaking incubator were co-transfected with these plasmids following the manufacturer's recommendations. The CR3022 monoclonal antibody was purified by affinity chromatography at 5 to 6 days post-transfection. Aliquots were snap-frozen in liquid nitrogen and stored at −80° C.

The secreted prefusion-stabilized form of SARS-CoV-2 S (2019-nCoV S-2P_dFurin_F3CH2S, aa 1-1208; (Wrapp et al., 2020)) was expressed in Expi293 cells from a plasmid generously provided by Drs. Barney Graham, Kizzmekia Corbett (Vaccine Research Center, NIAID, NIH), and Jason McLellan (University of Texas at Austin), and purified as described previously (Liu et al, PNAS under review). A fragment of the S protein containing the RBD region (aa 328-531) was expressed in Expi293 cells from a cDNA plasmid obtained from Dr. David Veesler through BEI Resources, NIAID, NIH (Walls et al., 2020). Aliquots were snap-frozen in liquid nitrogen and stored at −80° C.

Multicycle growth kinetics. Multicycle growth kinetics were performed on Vero and A549 cells. Sub-confluent wells of Vero or A549 cells in 6-well plates were inoculated in quadruplicate with APMV3 or APMV3/S-6P using an MOI of 0.01 PFU/cell. After 2 h incubation, Vero cells were washed once with Opti-MEM and replenished with Opti-MEM containing 1% L-glutamine and 2% TrypLE Select. A549 cells were washed with F-12K and replenished with F-12K containing 1% L-glutamine and 0.5% TrypLE Select. Two wells per time point and per virus were used to evaluate virus replication by scraping cells into the media. Cells in media were vortexed and subjected to short centrifugation to collect the clarified supernatant that was aliquoted and snap-frozen followed by storage at −80° C. Virus titers were determined in duplicate by immunoplaque assay on Vero cells at 37° C. as described above. The third replica wells from the growth curves were used to evaluate the expression of SARS-CoV-2 S and APMV3 N proteins by Western blotting, and the fourth wells were used to analyze the expression of SARS-CoV-2 S protein by flow cytometry as described below.

Western blotting. Vero or A549 cells seeded in 6-well plates were infected with APMV3 or APMV3/S-6P as described above (multicycle growth kinetics). At the indicated time points, cells from the third replica wells were washed once with PBS and lysed with Bolt LDS Sample Buffer (Thermo Fisher Scientific) containing Halt Protease Inhibitor Cocktail (Thermo Fisher Scientific). Then, lysates were passed through QIAshredder columns (Qiagen) and stored at −80° C. Parts of the clarified cell lysates were denatured with Bolt LDS Sample Buffer and Sample Reducing Agent (Thermo Fisher Scientific) at 90° C. for 5 min. Denatured proteins were resolved on 4-12% Bis-Tris gels (Thermo Fisher Scientific) and transferred to polyvinylidene difluoride membranes. Membranes were blocked with Intercept Blocking Buffer and probed with the following primary and secondary antibodies diluted in Intercept T20 Antibody Diluent (LI-COR): rabbit anti-APMV3 serum (1:2,000); goat anti-SARS-CoV-2 S serum (1:5,000); mouse anti-β-actin antibody (R&D Systems) (1:10,000) and IRDye 680RD donkey anti-rabbit IgG (1:10,000); IRDye 800CW donkey anti-goat IgG (LI-COR) (1:10,000); IRDye 800CW donkey anti-mouse IgG (LI-COR) (1:10,000). The membranes were washed and scanned using an Odyssey CLx scanner. Fluorescence signals of the SARS-CoV-2 S and APMV3 N proteins were background-corrected automatically by the Image Studio Lite software (LI-COR) and measured to quantify the intensity of each protein band. Values were normalized in each gel to β-actin intensity of the same sample.

Flow cytometry. Mock- or virus-infected Vero or A549 cells in the fourth replica of the 6-well plates from the multicycle growth kinetics were trypsinized with TrypLE Select and harvested at the indicated time points. Next, cells were stained with LIVE/DEAD Fixable Near-IR Dead Cell Stain Kit (Thermo Fisher Scientific) and fixed with Cytofix/Cytoperm (BD Biosciences). After permeabilization in Perm/Wash buffer (BD Biosciences), cells were incubated with a phycoerythrin-conjugated human anti-SARS-CoV-2 S monoclonal antibody (CR3022) (1:100) in Perm/Wash buffer for 20 min in the dark. Stained cells were washed five times with Perm/Wash buffer and resuspended in PBS until analyzed by a FACSymphony Flow Cytometer (BD Biosciences). At least 20,000 events were acquired for each sample. Data were analyzed with FlowJo (version 10.7). First, the quality control of each acquired sample was performed using the FlowAI plugin (Monaco et al., 2016). Then, compensation was performed automatically using beads for each antibody. Live/dead staining, forward scatter height, and forward scatter area were used to identify single live cells. The expression of the SARS-CoV-2 S protein was analyzed on single live cells.

Silver staining of sucrose-purified APMV3s. Preparations of egg-grown APMV3 and APMV3/S-6P viruses were subjected to centrifugation on 30%/60% sucrose step gradients at 28,000 rpm for 2 h at 4° C. The opaque virus bands were collected, diluted in Tris-EDTA-NaCl (TEN), and pelleted by centrifugation at 8,000 g for 2 h at 4° C. The pelleted virus was resuspended with TEN buffer, aliquoted, and stored at −80° C. Protein concentrations were determined by bicinchoninic acid (BCA) assay using a Micro BCA Protein Assay Kit (Thermo Fisher Scientific). Three micrograms of each preparation were denatured with Bolt LDS Sample Buffer and Sample Reducing Agent and boiled at 90° C. for 5 min. Proteins were separated on 4-12% Bis-Tris gels and stained using a Pierce Silver Stain Kit (Thermo Fisher Scientific) following the manufacturer's instructions.

Electron microscopy of sucrose-purified virus preparations. Two ml of egg-grown APMV3 or APMV3/S-6P were incubated with goat anti-SARS-CoV-2 S serum at 1:50 for 1 h under agitation. Mixtures were loaded on a 30% and 60% sucrose gradient in 5 mL thin-wall ultra-clear tubes (Beckman) and centrifugated at 30,000 rpm for 2 h. Virus bands were collected and fixed with 2% paraformaldehyde in PBS. For immunogold staining, 10 μl of samples were adsorbed, in saturated humid chambers at RT, to freshly glow-discharged 200 mesh Formvar/carbon-coated Ni grids for 30 min. Grids were washed in PBS, then blocked with 2% bovine serum albumin (BSA) in PBS followed by 0.1% acetylated BSA (BSA-c, Aurion) in PBS. All samples were labeled with donkey anti-goat antibody conjugated to 6 nm Au that was 1:50 diluted in 0.1% BSA-c in PBS as per manufacturer's instructions. The grids were washed with 0.1% BSA-c in PBS, then with PBS, and then in water followed by negative-staining with methylamine vanadate and electron microscopy observation.

Hamster studies. The hamster studies were approved by the NIAID Animal Care and Use Committee. All the animal experiments were carried out following the Guide for the Care and Use of Laboratory Animals by the NIH. Six-week-old golden Syrian hamsters (Mesocricetus auratus, Envigo Laboratories, Frederick, MD) were used in two separate experiments conducted in the BSL2 and BSL3 facilities approved by the USDA and CDC. Intranasal immunization or challenges were performed under light isoflurane anesthesia; at pre-determined endpoints, the animals were euthanized by CO₂ inhalation prior to necropsy.

In Experiment #1, six-week-old female Syrian hamsters that were randomly divided into two groups of 45 animals each were bled for serology and were immunized intranasally 11 days later with 6 log₁₀ PFU of either APMV3 or APMV3/S-6P diluted in Leibovitz's L-15 medium (50 μl per nostril). On days 3, 5 and 7 post-immunization, six animals per group were necropsied and brain, blood, nasal turbinate, lung, liver, kidney, spleen, and intestines were harvested. Tissues were weighed, mixed with L-15 medium (10 ml per g of tissue), homogenized using a gentleMACS Dissociator (Miltenyi Biotech), and clarified by centrifugation. Aliquots were snap-frozen to be stored at −80° C. Virus replication was evaluated by dual-staining immunoplaque assay, as described above. Two animals per group were harvested on days 3, 5, and 7 for IHC. Blood was collected for serology on day 26 post-immunization from the remaining animals (n=21 per group).

In Experiment #2, six-week-old male Syrian hamsters were randomly divided into two groups of 10 animals each. Animals were bled for serology on day −1. On day 0, the animals were immunized intranasally with 6 log₁₀ PFU of either APMV3 or APMV3/S-6P. On day 27 post-immunization, sera were collected, and animals were transferred to the ABSL3 facility. On day 30 post-immunization, animals were challenged intranasally with 4.5 log₁₀ TCID₅₀ of SARS-CoV-2 USA-WA1/2020 in 100 μl volumes per animal. Body weights and clinical symptoms were monitored daily. On days 3 and 5 post-challenge, five animals per group were necropsied, and nasal turbinates, lungs, and brains were collected, and processed as described above. The presence of the challenge virus in clarified tissue homogenates was evaluated by limiting dilution titration on Vero E6 cells. Titers were expressed as 50% tissue culture infectious dose (TCID₅₀) per g tissue, determined as described previously (Subbarao et al., 2004).

Immunohistopathology analysis. Lung tissue samples from hamsters were fixed in 10% neutral buffered formalin, processed through a Leica ASP6025 tissue processor (Leica Biosystems), and embedded in paraffin. Five micrometer tissue sections were stained with hematoxylin and eosin for routine histopathology. For IHC evaluation, sections were deparaffinized and rehydrated. After epitope retrieval, sections were incubated with goat hyperimmune serum to SARS-CoV-2 S (N25-154) at 1:1000, and rabbit polyclonal anti-APMV3 serum at 1:100. Chromogenic staining was carried out on the Bond RX platform (Leica Biosystems) according to manufacturer-supplied protocols. Detection with DAB chromogen was completed using the Bond Polymer Refine Detection kit (Leica Biosystems). The VisUCyte anti-goat horse radish peroxidase (HRP) polymer (R&D Systems) replaced the standard Leica anti-rabbit HRP polymer from the kit to bind the goat anti-SARS-CoV-2 S antibodies. Slides were finally cleared through gradient alcohol and xylene washes prior to mounting. Sections were examined by a board-certified veterinary pathologist using an Olympus BX51 light microscope and photomicrographs were taken using an Olympus DP73 camera.

SARS-CoV-2 neutralization assay. SARS-CoV-2 neutralizing antibody titers were determined in the BSL3 laboratory. Heat-inactivated sera were 2-fold serially diluted in Opti-MEM and mixed with an equal volume of SARS-CoV-2 (100 TCID₅₀) and incubated at 37° C. for 1 h. Mixtures were added to quadruplicate wells of Vero E6 cells in 96-well plates and incubated for four days. The 50% neutralizing dose (ND₅₀) was defined as the highest dilution of serum that completely prevented cytopathic effect in 50% of the wells and was expressed as a log₁₀ reciprocal value.

APMV3 neutralization assay. Heat-inactivated hamster sera were four-fold serially diluted and mixed with an equal volume of APMV3 (1,000 PFU) and incubated at 37° C. for 30 min. Then, the mixture was added to duplicate wells of Vero cells that were seeded on 24-well plates. After 1 h incubation at 37° C., 0.8% methylcellulose in Opti-MEM containing 1% L-glutamine, 2.5% penicillin-streptomycin, 0.1% gentamicin, 0.5% amphotericin B and 2% TrypLE Select was added in each well. Four days later, plates were fixed and immunostained for APMV3 as described above. The 60% plaque reduction neutralization titer (PRNT₆₀) was defined as the highest serum dilution that showed a 60% reduction in the number of plaques compared to the number counted in wells without serum and was expressed as a log₁₀ reciprocal value.

Enzyme-linked immunosorbent assay (ELISA). Nunc Maxisorp flat-bottom, 96-well plates (Thermo Fisher Scientific) were coated with purified antigens (SARS-CoV-2 S or SARS-CoV-2 S RBD) diluted in carbonate bicarbonate buffer (Sigma) at 4° C. overnight, and ELISAs were performed as previously described (Liu et al., under review). Plates were read at 450 nm using a microplate reader (Synergy Neo2, BioTek). For each serum, the average optical density (OD) at each dilution from duplicate wells was calculated with the average OD in blank wells subtracted. An end-point titer was defined as the highest dilution of each serum corresponding to the OD above the cutoff (average OD in blank wells plus three standard deviations) and was determined by interpolation using a model standard curve (Sigmoidal, 4PL, X is log [concentration]) in Prism 8 (GraphPad Software) and expressed as a log₁₀ reciprocal value. Serum IgA antibodies to the secreted form of S-2P or RBD were measured by DELFIA-TRF (Perkin Elmer) following the supplier's protocol.

Analysis of gene expression and quantification of SARS-CoV-2 RNA in lung tissues of hamsters. From 0.125 ml of lung homogenates (0.1 g of tissue per ml), total RNA was isolated using TRIzol Reagent, Phasemaker Tubes Complete System (Thermo Fisher Scientific), and PureLink RNA Mini Kit (Thermo Fisher Scientific) following the manufacturer's instructions. Using the High-Capacity RNA-to-cDNA Kit (Thermo Fisher Scientific), cDNA was amplified from 350 ng of each RNA. These cDNAs were used to quantify the level of expression of inflammatory/antiviral genes as well as SARS-CoV-2 RNA.

TaqMan low-density array cards (Thermo Fisher Scientific) were designed to contain TaqMan primers and probes for golden-Syrian hamsters inflammatory/antiviral genes and β-actin as a housekeeping gene (Bricker et al., 2021; Espitia et al., 2010; Sanchez-Felipe et al., 2021; Zivcec et al., 2011). Each cDNA was mixed with TaqMan Fast Advanced Master Mix (Thermo Fisher Scientific) and added into each fill port of the array cards for RT-qPCR with QuantStudio 7 Pro (Thermo Fisher Scientific). Results were analyzed using the comparative threshold cycle (AACT) method, normalized to β-actin, and expressed as fold changes over the average expression in three non-immunized, non-challenged hamsters. Heat maps were generated using the Gene Expression Similarity Investigation Suite (GENESIS program, release 1.8.1, genome.tugraz.at).

Quantification of genomic N (gN), E (gE), and subgenomic E mRNA (sgE) of the SARS-CoV-2 challenge virus USA-WA1/2020 was done in triplicate using TaqMan Fast Advanced Master Mix. The PCR strategy was taken from published work (Chandrashekar et al., 2020; Corman et al., 2020; Wolfel et al., 2020). The sgE assay used a forward PCR primer in the leader sequence and a reverse primer in the E gene, which thus made amplification specific to that subgenomic RNA, while the gE and gN assay used forward and reverse primers in the coding sequence, which thus made amplification specific to genomic RNA. Standard curves were generated using pcDNA3.1 plasmids containing gN, gE, or sgE sequences. The sensitivity of these TaqMan assays was 10 copies, which corresponds to a limit of detection of 5.2 log₁₀ copies per g of lung tissue.

Statistical analysis. Data sets were assessed for significance using one-way or two-way ANOVA with Ad-hoc or Sidak post-test using Prism 8 (GraphPad Software). Data were only considered significant at p≤0.05.

Virus recovery to generate material for clinical studies. To generate seed viruses for manufacture of clinical trial material, virus recovery was performed in WHO Vero cells, grown in OptiPro serum free medium (SFM) supplemented with 4 mM L-glutamine without animal-derived materials. Vero cells were electroporated using the Neon electroporation system (ThermoFisher) following the supplier's protocol. Programs 11 (1100 V, 40 ms, 1 pulse) or 17 (850 V, 30 ms, 2 pulses), and 19 (1050 V, 30 ms, 2 pulses) were selected based on pilot studies. 5×10⁵ Vero cells were transfected with plasmids pAPMV3/S-6P (2.5 μg), T7 spN3 (2.5 μg) expressing T7 RNA polymerase, and indicated pTM1 or pcDNA3.1 support plasmids (0.25 μg of APMV3 N support plasmid, 0.15 μg APMV3 P support plasmid, and 0.05 μg of APMV3 L support plasmid). To generate plasmids pTM1-N_{CO}, pTM1-P_{CO}, and pTM1-L_{CO}, the APMV3 ORFs had been optimized for human codon usage, Vero cells were transferred to individual wells of 12-well tissue culture plates pre-filled with 1 mL of OptiPro SFM with 4 mM L-glutamine. On day 2 after transfection, supernatants were harvested, clarified by centrifugation, and snap frozen. The titers of clarified supernatants were determined by dual-staining immunoplaque assay.

Table 1. Support plasmids for efficient virus recovery by electroporation of Vero cells. To generate seed viruses for clinical studies, virus recovery was performed in WHO Vero cells, grown in OptiPro serum free medium (SFM) supplemented with 4 mM L-glutamine without animal-derived materials. Vero cells were electroporated using the Neon electroporation system (ThermoFisher) following the supplier's protocol. Programs 11 (1100 V, 40 ms, 1 pulse), 17 (850 V, 30 ms, 2 pulses), and 19 (1050 V, 30 ms, 2 pulses) were selected based on pilot studies. To identify optimal conditions for virus recovery, two replica transfections were performed with each set of cDNAs using the specified electroporation programs (5×10⁵ Vero cells per transfection). After electroporation, Vero cells were transferred to individual wells of a 12-well tissue culture plate pre-filled with 1 mL of OptiPro SFM with 4 mM L-glutamine. On day 2 after transfection, supernatants were harvested, and the titers of clarified supernatants were determined by dual-staining immunoplaque assay. Wells positive for virus recovery (d) and APMV3/S-6P virus titers (e) are indicated. The APMV3 full length clone expressing the “6P” stabilized version of the SARS-CoV-2 S protein (pAPMV3/S-6P) and plasmid T7 spN3 expressing T7 RNA polymerase were used in each electroporation reaction, together with the indicated sets of support plasmids expressing the APMV3 N, P, and L proteins. The first set of support plasmids contained pTM-N, pTM1-P, and pcDNA3.1-L. The second set of support plasmids contained pTM1-N, pTM1-P, and pTM1-L. In the third set, pTM1 support plasmids contained APMV3 ORFs that had been optimized for human codon usage (pTM-N_{CO}, pTM-P_{CO}, pTM-L_{CO}). No APMV3/S-6P virus could be recovered by electroporation of Vero cells using support plasmids that included a pcDNA3.1-based plasmid expressing the APMV3 L protein. Transfection with pTM1 support plasmids containing non-codon optimized APMV3 sequences resulted in virus recovery from 2/6 replicas, while transfection with support plasmids expressing the N, P, and L proteins from codon-optimized ORFs resulted in virus recovery in 4/6 replicas. Thus, recovery using support plasmids with codon-optimized sequences was essential for efficient virus recovery in a system suitable for manufacture of material for clinical studies.

TABLE 1
Virus recovery in Vero cells by electroporation
using codon-optimized support plasmids.

| APMV3 N, P, L support | | | | |
|---|---|---|---|---|
| plasmids used with | Neon | | APMV3 and | |
| pAPMV3/S-6P (2.5 μg) | Electroproration | Independent | SARS-CoV-2 | Titer |
| and T7 spN3 (2.5 μg) ^{a} | Program^{b} | Replica^{c} | S positive^{d} | [PFU/ml]^{e} |
| pTM1-N (0.25 μg) | 11 | a | — | <5 |
| pTM1-P (0.15 μg) | 11 | b | — | <5 |
| pcDNA3.1-L (0.05 μg) | 17 | a | — | <5 |
| | 17 | b | — | <5 |
| | 19 | a | — | <5 |
| | 19 | b | — | <5 |
| pTM1-N (0.25 μg) | 11 | a | — | <5 |
| pTM1-P (0.15 μg) | 11 | b | — | <5 |
| pTM1-L (0.05 μg) | 17 | a | yes | 5 |
| | 17 | b | — | <5 |
| | 19 | a | yes | 10 |
| | 19 | b | — | <5 |
| pTM1-N_{CO} (0.25 μg) | 11 | a | — | <5 |
| pTM1-P_{CO} (0.15 μg) | 11 | b | yes | 10 |
| pTM1-L_{CO} (0.05 μg) | 17 | a | yes | 10 |
| | 17 | b | yes | 10 |
| | 19 | a | yes | 15 |
| | 19 | b | — | <5 |

^{a} Vero cells were electroporated using the Neon electroporation transfection system (ThermoFisher) following the supplier's protocol using indicated DNA plasmids. pAPMV3/S-6P, cDNA plasmid expressing the APMV3 genome with S-6P inserted as an additional gene between P and M; T7 spN3, plasmid expressing the RNA T7 polymerase; pTM1 and pcDNA3.1 support plasmids expressing the indicated APMV3 proteins under control of a T7 promoter; support plasmids contain an encephalomycarditis virus (ECMV) internal ribosomal entry site (IRES) to facilitate cap-independent translation. CO, optimized for human codon usage.
^{b}Neon electroporation programs 11 (1100 V, 40 ms, 1 pulse), 17 (850 V, 30 ms, 2 pulses), and 19 (1050 V, 30 ms, 2 pulses) were identified as most promising in prior pilot studies (data not shown).
^{c}Two replica transfections were performed with indicated cDNAs using the specified electroporation programs (5 × 10⁵ Vero cells per transfection). After electroporation, Vero cells were transferred to individual wells of 12-well tissue culture plates containing 1 mL of OptiProSFM medium with 4 mM L-glutamine.
^{d, e}On day 3 after transfection, 2% TrypLE Select was added, and cells were scaped into the supernatant. The mixture was vortexed for 30 seconds. The supernatant was clarified by centrifugation and snap-frozen. The titers of the clarified supernatants were determined later by dual-staining immunoplaque assav. Wells positive for virus recoverv (d) and titers (e) are indicated.

### Results

Generation of APMV3 expressing the SARS-CoV-2 S protein. APMV3 was used to express the SARS-CoV-2 S protein, the main protective antigen of SARS-CoV-2. The open reading frame (ORF) encoding the full-length SARS-CoV-2 S protein (1,273 aa) of the first available sequence [NCBI reference sequence NC_045512.2; (Wu et al., 2020a)] was codon-optimized for human expression. Three versions of the ORF were made encoding: (i) the unmodified full-length wild-type (WT) S protein (FIG. 1A, top construct); (ii) S protein stabilized in prefusion conformation by two proline substitutions at aa positions 986 and 987 (S-2P) (Wrapp et al., 2020); FIG. 1A, second from bottom); and (iii) S protein stabilized in the prefusion conformation by the above-mentioned two proline substitutions at aa positions 986 and 987 S-6P plus four additional proline mutations at aa positions 817, 892, 899, and 942 (S-6P), which were shown to confer increased stability to the prefusion conformation (Hsieh et al., 2020) (FIG. 1A, bottom). In addition, in the S-2P and S-6P versions, the S1/S2 polybasic furin cleavage motif “RRAR” was ablated by aa substitutions (RRAR-to-GSAS), as previously described (Wrapp et al., 2020). (Wrapp et al., 2020).

In addition, we are developing versions expressing stabilized versions of the S protein of the B.1.617.2 (Delta) or B.1.529 (Omicron) variants, providing for vaccine candidates with improved immunogenicity against currently circulating variants of concern.

Each of the three ORFs was designed to be framed by nucleotide adapters for insertion as an additional gene between the APMV3 P and M genes in the APMV3 genome (FIG. 1A and Materials and Methods). The design placed the S ORF under control of APMV3 gene start and gene end signals to direct transcription by the APMV3 polymerase complex into a separate mRNA. The three different versions of the S ORF were synthesized de novo and inserted into a cDNA clone encoding a full-length positive-sense copy of the APMV3 genome. These cDNAs were transfected with support plasmids into baby hamster kidney cells stably expressing T7 RNA polymerase (BSR T7/5; (Buchholz et al., 1999)), resulting in the recovery, by reverse genetics, of the viruses APMV3/S, APMV3/S-2P, and APMV3/S-6P. In parallel, we recovered the parental APMV3 empty vector from cDNA.

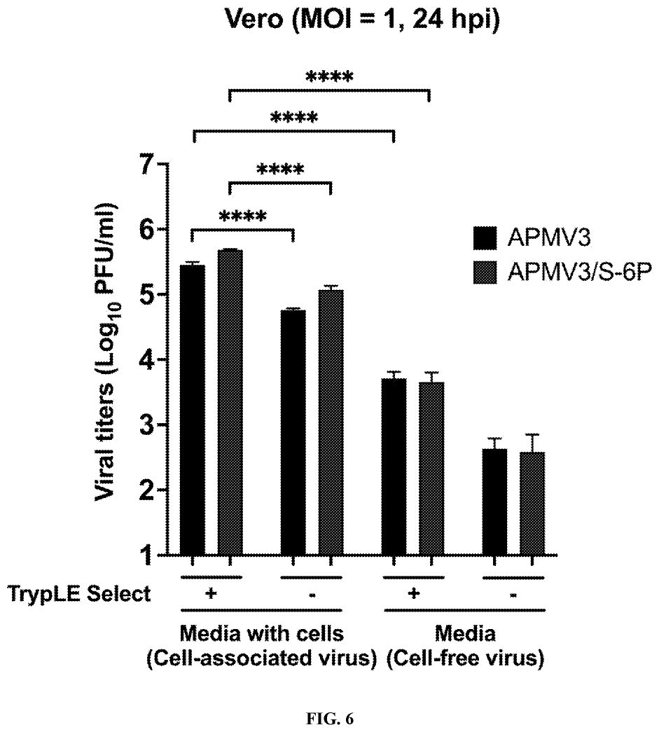

Viruses recovered from BSR T7/5 cells were passaged once or twice on Vero cells, a suitable substrate for vaccine manufacture. APMV3 replication on Vero cells is dependent on the addition of trypsin or allantoic fluid to provide the protease needed for cleavage activation of the APMV3 FO precursor (Kumar et al., 2008). In the present study, the addition of trypsin to the cell culture medium, as well as gentle manipulation of virus-infected Vero cells during the harvest by scraping and vortexing, significantly increased virus titers in clarified supernatants from the infected Vero cells (FIG. 6). Nonetheless, the recovered APMV3s replicated only to moderate titers in Vero cells, with final titers of virus stocks not exceeding 5.5 log₁₀ plaque-forming units (PFU) per mL (FIG. 6).

To generate high-titer virus stocks, the recombinant APMV3s were further amplified by inoculating Vero-grown material into the allantoic cavity of 11-day-old embryonated chicken eggs, which is another suitable substrate for vaccine manufacture. Two days after inoculation, allantoic fluids were harvested. The empty APMV3 vector replicated to high titers reaching a mean titer of 9.1 log₁₀ PFU/ml on day 2 post-infection (pi; from n=2 independent recoveries, FIG. 1). APMV3/S (n=2), APMV3/S-2P (n=2), and APMV3/S-6P (n=3) also replicated efficiently in embryonated chicken eggs, reaching mean titers of 8 log₁₀, 8.7 log₁₀, and 8.3 log₁₀ PFU/ml, respectively (FIG. 1).

Next, the stability of the S expression by Vero- and egg-derived APMV3 was evaluated by a dual-staining immunoplaque assay. This assay was designed to detect co-expression of S and APMV3 proteins by individual plaques that had developed on Vero cell monolayers under a methyl cellulose overlay. The monolayers were fixed and immunostained using a chicken hyperimmune serum to APMV3 and a human monoclonal antibody to the S protein. Following staining with species-specific secondary antibodies conjugated to infrared fluorescent dyes, infrared imaging revealed the presence of APMV3 proteins and S protein (pseudocolored in red and green, respectively): plaques expressing APMV3 proteins but not S would appear red and plaques co-expressing APMV3 protein and S protein would appear yellow (FIG. 1C). No significant differences in plaque morphology were observed between the empty APMV3 vector and APMV3 expressing any of the three versions of S protein (FIG. 1C). The expression of the three different versions of S by APMV3 vectors grown in Vero cells was stable, with dual-staining for both APMV3 and S protein detectable in >99% of plaques.

However, after amplification in embryonated chicken eggs, approximately 92% (mean from three independent virus recoveries) of APMV3/S-6P plaques were positive for S immunostaining, whereas only 32% (mean from two independent recoveries) and 25% (mean from two independent recoveries) of the APMV3/S and APMV3/S-2P plaques were positive for S immunostaining, respectively (FIG. 1D). Thus, substantial fractions of APMV3/S and APMV3/S-2P virions had lost the ability to express the SARS-CoV-2 S protein during propagation in chicken eggs. Sanger sequencing of the S ORF of two egg-grown APMV3/S stocks and one of the two egg-grown APMV3/S-2P stocks did not reveal any mutations. The second egg-grown APMV3/S-2P stock was generated by pooling allantoic fluid from 19 individual infected eggs. Prior to pooling, aliquots from six of the 19 eggs were used to extract viral RNA and sequence the S ORF by Sanger sequencing. No mutation in the S ORF was identified in the viruses harvested from three of these six eggs. However, nonsense mutations were identified at codon 129 (virus harvested from two eggs) or codon 291 of S ORF (virus from one egg). Complete-genome sequencing of one egg-grown APMV3 stock and three egg-grown APMV3/S-6P stocks were also performed, and did not reveal any mutations. Thus, the introduction of the six-proline substitutions provided increased stability of S expression from the APMV3 vector during replication in embryonated chicken eggs. Due to their low yields in Vero cells and genetic instability in embryonated chicken eggs, APMV3/S and APMV3/S-2P were not evaluated further, while AMPV3/S-6P was further investigated in vitro and in the hamster model.

In-vitro characterization of APMV3/S-6P. To compare the multicycle replication of APMV3/S-6P and APMV3, Vero or A549 cells were infected with the egg-grown virus stocks at a low multiplicity of infection (MOI) of 0.01 at 37° C. in the presence of trypsin. In Vero cells, both viruses replicated with similar kinetics and reached peak titers of around 5.3 log₁₀ PFU/ml at 64 hpi (FIG. 1E). APMV3/S-6P and APMV3 also replicated in human lung A549 cells, albeit less efficiently than in Vero cells reaching peak titers at 32 hpi of only 3.9 log₁₀ and 4.0 log₁₀ PFU/ml, respectively (FIG. 1F).

To evaluate if S was stably expressed by APMV3/S-6P in Vero and A549 cells, the percentage of plaques that expressed S over the multicycle replication experiments was determined by the dual-staining immunoplaque assay (FIG. 1G). Following passage in Vero or A549 cells, 83 to 98% of APMV3/S-6P plaques were S-positive, with the percentages being very similar between the two cell lines. In addition, the percentage of S-expressing plaques did not decrease over the time course, suggesting that S expression by APMV3/S-6P was stable in both cell lines.

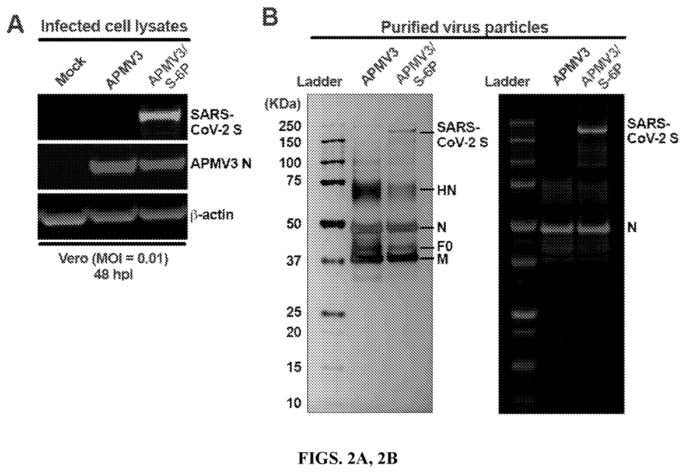

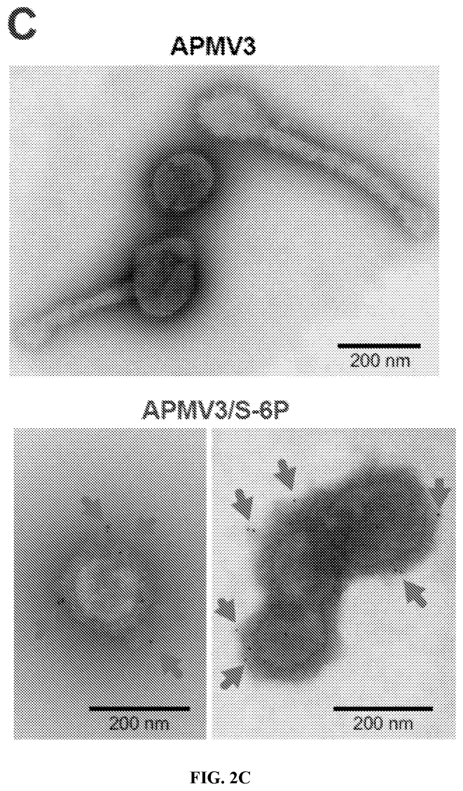

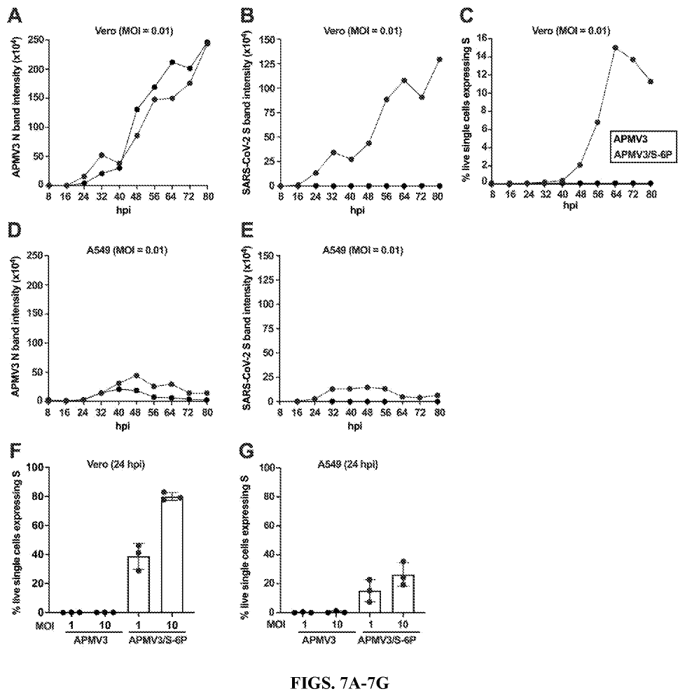

Additional wells were used from the multicycle replication experiment described in FIGS. 1E and 1F to characterize the expression of viral proteins in Vero and A549 cells by Western blotting and flow cytometry (FIG. 7A). Cell lysates were prepared and analyzed under reducing and denaturing conditions by SDS polyacrylamide gel electrophoresis (PAGE) and Western blotting using antisera against APMV3 and against SARS-CoV-2 S. A representative Western blot of Vero cells harvested 48 hpi is shown in FIG. 2A. In lysates of APMV3- and APMV3/S-6P-infected Vero cells, we detected a strong band consistent in size with APMV3 N. In Vero cells, the APMV3 N protein accumulated similarly in APMV3- and APMV3/S-6P-infected cells from 24 to 80 hpi (FIG. 7A). In the APMV3/S-6P infected-cell lysate only, we additionally detected a single strong band consistent in size with the uncleaved SARS-CoV-2 S0 protein (FIG. 2A). The SARS-CoV-2 S protein also accumulated in APMV3/S-6P-infected Vero cells from 24 to 80 hpi (FIG. 7B). Using replicate wells from the same experiment, the rate of S expressing cells over time was determined by flow cytometry (FIG. 7B). This showed that, at 64 hpi, about 15% of Vero cells infected with APMV3/S-6P were expressing SARS-CoV-2 S (FIG. 7C). This percentage declined from 64 to 80 hpi. As described above, about 98% of the plaques produced by APMV3/S-6P expressed S in Vero cells at 64 hpi (FIG. 1G). Thus, S was an appropriate marker to evaluate the rate of APMV3/S-6P-infected cells. In A549 cells, the expression level of APMV3 N and SARS-CoV-2 S was five- and eight-fold lower than in Vero cells, respectively (FIGS. 7D and 7E).

In an additional set of three independent experiments, Vero and A549 cells were infected with APMV3 or APMV3/S-6P using higher MOIs of 1 or 10 PFU/cell (FIGS. 7F and 7G). Cells were harvested at 24 hpi, and the proportion of cells expressing SARS-CoV-2 S protein was analyzed by flow cytometry. About 40% and 80% of the live single permeabilized Vero cells were positive for S protein at 24 hours with an MOI of 1 and 10 PFU/cell, respectively (FIG. 7F), while only about 15% and 26% of A549 cells had the S protein detectable at an MOI of 1 or 10 PFU/cell (FIG. 7G). Thus, A549 cells were susceptible to APMV3/S-6P infection, but their permissiveness was lower than that of Vero cells.

Incorporation of SARS-CoV-2 S in APMV3/S-6P virus particles. To investigate whether SARS-CoV-2 S was incorporated in APMV3/S-6P virions, we purified APMV3 and APMV3/S-6P that had been grown to high titers in embryonated chicken eggs by ultracentrifugation through discontinuous sucrose gradients. To analyze the protein composition, virus preparations were subjected to SDS-PAGE followed by silver staining (FIG. 2B, left panel) or Western blotting (FIG. 2B, right panel). A band with a molecular size corresponding to that of SARS-CoV-2 S was apparent on the silver-stained gel in the APMV3/S-6P preparation, but not in the APMV3 preparation. Immunostaining confirmed the presence of SARS-CoV-2 S in sucrose-purified preparations of APMV3/S-6P (FIG. 2B, right panel). Silver staining revealed additional protein bands in both the APMV3 and APMV3/S-6P preparations (FIG. 2B, left panel). These protein bands were identified by mass spectrometry of gel bands excised from a Coomassie blue-stained gel ran in parallel, as APMV3 HN (estimated molecular weight: 70 kDa), N (45-50 kDa), FO (40-45 kDa), and M (40 kDa). HN in particular was somewhat more abundant in the APMV3 preparation than in the APMV3/S-6P preparation. Western blotting confirmed that APMV3 N was present in similar amounts in both APMV3 and APMV3/S-6P preparations.

The incorporation of S protein in APMV3/S-6P particles was also evaluated by immune-electron microscopy (FIG. 2C). Egg-grown APMV3 and APMV3/S-6P were incubated with goat polyclonal antibodies raised against a recombinantly-expressed secreted form of S-2P (Liu et al., under review), followed by purification by ultracentrifugation through a discontinuous sucrose gradient. Purified preparations were labeled with a secondary anti-goat antibody conjugated with gold particles, followed by negative staining. Electron microscopy revealed the presence of abundant pleomorphic particles surrounded by envelope glycoproteins, consistent with APMV3 virions. No obvious differences in morphology between wt APMV3 and APMV3/S-6P were observed. Immunogold-labeling was associated with the glycoprotein layer surrounding the APMV3/S-6P particles (FIG. 2C, bottom panels), indicating the presence of S protein on the APMV3/S-6P envelope. No immunogold labeling was detected on APMV3 virus particles (FIG. 2C top panel). This further confirmed that SARS-CoV-2 S was incorporated in the APMV3/S-6P particles.

Replication and genetic stability of APMV3/S-6P in hamsters. APMV3 replicates to moderate levels (˜3 log₁₀ PFU/g lung tissue) in the respiratory tract of golden Syrian hamsters without any gross clinical disease (Samuel et al., 2011). The hamster ACE2 sequence shares a high similarity with human ACE2, and SARS-CoV-2 replicates to higher titers than APMV3 in hamsters (˜7 log₁₀ PFU/g lung tissue) and induces moderate clinical disease (Chan et al., 2020; Imai et al., 2020; Sia et al., 2020). Thus, golden Syrian hamsters are an appropriate animal model to evaluate both the replication and immunogenicity of APMV3 vectors, and protection against SARS-CoV-2 challenge.

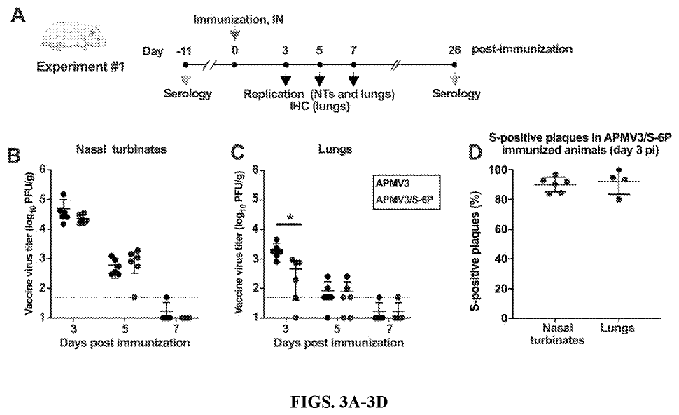

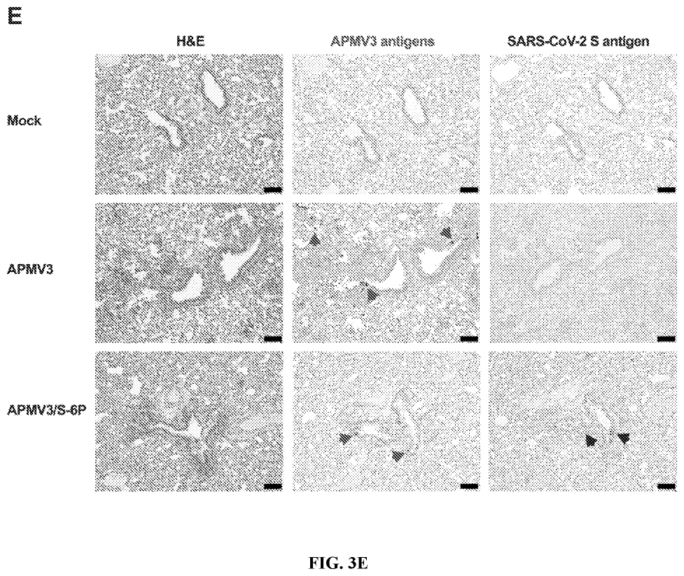

To evaluate virus replication and immunogenicity, six-week-old hamsters in groups of 45 were inoculated intranasally with a dose of 6 log₁₀ PFU of APMV3/S-6P or the empty APMV3 vector per animal (Experiment #1, see FIG. 3A for the timeline). On days 3, 5 and 7 pi, six hamsters per group were euthanized, and virus replication was evaluated in the nasal turbinates (NT) and lungs (FIGS. 3B-3D), as well as in brain tissue, kidneys, spleen, liver, intestines, and blood. It was found that APMV3 and APMV3/S-6P replicated similarly well in the NT reaching 4.5 log₁₀ and 4.3 log₁₀ PFU/g, respectively on day 3 pi (FIG. 3B). Titers of both viruses were lower by day 5 pi (2.7 log₁₀ and 3.0 log₁₀ PFU/g for APMV3 and APMV3/S-6P, respectively), and no replication was detected on day 7 in both groups, except for one animal in the APMV3 group in which replication in the NT was detected at the limit of detection.

Both viruses replicated to lower titers in the lungs than in the NTs (FIG. 3C); the peak titers of APMV3 and APMV3/S-6P on day 3 pi reached 3.3 log₁₀ PFU/g (70-fold lower compared to NTs) and 2.7 log₁₀ PFU/g (20-fold lower compared to NTs), respectively. The mean peak titer of APMV3/S-6P was four-fold lower compared to the APMV3 empty vector control, suggesting that the insertion of the S gene reduced APMV3 replication in the lungs. Lung titers of both viruses were lower by day 5. On day 7, only one animal from each group had virus detectable in the lungs, with titers at the limit of detection.

The stability of S expression by APMV3/S-6P was evaluated by dual-staining immunoplaque assay from animals necropsied on day 3 pi with virus detectable by titration in respiratory tissues (NTs and lungs from six and four hamsters, respectively; FIG. 3D). About 90% and 92% of the APMV3/S-6P plaques from the NT and lungs, respectively, expressed S (FIG. 3D), providing evidence that S expression by APMV3/S-6P was stable in hamsters. No replication of APMV3 and APMV3/S-6P was detected in the spleen, intestine, brain, kidney, liver and blood, confirming that APMV3 replication was restricted to the respiratory tract and was not altered by the introduction of S in its prefusion form.

On days 3, 5 and 7, lung tissues were harvested from two additional animals per group and processed for immunohistochemistry (IHC) analysis to detect the expression of APMV3 and SARS-CoV-2 S antigen. Representative hematoxylin and eosin (H&E) staining and IHC images from day 5 are shown in FIG. 3E. APMV3 and SARS-CoV-2 antigen was detected (indicated by colored arrows) in columnar bronchial epithelial cells. Antigen-positive cells were largely limited to the bronchial and bronchiolar airway epithelium, and only present in a few locations and only at day 5 post-immunization.

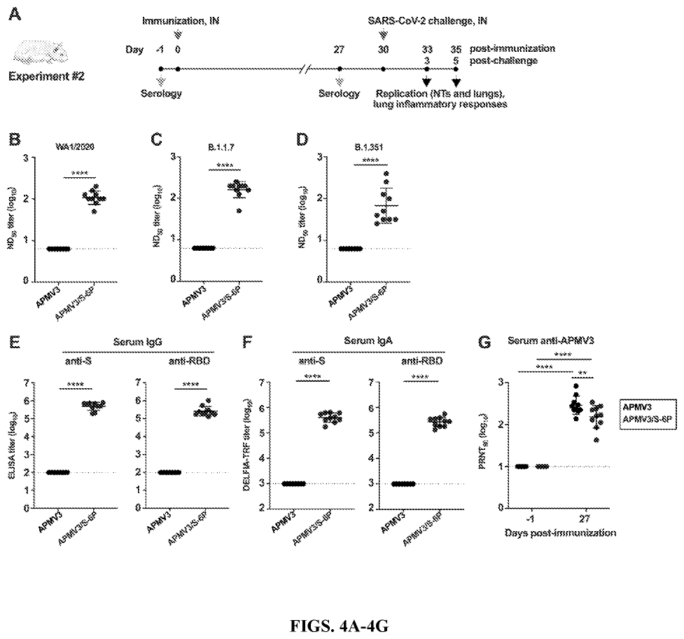

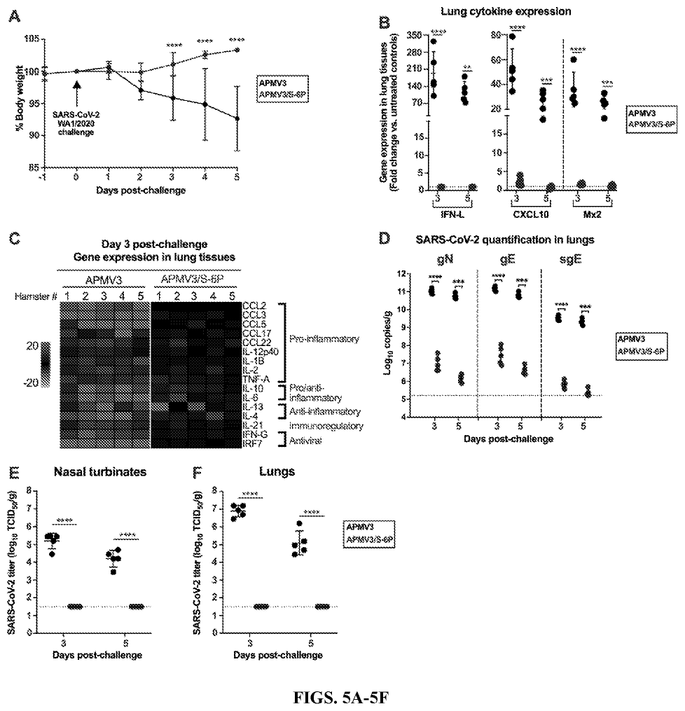

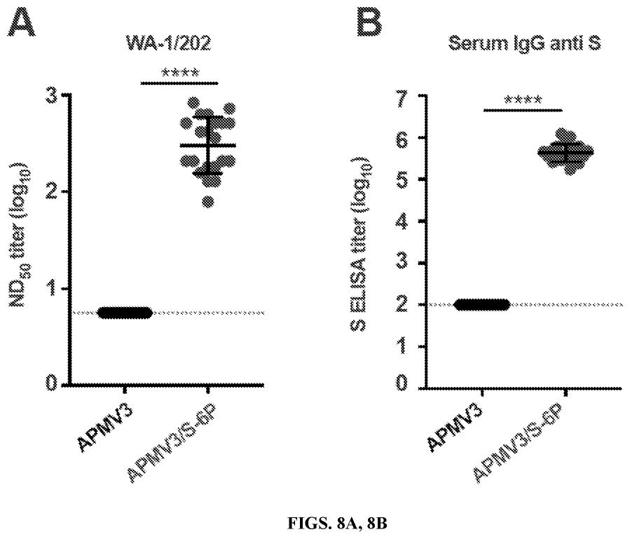

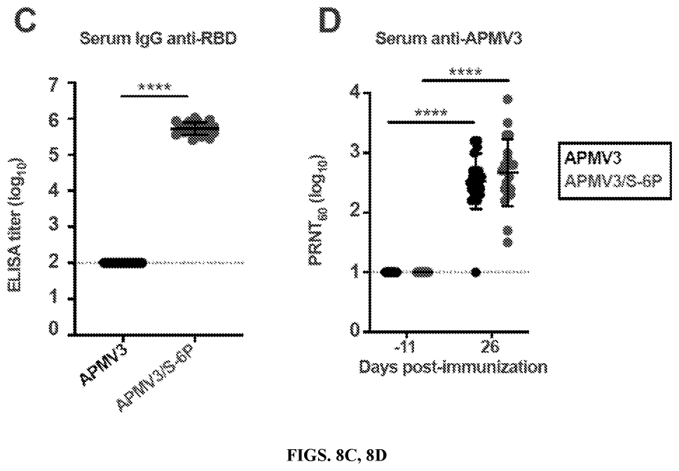

Immunogenicity in hamsters. In a continuation of Experiment #1 described above (see FIG. 3A for the timeline), we collected sera on day 26 pi from the remaining 21 animals per group. In addition, a second study was performed in hamsters (see FIG. 4A for the time line), also six-week-old hamsters, in which two groups of 10 hamsters each were immunized intranasally with the same inocula as in Experiment #1 (6 log₁₀ PFU of APMV3/S-6P or empty APMV3 vector). Sera were obtained on day 27 after immunization. Analyses of the sera from Experiments #1 and #2 are shown in FIGS. 4B-4G and FIGS. 8A-8D, respectively, and described below.

The SARS-CoV-2 neutralizing antibody titers were measured by 50% neutralizing dose (ND₅₀) assays. In both experiments, hamsters immunized with APMV3/S-6P developed high levels of serum neutralizing antibodies against the homologous SARS-CoV-2 WA1/2020 isolate (mean ND₅₀ titers of 2.5 and 2.0 log₁₀ in experiments #1 and #2, respectively; FIGS. 4B and 8A). In addition, using sera from Experiment #2 alone, the serum neutralizing titers were evaluated against two variants of concern, lineages B.1.1.7 and B.1.351. Serum antibodies from APMV3/S-6P-immunized hamsters efficiently neutralized the SARS-CoV-2 B.1.1.7 lineage (mean ND₅₀ titer of 2.2 log₁₀, FIG. 4C), as well as the SARS-CoV-2 B.1.351 lineage, although the titers against the latter lineage were 1.6-fold lower (mean ND₅₀ titer of 1.8 log₁₀), and more variable among the individual animals (ND₅₀ titers ranging from 1.4 to 2.6 log₁₀, FIG. 4D).

In both experiments, the levels of serum IgG against a recombinantly-expressed secreted form of the SARS-CoV-2 S protein (aa 1-1208; FIGS. 4E, left panel, and 8B) and the RBD of S (FIGS. 4E, right panel, and 8C) were also measured by ELISA. In both studies, APMV3/S-6P induced a strong antibody response against the S protein (mean titers of 5.6 and 5.7 log₁₀ in Experiment #1 and #2, respectively) and against the RBD (mean titer=5.7 and 5.4 log₁₀, in Experiment #1 and #2, respectively). The similarity in titers between the anti-S and anti-RBD responses suggested that most of the anti-S response was directed against the RBD. In addition, in Experiment #2, the serum IgA titers against S and RBD were evaluated by dissociation-enhanced lanthanide time-resolved fluorescent immunoassay (DELFIA-TRF) (FIG. 4F). This showed that APMV3/S-6P induced strong anti-S and anti-RBD IgA responses (mean titers of 5.6 and 5.4 log₁₀, respectively). As expected, the SARS-CoV-2 neutralizing antibody titers and the anti-S and anti-RBD IgG ELISA titers of the sera collected from the APMV3-empty-vector-immunized groups in both experiments were below the limit of detection (FIGS. 4B-4F and 8A-8C).

Finally, in both experiments, there were strong serum neutralizing antibody responses against the APMV3 vector: in Experiment #1, the responses were not significantly different between the two groups (FIG. 8D), whereas in Experiment #2 the response was modestly but significantly reduced in the APMV3/S-6P group (FIG. 4G).

APMV3/S-6P protects hamsters from a SARS-CoV-2 challenge. In a continuation of Experiment #2 described above (see FIG. 4A for the timeline), the protective efficacy against SARS-CoV-2 was assessed by challenging the hamsters on day 30 with 4.5 log₁₀ TCID₅₀ of SARS-CoV-2 USA-WA1/2020 (FIGS. 4A and 5A-5F).

The body weights of the APMV3- and APMV3/S-6P-immunized hamsters were monitored from day −1 to 5 post-challenge (FIG. 5A). Hamsters immunized with APMV3 lost weight from day 2 to 5 following SARS-CoV-2 challenge (7.3% average loss from their initial weights on day 0), while APMV3/S-6P-immunized hamsters continued to gain weight (3.3% average gain from their initial weight on day 0). No other clinical symptoms were observed in either group.

To compare inflammatory responses following SARS-CoV-2 challenge, the expression of 19 selected inflammatory/antiviral genes was analyzed by TaqMan real-time quantitative PCR (RT-qPCR) assays of total RNA extracted from lung homogenates (FIGS. 5B and 5C). qPCR data were normalized to 3-actin and expressed as the fold-increase over the average value from three unimmunized and unchallenged hamsters. IFN-L and two IFN-regulated genes, CXCL10 and Mx2, were the three most strongly up-regulated genes on days 3 and 5 post-challenge in the lungs of APMV3-immunized animals (194-, 52- and 36-fold increase on average on day 3, respectively, FIG. 5B). In contrast, these three genes were barely up-regulated post-challenge in APMV3/S-6P-immunized hamsters (IFN-L did not have a detectable increase, and the average increases for CXCL10 and Mx2 were 2.2 and 1.6-fold, respectively). Analysis of the other 16 inflammatory/antiviral genes (FIG. 5C) similarly showed a strong induction of an inflammatory response post-challenge in the lungs of the APMV3-immunized hamsters, whereas the response post-challenge was much lower in the lungs of hamsters that had been immunized with APMV3/S-6P.

The presence of SARS-CoV-2 RNA was next evaluated in hamsters following challenge. SARS-CoV-2 genomic and subgenomic RNA were detected by RT-qPCR assays of RNA extracted from lung homogenates in the experiment described above (FIG. 5D). TaqMan assays were used to separately quantify the genomic N (gN), genomic E (gE), and subgenomic E (sgE) mRNA. The presence of sgE mRNA reflects active SARS-CoV-2 transcription, indicative of effective SARS-CoV-2 replication. In APMV3/S-6P-immunized hamsters, the levels of SARS-CoV-2 gN and gE RNA were 4 log₁₀ lower on days 3 and 5 post-challenge, respectively, compared to hamsters previously immunized with APMV3 empty-vector control. The level of SARS-CoV-2 sgE mRNA also was much lower in APMV3/S-6P-immunized hamsters compared to APMV3-immunized hamsters (4 log₁₀ difference on day 3), and close to the limit of detection (5.2 log₁₀ copies/g). Thus, intranasal immunization of hamsters with APMV3/S-6P strongly restricted SARS-CoV-2 replication in the lungs.

Next, SARS-CoV-2 replication was evaluated by a limiting dilution assay on Vero E6 cells of NT and lung tissue homogenates collected on day 3 and 5 post-challenge (FIGS. 5E-5F). In the NTs of hamsters immunized with the APMV3 empty vector, SARS-CoV-2 titers reached 5.2 log₁₀ TCID₅₀/g and 4.2 log₁₀ TCID₅₀/g on days 3 and 5 post-challenge, respectively (FIG. 5E), indicating that SARS-CoV-2 replicated efficiently. In contrast, no infectious SARS-CoV-2 was detected in the NT of APMV3/S-6P-immunized hamsters on days 3 and 5 post-challenge. In the lungs of APMV3 empty-vector immunized hamsters, SARS-CoV-2 titers reached 6.9 log₁₀ TCID₅₀/g and 5.1 log₁₀ TCID₅₀/g on day 3 and 5 post-challenge, respectively (FIG. 5F), indicating that the challenge virus replicated efficiently. However, SARS-CoV-2 was not detected by titration in the lungs of APMV3/S-6P-immunized hamsters at either day 3 or 5 post-challenge. In addition, no replication of SARS-CoV-2 was detected by titration from the brains of hamsters in any groups (data not shown). Therefore, a single intranasal dose of APMV3/S-6P successfully prevented replication of SARS-CoV-2 in the upper and lower respiratory tract of hamsters, and immunized hamsters were protected from severe inflammation in the lungs and weight loss.

APMV3/S-6P recovery in WHO Vero cells without animal derived materials is facilitated by codon optimization of APMV3 support plasmids. To generate material for clinical studies, it is essential that viruses can be recovered from cDNA in a suitable cell substrate, for example in World Health Organization (WHO) Vero cells, in absence of animal derived materials. Recovery of RNA viruses from cDNA in serum-free systems is very inefficient. To recover APMV3/S-6P, World Health Organization (WHO) Vero cells grown in serum-free media were electroporated with pAPMV3/S-6P and support plasmids using the Neon electroporation method (see Materials and Methods). Expression from cDNA plasmids of the full-length APMV3/S-6P antigenome and the APMV3 N, P, and L proteins is driven by the T7 RNA polymerase promoter (T7 promoter) and is dependent on co-expression of T7 RNA polymerase from an additional plasmid (T7 spN3). Recovery of APMV3/S-6P in Vero cells from this plasmid-driven system was not successful if a pcDNA3.1 support plasmid expressing the APMV3 L protein was included in the electroporation reactions (Table 1). Since pTM1 plasmids had been successfully used in recovery systems for other negative-strand RNA viruses, the APMV3 L ORF was transferred to a pTM1-based support plasmid. This pTM1-based plasmid was used to replace the pcDNA3.1-L support plasmid in electroporations of Vero cells. Using pTM1-N and pTM1-P, together with pTM1-L, APMV3/S-6P recovery was successful from 2 of 6 electroporation reactions. To increase the efficiency of virus recovery in Vero cells, APMV3 N, P, and L ORFs expressed by support plasmids were optimized for human codon usage. In electroporation experiments using these codon-optimized versions of pTM1-N_{CO}, pTM1-P_{CO}, and pTM1-L_{CO} support plasmids, APMV3/S-6P was recovered from 4 of 6 electroporation reactions, suggesting that the codon optimization increased the efficiency of virus recovery in an animal product free system based on WHO Vero cells (Table 1). Recovery of infectious APMV3 from plasmids depends on efficient co-expression of the APMV3 N, P, and L proteins from support plasmids.

### Discussion

A live attenuated APMV3-based vaccine candidate was developed against SARS-CoV-2 for intranasal delivery. The best-characterized, APMV1 (also known as NDV), was previously shown to be well-tolerated in humans when used at high doses (usually administered parenterally) in cancer immunotherapy or as an experimental oncolytic agent (Burman et al., 2020). Previous evaluation of NDV as a vaccine vector administered by the respiratory tract showed that it was highly attenuated in non-human primates due to its strong host-range restriction but nonetheless was highly immunogenic (Bukreyev and Collins, 2008; DiNapoli et al., 2009). However, NDV is an important pathogen for poultry, and the use of the more-immunogenic NDV strains as vaccine vectors is not practical due to animal health concerns. APMV3 represents an alternative as a vector platform. Unlike NDV, APMV3 is only mildly pathogenic in poultry (Kumar et al., 2010). APMV3 also is highly attenuated in mammals due to strong host-range restriction (Samuel et al., 2011). Similar to other APMVs, APMV3 has a tropism for respiratory epithelial cells, and our results show that it readily infects and replicates in human airway A549 cells.

Preferably, a viral vector should be based on a virus for which humans have low seroprevalence. Otherwise, pre-existing immunity to the vector will likely restrict its infection and replication, thereby reducing its antigenic load and immunogenicity. For example, the use of adenoviruses as vectors depends on the availability of human or simian adenovirus serotypes that exhibit low immunity in the general human population (Sadoff et al., 2021; Voysey et al., 2021). APMV3 is very distinct from human viruses, and it is anticipated to lack antigenic relatedness. In addition, human infection with APMV3 is generally unknown and likely resembles that of NDV, which occasionally infects individuals who have close contact with birds causing a restricted and mild infection. Therefore, most humans have not been infected with APMV3 and are free of any pre-existing direct or cross-reacting immunity to APMV3.

In other work, a chimeric bovine/human parainfluenza type 3 (B/HPIV3) vector a expressing the SARS-CoV-2 S protein was recently developed as a pediatric vaccine candidate for intranasal immunization (Liu et al., PNAS, under review). Like APMV3, B/HPIV3 is a replication-competent virus. However, an important difference is that B/HPIV3 is a vaccine candidate for the human pediatric pathogen HPIV3, and has been shown to be well-tolerated and immunogenic against HPIV3 in pediatric clinical trials (Bernstein et al., 2012; Karron et al., 2012). Thus, B/HPIV3 expressing SARS-CoV-2 provides a bivalent candidate vaccine against HPIV3 and COVID-19. However, the replication and immunogenicity of the B/HPIV3 vector would likely be restricted in HPIV3-immune individuals. Thus, the B/HPIV3 vector might not be effective in most adults, given the high seroprevalence to HPIV3, but should be suitable for pediatric use. In contrast, the APMV3 vector has potential use in both pediatric and adult populations regardless of exposure histories to human viruses.

In the present study, the full-length version (aa 1-1,273) of the S protein of the first available SARS-CoV-2 sequence from an ORF that was codon-optimized for human expression. The amino acid sequence of this S protein was identical to that of the challenge virus USA-WA1/2020 that was used in this study. The S protein was engineered to be stabilized in the prefusion conformation in two versions (S-2P and S-6P) by the introduction of two (K986P and V987P) or six (K986P and V987P plus F817P, A892P, A899P, and A942P) proline substitutions into the S2 domain, as previously described (Corbett et al., 2020; Hsieh et al., 2020; Mercado et al., 2020; Walsh et al., 2020). In addition, in each version, four amino acid residues of the furin cleavage site, 682-RRAR-685, were replaced with GSAS, as was previously described for secreted prefusion-stabilized versions (Hsieh et al., 2020; Wrapp et al., 2020). The S-2P protein is the form used in most of the SARS-CoV-2 vaccines in human use (Corbett et al., 2020; Mercado et al., 2020; Walsh et al., 2020) or in development (Tian et al., 2021) and was shown to induce a higher level of neutralizing antibodies than the WT S (Mercado et al., 2020). The further inclusion of the four additional proline substitutions (to create S-6P) resulted in eight-fold higher production in eukaryotic cells in vitro and greater stability in the prefusion conformation, compared to S-2P (Hsieh et al., 2020). Removing the furin cleavage site may further optimize the stabilization of the S protein in its prefusion conformation (Bos et al., 2020; Wrobel et al., 2020), and was previously shown to increase the efficacy of a recombinant spike-based SARS-CoV-2 vaccine in the mouse model (Amanat et al., 2021). Furthermore, the S protein with the GSAS cleavage site instead of RRAR did not induce cell-cell fusion and syncytia formation (Papa et al., 2021), as would be expected for a protein that is largely immobilized in the prefusion conformation. Thus, ablation of the furin cleavage site provides for an additional safeguard by rendering the SARS-CoV-2 S protein non-functional for mediating fusion in a live viral vector. In our study, we did not detect any replication of APMV3 or APMV3/S-6P outside of the respiratory tract in hamsters, indicating that the tissue tropism of the APMV3 vector expressing S did not differ from that of APMV3 in this model.

APMV3 vectors were constructed to express the WT S, S-2P, and S-6P proteins, and all viruses were efficiently recovered and expressed the corresponding S protein when passaged in Vero cells. However, when evaluated by plaque assay following replication to high titer in chicken embryonated eggs, a high proportion of APMV3/S and APMV3/S-2P virions had lost the expression of WT S and S-2P proteins, respectively. In contrast, expression of S-6P by APMV3/S-6P was maintained in a high percentage of virions, even though this virus replicated similarly to high titers in eggs. The genetic instability of the APMV3/S and APMV3/S-2P stocks precluded direct comparison with APMV3/S-6P. The reasons for the greater genetic stability of APMV3/6P remain to be elucidated. As one possibility, since the S-6P protein was incorporated in the APMV3/S-6P virion (and the S-2P form of S likely was also incorporated into the virions), it may be that incorporation of the less-stable S-2P protein was more detrimental to APMV3 virion formation than the more-stable S-6P protein, and thus conferred a greater selective pressure for loss of expression of its gene.

The characterization of APMV3/S-6P was further pursued in vitro. The replication kinetics of APMV3/S-6P were comparable to those of wild type APMV3 in Vero and A549 cells, but the overall replication in A549 cells was lower. However, the ability to grow APMV3/S-6P to high titers in embryonated chicken eggs, an approved vaccine substrate, would facilitate vaccine manufacture. Assuming that 6 log₁₀ PFU would be selected as a vaccine dose and that five to 10 ml of virus can be harvested from one egg, about 500 to 1000 doses per egg could be generated in two days.

As noted, the S-6P protein was incorporated into the B/HPIV3 virion. This likely did not confer any advantage to the vector, since the S-6P protein should be largely non-functional. However, in a previous study, we found that efficient incorporation of the respiratory syncytial virus (RSV) fusion (F) protein into the B/HPIV3 virion resulted in an increase in the quantity and quality of RSV-neutralizing antibodies that was comparable to that provided by stabilization in the prefusion conformation without incorporation (Liang et al., 2016). The increased immunogenicity associated with incorporation into the vector particle might be due to more efficient presentation to the immune system of a protein that is in a multimeric array and/or is in a particle (Liang et al., 2016). In any event, the incorporation of S-6P into the APMV3 vector particles that occurred similarly might increase its immunogenicity.

In the hamster model, APMV3/S-6P replicated as efficiently as APMV3 in the upper respiratory tract, but was slightly reduced in the lungs on day 3 pi, suggesting that the supernumerary gene marginally reduced the efficiency of replication in this model. Despite this mild restriction in the lung, a single intranasal delivery of APMV3/S-6P was highly immunogenic in hamsters. Indeed, 6 log₁₀ PFU of APMV3/S-6P induced robust serum neutralizing antibody titers against the lineage A isolate WA1/2020 in all immunized hamsters. A strong serum IgG response to the secreted version of prefusion S and to its RBD domain (with mean titers above 5.4 log₁₀ in two independent studies) was detected. This compares well with, and is considered to be at least as potent as, responses observed in animals that had been vaccinated intranasally or intramuscularly with adenovirus-vectored SARS-CoV-2 vaccines (Feng et al., 2020; Tostanoski et al., 2020; Wu et al., 2020b). The APMV3/S-6P-induced antibodies also neutralized two variants of concern of lineage B.1.1.7 and B.1.351, although the neutralizing titers to a representative of lineage B.1.351 were reduced and more variable among hamsters, similar to findings with other vaccine candidates expressing S proteins of representatives of the SARS-CoV-2 A lineage (Corbett et al., 2021). Thus APMV3/S-6P would be expected to generate at least some level of protection against these variants of concern.

APMV3/S-6P efficiently protected hamsters against challenge with SARS-CoV-2. Unlike hamsters previously immunized with the empty APMV3 vector, APMV3/S-6P-immunized hamsters were protected from weight loss after SARS-CoV-2 challenge. The low or absent inflammatory response in the lungs of APMV3/S-6P immunized hamsters following challenge further confirmed efficient protection against challenge. Furthermore, the absent or very low levels of infectious SARS-CoV-2 in the NTs and lungs suggested that intranasal inoculation with a single dose of APMV3/S-6P induced near-sterilizing immunity in the hamster model. Correlation of SARS-CoV-2 neutralizing serum antibodies with protective efficacy was described in an earlier report (Mercado et al., 2020). However, since the present vaccine candidate was live and was administered by the intranasal route, it is reasonable to suggest that a local airway IgA response as well as systemic and local T cell-mediated responses also contributed to protection in the respiratory tract. Since it is technically challenging to reliably determine antibody titers in the mucosal lining fluid in hamsters, the serum IgA titers to the SARS-CoV-2 S protein were measured by an enhanced IgA ELISAs, showing that a single intranasal dose of APMV3/S-6P induced a potent IgA response. A more extensive comparison of the mucosal and the serum IgA response to immunization with APMV3 vectors via the respiratory route will be performed in future studies in rhesus macaque studies.

Intranasal vaccination stimulates local mucosal immunity in addition to systemic immunity (Riese et al., 2014). In contrast, all currently approved human COVID-19 vaccines are administered parenterally and thus do not directly stimulate respiratory tract immunity. It is generally recognized that local mucosal immunity is particularly effective in restricting virus replication and re-infection in the respiratory tract. For example, studies with experimental vaccines for RSV in animal models showed that live-attenuated vaccines administered by the intranasal route provided greater protection in the upper respiratory tract compared to parenterally-administered vaccinia virus vectors or RSV subunits (Crowe et al., 1993; Murphy et al., 1990). Systemic immune effectors may not efficiently access the respiratory tract, especially the upper respiratory tract. For example, while serum IgG accesses the respiratory tract by transudation, for IgG specific to influenza A virus there was a gradient of approximately 350:1 between titers in the serum versus the upper respiratory tract (Wagner et al., 1987). Immunization in the respiratory tract induces local B cell responses in nasal- and bronchial-associated lymphoid tissue (NALT and BALT), resulting in the induction of mucosally-secreted and serum IgA and IgG, as observed in recent studies with adenovirus-vectored SARS-CoV-2 vaccine candidates (Feng et al., 2020; Wu et al., 2020b). Respiratory immunization also induces local and systemic T cell responses whereas parenteral immunization primarily induces systemic responses. For example, respiratory immunization with a parainfluenza virus vector expressing the Ebola virus GP efficiently induced lung-resident CD8+ and CD4+ T cells, compared to a predominantly systemic response to Ebola virus GP expressed by an alphavirus replicon given intramuscularly (Meyer et al., 2015). Similarly, an intranasal vectored SARS-CoV-2 vaccine induced tissue-resident T cell responses, thereby protecting the upper and lower respiratory tract against SARS-CoV-2 (Hassan et al., 2020). In a recent study (Meyer et al., 2021), immunization of hamsters by the parenteral route with a prime-boost regimen of Moderna mRNA-1273 SARS-CoV-2 vaccine, followed by challenge with SARS-CoV-2, resulted in a high level of restriction of challenge SARS-CoV-2 replication in the lungs, whereas there was substantial replication of challenge SARS-CoV-2 in the upper respiratory tract. Thus, while replication in the lung was efficiently restricted by parenteral vaccination, replication in the upper respiratory tract was not efficiently restricted, which would increase shedding and virus transmission (Meyer et al., 2021). Therefore, SARS-CoV-2 vaccines capable of direct immunization of the respiratory tract are of interest

In summary, an attenuated, replication-competent APMV3-vectored vaccine was generated that stably expresses a version of the SARS-CoV-2 S protein that has been stabilized in the prefusion conformation by six proline substitutions. The vaccine virus replicates to high titers in embryonated chicken eggs, which are a suitable substrate for vaccine manufacture. This vector provides for direct immunization of the respiratory tract using a virus for which humans generally lack immunity that otherwise could interfere with vaccination. A single intranasal dose of this vaccine candidate in hamsters elicited robust serum IgG and IgA antibody responses against SARS-CoV-2 S and conferred full protection of both the upper and lower respiratory tract of hamsters against SARS-CoV-2 challenge. These results support the further clinical development of this intranasal vaccine candidate. This candidate could be used as a stand-alone vaccine in a heterologous prime-boost with any of the vaccines presently in human use and in development.

### REFERENCES

Alexander, D. J. (2000). Newcastle disease and other avian paramyxoviruses. Rev Sci Tech 19, 443-462.
Amanat, F., Strohmeier, S., Rathnasinghe, R., Schotsaert, M., Coughlan, L., Garcia-Sastre, A., and Krammer, F. (2021). Introduction of Two Prolines and Removal of the Polybasic Cleavage Site Lead to Higher Efficacy of a Recombinant Spike-Based SARS-CoV-2 Vaccine in the Mouse Model. mBio 12.
Bos, R., Rutten, L., van der Lubbe, J. E. M., Bakkers, M. J. G., Hardenberg, G., Wegmann, F., Zuijdgeest, D., de Wilde, A. H., Koornneef, A., Verwilligen, A., et al. (2020). Ad26 vector-based COVID-19 vaccine encoding a prefusion-stabilized SARS-CoV-2 Spike immunogen induces potent humoral and cellular immune responses. NPJ Vaccines 5, 91.
Braun, J., Loyal, L., Frentsch, M., Wendisch, D., Georg, P., Kurth, F., Hippenstiel, S., Dingeldey, M., Kruse, B., Fauchere, F., et al. (2020). SARS-CoV-2-reactive T cells in healthy donors and patients with COVID-19. Nature 587, 270-274.
Bricker, T. L., Darling, T. L., Hassan, A. O., Harastani, H. H., Soung, A., Jiang, X., Dai, Y. N., Zhao, H., Adams, L. J., Holtzman, M. J., et al. (2021). A single intranasal or intramuscular immunization with chimpanzee adenovirus-vectored SARS-CoV-2 vaccine protects against pneumonia in hamsters. Cell Rep 36, 109400.
Buchholz, U. J., Finke, S., and Conzelmann, K. K. (1999). Generation of bovine respiratory syncytial virus (BRSV) from cDNA: BRSV NS2 is not essential for virus replication in tissue culture, and the human RSV leader region acts as a functional BRSV genome promoter. J Virol 73, 251-259.
Bukreyev, A., and Collins, P. L. (2008). Newcastle disease virus as a vaccine vector for humans. Curr Opin Mol Ther 10, 46-55.
Bukreyev, A., Huang, Z., Yang, L., Elankumaran, S., St Claire, M., Murphy, B. R., Samal, S. K., and Collins, P. L. (2005). Recombinant newcastle disease virus expressing a foreign viral antigen is attenuated and highly immunogenic in primates. J Virol 79, 13275-13284.
Burman, B., Pesci, G., and Zamarin, D. (2020). Newcastle Disease Virus at the Forefront of Cancer Immunotherapy. Cancers (Basel) 12.
Chan, J. F., Zhang, A. J., Yuan, S., Poon, V. K., Chan, C. C., Lee, A. C., Chan, W. M., Fan, Z., Tsoi, H. W., Wen, L., et al. (2020). Simulation of the clinical and pathological manifestations of Coronavirus Disease 2019 (COVID-19) in golden Syrian hamster model: implications for disease pathogenesis and transmissibility. Clin Infect Dis.
Chandrashekar, A., Liu, J., Martinot, A. J., McMahan, K., Mercado, N. B., Peter, L., Tostanoski, L. H., Yu, J., Maliga, Z., Nekorchuk, M., et al. (2020). SARS-CoV-2 infection protects against rechallenge in rhesus macaques. Science 369, 812-817.
Clemons, D. J., Besch-Williford, C., Steffen, E. K., Riley, L. K., and Moore, D. H. (1992). Evaluation of a subcutaneously implanted chamber for antibody production in rabbits. Lab Anim Sci 42, 307-311.
Corbett, K. S., Edwards, D. K., Leist, S. R., Abiona, O. M., Boyoglu-Barnum, S., Gillespie, R. A., Himansu, S., Schafer, A., Ziwawo, C. T., DiPiazza, A. T., et al. (2020a). SARS-CoV-2 mRNA vaccine design enabled by prototype pathogen preparedness. Nature 586, 567-571.
Corbett, K. S., Flynn, B., Foulds, K. E., Francica, J. R., Boyoglu-Barnum, S., Werner, A. P., Flach, B., O'Connell, S., Bock, K. W., Minai, M., et al. (2020b). Evaluation of the mRNA-1273 Vaccine against SARS-CoV-2 in Nonhuman Primates. N Engl J Med.
Corbett, K. S., Werner, A. P., Connell, S. O., Gagne, M., Lai, L., Moliva, J. I., Flynn, B., Choi, A., Koch, M., Foulds, K. E., et al. (2021). mRNA-1273 protects against SARS-CoV-2 beta infection in nonhuman primates. Nat Immunol.
Corman, V. M., Landt, O., Kaiser, M., Molenkamp, R., Meijer, A., Chu, D. K., Bleicker, T., Brunink, S., Schneider, J., Schmidt, M. L., et al. (2020). Detection of 2019 novel coronavirus (2019-nCoV) by real-time RT-PCR. Euro Surveill 25.
DiNapoli, J. M., Kotelkin, A., Yang, L., Elankumaran, S., Murphy, B. R., Samal, S. K., Collins, P. L., and Bukreyev, A. (2007). Newcastle disease virus, a host range-restricted virus, as a vaccine vector for intranasal immunization against emerging pathogens. Proc Natl Acad Sci USA 104, 9788-9793.
DiNapoli, J. M., Nayak, B., Yang, L., Finneyfrock, B. W., Cook, A., Andersen, H., Torres-Velez, F., Murphy, B. R., Samal, S. K., Collins, P. L., et al. (2010a). Newcastle disease virus-vectored vaccines expressing the hemagglutinin or neuraminidase protein of H5N1 highly pathogenic avian influenza virus protect against virus challenge in monkeys. J Virol 84, 1489-1503.
DiNapoli, J. M., Ward, J. M., Cheng, L., Yang, L., Elankumaran, S., Murphy, B. R., Samal, S. K., Collins, P. L., and Bukreyev, A. (2009). Delivery to the lower respiratory tract is required for effective immunization with Newcastle disease virus-vectored vaccines intended for humans. Vaccine 27, 1530-1539.
DiNapoli, J. M., Yang, L., Samal, S. K., Murphy, B. R., Collins, P. L., and Bukreyev, A. (2010b). Respiratory tract immunization of non-human primates with a Newcastle disease virus-vectored vaccine candidate against Ebola virus elicits a neutralizing antibody response. Vaccine 29, 17-25.
Espitia, C. M., Zhao, W., Saldarriaga, O., Osorio, Y., Harrison, L. M., Cappello, M., Travi, B. L., and Melby, P. C. (2010). Duplex real-time reverse transcriptase PCR to determine cytokine mRNA expression in a hamster model of New World cutaneous leishmaniasis. BMC Immunol 11, 31.
Feng, L., Wang, Q., Shan, C., Yang, C., Feng, Y., Wu, J., Liu, X., Zhou, Y., Jiang, R., Hu, P., et al. (2020). An adenovirus-vectored COVID-19 vaccine confers protection from SARS-COV-2 challenge in rhesus macaques. Nat Commun 11, 4207.
Freeman, A. I., Zakay-Rones, Z., Gomori, J. M., Linetsky, E., Rasooly, L., Greenbaum, E., Rozenman-Yair, S., Panet, A., Libson, E., Irving, C. S., et al. (2006). Phase I/II trial of intravenous NDV-HUJ oncolytic virus in recurrent glioblastoma multiforme. Molecular Therapy 13, 221-228.
Garcia-Sastre, A., and Mena, I. (2013). Novel vaccine strategies against emerging viruses. Curr Opin Virol 3, 210-216.
Hassan, A. O., Kafai, N. M., Dmitriev, I. P., Fox, J. M., Smith, B. K., Harvey, I. B., Chen, R. E., Winkler, E. S., Wessel, A. W., Case, J. B., et al. (2020). A Single-Dose Intranasal ChAd Vaccine Protects Upper and Lower Respiratory Tracts against SARS-CoV-2. Cell 183, 169-184 el 13.
Hoffmann, M., Kleine-Weber, H., Schroeder, S., Kruger, N., Herrler, T., Erichsen, S., Schiergens, T. S., Herrler, G., Wu, N. H., Nitsche, A., et al. (2020). SARS-CoV-2 Cell Entry Depends on ACE2 and TMPRSS2 and Is Blocked by a Clinically Proven Protease Inhibitor. Cell 181, 271-280 e278.
Hsieh, C. L., Goldsmith, J. A., Schaub, J. M., DiVenere, A. M., Kuo, H. C., Javanmardi, K., Le, K. C., Wrapp, D., Lee, A. G., Liu, Y., et al. (2020). Structure-based design of prefusion-stabilized SARS-CoV-2 spikes. Science 369, 1501-1505.
Hu, Z., Ni, J., Cao, Y., and Liu, X. (2020). Newcastle Disease Virus as a Vaccine Vector for 20 Years: A Focus on Maternally Derived Antibody Interference. Vaccines (Basel) 8.
Imai, M., Iwatsuki-Horimoto, K., Hatta, M., Loeber, S., Halfmann, P. J., Nakajima, N., Watanabe, T., Ujie, M., Takahashi, K., Ito, M., et al. (2020). Syrian hamsters as a small animal model for SARS-CoV-2 infection and countermeasure development. Proc Natl Acad Sci USA 117, 16587-16595.
Ju, B., Zhang, Q., Ge, J., Wang, R., Sun, J., Ge, X., Yu, J., Shan, S., Zhou, B., Song, S., et al. (2020). Human neutralizing antibodies elicited by SARS-CoV-2 infection. Nature 584, 115-119. Kolakofsky, D., Pelet, T., Garcin, D., Hausmann, S., Curran, J., and Roux, L. (1998). Paramyxovirus RNA synthesis and the requirement for hexamer genome length: the rule of six revisited. J Virol 72, 891-899.
Kumar, S., Militino Dias, F., Nayak, B., Collins, P. L., and Samal, S. K. (2010). Experimental avian paramyxovirus serotype-3 infection in chickens and turkeys. Vet Res 41, 72.
Kumar, S., Nayak, B., Collins, P. L., and Samal, S. K. (2008). Complete genome sequence of avian paramyxovirus type 3 reveals an unusually long trailer region. Virus Res 137, 189-197.
Liang, B., Ngwuta, J. O., Herbert, R., Swerczek, J., Dorward, D. W., Amaro-Carambot, E., Mackow, N., Kabatova, B., Lingemann, M., Surman, S., et al. (2016). Packaging and Prefusion Stabilization Separately and Additively Increase the Quantity and Quality of Respiratory Syncytial Virus (RSV)-Neutralizing Antibodies Induced by an RSV Fusion Protein Expressed by a Parainfluenza Virus Vector. J Virol 90, 10022-10038.
Logunov, D. Y., Dolzhikova, I. V., Shcheblyakov, D. V., Tukhvatulin, A. I., Zubkova, O. V., Dzharullaeva, A. S., Kovyrshina, A. V., Lubenets, N. L., Grousova, D. M., Erokhova, A. S., et al. (2021). Safety and efficacy of an rAd26 and rAd5 vector-based heterologous prime-boost COVID-19 vaccine: an interim analysis of a randomised controlled phase 3 trial in Russia. Lancet.
Martinez-Sobrido, L., Gitiban, N., Fernandez-Sesma, A., Cros, J., Mertz, S. E., Jewell, N. A., Hammond, S., Flano, E., Durbin, R. K., Garcia-Sastre, A., et al. (2006). Protection against respiratory syncytial virus by a recombinant Newcastle disease virus vector. J Virol 80, 1130-1139.
Mercado, N. B., Zahn, R., Wegmann, F., Loos, C., Chandrashekar, A., Yu, J., Liu, J., Peter, L., McMahan, K., Tostanoski, L. H., et al. (2020). Single-shot Ad26 vaccine protects against SARS-CoV-2 in rhesus macaques. Nature 586, 583-588.
Meyer, M., Wang, Y., Edwards, D., Smith, G. R., Rubenstein, A. B., Ramanathan, P., Mire, C. E., Pietzsch, C., Chen, X., Ge, Y., et al. (2021). Attenuated activation of pulmonary immune cells in mRNA-1273 vaccinated hamsters after SARS-CoV-2 infection. J Clin Invest.
Monaco, G., Chen, H., Poidinger, M., Chen, J., de Magalhaes, J. P., and Larbi, A. (2016). flowAI: automatic and interactive anomaly discerning tools for flow cytometry data. Bioinformatics 32, 2473-2480.
Pallesen, J., Wang, N., Corbett, K. S., Wrapp, D., Kirchdoerfer, R. N., Turner, H. L., Cottrell, C. A., Becker, M. M., Wang, L., Shi, W., et al. (2017). Immunogenicity and structures of a rationally designed prefusion MERS-CoV spike antigen. Proc Natl Acad Sci USA 114, E7348-E7357.
Papa, G., Mallery, D. L., Albecka, A., Welch, L. G., Cattin-Ortola, J., Luptak, J., Paul, D., McMahon, H. T., Goodfellow, I. G., Carter, A., et al. (2021). Furin cleavage of SARS-CoV-2 Spike promotes but is not essential for infection and cell-cell fusion. PLoS Pathog 17, e1009246. Pecora, A. L., Rizvi, N., Cohen, G. I., Meropol, N.J., Sterman, D., Marshall, J. L., Goldberg, S., Gross, P., O'Neil, J. D., Groene, W. S., et al. (2002). Phase I trial of intravenous administration of PV701, an oncolytic virus, in patients with advanced solid cancers. J Clin Oncol 20, 2251-2266. Riese, P., Sakthivel, P., Trittel, S., and Guzman, C. A. (2014). Intranasal formulations: promising strategy to deliver vaccines. Expert Opin Drug Deliv 11, 1619-1634.
Rima, B., Balkema-Buschmann, A., Dundon, W. G., Duprex, P., Easton, A., Fouchier, R., Kurath, G., Lamb, R., Lee, B., Rota, P., et al. (2019). ICTV Virus Taxonomy Profile: Paramyxoviridae. Journal of General Virology 100, 1593-1594.
Sadoff, J., Le Gars, M., Shukarev, G., Heerwegh, D., Truyers, C., de Groot, A. M., Stoop, J., Tete, S., Van Damme, W., Leroux-Roels, I., et al. (2021). Interim Results of a Phase 1-2a Trial of Ad26. COV2. S Covid-19 Vaccine. N Engl J Med.
Samuel, A. S., Subbiah, M., Shive, H., Collins, P. L., and Samal, S. K. (2011). Experimental infection of hamsters with avian paramyxovirus serotypes 1 to 9. Vet Res 42, 38.
Sanchez-Felipe, L., Vercruysse, T., Sharma, S., Ma, J., Lemmens, V., Van Looveren, D., Arkalagud Javarappa, M. P., Boudewijns, R., Malengier-Devlies, B., Liesenborghs, L., et al. (2021). A single-dose live-attenuated YF17D-vectored SARS-CoV-2 vaccine candidate. Nature 590, 320-325.
Shirvani, E., Varghese, B. P., Paldurai, A., and Samal, S. K. (2020). A recombinant avian paramyxovirus serotype 3 expressing the hemagglutinin protein protects chickens against H5N1 highly pathogenic avian influenza virus challenge. Sci Rep 10, 2221.
Sia, S. F., Yan, L. M., Chin, A. W. H., Fung, K., Choy, K. T., Wong, A. Y. L., Kaewpreedee, P., Perera, R. A. P. M., Poon, L. L. M., Nicholls, J. M., et al. (2020). Pathogenesis and transmission of SARS-CoV-2 in golden hamsters. Nature 583, 834-838.
Subbarao, K., McAuliffe, J., Vogel, L., Fahle, G., Fischer, S., Tatti, K., Packard, M., Shieh, W. J., Zaki, S., and Murphy, B. (2004). Prior infection and passive transfer of neutralizing antibody prevent replication of severe acute respiratory syndrome coronavirus in the respiratory tract of mice. J Virol 78, 3572-3577.
Sun, W., McCroskery, S., Liu, W. C., Leist, S. R., Liu, Y., Albrecht, R. A., Slamanig, S., Oliva, J., Amanat, F., Schafer, A., et al. (2020). A Newcastle Disease Virus (NDV) Expressing a Membrane-Anchored Spike as a Cost-Effective Inactivated SARS-CoV-2 Vaccine. Vaccines (Basel) 8.
Tian, J. H., Patel, N., Haupt, R., Zhou, H., Weston, S., Hammond, H., Logue, J., Portnoff, A. D., Norton, J., Guebre-Xabier, M., et al. (2021). SARS-CoV-2 spike glycoprotein vaccine candidate NVX-CoV2373 immunogenicity in baboons and protection in mice. Nat Commun 12, 372.
Tostanoski, L. H., Wegmann, F., Martinot, A. J., Loos, C., McMahan, K., Mercado, N. B., Yu, J., Chan, C. N., Bondoc, S., Starke, C. E., et al. (2020). Ad26 vaccine protects against SARS-CoV-2 severe clinical disease in hamsters. Nat Med 26, 1694-1700.
Voysey, M., Clemens, S. A. C., Madhi, S. A., Weckx, L. Y., Folegatti, P. M., Aley, P. K., Angus, B., Baillie, V. L., Barnabas, S. L., Bhorat, Q. E., et al. (2021). Safety and efficacy of the ChAdOxI nCoV-19 vaccine (AZD1222) against SARS-CoV-2: an interim analysis of four randomised controlled trials in Brazil, South Africa, and the UK. Lancet 397, 99-111.
Walls, A. C., Park, Y. J., Tortorici, M. A., Wall, A., McGuire, A. T., and Veesler, D. (2020). Structure, Function, and Antigenicity of the SARS-CoV-2 Spike Glycoprotein. Cell 181, 281-292 e286.
Walsh, E. E., Frenck, R. W., Jr., Falsey, A. R., Kitchin, N., Absalon, J., Gurtman, A., Lockhart, S., Neuzil, K., Mulligan, M. J., Bailey, R., et al. (2020). Safety and Immunogenicity of Two RNA-Based Covid-19 Vaccine Candidates. N Engl J Med 383, 2439-2450.
Wolfel, R., Corman, V. M., Guggemos, W., Seilmaier, M., Zange, S., Muller, M. A., Niemeyer, D., Jones, T. C., Vollmar, P., Rothe, C., et al. (2020). Virological assessment of hospitalized patients with COVID-2019. Nature 581, 465-469.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
Wrobel, A. G., Benton, D. J., Xu, P., Roustan, C., Martin, S. R., Rosenthal, P. B., Skehel, J. J., and Gamblin, S. J. (2020). SARS-CoV-2 and bat RaTG13 spike glycoprotein structures inform on virus evolution and furin-cleavage effects. Nat Struct Mol Biol 27, 763-767.
Wu, F., Zhao, S., Yu, B., Chen, Y. M., Wang, W., Song, Z. G., Hu, Y., Tao, Z. W., Tian, J. H., Pei, Y. Y., et al. (2020a). A new coronavirus associated with human respiratory disease in China. Nature 579, 265-269.
Wu, S., Zhong, G., Zhang, J., Shuai, L., Zhang, Z., Wen, Z., Wang, B., Zhao, Z., Song, X., Chen, Y., et al. (2020b). A single dose of an adenovirus-vectored vaccine provides protection against SARS-CoV-2 challenge. Nat Commun 11, 4081.
Yoshida, A., Kim, S. H., Manoharan, V. K., Varghese, B. P., Paldurai, A., and Samal, S. K. (2019). Novel avian paramyxovirus-based vaccine vectors expressing the Ebola virus glycoprotein elicit mucosal and humoral immune responses in guinea pigs. Sci Rep 9, 5520.
Yoshida, A., and Samal, S. K. (2017). Avian Paramyxovirus Type-3 as a Vaccine Vector: Identification of a Genome Location for High Level Expression of a Foreign Gene. Front Microbiol 8, 693.
Zhou, P., Yang, X. L., Wang, X. G., Hu, B., Zhang, L., Zhang, W., Si, H. R., Zhu, Y., Li, B., Huang, C. L., et al. (2020). A pneumonia outbreak associated with a new coronavirus of probable bat origin. Nature 579, 270-273.
Zhu, F. C., Guan, X. H., Li, Y. H., Huang, J. Y., Jiang, T., Hou, L. H., Li, J. X., Yang, B. F., Wang, L., Wang, W. J., et al. (2020). Immunogenicity and safety of a recombinant adenovirus type-5-vectored COVID-19 vaccine in healthy adults aged 18 years or older: a randomised, double-blind, placebo-controlled, phase 2 trial. Lancet 396, 479-488.
Zivcec, M., Safronetz, D., Haddock, E., Feldmann, H., and Ebihara, H. (2011). Validation of assays to monitor immune responses in the Syrian golden hamster (Mesocricetus auratus). J Immunol Methods 368, 24-35.

### Full-Length Plasmids:

pAPMV3_SARS_CoV-2-S_wt
(S sequence of NC 045512.2), SEQ ID NO: 1:
ACTAAACAGAAAATTAATACTTGTTAGTAGTCGCCGATTAATCCG
CTAATATATTAGGAGCGGAAGTCCTACACTCCGCCTCCGACCACG
AATTGAAATCATCATGGCTGGAATTTTTAATACTTATGAGCTCTT
TGTTAAGGATCAGACATGTATGCACAAGAGGGCTGCAAGCCTAAT
ATCGGGAGGTCAGCTTAAAAGCAATATACCAGTCTTCATCACCAC
AAAAGATGATCCAGCTGTCCGATGGGACCTTGTATGTTTTAATCT
CCGACTTGTTGTTAGTGAATCATCTACCTCGGTGATCCGGCAGGG
GGCTCTTATATCATTGCTTTCCATTACTGCTAGCAATATGAGGGC
TCTTGCAGCAATTGCTGGGCAGACTGATGAATCGATGATTAATAT
TATAGAAGTTGTGGACTTTGATGGACTTGAGCCCCAGTGTGACAC
TCGCAGTGGCTTAGACGCTCAGAAGCAGGAGATGTTTAAGGATAT
TGCCCGTGATATGCCGAGGGCACTATCCAGCGGGACCCCGTTCCA
GCACGGAAATGCGGAAGCGAATGGGCCTGAAGACACACACATGTT
CCTCCGATCCGCAATTAGCGTTCTGACACAGATCTGGATCCTGGT
TGCAAAAGCCATGACTAACATTGATGGCAGCAACGAGGCAAGCGA
TCGCCGCCTTGCCAAGTATATTCAACAGAATAGAATTGACAGGCG
ATTCATGCTTGCTCAAGCTACCAGGACTGTATGTCAGCAGGTCAT
AAAGGATTCTTTGACCTTGAGAAGGTTCCTTGTAACTGAGCTTCG
AAAGTCGCGGGGGGCATTGCATAGCGGGTCATCTTACTATGCTAT
GATTGGAGACATGCAGGCATACATATTTAATGCAGGCCTTACACC
ATTCCTCACTACCCTCAGGTACGGGATCGGGACTAAATACCATGC
CCTTGCAATTAGCTCACTTGCAGGTGACCTGAATAAGATTAAGGG
GTTACTGACTCTATACAAAGAGAAAGGGAGTGATGCGGGCTATAT
GGCACTTCTAGAGGATGCTGACTGCATGCAATTTGCGCCAGGTAA
TTACTCACTATTATATTCATATGCGATGGGAGTAGCAAGTGTACA
TGATGAAGGCATGAGGAATTACCAATACGCCCGGCGCTTCCTCCA
TAAGGGGATGTACCAATTTGGACGTGATATCGCAACACAACAGCA
ACATGCACTTGATGAATCCCTCGCCCAGGAGATGAGAATCACAGA
GGCTGACAGGGCAAATCTCAAGGTAATGATGGCAAACATTGGGGA
AGCATCAAGCTTCAGTGATATGCCTAATCCAGGAGCTAGCGGCAT
TCCTGCCTTCAATGACCGCCCTGATGAGATCTTCTCTGAGCCAGG
CTATCGCAAGCCCCCTCAAGATCAGCCGCTGATAAAGCTCCCAGA
TCTCGAGGAAGAAGAGCAGGACGAATTCGACATGTAGAGAGCCGC
CCACCACCCAGAGCCAGCGACAAACCCAGGCAAAACAACCCACCA
TCTCCGACCCGGAACACAACGCAACATCGAGGACCCCGGGCAGCC
GACACCGCAGCCGCAAGACCTCCGCATCATGAACGACCAGCATCC
AAACATCTGTCCTCCCTATTCTATCAGTTTAATAAAAAAGGCTTT
ACGTGCACAGACAGAGATCCTGACATAATTCAGCCGGGGGGATTC
AACTTAGGAGCGGAAGTATCCCTCCTAACCTGCACCTGCATTTCA
ATATGGATCTTGAGTTTAGTAGTGAGGAAGCAGTCGCAGCCCTCT
TGGATGTGAGTTCCTCAACCATAACCGAGGTTCTGAGCAAGCAAA
GTATTCCTGACCCTAGCTTTCTCACGTCCACGGAGGCATCCGGTG
AGCAAGTTCCAGATTCGAAGGTCGCCAATCACAAAGCCAGTGTCG
ATAAGACCCCAGATCAAGGCCAACCATCGGCAACGCCTTCGGCTC
CTCCTGAGACTGCCGAAAACATCAATACACCATCTTGCGAGGACG
GTTTACCCCCTAACTTCTTCATCCCAAGGGTAGAGAGTTACCACA
CTAACCTTTTTAAAGGGGGCCCCCAATTGGACTCAACGGAGCGGC
CGCCTGGTCACCAAATAGTTTGCGGAGATCAAGACGCGGACCTCT
CCCGAGCCGGGATAGCGAGAAAGAAGAAGAAGCAGAAGCATTGCA
AAGCGCTCAACTTTTCGGAATCCCCACTCACAGACAATCAGGCCA
TAGAAGGGAGTATTCAATCTCATGGAGCCCTGGATCAGGAACCTT
CCAGACAGAGACCTGGTGCAACCCAGCCTGCTCTCCAGTCACCGC
CTTCCCAAAACAATACAAGTGTGCATGCAGACAGTGCCCAAGATT
CTGCGATCTCTGTTTCAATCCCGCTGACTATGGTGGAATCATTGA
TCTCTCAAGTGTCAAAGTTGTCGGACCAAGTCTCGCAGATACAGA
AGTTGGTGAGTACCCTTCCTCAAATTAAGACTGATATTGCTTCGA
TTCGGAACATGCAAGCAGCTTTGGAAGGACAGCTCAGTATGATTC
GTATATTGGATCCAGGAAACTGTTCGGAATCATCACTCAATGCGC
TGAGAGGGTTCAGTGGTAAGGCACCTGTTATTGTGAGTGGTCCAG
GAAATCCCAATCAATGCATCAGCCAAGGCTCATCCACCACAATTT
GCCTGGATGAGTTAGCCCGGCCTGTGCCAAATCCTATACAGGTGA
AACTTCAAGATGATCAGAAGGACCTATCTGCACAGCGACATGCTG
TCATTGCATTACTCGAAACCAGAATCCCCCCGGGACCAAAACGTG
ACAAATTACTGTCATTGGTTGCAATTGCAAAGACTGCAAGTGATT
TAATCAAGATTAAGAGAATGGCAGTCCTCGGCCAGTGATATCACC
CTATGGCAACCAGGCTACAAATGTAAGCAGTTTCCGATATGTACC
GAATCAATAACACGACCCCTTCCGCAAAAGCAAGCGCGAGCATGA
CACACTCAGCTTCAGCCAATTCTAGCTTCACTAAGCAGGTGATCT
GACCATCTGGCTTTACTTCCGCTTTATAAAAAACTCCTAGATCTA
ACATGGAGGCAGACCGAGCTAATGCGGAGGGAGTACACAAGGGGA
ATCAAAGGAGCGGAAGGTACTAAGTCACTCCACCGCCTGCCCTCG
CCCATCCGCAGCCTCCCCCAACCCGCGGCGCCGCCACCATGTTTG
TTTTTCTTGTTTTATTGCCACTAGTCTCTAGTCAGTGTGTTAATC
TTACAACCAGAACTCAATTACCCCCTGCATACACTAATTCTTTCA
CACGTGGTGTTTATTACCCTGACAAAGTTTTCAGATCCTCAGTTT
TACATTCAACTCAGGACTTGTTCTTACCTTTCTTTTCCAATGTTA
CTTGGTTCCATGCTATACATGTCTCTGGGACCAATGGTACTAAGA
GGTTTGATAACCCTGTCCTACCATTTAATGATGGTGTTTATTTTG
CTTCCACTGAGAAGTCTAACATAATAAGAGGCTGGATTTTTGGTA
CTACTTTAGATTCGAAGACCCAGTCCCTACTTATTGTTAATAACG
CTACTAATGTTGTTATTAAAGTCTGTGAATTTCAATTTTGTAATG
ATCCATTTTTGGGTGTTTATTACCACAAAAACAACAAAAGTTGGA
TGGAAAGTGAGTTCAGAGTTTATTCTAGTGCGAATAATTGCACTT
TTGAATATGTCTCTCAGCCTTTTCTTATGGACCTTGAAGGAAAAC
AGGGTAATTTCAAAAATCTTAGGGAATTTGTGTTTAAGAATATTG
ATGGTTATTTTAAAATATATTCTAAGCACACGCCTATTAATTTAG
TGCGTGATCTCCCTCAGGGTTTTTCGGCTTTAGAACCATTGGTAG
ATTTGCCAATAGGTATTAACATCACTAGGTTTCAAACTTTACTTG
CTTTACATAGAAGTTATTTGACTCCTGGTGATTCTTCTTCAGGTT
GGACAGCTGGTGCTGCAGCTTATTATGTGGGTTATCTTCAACCTA
GGACTTTTCTATTAAAATATAATGAAAATGGAACCATTACAGATG
CTGTAGACTGTGCACTTGACCCTCTCTCAGAAACAAAGTGTACGT
TGAAATCCTTCACTGTAGAAAAAGGAATCTATCAAACTTCTAACT
TTAGAGTCCAACCAACAGAATCTATTGTTAGATTTCCTAATATTA
CAAACTTGTGCCCTTTTGGTGAAGTTTTTAACGCCACCAGATTTG
CATCTGTTTATGCTTGGAACAGGAAGAGAATCAGCAACTGTGTTG
CTGATTATTCTGTCCTATATAATTCCGCATCATTTTCCACTTTTA
AGTGTTATGGAGTGTCTCCTACTAAATTAAATGATCTCTGCTTTA
CTAATGTCTATGCAGATTCATTTGTAATTAGAGGTGATGAAGTCA
GACAAATCGCTCCAGGGCAAACTGGAAAGATTGCTGATTATAATT
ATAAATTACCAGATGATTTTACAGGCTGCGTTATAGCTTGGAATT
CTAACAATCTTGATTCTAAGGTTGGTGGTAATTATAATTACCTGT
ATAGATTGTTTAGGAAGTCTAATCTCAAACCTTTTGAGAGAGATA
TTTCAACTGAAATCTATCAGGCCGGTAGCACACCTTGTAATGGTG
TTGAAGGTTTTAATTGTTACTTTCCTTTACAATCATATGGTTTCC
AACCCACTAATGGTGTTGGTTACCAACCATACAGAGTAGTAGTAC
TTTCTTTTGAACTTCTACATGCACCAGCAACTGTTTGTGGACCTA
AAAAGTCTACTAATTTGGTTAAAAACAAATGTGTCAATTTCAACT
TCAATGGTTTAACAGGCACAGGTGTTCTTACTGAGTCTAACAAAA
AGTTTCTGCCTTTCCAACAATTTGGCAGAGACATTGCTGACACTA
CTGATGCTGTCCGTGATCCACAGACACTTGAGATTCTTGACATTA
CACCATGTTCTTTTGGTGGTGTCAGTGTTATAACACCAGGAACAA
ATACTTCTAACCAGGTTGCTGTTCTTTATCAGGATGTTAACTGCA
CAGAAGTCCCTGTTGCTATTCATGCAGATCAACTTACTCCTACTT
GGCGTGTTTATTCTACAGGTTCTAATGTTTTTCAAACACGTGCAG
GCTGTTTAATAGGGGCTGAACATGTCAACAACTCATATGAGTGTG
ACATACCCATTGGTGCAGGTATATGCGCTAGTTATCAGACTCAGA
CTAATTCTCCTCGGCGGGCACGTAGTGTAGCTAGTCAATCCATCA
TTGCCTACACTATGTCACTTGGTGCAGAAAATTCAGTTGCTTACT
CTAATAACTCTATTGCCATACCCACAAATTTTACTATTAGTGTTA
CCACAGAAATTCTACCAGTGTCTATGACCAAGACATCAGTAGATT
GTACAATGTACATTTGTGGTGATTCAACTGAATGCAGCAATCTTT
TGTTGCAATATGGCAGTTTTTGTACACAATTAAACCGTGCTTTAA
CTGGAATAGCTGTTGAACAAGACAAAAACACCCAAGAAGTTTTTG
CACAAGTCAAACAAATTTACAAAACACCACCAATTAAAGATTTTG
GTGGTTTTAATTTTTCACAAATATTACCAGATCCATCAAAACCAA
GCAAGAGGTCATTTATTGAAGATCTACTTTTCAACAAAGTGACAC
TTGCAGATGCTGGCTTCATCAAACAATATGGTGATTGCCTTGGTG
ATATTGCTGCTAGAGACCTCATTTGTGCACAAAAGTTTAACGGCC
TTACTGTTTTGCCACCTTTGCTCACAGATGAAATGATTGCTCAAT
ACACTTCTGCACTGTTAGCGGGTACAATCACTTCTGGTTGGACCT
TTGGTGCAGGTGCTGCATTACAAATACCATTTGCTATGCAAATGG
CTTATAGGTTTAATGGTATTGGAGTTACACAGAATGTTCTCTATG
AGAACCAAAAATTGATTGCCAACCAATTTAATAGTGCTATTGGCA
AAATTCAAGACTCACTTTCTTCCACAGCAAGTGCACTTGGAAAAC
TTCAAGATGTGGTCAACCAAAATGCACAAGCTTTAAACACGCTTG
TTAAACAACTTAGCTCCAATTTTGGTGCAATTTCAAGTGTTTTAA
ATGATATCCTTTCACGTCTTGACAAAGTTGAGGCTGAAGTGCAAA
TTGATAGGTTGATCACAGGCAGACTTCAAAGTTTGCAGACATATG
TGACTCAACAATTAATTAGAGCTGCAGAAATCAGAGCTTCTGCTA
ATCTTGCTGCTACTAAAATGTCAGAGTGTGTACTTGGACAATCAA
AAAGAGTTGATTTTTGTGGAAAGGGCTATCATCTTATGTCCTTCC
CTCAGTCAGCACCTCATGGTGTAGTCTTCTTGCATGTGACTTATG
TCCCTGCACAAGAAAAGAACTTCACAACTGCTCCTGCCATTTGTC
ATGATGGAAAAGCACACTTTCCTCGTGAAGGTGTCTTTGTTTCAA
ATGGCACACACTGGTTTGTAACACAAAGGAATTTTTATGAACCAC
AAATCATTACTACAGACAACACATTTGTGTCTGGTAACTGTGATG
TTGTAATAGGAATTGTCAACAACACAGTTTATGATCCTTTGCAAC
CTGAATTAGACTCATTCAAGGAGGAGTTAGATAAATATTTTAAGA
ATCATACATCACCAGATGTTGATTTAGGTGACATCTCTGGCATTA
ATGCTTCAGTTGTAAACATTCAAAAAGAAATTGACCGCCTCAATG
AGGTTGCCAAGAATTTAAATGAATCTCTCATCGATCTCCAAGAAC
TTGGAAAGTATGAGCAGTACATCAAGTGGCCATGGTACATTTGGC
TAGGTTTTATAGCTGGCTTGATTGCCATAGTAATGGTGACAATTA
TGCTTTGCTGTATGACCAGTTGCTGTAGTTGTCTCAAGGGCTGTT
GTTCTTGTGGATCCTGCTGCAAATTTGATGAAGACGACTCTGAGC
CAGTGCTCAAAGGAGTCAAATTACATTACACATAATGATTTATAA
AAAACATTGTGAGAATGCAATTTAAGGACGAAAGTAGGAGCGGAA
GCCGCGGCCCAGGCTCCATTCAAAAACCATGGCCGCTGCTCTCGG
GAACATGCACCCGTCATCGTCGATTACTCTTATGCATGATGACCC
GTCCATCCAGACGCAGTTGCTAGCCTTCCCTCTGATAAGTGAGAA
GACAGAGACAGGGACGACAAAGCTGCAGCCTCAAGTGAGAATGCA
ATCTTTCCTATCAACCGATAGCCAGAAGTATCATCTGGTCTTTAT
CAACACATATGGTTTCATTGCGGAGGATTTTAATTGTAGTCCAGC
AAACGGATTTGTGCCTGCACTATTTCAACCCAAGTCTAAAGTCTT
ATCGTCAGCGATGGTTACACTTGGGGCTGTCCCTGCTGACACTGT
TCTCCAGGACCTACAACGGGACTTAATCGCAATGAGATTTAAAGT
CCGAAAGAGTGCGTCAGCGAAGGAGCTAATATTATTCTCCACAGA
CAATGTCCCTGCAACATTAACAGGGTCATCAGTCTGGAAGAATAA
AGGAGTAATAGCTGATACAGCAACTGCAGTTAAAGCGCCCGGTCG
CATCTCATGCGATGCGGTATGCAGTTATTGCATTACATTCATCTC
TTTTTGCTTCTTCCACTCATCTGCTCTTTTTAAGGTGCCCAAGCC
TCTGCTGAATTTCGAGACGGCCATAGCATATTCCCTTGTACTCCA
GGTCGAGCTTGAGTTCCCAAATATCAAAGATACCTTACACGAGAA
GTATCTCAAAGTTAAAGACTCGAAGTGGTATTGTACAATTGACAT
CCATATTGGGAATCTTTTAAAGAGAACCGCCAAGCAACGAAGGAG
GACACCTTCAGAGATAATGCAGAAAGTCCGTAAGATGGGTTTCAG
GATTGGGTTATACGATTTGTGGGGTCCAACAATTGTGGTTGAATT
AACTGGGTCCTCAAGTAAGTCCCTACACGGATTCTTCTCGAGTGA
GCGGCTGGCCTGCCACCCGGTATCACAGTATAATCCTCATGTTGG
ACAGCTTATCTGGGCCCACGATGTATCCATCACGGGGTGCCACAT
GATTATATCTGAACTTGAGAAAAAGAAGGCACTTGCAATGGCTGA
CCTGACCGTGAGTGATGCAGTAGCTGTCAACACGAGCATCAAGAG
CTTATCGCCTTTCAGACTGTTCAAAAAATATTAGGGCTAGACAAC
TCCTAGGGTGTGTGCTTATAATTTGCATATTCTAGATTATAATCA
TTAACCGCGTCCAAATGATCAGACCGCATAATTAACAGCTCAGCC
AGGACTCGCAAATCCTGCATAGGCTCAAATAACAAGACGACATAT
ATACTCACACAACAAGGTGTAATATCTAGAAGGAATCAGAATTGA
GACAATATCTAGGTTATCAGCGCTACTGCCCTGCAACTCACTATG
TATGCTTGGCATTAATAAAAAACATTGTGAGAATGCAATTTAAGG
ACGAAAGTAGGAGCGGAACACGCTCAACTGAACCCAAGCGCACAC
CAGCCCCCCGGCACCGCACCGGAAGGACGCACAACAGCCGACCGC
CCCCACCAGGGCAGAGGCCATGCAACCAGGCTCGGCACTCCACCT
CCCGCACCTGTACATAATAATTGCCTTAGTCAGCGATGGCACTTT
AGGCCAAACAGCTAAAATTGATAGGCTGATACAGGCAGGGATTGT
CTTGGGTAGTGGAAAGGAGCTCCATATCTCACAAGATTCAGGGAC
ACTGGATTTATTCGTGAGGTTGTTACCAGTACTCCCTTCGAATTT
GTCCCATTGCCAGCTTGAAGCAATAACACAGTATAATAAGACGGT
GACTAGGTTATTGGCCCCAATAGGGAAGAACCTAGAACAGGTACT
ACAAGCGAGACCGCGTGGGAGGCTGTTCGGAGCAATAATAGGGTC
CATCGCGCTTGGTGTTGCCACATCAGCACAGATTACCGCTGCAAT
TGCACTCGTCCGTGCACAGCAAAATGCTAATGATATATTAGCACT
TAAAAACGCTCTCCAATCCAGCAATGAGGCAATCCGTCAACTCAC
GTATGGCCAGGACAAACAGTTACTTGCAATTTCTAAAATACAAAA
AGCTGTGAATGAGCAGATCCTCCCTGCACTCGATCAGCTGGACTG
TGCTGTCCTTGGAACCAAACTAGCTGTGCAGCTTAATCTGTATCT
CATTGAGATGACTACCATTTTCGGCGAGCAAATCAATAATCCAGT
CTTGGCGACCATCCCATTAAGTTACATTTTACGCCTGACCGGTGC
GGAGTTGAACAACGTCCTCATGAAGCAAGCCCGATCGTCCTTAAG
CCTTGTACAGTTAGTTTCAAAAGGCTTACTTAGTGGACAAGTAAT
TGGCTATGACCCATCTGTTCAAGGTCTGATAATAAGGGTGAATCT
GATGCGAACGCAGAAGATCGATAGAGCACTAGTTTATCAGCCGTA
TGTATTACCAATCACTCTTAACTCTAACATAGTAACACCAATCGC
CCCAGAATGCGTAATTCAGAAAGGGACGATCATAGAGGGTATGTC
ACGGAAGGATTGTACAGAGTTGGAGCAAGACATAATTTGTAGGAC
AGTTACGACATACACGCTTGCCAGAGACACCAGATTGTGTTTACA
AGGCAATATCTCTTCTTGTAGGTATCAACAGTCAGGCACCCAACT
ACACACCCCATTTATTACGTACAACGGGGCAGTTATCGCAAACTG
TGATTTAGTCTCTTGTCGGTGTCTCCGTCCTCCTATGATTATCAC
CCAAGTAAAAGGGTACCCTCTTACCATAATCACAAGGTCCGTATG
TCAAGAATTATCCGTTGACAACTTAGTCCTGAATATTGAGACACA
CCATAATTTCTCCCTTAATCCAACAATTATCGACCCTCTAACAAG
AGTAATAGCCACTACACCGTTAGAGATAGATTCCTTAATCCAAGA
GGCACAAGATCATGCGAATGCAGCGCTAGCTAAAGTGGAAGAGAG
TGATAAATATCTCAGGGCAGTTACTGGCGGTAATTATTCGAATTG
GTATATCGTTCTTGTCATCGTCTTACTATTTGGGAATTTGGGCTG
GTCGCTGTTGTTAACAGTACTTCTATGTAGGTCACGGAAACAACA
GCGACGTTATCAACAAGATGATTCCGTCGGGTCAGAAAGAGGTGT
TGGGGTTGGGACCATCCAGTATATGTCATAAGTGGTGTCAGACAT
CACAATCCAAGTCACTCAGTTATCTACTCCGCTGGCTCGGAGCCA
TCGACAACGGCGTGAGCCCTCATCCGGGGACAATCCACAACCTGA
TACTACCAGACAGTGGCTCCGCACGATGGCAGCAGCCCACAACGT
CCTGCATGTAAATATCATTGCGTTACTTTAATTAAAAAACCTCAA
GGTGACCCATCCTGGCCCATCCGATTGGAGGAGCGGAAGGTTTTA
AGGTAATCAGGCAAATTGTTTCCAACATGGAATCCCCTCCTTCTG
GCAAGGATGCGCCAGCCTTCCGCGAGCCTAAGCGAACTTGCAGAC
TCTGTTACAGGGCGACGACTCTCTCCCTTAATCTCACCATCGTTG
TATTATCAATTATAAGCATTTATGTATCAACTCAAACTGGGGCAA
ACAACTCTTGTGTCAATCCCACAATCGTAACTCCTGATTATTTAA
CTGGCAGCACGACAGGCTCAGTTGAAGATCTTGCTGACTTAGAGA
GTCAACTCCGGGAGATACGTAGAGACACAGGGATTAATCTCCCAG
TTCAGATCGATAATACAGAAAATCTAATACTGACAACTCTGGCGA
GTATCAACTCGAATTTACGCTTTCTGCAAAATGCAACAACTGAGA
GCCAGACCTGCCTATCTCCTGTCAATGACCCTCGCTTTGTAGCTG
GCATAAATAGGATCCCAGCAGGGTCGATGGCATATAATGACTTCA
GCAACCTCATTGAGCACGTAAATTTCATACCGTCGCCGACAACGT
TGTCAGGCTGCACCAGGATACCATCATTTTCACTCTCTAAGACGC
ACTGGTGTTATACCCACAATGTTATATCAAACGGGTGCCTTGACC
ATGCTGCAAGTTCACAATACATCTCAATAGGGATCGTTGATACCG
GCCTAAATAATGAGCCCTATTTTAGAACAATGTCTTCAAAGTCAC
TGAATGACGGATTAAATAGAAAGAGTTGCTCTGTGACCGCAGCCG
CTAATGCATGTTGGTTACTCTGTAGCGTAGTAACAGAATACGAAG
CTGCGGACTATCGATCACGAACTCCTACTGCTATGGTTCTGGGGC
GATTCGATTTTAATGGTGAATACACAGAAATAGCTGTCCCCTCCT
CACTATTCGATGGCCGGTTTGCATCTAACTATCCAGGTGTGGGAT
CGGGAACTCAAGTCAATGGAACATTATATTTCCCTTTATATGGAG
GGGTCCTTAATGGGTCAGATATAGAAACGGCAAACAAGGGGAAAT
CCTTCAGGCCTCAGAATCCTAAGAATAGGTGTCCAGACTCGGAGG
CGATCCAAAGCTTTAGAGCGCAAGATAGCTATTACCCGACGAGAT
TTGGGAAGGTGCTGATCCAACAGGCAATCATTGCATGTAGGATCA
GTAACAAAAGTTGTACTGATTTCTATCTCCTGTACTTCGACAACA
ACAGAGTGATGATGGGTGCAGAGGCACGGCTATATTATCTTAATA
ACCAATTATATCTATATCAACGCTCGTCGAGCTGGTGGCCGCATC
CTTTGTTCTATAGCATCTCATTACCGTCATGTCAAGCCCTTGCTG
TCTGTCAAATTACAGAGGCCCATCTGACACTGACTTACGCCACGT
CGAGGCCAGGAATGTCTATCTGTACAGGAGCATCAAGGTGTCCAA
ATAATTGCGTAGACGGTGTATATACTGATGTATGGCCATTGACAA
AGAACGATGCTCAGGACCCCAATCTCTTCTACACTGTATACTTAA
ATAATTCAACTCGGCGGATTAGCCCAACCATAAGTCTATACACTT
ATGACCGCAGGATCAAATCTAAGCTGGCAGTGGGTAGTGATATAG
GAGCAGCTTATACCACGAGTACCTGTTTCGGCCGATCAGATACAG
GGGCGGTTTATTGCTTAACTATAATGGAGACCGTTAACACAATAT
TTGGTCAGTACCGGATCGTGCCAATATTACTTAGAGTGACAAGCC
GGTAATGGATTGTTCCTAGTGAAGTGGGGCCTATGGTTACTCCGT
AACGGAGATGACCTGATTTATGCTATGTAACTAATTTTGCTCAAT
CGGGAGGGTGATCGGCCGGGTATGTCCACAATGACAATGACTCCT
ATTCCCTTGCTAGAGCTAGCTTCCTCTTTAATCCAATACAATCCC
TACCTGTAGTTTAATAAAAAAATCCTCTATATAAGATCCAGACGC
GTGCTATATAAGCTAATAGCTTAGGATGGAAACGACAGGAGCGGA
ACATATCCCTCCTACCATGTCTTCTCATAATATTATCCTCCCTGA
CCACCACTTAAATTCCCCTATTGTACTAAATAAATTGATGTACTA
TTGTAAACTCCTTAATATATTGCCTGACCCTAAGTCGCCATGGTA
CGAGAAGATTAAGTCTTGGACTAATTGCTGCATCCGTGTGTCAGA
CAGCAATCGCATGACTTTGTCACGCGCCTCCAAGCTACGAGAACT
ATTGGCAACGTACGGCGTATATTCGAAGAACCACCAGCAATGTTA
TACCACCATAATCCATCCCCAATCCCTATCACCCATCATGCAGAC
TGTCTCTCAACTGGGACGCTCAATCCCGACCTGGGCCAGGATCCG
AAAGGAGATCACACACAGTATCATAGCCCAAACGAATAAATTTGA
ATCCTTATTCCACAACATCTCAAAGGACCTGACAGGGAAAACCAA
CTTGTTTGGCGGATTCTGTGATTTACATGGGAGCATAAGCACTGC
TGCAAAGCGCTTAATGAATCAACCGGGTCTCTACTTGGATTCACC
TGATGCCCATGCATGTCAATTTTTGTTCCAGCTTAAAACCTGTCA
ACGGGAATTGATTCTACTTATGAGGCAGAATGCCACAACAGAGCT
GATTAGGGTTTTTGACTATCCTGAGCTACGGGTTCTTGTGACACC
TGAATATTCGGTCTGGGTTTTTACACGGACCAAACAAGTTACATT
ACTAACATTCGATTGCTTGTTAATGTACTGCGATCTTTCAGACGG
CCGGCACAACATCCTATTCACTTGTAAGCTTTTGTCTCACTTAAA
CCACCTCGGGTGTCGGATAAGGGACCTGCTGGTGCTAATTGATTC
ACTTGCCGAGAAACACCCCTTAATAGTTTATGATGTAGTTGCAAG
TCTGGAGTCGTTATCATATGGTGCTATACAGCTACATGACAAGGT
AGCTGGTTATGCTGGAACATTTTTCTCATTTATCCTTGCAGAAAT
ACAGGATTCCTTAGAAACAGTGTTGGATCAAGGTAATAGTGAATC
TATCATATCCCAAATAAGGAACATATACTCAGGTCTCACAGTAAA
CGAGGCTGCAGAATTGCTATGTGTTATGAGGCTTTGGGGCCACCC
TGCCCTAAACAGTGTAGACGCTGCAAGCAAGGTGAGACAGAGTAT
GTGCGCTGGCAAGCTGCTTAAATTTGATACGATCCAGTTAGTCCT
TGCATTCTTCAATACACTAATAATTAATGGATATAGGAGAAAGCA
CCATGGGCGATGGCCGAATGTTTGTAGTGACTCTATAATTGGGAA
TGAACTCAAGCGGATGTATTTTGATCAATGTGAGATCCCCCACGA
CTTCACATTAAAGAACTACCGGGAATTAAGTCTTCTTGAATTCGA
GTGCACATTTCCCATAGAATTATCAGATAAACTGAACATATTTTT
AAAAGATAAGGCAATAGCATTCCCGAAGTCCAGGTGGACATCTCC
TTTCAAAGCAGATATCACACCGCGCCATTTGCTGCAAGCGCCTGA
GTTTAAAACCCGTGCCAACAGACTACTGCTATCATTCTTACAACT
CGAGGAATTCTCCATCGAATCAGAACTAGAATATGTGACTACCCT
GGCTTACCTGGATGATGATGAGTTCAACGTATCTTACTCCCTCAA
AGAGAAGGAGGTAAAGACAGATGGTCGAATCTTCGCTAAGCTCAC
AAGGAAAATGCGGAGCTGTCAGGTGATATTCGAAGAATTGCTAGC
AGAGCATGTCTCTCCATTATTTAAAGACAACGGCGTTACTATGGC
CGAATTGTCACTTACTAAGAGCCTACTCGCTATTAGCAACCTAAG
CTCCACCTTATTTGAGACGCAAACAAGGCAAGGCGATCGGAATGC
AAGATTCACGCATGCGCACTTCATAACAACTGACCTGCAAAAGTA
CTGTTTAAACTGGAGGTACCAGAGTGTAAAACTGTTCGCACGTCA
ATTGAACCGGTTATTTGGTCTGCAGCATGGGTTTGAATGGATCCA
TTGCATACTAATGAAGTCCACTATGTATGTCGCAGACCCCTTTAA
CCCTCCACATAGCAATGCACGTGAGGCACTCGATGACAACTTAAA
TAGCGACATATTCATTGTATCACCTAGGGGGGCCATCGAGGGACT
ATGCCAAAAAATGTGGACAATCATCTCAATCTCGGCGATCCATGC
ATCCGCTGCTGTGGCTGGGCTACGGGTGGCATCTATGGTCCAAGG
TGATAACCAAGTTATTGGGGTAACACGCGAGTTCCTAGCTGGGCA
TGATCAAACTTTTGTAGACGATCAATTGACGGTTTCACTTGAGAA
CTTCACTCAGATATTCAAGGAAGTCAACTACGGGETAGGTCATAA
TCTCAAGCTGAGGGAAACAATCAAGTCGAGCCACATGTTCATCTA
CTCAAAGAGGATCTTCTACGACGGCCGAGTGCTTCCACAACTTCT
AAAGAACATAAGTAAGTTAACCCTATCAGCTACAACTACAGGGGA
GAACTGTTTAACGAGTTGCGGGGACCTATCATCATGCATCACACG
ATGTATAGAGAATGGGTTCCCTAAAGATGCAGCATTCGTTTTGAA
TCAATTAGTGATTAGGATCCAGATACTTGCCGATCACTTTTATTC
CATCCTTGGAGGATGTTTCTCTGGGTTGAACCAGGCGGATATCCG
TCTCCTTCTATATGAGGGTGCAATTTTACCGGCACAATTAGGAGG
GTTCAACAACCTCAACATGTCACGGCTATTCTGTCGGAATATTGG
TGACCCATTAGTCGCGTCCATCGCAGACACCAAGAGATTTGTGAA
GTGCCAACTGCTCGTTCCTCACATTTTAGACTCTGTCGTTGCTAT
TACCGATAGGAAGGGGTCTTTTACGACACTGATGATGGATCCATA
TTCAATAAACTTGGATTATATCCAGCAACCAGAGACACGATTGAA
GCGACATGTGCAAAAGGTTTTACTACAAGAGTCAGTGAATCCGCT
ACTCCAAGGGGTCTTCCTAGAGTCTCAACAAGAGGAAGAGGAACA
ACTTGCAGCGTTCTTGCTGGACCGAGAAGTCGTAATGCCAAGGGT
TGCGCACGCTATATTTGAGTGTACCAGCTTAGGTAGGCGCCGACA
TATCCAGGGCCTCATCGACACGACAAAGACCATTATTGCACTTGC
GTTGGATACACAGCAGTTAAGTTATACTAAACGTGAACAGATTGT
GACGTACAATGCAACTTATATGAGGTCCTTAGCAAGCATGCTCAG
TTCAAGCCATAATCAGACACGTCGGTCTGTGATTGGTCACGCATC
CTTCAATATCACCGACTGCTCGGTCATCCTGGCTCAACAAGTGAG
ACGTGCAAGCTGGGCGCCCTTGCTGAATTGGCGGTCATTAGAAGG
CTTAGAAGTACCCGACCCTATTGAGTCAGTAGCCGGCTACCTGGG
TTTGGATTCTAACAACTGTTTCTTATGCTGCCACGAGCAGAACAG
CTATTCCTGGTTTTTCCTTCCCCGAATGTGCCATTTTGATGATTC
GAGACAATCTAACTCCATACAAAGGGTACCTTACATTGGTTCTAA
GACTGACGAGCGCCAGATGTCTACGATAAATCTACTTGAGAAAAC
TACCTGTCATGCTAGAGCAGCTACGAGGCTTGCATCATTATACAT
TTGGGCCTTTGGGGACTCAGACGATAGTTGGGATGCAGTCGAGAC
TCTATCTAACAGTCGATGCCAGATCACCCGAGAGCACTTGCAAGC
TCTGTGCCCTATGCCCTCTTCAGTGAATCTCCACCATCGATTGAA
CGACGGAATAACACAAGTGAAGTTTATGCCATCGACCAATAGCAG
GGTCTCTCGATTTGTTCATATTTCCAATGACCGGCAGAATTACGT
ATTGGATGACTCCGTTACTGACAGTAATCTGATTTACCAACAAGT
CATGTTGCTAGGACTTAGTATACTTGAAACATATTTCAGAGAACC
TACAGCGACGAACTTATCAAGCTTGGTGCTGCACCTTCACACTGA
TGTATCTTGTTGCTTACGAGAATGCCCGATGACACAATATGCGCC
CCCCCTGCGAGACATCCCAGAGTTAACTATAACTGCGTCAAATCC
TTTCTTATTTGATCAAGAACCTATTAGCGAAGCAGACCTGTGTAG
ACTCTCAAAGATAGCATTCCGTAGAGCAGGAGACAACTACGATGC
ATACGACCAGTTTCAACTAAGAGCAACACTGGCCGCAACTACAGG
TAAGGATGTGGCAGCAACAATCTTTGGTCCTCTAGCTGCAGTATC
AGCAAAAAATGATGCAATAGTCACAAATGACTATAGCGGGAATTG
GATTTCCGAGTTTAGGTATAGTGATCTCTATCTTTTGAGTGTTAG
CCTGGGCTACGAGATCCTTTTGATTTTCGCGTATCAACTTTACTA
CCTACGGATACGGTACCGGCAAAATATTGTATGCTATATGGAATC
TGTATTCCGACGTTGTCATTCTTTATGCTTGGGCGACTTAATCCA
GACTATTTCGCACTCTGAAATACTGGTTGGGTTAAATGCAGCGGG
CTTTAATCTTACCTTGGATCGAAGTGATTTGAAGGAGAATCAACT
CTCGCGGCTTGCTGTGAAATACCTCACATTGTGTGTCCAGACTGC
AATAAGTAATTTGGAGGTAGGATCAGAACCCCTTTGTATTATTGG
AGGTCAATTAGATGATGACATATCATTCCAAGTTGCCCACTTCCT
CTGTCGACGACTGTGCATCATCAGCTTGGTCCACTCAAATGTACA
GAACTTACCCCCGATTAGGGATAATGAAGTTGATGTAAAATCCAA
GTTGATCTACGACCATCTTAAGCTAGTTGCTACCACACTCAATAA
TAGGGACCAGTCGTATCTGCTAACACTACTGAATAAGCCTAACAT
CGAGCTTCATACTCCACAGGTTTACTTCATTATGAGGAAGTGTCT
GGGATTATTAAAAGTCTATGGACCTGTACCTCAAAAGCAACCACT
TCCAACCTCCCCAGTAGTATCACTACCAAATAAGTGCAAATCGAA
ATGGAAGCTTGAGGAGGTAATTGACAGCATTGAGTCACCTAAGTC
TTTTAACTGGGTTCCTGATACGACGATTCCATTGGATGCTGAGCA
AAATCCCCCAAATCCTAACAGGGTCATCGATAAGATAAACATTCT
AAGATCTCTGAGCCCGCGTCACTCAGTCTGGTACCGTAACCGGCA
ATACCGGTACGTGTTGAGCCAGTTAGGGCATGACCAGCTAGGTGG
TGCAACCCTATATCTTGGTGAAGGGAGCGGCTCCACAATCTTGAA
CATAGAGCCAAAAGTCAGGAGTGATAAGATATATTATCATACCTA
TTTCTCTGCAGACAAAAGCCCTGCACAGCGCAATTTCATCCCCCA
GCCAACTACTTTTCTGCGATCTAACTTTTATCACTACGAATTAGA
GCCGTCTGGATGTGAGTTCATCAATTGTTGGTCCGAGGATGTAAA
TGCAACGAATCTTACGGAACTGCGTTGCATAAATCACATATTAAC
TGTGATTCCAGTCGGATCTCTAACACGTATTATTTGTGACGTGGA
ATTGGCAAATGACACGTCAATACAATCAGTGGCCACCGCATACAT
GAACTTAAGCATTCTTGCACATGCTCTCCTCAACCAGGGAGGCGT
YTGCATCTGTAGGTGCCACCTGTTAGACACGTCCCATCTTGCGAT
TGTTTCGTTTGTACTTAAAACATTATCAAGCCAGCTGGCGATTTC
GTTTTCAGGATATTGTGGAACCAATGACCCATCTTGTGTCATAGG
AGTCACGAAAGAGTGCTCCATTAGCCTGGATGTCTTAAGTTCGAT
TGCAGCTGCATTTATAAATGAATTGCCCTCAAATGAGCTGCCGGT
CCCTCAATCATTATTAACGCTACTAGGGTGTTATCAAGACCAGTT
AGAGAGCCTTAGTGCACTCATTGAAAAAACATGGATCCAGGAGAT
CCGGAGGCCTCATTTAATGCAATGTGAGATGGAGTGGATAGGACT
TTTGGGAACAGATGCGCTGGGCGATGTCGACACATTTATCAGTTA
TCGCAATGATTCGTGTAATTCCATCCCTGATCTACTGACGCCAGC
TGTGAGTGCCCTCTTATTTGAATTAATCAGCCTGACACCTGAGCT
TCCTGGTACGGAGGACCAGCCGAGTAGGCGTGTAATCAGCATTGG
CCAAGCTTTTAACCTGACTATTTCGGGAAAAGTTAATACGATGAT
CAGATCATGCTGTGAGCAGTGCATTAAGTTATTAGCTGCCAATGT
CTCCATCCTTAGTGATGTAGATTTGTCGTATTTTGTGAGGGGGAT
TAGAGATGGTTCTTTTGCTTTGGGGTTGTTAATCACCCAGCGTCA
AATACTCAAGGCATCGAGAGCACCAAAGTACCTCAAAACGCATCA
GGTCCAGAACTGGATTGCGTTGTTACTGGAGGTCAAACTAGAGGA
AATCTTCTCTCGCCACTACCGGAAAGTGCTATTGAGAACTCTACG
GCTGTTATCCCTCTACGGGCTCCTGCAGCAGAAGGAAAGTTGATC
ATGATAGATGTCTCTCTGATTTCGCCTCAGCCCTATATACAGATA
CCTGCTACTGCTGTTGTAACTATGCAAATATAGGCTAACCTCGGT
CCGGAGTATTGAGGTAATGGGGAGACTCGTAATTGGAGAAAGAAG
AAGAACGGGGCAGATACCAGACCCCGGGATACACTACTATAAGCT
ACTTGGGTGACTCAGGACGTCACTAATAAGAGTACACTGGTTTTA
ACAATAACACTGATAGTGTAACACTGAATATTGTAAACAGTCTCA
GGGATTATTTAATTAAAAAGACATTGCGGATCATCATGTCAGGCC
TGAACCAATCAGAAACCCATCCTACAGGCATGAGTCGATTAATCC
AGTTTAATTTAGTCCAAGATGATTAGTACTCTTAGATGTTACTAA
GTATCAGGATTTAGCGTACTAGATTAAACGTCTTTGAAAGTCATT
TTACCCTGCAAGGTGCATAAGCATATGCAGGGGAATACCCACCAC
TGGTTCGTTAGTCTAATTTATTCAAAGATGCCACCAACTGAGTGC
ACTGGGAGTCTGATTGAGCTGAATCAACCTAAAGCTCAAGCTTTC
CTGTGATGTAGCATGCAGTGTGCTACTGCTGCATAGAGCCAATCA
GGGGCGTCTTGAAGGCCTCCACCTTCCTCAGGCAACGATAAAGCA
GCTCACTGCTACACCCACCATGCAAATACAAGGATATGTAGGTCC
ATCACCCTGCTTGCTAATGTGGTCGGCTCCAGGACCATGGCCATA
GCGCTATCCGTCTAATTCAATCCGCCTCATCATATGCGTATACAA
TCCTCCACTGAGAAATGCACCTGCTCAATCCAATGACAAACTACA
ACAACACGAGATCATTATGGGGTCGATTCCCTTGAATCAGAAAGG
TAAAAACGGCAGCGGAGATGGAGAATACCAGACCTAATCGGCAAA
GAACAAAAGGTTGAAGACTGATTTAAAAATCATTATAACTTTTTG
TTTAGTGGGTCGGCATGGCATCTCCACCTCCTCGCGGTCCGGACC
TGGGCATCCGAAGGAGGACGCACGTCCACTCGGATGGCTAAGGGA
GAGCCTGCAGTAGCATAACCCCTTGGGGCCTCTAAACGGGTCTTG
AGGGGTTTTTTGGTGAAAGGAGGAACTATAGGCCGGCCCTGCAGC
AATGGCAACAACGTTGCGCAAACTATTAACTGGCGAACTACTTAC
TCTAGCTTCCCGGCAACAATTAATAGACTGGATGGAGGCGGATAA
AGTTGCAGGACCACTTCTGCGCTCGGCCCTTCCGGCTGGCTGGTT
TATTGCTGATAAATCTGGAGCCGGTGAGCGTGGGTCTCGCGGTAT
CATTGCAGCACTGGGGCCAGATGGTAAGCCCTCCCGTATCGTAGT
TATCTACACGACGGGGAGTCAGGCAACTATGGATGAACGAAATAG
ACAGATCGCTGAGATAGGTGCCTCACTGATTAAGCATTGGTAACT
GTCAGACCAAGTTTACTCATATATACTTTAGATTGATTTAAAACT
TCATTTTTAATTTAAAAGGATCTAGGTGAAGATCCTTTTTGATAA
TCTCATGACCAAAATCCCTTAACGTGAGTTTTCGTTCCACTGAGC
GTCAGACCCCGTAGAAAAGATCAAAGGATCTTCTTGAGATCCTTT
TTTTCTGCGCGTAATCTGCTGCTTGCAAACAAAAAAACCACCGCT
ACCAGCGGTGGTTTGTTTGCCGGATCAAGAGCTACCAACTCTTTT
TCCGAAGGTAACTGGCTTCAGCAGAGCGCAGATACCAAATACTGT
CCTTCTAGTGTAGCCGTAGTTAGGCCACCACTTCAAGAACTCTGT
AGCACCGCCTACATACCTCGCTCTGCTAATCCTGTTACCAGTGGC
TGCTGCCAGTGGCGATAAGTCGTGTCTTACCGGGTTGGACTCAAG
ACGATAGTTACCGGATAAGGCGCAGCGGTCGGGCTGAACGGGGGG
TTCGTGCACACAGCCCAGCTTGGAGCGAACGACCTACACCGAACT
GAGATACCTACAGCGTGAGCTATGAGAAAGCGCCACGCTTCCCGA
AGGGAGAAAGGCGGACAGGTATCCGGTAAGCGGCAGGGTCGGAAC
AGGAGAGCGCACGAGGGAGCTTCCAGGGGGAAACGCCTGGTATCT
TTATAGTCCTGTCGGGTTTCGCCACCTCTGACTTGAGCGTCGATT
TTTGTGATGCTCGTCAGGGGGGCGGAGCCTATGGAAAAACGCCAG
CAACGCGGCCTTTTTACGGTTCCTGGCCTTTTGCTGGCCTTTTGC
TCACATGTTCTTTCCTGCGTTATCCCCTGATTCTGTGGATAACCG
TATTACCGCCTTTGAGTGAGCTGATACCGCTCGCCGCAGCCGAAC
GACCGAGCGCAGCGAGTCAGTGAGCGAGGAAGCGGAAGAGCGCCT
GATGCGGTATTTTCTCCTTACGCATCTGTGCGGTATTTCACACCG
CATATGGTGCACTCTCAGTACAATCTGCTCTGATGCCGCATAGTT
AAGCCAGTATACACTCCGCTATCGCTACGTGACTGGGTCATGGCT
GCGCCCCGACACCCGCCAACACCCGCTGACGCGCCCTGACGGGCT
TGTCTGCTCCCGGCATCCGCTTACAGACAAGCTGTGACCGTCTCC
GGGAGCTGCATGTGTCAGAGGTTTTCACCGTCATCACCGAAACGC
GCGAGGCAGCTGCGGTAAAGCTCATCAGCGTGGTCGTGAAGCGAT
TCACAGATGTCTGCCTGTTCATCCGCGTCCAGCTCGTTGAGTTTC
TCCAGAAGCGTTAATGTCTGGCTTCTGATAAAGCGGGCCATGTTA
AGGGCGGTTTTTTCCTGTTTGGTCACTTGATGCCTCCGTGTAAGG
GGGAATTTCTGTTCATGGGGGTAATGATACCGATGAAACGAGAGA
GGATGCTCACGATACGGGTTACTGATGATGAACATGCCCGGTTAC
TGGAACGTTGTGAGGGTAAACAACTGGCGGTATGGATGCGGCGGG
ACCAGAGAAAAATCACTCAGGGTCAATGCCAGCGCTTCGTTAATA
CAGATGTAGGTGTTCCACAGGGTAGCCAGCAGCATCCTGCGATGC
AGATCCGGAACATAATGGTGCAGGGCGCTGACTTCCGCGTTTCCA
GACTTTACGAAACACGGAAACCGAAGACCATTCATGTTGTTGCTC
AGGTCGCAGACGTTTTGCAGCAGCAGTCGCTTCACGTTCGCTCGC
GTATCGGTGATTCATTCTGCTAACCAGTAAGGCAACCCCGCCAGC
CTAGCCGGGTCCTCAACGACAGGAGCACGATCATGCGCACCCGTG
GCCAGGACCCAACGCTGCCCGAGATGCGCCGCGTGCGGCTGCTGG
AGATGGCGGACGCGATGGATATGTTCTGCCAAGGGTTGGTTTGCG
CATTCACAGTTCTCCGCAAGAATTGATTGGCTCCAATTCTTGGAG
TGGTGAATCCGTTAGCGAGGTGCCGCCGGCTTCCATTCAGGTCGA
GGTGGCCCGGCTCCATGCACCGCGACGCAACGCGGGGAGGCAGAC
AAGGTATAGGGCGGCGCCTACAATCCATGCCAACCCGTTCCATGT
GCTCGCCGAGGCGGCATAAATCGCCGTGACGATCAGCGGTCCAGT
GATCGAAGTTAGGCTGGTAAGAGCCGCGAGCGATCCTTGAAGCTG
TCCCTGATGGTCGTCATCTACCTGCCTGGACAGCATGGCCTGCAA
CGCGGGCATCCCGATGCCGCCGGAAGCGAGAAGAATCATAATGGG
GAAGGCCATCCAGCCTCGCGTCGCGAACGCCAGCAAGACGTAGCC
CAGCGCGTCGGCCGCCATGCCGGCGATAATGGCCTGCTTCTCGCC
GAAACGTTTGGTGGCGGGACCAGTGACGAAGGCTTGAGCGAGGGC
GTGCAAGATTCCGAATACCGCAAGCGACAGGCCGATCATCGTCGC
GCTCCAGCGAAAGCGGTCCTCGCCGAAAATGACCCAGAGCGCTGC
CGGCACCTGTCCTACGAGTTGCATGATAAAGAAGACAGTCATAAG
TGCGGCGACGATAGTCATGCCCCGCGCCCACCGGAAGGAGCTGAC
TGGGTTGAAGGCTCTCAAGGGCATCGGTCGACGCTCTCCCTTATG
CGACTCCTGCATTAGGAAGCAGCCCAGTAGTAGGTTGAGGCCGTT
GAGCACCGCCGCCGCAAGGAATGGTGCATGCAAGGAGATGGCGCC
CAACAGTCCCCCGGCCACGGGGCCTGCCACCATACCCACGCCGAA
ACAAGCGCTCATGAGCCCGAAGTGGCGAGCCCGATCTTCCCCATC
GGTGATGTCGGCGATATAGGCGCCAGCAACCGCACCTGTGGCGCC
GGTGATGCCGGCCACGATGCGTCCGGCGTAGAGGATCCACAGGAC
GGGTGTGGTCGCCATGATCGCGTAGTCGATAGTGGCTCCAAGTAG
CGAAGCGAGCAGGACTGGGCGGCGGCCAAAGCGGTCGGACAGTGC
TCCGAGAACGGGTGCGCATAGAAATTGCATCAACGCATATAGCGC
TAGCAGCACGCCATAGTGACTGGCGATGCTGTCGGAATGGACGAT
ATCCCGCAAGAGGCCCGGCAGTACCGGCATAACCAAGCCTATGCC
TACAGCATCCAGGGTGACGGTGCCGAGGATGACGATGAGCGCATT
GTTAGATTTCATACACGGTGCCTGACTGCGTTAGCAATTTAACTG
TGATAAACTACCGCATTAAAGCTTATCGATGATAAGCTGTCAAAC
ATGAGAATTCGGCGCGCCTAATACGACTCACTATAGGG
pAPMV3_SARS_CoV-2-S_GSopt
(S ORF optimized for human codon usage),
SEQ ID NO: 2:
ACTAAACAGAAAATTAATACTTGTTAGTAGTCGCCGATTAATCCG
CTAATATATTAGGAGCGGAAGTCCTACACTCCGCCTCCGACCACG
AATTGAAATCATCATGGCTGGAATTTTTAATACTTATGAGCTCTT
TGTTAAGGATCAGACATGTATGCACAAGAGGGCTGCAAGCCTAAT
ATCGGGAGGTCAGCTTAAAAGCAATATACCAGTCTTCATCACCAC
AAAAGATGATCCAGCTGTCCGATGGGACCTTGTATGTTTTAATCT
CCGACTTGTTGTTAGTGAATCATCTACCTCGGTGATCCGGCAGGG
GGCTCTTATATCATTGCTTTCCATTACTGCTAGCAATATGAGGGC
TCTTGCAGCAATTGCTGGGCAGACTGATGAATCGATGATTAATAT
TATAGAAGTTGTGGACTTTGATGGACTTGAGCCCCAGTGTGACAC
TCGCAGTGGCTTAGACGCTCAGAAGCAGGAGATGTTTAAGGATAT
TGCCCGTGATATGCCGAGGGCACTATCCAGCGGGACCCCGTTCCA
GCACGGAAATGCGGAAGCGAATGGGCCTGAAGACACACACATGTT
CCTCCGATCCGCAATTAGCGTTCTGACACAGATCTGGATCCTGGT
TGCAAAAGCCATGACTAACATTGATGGCAGCAACGAGGCAAGCGA
TCGCCGCCTTGCCAAGTATATTCAACAGAATAGAATTGACAGGCG
ATTCATGCTTGCTCAAGCTACCAGGACTGTATGTCAGCAGGTCAT
AAAGGATTCTTTGACCTTGAGAAGGTTCCTTGTAACTGAGCTTCG
AAAGTCGCGGGGGGCATTGCATAGCGGGTCATCTTACTATGCTAT
GATTGGAGACATGCAGGCATACATATTTAATGCAGGCCTTACACC
ATTCCTCACTACCCTCAGGTACGGGATCGGGACTAAATACCATGC
CCTTGCAATTAGCTCACTTGCAGGTGACCTGAATAAGATTAAGGG
GTTACTGACTCTATACAAAGAGAAAGGGAGTGATGCGGGCTATAT
GGCACTTCTAGAGGATGCTGACTGCATGCAATTTGCGCCAGGTAA
TTACTCACTATTATATTCATATGCGATGGGAGTAGCAAGTGTACA
TGATGAAGGCATGAGGAATTACCAATACGCCCGGCGCTTCCTCCA
TAAGGGGATGTACCAATTTGGACGTGATATCGCAACACAACAGCA
ACATGCACTTGATGAATCCCTCGCCCAGGAGATGAGAATCACAGA
GGCTGACAGGGCAAATCTCAAGGTAATGATGGCAAACATTGGGGA
AGCATCAAGCTTCAGTGATATGCCTAATCCAGGAGCTAGCGGCAT
TCCTGCCTTCAATGACCGCCCTGATGAGATCTTCTCTGAGCCAGG
CTATCGCAAGCCCCCTCAAGATCAGCCGCTGATAAAGCTCCCAGA
TCTCGAGGAAGAAGAGCAGGACGAATTCGACATGTAGAGAGCCGC
CCACCACCCAGAGCCAGCGACAAACCCAGGCAAAACAACCCACCA
TCTCCGACCCGGAACACAACGCAACATCGAGGACCCCGGGCAGCC
GACACCGCAGCCGCAAGACCTCCGCATCATGAACGACCAGCATCC
AAACATCTGTCCTCCCTATTCTATCAGTTTAATAAAAAAGGCTTT
ACGTGCACAGACAGAGATCCTGACATAATTCAGCCGGGGGGATTC
AACTTAGGAGCGGAAGTATCCCTCCTAACCTGCACCTGCATTTCA
ATATGGATCTTGAGTTTAGTAGTGAGGAAGCAGTCGCAGCCCTCT
TGGATGTGAGTTCCTCAACCATAACCGAGGTTCTGAGCAAGCAAA
GTATTCCTGACCCTAGCTTTCTCACGTCCACGGAGGCATCCGGTG
AGCAAGTTCCAGATTCGAAGGTCGCCAATCACAAAGCCAGTGTCG
ATAAGACCCCAGATCAAGGCCAACCATCGGCAACGCCTTCGGCTC
CTCCTGAGACTGCCGAAAACATCAATACACCATCTTGCGAGGACG
GTTTACCCCCTAACTTCTTCATCCCAAGGGTAGAGAGTTACCACA
CTAACCTTTTTAAAGGGGGCCCCCAATTGGACTCAACGGAGCGGC
CGCCTGGTCACCAAATAGTTTGCGGAGATCAAGACGCGGACCTCT
CCCGAGCCGGGATAGCGAGAAAGAAGAAGAAGCAGAAGCATTGCA
AAGCGCTCAACTTTTCGGAATCCCCACTCACAGACAATCAGGCCA
TAGAAGGGAGTATTCAATCTCATGGAGCCCTGGATCAGGAACCTT
CCAGACAGAGACCTGGTGCAACCCAGCCTGCTCTCCAGTCACCGC
CTTCCCAAAACAATACAAGTGTGCATGCAGACAGTGCCCAAGATT
CTGCGATCTCTGTTTCAATCCCGCTGACTATGGTGGAATCATTGA
TCTCTCAAGTGTCAAAGTTGTCGGACCAAGTCTCGCAGATACAGA
AGTTGGTGAGTACCCTTCCTCAAATTAAGACTGATATTGCTTCGA
TTCGGAACATGCAAGCAGCTTTGGAAGGACAGCTCAGTATGATTC
GTATATTGGATCCAGGAAACTGTTCGGAATCATCACTCAATGCGC
TGAGAGGGTTCAGTGGTAAGGCACCTGTTATTGTGAGTGGTCCAG
GAAATCCCAATCAATGCATCAGCCAAGGCTCATCCACCACAATTT
GCCTGGATGAGTTAGCCCGGCCTGTGCCAAATCCTATACAGGTGA
AACTTCAAGATGATCAGAAGGACCTATCTGCACAGCGACATGCTG
TCATTGCATTACTCGAAACCAGAATCCCCCCGGGACCAAAACGTG
ACAAATTACTGTCATTGGTTGCAATTGCAAAGACTGCAAGTGATT
TAATCAAGATTAAGAGAATGGCAGTCCTCGGCCAGTGATATCACC
CTATGGCAACCAGGCTACAAATGTAAGCAGTTTCCGATATGTACC
GAATCAATAACACGACCCCTTCCGCAAAAGCAAGCGCGAGCATGA
CACACTCAGCTTCAGCCAATTCTAGCTTCACTAAGCAGGTGATCT
GACCATCTGGCTTTACTTCCGCTTTATAAAAAACTCCTAGATCTA
ACATGGAGGCAGACCGAGCTAATGCGGAGGGAGTACACAAGGGGA
ATCAAAGGAGCGGAAGGTACTAAGTCACTCCACCGCCTGCCCTCG
CCCATCCGCAGCCTCCCCCAACCCGCGGCGCCGCCACCATGTTCG
TGTTTCTGGTGCTGCTGCCTCTGGTGAGCTCCCAGTGCGTGAACC
TGACCACAAGGACCCAGCTGCCCCCTGCCTATACCAATTCCTTCA
CACGGGGCGTGTACTATCCCGACAAGGTGTTTAGATCTAGCGTGC
TGCACTCCACACAGGATCTGTTTCTGCCTTTCTTTTCTAACGTGA
CCTGGTTCCACGCCATCCACGTGAGCGGCACCAATGGCACAAAGC
GGTTCGACAATCCAGTGCTGCCCTTTAACGATGGCGTGTACTTCG
CCTCCACCGAGAAGTCTAACATCATCAGAGGCTGGATCTTTGGCA
CCACACTGGACAGCAAGACACAGTCCCTGCTGATCGTGAACAATG
CCACCAACGTGGTCATCAAGGTGTGCGAGTTCCAGTTTTGTAATG
ATCCATTCCTGGGCGTGTACTATCACAAGAACAATAAGTCTTGGA
TGGAGAGCGAGTTTCGCGTGTATTCCTCTGCCAACAATTGCACAT
TTGAGTACGTGTCCCAGCCCTTCCTGATGGACCTGGAGGGCAAGC
AGGGCAATTTCAAGAACCTGAGGGAGTTCGTGTTTAAGAATATCG
ATGGCTACTTCAAGATCTACTCCAAGCACACCCCAATCAACCTGG
TGCGCGACCTGCCACAGGGCTTCTCTGCCCTGGAGCCACTGGTGG
ATCTGCCCATCGGCATCAACATCACCCGGTTTCAGACACTGCTGG
CCCTGCACAGAAGCTACCTGACACCAGGCGACAGCTCCTCTGGAT
GGACCGCAGGAGCTGCCGCCTACTATGTGGGCTATCTGCAGCCCA
GGACCTTCCTGCTGAAGTACAACGAGAATGGCACCATCACAGACG
CCGTGGATTGCGCCCTGGATCCCCTGTCTGAGACCAAGTGTACAC
TGAAGAGCTTTACCGTGGAGAAGGGCATCTATCAGACAAGCAATT
TCAGGGTGCAGCCTACCGAGTCCATCGTGCGCTTTCCCAATATCA
CAAACCTGTGCCCTTTTGGCGAGGTGTTCAACGCAACCCGCTTCG
CCAGCGTGTACGCCTGGAATAGGAAGCGCATCTCCAACTGCGTGG
CCGACTATTCTGTGCTGTACAACAGCGCCTCCTTCTCTACCTTTA
AGTGCTATGGCGTGAGCCCCACAAAGCTGAATGACCTGTGCTTTA
CCAACGTGTACGCCGATTCCTTCGTGATCAGGGGCGACGAGGTGC
GCCAGATCGCCCCTGGCCAGACAGGCAAGATCGCCGACTACAATT
ATAAGCTGCCTGACGATTTCACCGGCTGCGTGATCGCCTGGAACT
CTAACAATCTGGATAGCAAAGTGGGCGGCAACTACAATTATCTGT
ACCGGCTGTTTAGAAAGTCTAATCTGAAGCCATTCGAGAGGGACA
TCTCCACAGAGATCTACCAGGCCGGCTCTACCCCCTGCAATGGCG
TGGAGGGCTTTAACTGTTATTTCCCTCTGCAGAGCTACGGCTTCC
AGCCAACAAACGGCGTGGGCTATCAGCCCTACCGCGTGGTGGTGC
TGTCTTTTGAGCTGCTGCACGCACCTGCAACAGTGTGCGGACCAA
AGAAGAGCACCAATCTGGTGAAGAACAAGTGCGTGAACTTCAACT
TCAACGGACTGACCGGCACAGGCGTGCTGACCGAGTCCAACAAGA
AGTTCCTGCCTTTTCAGCAGTTCGGCAGGGACATCGCAGATACCA
CAGACGCCGTGCGCGACCCTCAGACCCTGGAGATCCTGGATATCA
CACCATGCTCCTTCGGCGGCGTGTCTGTGATCACACCAGGCACCA
ATACAAGCAACCAGGTGGCCGTGCTGTATCAGGACGTGAATTGTA
CCGAGGTGCCCGTGGCAATCCACGCAGATCAGCTGACCCCTACAT
GGCGGGTGTACTCTACCGGCAGCAACGTGTTCCAGACAAGAGCCG
GATGCCTGATCGGAGCCGAGCACGTGAACAATAGCTATGAGTGCG
ACATCCCTATCGGCGCCGGCATCTGTGCCTCCTACCAGACCCAGA
CAAACTCCCCACGGAGAGCCCGGTCTGTGGCCAGCCAGTCCATCA
TCGCCTATACCATGAGCCTGGGCGCCGAGAATTCCGTGGCCTACT
CCAACAATTCTATCGCCATCCCTACCAACTTCACAATCTCCGTGA
CCACAGAGATCCTGCCAGTGAGCATGACCAAGACATCCGTGGACT
GCACAATGTATATCTGTGGCGATTCCACCGAGTGCTCTAACCTGC
TGCTGCAGTACGGCTCTTTTTGTACCCAGCTGAATAGAGCCCTGA
CAGGCATCGCCGTGGAGCAGGACAAGAACACACAGGAGGTGTTCG
CCCAGGTGAAGCAGATCTACAAGACCCCACCCATCAAGGACTTTG
GCGGCTTCAACTTCAGCCAGATCCTGCCCGATCCTAGCAAGCCAT
CCAAGCGGTCTTTTATCGAGGACCTGCTGTTCAACAAGGTGACCC
TGGCCGATGCCGGCTTCATCAAGCAGTATGGCGATTGCCTGGGCG
ACATCGCCGCCAGAGACCTGATCTGTGCCCAGAAGTTTAATGGCC
TGACCGTGCTGCCTCCACTGCTGACAGATGAGATGATCGCCCAGT
ACACATCTGCCCTGCTGGCCGGCACCATCACAAGCGGATGGACCT
TCGGCGCAGGAGCCGCCCTGCAGATCCCCTTTGCCATGCAGATGG
CCTATCGGTTCAACGGCATCGGCGTGACCCAGAATGTGCTGTACG
AGAACCAGAAGCTGATCGCCAATCAGTTTAACTCCGCCATCGGCA
AGATCCAGGACTCTCTGAGCTCCACAGCCAGCGCCCTGGGCAAGC
TGCAGGATGTGGTGAATCAGAACGCCCAGGCCCTGAATACCCTGG
TGAAGCAGCTGTCTAGCAACTTCGGCGCCATCTCCTCTGTGCTGA
ATGATATCCTGAGCAGGCTGGACAAGGTGGAGGCAGAGGTGCAGA
TCGACCGGCTGATCACAGGCAGACTGCAGTCCCTGCAGACCTACG
TGACACAGCAGCTGATCAGGGCAGCAGAGATCAGGGCCTCTGCCA
ATCTGGCCGCCACCAAGATGAGCGAGTGCGTGCTGGGCCAGTCCA
AGAGAGTGGACTTTTGTGGCAAGGGCTATCACCTGATGAGCTTCC
CACAGTCCGCCCCTCACGGAGTGGTGTTTCTGCACGTGACCTACG
TGCCAGCCCAGGAGAAGAACTTCACCACAGCACCAGCAATCTGCC
ACGATGGCAAGGCACACTTTCCTAGGGAGGGCGTGTTCGTGAGCA
ACGGCACCCACTGGTTTGTGACACAGCGCAATTTCTACGAGCCAC
AGATCATCACCACAGACAATACATTCGTGTCCGGCAACTGTGACG
TGGTCATCGGCATCGTGAACAATACCGTGTATGATCCTCTGCAGC
CAGAGCTGGACTCTTTTAAGGAGGAGCTGGATAAGTACTTCAAGA
ATCACACCAGCCCCGACGTGGATCTGGGCGACATCTCTGGCATCA
ATGCCAGCGTGGTGAACATCCAGAAGGAGATCGACAGGCTGAACG
AGGTGGCCAAGAATCTGAACGAGTCCCTGATCGATCTGCAGGAGC
TGGGCAAGTATGAGCAGTACATCAAGTGGCCCTGGTATATCTGGC
TGGGCTTCATCGCCGGCCTGATCGCCATCGTGATGGTGACCATCA
TGCTGTGCTGTATGACAAGCTGCTGTTCCTGCCTGAAGGGCTGCT
GTTCTTGTGGCAGCTGCTGTAAGTTTGATGAGGACGATAGCGAGC
CTGTGCTGAAGGGCGTGAAGCTGCACTACACCTGATAGTTTATAA
AAAACATTGTGAGAATGCAATTTAAGGACGAAAGTAGGAGCGGAA
GCCGCGGCCCAGGCTCCATTCAAAAACCATGGCCGCTGCTCTCGG
GAACATGCACCCGTCATCGTCGATTACTCTTATGCATGATGACCC
GTCCATCCAGACGCAGTTGCTAGCCTTCCCTCTGATAAGTGAGAA
GACAGAGACAGGGACGACAAAGCTGCAGCCTCAAGTGAGAATGCA
ATCTTTCCTATCAACCGATAGCCAGAAGTATCATCTGGTCTTTAT
CAACACATATGGTTTCATTGCGGAGGATTTTAATTGTAGTCCAGC
AAACGGATTTGTGCCTGCACTATTTCAACCCAAGTCTAAAGTCTT
ATCGTCAGCGATGGTTACACTTGGGGCTGTCCCTGCTGACACTGT
TCTCCAGGACCTACAACGGGACTTAATCGCAATGAGATTTAAAGT
CCGAAAGAGTGCGTCAGCGAAGGAGCTAATATTATTCTCCACAGA
CAATGTCCCTGCAACATTAACAGGGTCATCAGTCTGGAAGAATAA
AGGAGTAATAGCTGATACAGCAACTGCAGTTAAAGCGCCCGGTCG
CATCTCATGCGATGCGGTATGCAGTTATTGCATTACATTCATCTC
TTTTTGCTTCTTCCACTCATCTGCTCTTTTTAAGGTGCCCAAGCC
TCTGCTGAATTTCGAGACGGCCATAGCATATTCCCTTGTACTCCA
GGTCGAGCTTGAGTTCCCAAATATCAAAGATACCTTACACGAGAA
GTATCTCAAAGTTAAAGACTCGAAGTGGTATTGTACAATTGACAT
CCATATTGGGAATCTTTTAAAGAGAACCGCCAAGCAACGAAGGAG
GACACCTTCAGAGATAATGCAGAAAGTCCGTAAGATGGGTTTCAG
GATTGGGTTATACGATTTGTGGGGTCCAACAATTGTGGTTGAATT
AACTGGGTCCTCAAGTAAGTCCCTACACGGATTCTTCTCGAGTGA
GCGGCTGGCCTGCCACCCGGTATCACAGTATAATCCTCATGTTGG
ACAGCTTATCTGGGCCCACGATGTATCCATCACGGGGTGCCACAT
GATTATATCTGAACTTGAGAAAAAGAAGGCACTTGCAATGGCTGA
CCTGACCGTGAGTGATGCAGTAGCTGTCAACACGAGCATCAAGAG
CTTATCGCCTTTCAGACTGTTCAAAAAATATTAGGGCTAGACAAC
TCCTAGGGTGTGTGCTTATAATTTGCATATTCTAGATTATAATCA
TTAACCGCGTCCAAATGATCAGACCGCATAATTAACAGCTCAGCC
AGGACTCGCAAATCCTGCATAGGCTCAAATAACAAGACGACATAT
ATACTCACACAACAAGGTGTAATATCTAGAAGGAATCAGAATTGA
GACAATATCTAGGTTATCAGCGCTACTGCCCTGCAACTCACTATG
TATGCTTGGCATTAATAAAAAACATTGTGAGAATGCAATTTAAGG
ACGAAAGTAGGAGCGGAACACGCTCAACTGAACCCAAGCGCACAC
CAGCCCCCCGGCACCGCACCGGAAGGACGCACAACAGCCGACCGC
CCCCACCAGGGCAGAGGCCATGCAACCAGGCTCGGCACTCCACCT
CCCGCACCTGTACATAATAATTGCCTTAGTCAGCGATGGCACTTT
AGGCCAAACAGCTAAAATTGATAGGCTGATACAGGCAGGGATTGT
CTTGGGTAGTGGAAAGGAGCTCCATATCTCACAAGATTCAGGGAC
ACTGGATTTATTCGTGAGGTTGTTACCAGTACTCCCTTCGAATTT
GTCCCATTGCCAGCTTGAAGCAATAACACAGTATAATAAGACGGT
GACTAGGTTATTGGCCCCAATAGGGAAGAACCTAGAACAGGTACT
ACAAGCGAGACCGCGTGGGAGGCTGTTCGGAGCAATAATAGGGTC
CATCGCGCTTGGTGTTGCCACATCAGCACAGATTACCGCTGCAAT
TGCACTCGTCCGTGCACAGCAAAATGCTAATGATATATTAGCACT
TAAAAACGCTCTCCAATCCAGCAATGAGGCAATCCGTCAACTCAC
GTATGGCCAGGACAAACAGTTACTTGCAATTTCTAAAATACAAAA
AGCTGTGAATGAGCAGATCCTCCCTGCACTCGATCAGCTGGACTG
TGCTGTCCTTGGAACCAAACTAGCTGTGCAGCTTAATCTGTATCT
CATTGAGATGACTACCATTTTCGGCGAGCAAATCAATAATCCAGT
CTTGGCGACCATCCCATTAAGTTACATTTTACGCCTGACCGGTGC
GGAGTTGAACAACGTCCTCATGAAGCAAGCCCGATCGTCCTTAAG
CCTTGTACAGTTAGTTTCAAAAGGCTTACTTAGTGGACAAGTAAT
TGGCTATGACCCATCTGTTCAAGGTCTGATAATAAGGGTGAATCT
GATGCGAACGCAGAAGATCGATAGAGCACTAGTTTATCAGCCGTA
TGTATTACCAATCACTCTTAACTCTAACATAGTAACACCAATCGC
CCCAGAATGCGTAATTCAGAAAGGGACGATCATAGAGGGTATGTC
ACGGAAGGATTGTACAGAGTTGGAGCAAGACATAATTTGTAGGAC
AGTTACGACATACACGCTTGCCAGAGACACCAGATTGTGTTTACA
AGGCAATATCTCTTCTTGTAGGTATCAACAGTCAGGCACCCAACT
ACACACCCCATTTATTACGTACAACGGGGCAGTTATCGCAAACTG
TGATTTAGTCTCTTGTCGGTGTCTCCGTCCTCCTATGATTATCAC
CCAAGTAAAAGGGTACCCTCTTACCATAATCACAAGGTCCGTATG
TCAAGAATTATCCGTTGACAACTTAGTCCTGAATATTGAGACACA
CCATAATTTCTCCCTTAATCCAACAATTATCGACCCTCTAACAAG
AGTAATAGCCACTACACCGTTAGAGATAGATTCCTTAATCCAAGA
GGCACAAGATCATGCGAATGCAGCGCTAGCTAAAGTGGAAGAGAG
TGATAAATATCTCAGGGCAGTTACTGGCGGTAATTATTCGAATTG
GTATATCGTTCTTGTCATCGTCTTACTATTTGGGAATTTGGGCTG
GTCGCTGTTGTTAACAGTACTTCTATGTAGGTCACGGAAACAACA
GCGACGTTATCAACAAGATGATTCCGTCGGGTCAGAAAGAGGTGT
TGGGGTTGGGACCATCCAGTATATGTCATAAGTGGTGTCAGACAT
CACAATCCAAGTCACTCAGTTATCTACTCCGCTGGCTCGGAGCCA
TCGACAACGGCGTGAGCCCTCATCCGGGGACAATCCACAACCTGA
TACTACCAGACAGTGGCTCCGCACGATGGCAGCAGCCCACAACGT
CCTGCATGTAAATATCATTGCGTTACTTTAATTAAAAAACCTCAA
GGTGACCCATCCTGGCCCATCCGATTGGAGGAGCGGAAGGTTTTA
AGGTAATCAGGCAAATTGTTTCCAACATGGAATCCCCTCCTTCTG
GCAAGGATGCGCCAGCCTTCCGCGAGCCTAAGCGAACTTGCAGAC
TCTGTTACAGGGCGACGACTCTCTCCCTTAATCTCACCATCGTTG
TATTATCAATTATAAGCATTTATGTATCAACTCAAACTGGGGCAA
ACAACTCTTGTGTCAATCCCACAATCGTAACTCCTGATTATTTAA
CTGGCAGCACGACAGGCTCAGTTGAAGATCTTGCTGACTTAGAGA
GTCAACTCCGGGAGATACGTAGAGACACAGGGATTAATCTCCCAG
TTCAGATCGATAATACAGAAAATCTAATACTGACAACTCTGGCGA
GTATCAACTCGAATTTACGCTTTCTGCAAAATGCAACAACTGAGA
GCCAGACCTGCCTATCTCCTGTCAATGACCCTCGCTTTGTAGCTG
GCATAAATAGGATCCCAGCAGGGTCGATGGCATATAATGACTTCA
GCAACCTCATTGAGCACGTAAATTTCATACCGTCGCCGACAACGT
TGTCAGGCTGCACCAGGATACCATCATTTTCACTCTCTAAGACGC
ACTGGTGTTATACCCACAATGTTATATCAAACGGGTGCCTTGACC
ATGCTGCAAGTTCACAATACATCTCAATAGGGATCGTTGATACCG
GCCTAAATAATGAGCCCTATTTTAGAACAATGTCTTCAAAGTCAC
TGAATGACGGATTAAATAGAAAGAGTTGCTCTGTGACCGCAGCCG
CTAATGCATGTTGGTTACTCTGTAGCGTAGTAACAGAATACGAAG
CTGCGGACTATCGATCACGAACTCCTACTGCTATGGTTCTGGGGC
GATTCGATTTTAATGGTGAATACACAGAAATAGCTGTCCCCTCCT
CACTATTCGATGGCCGGTTTGCATCTAACTATCCAGGTGTGGGAT
CGGGAACTCAAGTCAATGGAACATTATATTTCCCTTTATATGGAG
GGGTCCTTAATGGGTCAGATATAGAAACGGCAAACAAGGGGAAAT
CCTTCAGGCCTCAGAATCCTAAGAATAGGTGTCCAGACTCGGAGG
CGATCCAAAGCTTTAGAGCGCAAGATAGCTATTACCCGACGAGAT
TTGGGAAGGTGCTGATCCAACAGGCAATCATTGCATGTAGGATCA
GTAACAAAAGTTGTACTGATTTCTATCTCCTGTACTTCGACAACA
ACAGAGTGATGATGGGTGCAGAGGCACGGCTATATTATCTTAATA
ACCAATTATATCTATATCAACGCTCGTCGAGCTGGTGGCCGCATC
CTTTGTTCTATAGCATCTCATTACCGTCATGTCAAGCCCTTGCTG
TCTGTCAAATTACAGAGGCCCATCTGACACTGACTTACGCCACGT
CGAGGCCAGGAATGTCTATCTGTACAGGAGCATCAAGGTGTCCAA
ATAATTGCGTAGACGGTGTATATACTGATGTATGGCCATTGACAA
AGAACGATGCTCAGGACCCCAATCTCTTCTACACTGTATACTTAA
ATAATTCAACTCGGCGGATTAGCCCAACCATAAGTCTATACACTT
ATGACCGCAGGATCAAATCTAAGCTGGCAGTGGGTAGTGATATAG
GAGCAGCTTATACCACGAGTACCTGTTTCGGCCGATCAGATACAG
GGGCGGTTTATTGCTTAACTATAATGGAGACCGTTAACACAATAT
TTGGTCAGTACCGGATCGTGCCAATATTACTTAGAGTGACAAGCC
GGTAATGGATTGTTCCTAGTGAAGTGGGGCCTATGGTTACTCCGT
AACGGAGATGACCTGATTTATGCTATGTAACTAATTTTGCTCAAT
CGGGAGGGTGATCGGCCGGGTATGTCCACAATGACAATGACTCCT
ATTCCCTTGCTAGAGCTAGCTTCCTCTTTAATCCAATACAATCCC
TACCTGTAGTTTAATAAAAAAATCCTCTATATAAGATCCAGACGC
GTGCTATATAAGCTAATAGCTTAGGATGGAAACGACAGGAGCGGA
ACATATCCCTCCTACCATGTCTTCTCATAATATTATCCTCCCTGA
CCACCACTTAAATTCCCCTATTGTACTAAATAAATTGATGTACTA
TTGTAAACTCCTTAATATATTGCCTGACCCTAAGTCGCCATGGTA
CGAGAAGATTAAGTCTTGGACTAATTGCTGCATCCGTGTGTCAGA
CAGCAATCGCATGACTTTGTCACGCGCCTCCAAGCTACGAGAACT
ATTGGCAACGTACGGCGTATATTCGAAGAACCACCAGCAATGTTA
TACCACCATAATCCATCCCCAATCCCTATCACCCATCATGCAGAC
TGTCTCTCAACTGGGACGCTCAATCCCGACCTGGGCCAGGATCCG
AAAGGAGATCACACACAGTATCATAGCCCAAACGAATAAATTTGA
ATCCTTATTCCACAACATCTCAAAGGACCTGACAGGGAAAACCAA
CTTGTTTGGCGGATTCTGTGATTTACATGGGAGCATAAGCACTGC
TGCAAAGCGCTTAATGAATCAACCGGGTCTCTACTTGGATTCACC
TGATGCCCATGCATGTCAATTTTTGTTCCAGCTTAAAACCTGTCA
ACGGGAATTGATTCTACTTATGAGGCAGAATGCCACAACAGAGCT
GATTAGGGTTTTTGACTATCCTGAGCTACGGGTTCTTGTGACACC
TGAATATTCGGTCTGGGTTTTTACACGGACCAAACAAGTTACATT
ACTAACATTCGATTGCTTGTTAATGTACTGCGATCTTTCAGACGG
CCGGCACAACATCCTATTCACTTGTAAGCTTTTGTCTCACTTAAA
CCACCTCGGGTGTCGGATAAGGGACCTGCTGGTGCTAATTGATTC
ACTTGCCGAGAAACACCCCTTAATAGTTTATGATGTAGTTGCAAG
TCTGGAGTCGTTATCATATGGTGCTATACAGCTACATGACAAGGT
AGCTGGTTATGCTGGAACATTTTTCTCATTTATCCTTGCAGAAAT
ACAGGATTCCTTAGAAACAGTGTTGGATCAAGGTAATAGTGAATC
TATCATATCCCAAATAAGGAACATATACTCAGGTCTCACAGTAAA
CGAGGCTGCAGAATTGCTATGTGTTATGAGGCTTTGGGGCCACCC
TGCCCTAAACAGTGTAGACGCTGCAAGCAAGGTGAGACAGAGTAT
GTGCGCTGGCAAGCTGCTTAAATTTGATACGATCCAGTTAGTCCT
TGCATTCTTCAATACACTAATAATTAATGGATATAGGAGAAAGCA
CCATGGGCGATGGCCGAATGTTTGTAGTGACTCTATAATTGGGAA
TGAACTCAAGCGGATGTATTTTGATCAATGTGAGATCCCCCACGA
CTTCACATTAAAGAACTACCGGGAATTAAGTCTTCTTGAATTCGA
GTGCACATTTCCCATAGAATTATCAGATAAACTGAACATATTTTT
AAAAGATAAGGCAATAGCATTCCCGAAGTCCAGGTGGACATCTCC
TTTCAAAGCAGATATCACACCGCGCCATTTGCTGCAAGCGCCTGA
GTTTAAAACCCGTGCCAACAGACTACTGCTATCATTCTTACAACT
CGAGGAATTCTCCATCGAATCAGAACTAGAATATGTGACTACCCT
GGCTTACCTGGATGATGATGAGTTCAACGTATCTTACTCCCTCAA
AGAGAAGGAGGTAAAGACAGATGGTCGAATCTTCGCTAAGCTCAC
AAGGAAAATGCGGAGCTGTCAGGTGATATTCGAAGAATTGCTAGC
AGAGCATGTCTCTCCATTATTTAAAGACAACGGCGTTACTATGGC
CGAATTGTCACTTACTAAGAGCCTACTCGCTATTAGCAACCTAAG
CTCCACCTTATTTGAGACGCAAACAAGGCAAGGCGATCGGAATGC
AAGATTCACGCATGCGCACTTCATAACAACTGACCTGCAAAAGTA
CTGTTTAAACTGGAGGTACCAGAGTGTAAAACTGTTCGCACGTCA
ATTGAACCGGTTATTTGGTCTGCAGCATGGGTTTGAATGGATCCA
TTGCATACTAATGAAGTCCACTATGTATGTCGCAGACCCCTTTAA
CCCTCCACATAGCAATGCACGTGAGGCACTCGATGACAACTTAAA
TAGCGACATATTCATTGTATCACCTAGGGGGGCCATCGAGGGACT
ATGCCAAAAAATGTGGACAATCATCTCAATCTCGGCGATCCATGC
ATCCGCTGCTGTGGCTGGGCTACGGGTGGCATCTATGGTCCAAGG
TGATAACCAAGTTATTGGGGTAACACGCGAGTTCCTAGCTGGGCA
TGATCAAACTTTTGTAGACGATCAATTGACGGTTTCACTTGAGAA
CTTCACTCAGATATTCAAGGAAGTCAACTACGGGCTAGGTCATAA
TCTCAAGCTGAGGGAAACAATCAAGTCGAGCCACATGTTCATCTA
CTCAAAGAGGATCTTCTACGACGGCCGAGTGCTTCCACAACTTCT
AAAGAACATAAGTAAGTTAACCCTATCAGCTACAACTACAGGGGA
GAACTGTTTAACGAGTTGCGGGGACCTATCATCATGCATCACACG
ATGTATAGAGAATGGGTTCCCTAAAGATGCAGCATTCGTTTTGAA
TCAATTAGTGATTAGGATCCAGATACTTGCCGATCACTTTTATTC
CATCCTTGGAGGATGTTTCTCTGGGTTGAACCAGGCGGATATCCG
TCTCCTTCTATATGAGGGTGCAATTTTACCGGCACAATTAGGAGG
GTTCAACAACCTCAACATGTCACGGCTATTCTGTCGGAATATTGG
TGACCCATTAGTCGCGTCCATCGCAGACACCAAGAGATTTGTGAA
GTGCCAACTGCTCGTTCCTCACATTTTAGACTCTGTCGTTGCTAT
TACCGATAGGAAGGGGTCTTTTACGACACTGATGATGGATCCATA
TTCAATAAACTTGGATTATATCCAGCAACCAGAGACACGATTGAA
GCGACATGTGCAAAAGGTTTTACTACAAGAGTCAGTGAATCCGCT
ACTCCAAGGGGTCTTCCTAGAGTCTCAACAAGAGGAAGAGGAACA
ACTTGCAGCGTTCTTGCTGGACCGAGAAGTCGTAATGCCAAGGGT
TGCGCACGCTATATTTGAGTGTACCAGCTTAGGTAGGCGCCGACA
TATCCAGGGCCTCATCGACACGACAAAGACCATTATTGCACTTGC
GTTGGATACACAGCAGTTAAGTTATACTAAACGTGAACAGATTGT
GACGTACAATGCAACTTATATGAGGTCCTTAGCAAGCATGCTCAG
TTCAAGCCATAATCAGACACGTCGGTCTGTGATTGGTCACGCATC
CTTCAATATCACCGACTGCTCGGTCATCCTGGCTCAACAAGTGAG
ACGTGCAAGCTGGGCGCCCTTGCTGAATTGGCGGTCATTAGAAGG
CTTAGAAGTACCCGACCCTATTGAGTCAGTAGCCGGCTACCTGGG
TTTGGATTCTAACAACTGTTTCTTATGCTGCCACGAGCAGAACAG
CTATTCCTGGTTTTTCCTTCCCCGAATGTGCCATTTTGATGATTC
GAGACAATCTAACTCCATACAAAGGGTACCTTACATTGGTTCTAA
GACTGACGAGCGCCAGATGTCTACGATAAATCTACTTGAGAAAAC
TACCTGTCATGCTAGAGCAGCTACGAGGCTTGCATCATTATACAT
TTGGGCCTTTGGGGACTCAGACGATAGTTGGGATGCAGTCGAGAC
TCTATCTAACAGTCGATGCCAGATCACCCGAGAGCACTTGCAAGC
TCTGTGCCCTATGCCCTCTTCAGTGAATCTCCACCATCGATTGAA
CGACGGAATAACACAAGTGAAGTTTATGCCATCGACCAATAGCAG
GGTCTCTCGATTTGTTCATATTTCCAATGACCGGCAGAATTACGT
ATTGGATGACTCCGTTACTGACAGTAATCTGATTTACCAACAAGT
CATGTTGCTAGGACTTAGTATACTTGAAACATATTTCAGAGAACC
TACAGCGACGAACTTATCAAGCTTGGTGCTGCACCTTCACACTGA
TGTATCTTGTTGCTTACGAGAATGCCCGATGACACAATATGCGCC
CCCCCTGCGAGACATCCCAGAGTTAACTATAACTGCGTCAAATCC
TTTCTTATTTGATCAAGAACCTATTAGCGAAGCAGACCTGTGTAG
ACTCTCAAAGATAGCATTCCGTAGAGCAGGAGACAACTACGATGC
ATACGACCAGTTTCAACTAAGAGCAACACTGGCCGCAACTACAGG
TAAGGATGTGGCAGCAACAATCTTTGGTCCTCTAGCTGCAGTATC
AGCAAAAAATGATGCAATAGTCACAAATGACTATAGCGGGAATTG
GATTTCCGAGTTTAGGTATAGTGATCTCTATCTTTTGAGTGTTAG
CCTGGGCTACGAGATCCTTTTGATTTTCGCGTATCAACTTTACTA
CCTACGGATACGGTACCGGCAAAATATTGTATGCTATATGGAATC
TGTATTCCGACGTTGTCATTCTTTATGCTTGGGCGACTTAATCCA
GACTATTTCGCACTCTGAAATACTGGTTGGGTTAAATGCAGCGGG
CTTTAATCTTACCTTGGATCGAAGTGATTTGAAGGAGAATCAACT
CTCGCGGCTTGCTGTGAAATACCTCACATTGTGTGTCCAGACTGC
AATAAGTAATTTGGAGGTAGGATCAGAACCCCTTTGTATTATTGG
AGGTCAATTAGATGATGACATATCATTCCAAGTTGCCCACTTCCT
CTGTCGACGACTGTGCATCATCAGCTTGGTCCACTCAAATGTACA
GAACTTACCCCCGATTAGGGATAATGAAGTTGATGTAAAATCCAA
GTTGATCTACGACCATCTTAAGCTAGTTGCTACCACACTCAATAA
TAGGGACCAGTCGTATCTGCTAACACTACTGAATAAGCCTAACAT
CGAGCTTCATACTCCACAGGTTTACTTCATTATGAGGAAGTGTCT
GGGATTATTAAAAGTCTATGGACCTGTACCTCAAAAGCAACCACT
TCCAACCTCCCCAGTAGTATCACTACCAAATAAGTGCAAATCGAA
ATGGAAGCTTGAGGAGGTAATTGACAGCATTGAGTCACCTAAGTC
TTTTAACTGGGTTCCTGATACGACGATTCCATTGGATGCTGAGCA
AAATCCCCCAAATCCTAACAGGGTCATCGATAAGATAAACATTCT
AAGATCTCTGAGCCCGCGTCACTCAGTCTGGTACCGTAACCGGCA
ATACCGGTACGTGTTGAGCCAGTTAGGGCATGACCAGCTAGGTGG
TGCAACCCTATATCTTGGTGAAGGGAGCGGCTCCACAATCTTGAA
CATAGAGCCAAAAGTCAGGAGTGATAAGATATATTATCATACCTA
TTTCTCTGCAGACAAAAGCCCTGCACAGCGCAATTTCATCCCCCA
GCCAACTACTTTTCTGCGATCTAACTTTTATCACTACGAATTAGA
GCCGTCTGGATGTGAGTTCATCAATTGTTGGTCCGAGGATGTAAA
TGCAACGAATCTTACGGAACTGCGTTGCATAAATCACATATTAAC
TGTGATTCCAGTCGGATCTCTAACACGTATTATTTGTGACGTGGA
ATTGGCAAATGACACGTCAATACAATCAGTGGCCACCGCATACAT
GAACTTAAGCATTCTTGCACATGCTCTCCTCAACCAGGGAGGCGT
YTGCATCTGTAGGTGCCACCTGTTAGACACGTCCCATCTTGCGAT
TGTTTCGTTTGTACTTAAAACATTATCAAGCCAGCTGGCGATTTC
GTTTTCAGGATATTGTGGAACCAATGACCCATCTTGTGTCATAGG
AGTCACGAAAGAGTGCTCCATTAGCCTGGATGTCTTAAGTTCGAT
TGCAGCTGCATTTATAAATGAATTGCCCTCAAATGAGCTGCCGGT
CCCTCAATCATTATTAACGCTACTAGGGTGTTATCAAGACCAGTT
AGAGAGCCTTAGTGCACTCATTGAAAAAACATGGATCCAGGAGAT
CCGGAGGCCTCATTTAATGCAATGTGAGATGGAGTGGATAGGACT
TTTGGGAACAGATGCGCTGGGCGATGTCGACACATTTATCAGTTA
TCGCAATGATTCGTGTAATTCCATCCCTGATCTACTGACGCCAGC
TGTGAGTGCCCTCTTATTTGAATTAATCAGCCTGACACCTGAGCT
TCCTGGTACGGAGGACCAGCCGAGTAGGCGTGTAATCAGCATTGG
CCAAGCTTTTAACCTGACTATTTCGGGAAAAGTTAATACGATGAT
CAGATCATGCTGTGAGCAGTGCATTAAGTTATTAGCTGCCAATGT
CTCCATCCTTAGTGATGTAGATTTGTCGTATTTTGTGAGGGGGAT
TAGAGATGGTTCTTTTGCTTTGGGGTTGTTAATCACCCAGCGTCA
AATACTCAAGGCATCGAGAGCACCAAAGTACCTCAAAACGCATCA
GGTCCAGAACTGGATTGCGTTGTTACTGGAGGTCAAACTAGAGGA
AATCTTCTCTCGCCACTACCGGAAAGTGCTATTGAGAACTCTACG
GCTGTTATCCCTCTACGGGCTCCTGCAGCAGAAGGAAAGTTGATC
ATGATAGATGTCTCTCTGATTTCGCCTCAGCCCTATATACAGATA
CCTGCTACTGCTGTTGTAACTATGCAAATATAGGCTAACCTCGGT
CCGGAGTATTGAGGTAATGGGGAGACTCGTAATTGGAGAAAGAAG
AAGAACGGGGCAGATACCAGACCCCGGGATACACTACTATAAGCT
ACTTGGGTGACTCAGGACGTCACTAATAAGAGTACACTGGTTTTA
ACAATAACACTGATAGTGTAACACTGAATATTGTAAACAGTCTCA
GGGATTATTTAATTAAAAAGACATTGCGGATCATCATGTCAGGCC
TGAACCAATCAGAAACCCATCCTACAGGCATGAGTCGATTAATCC
AGTTTAATTTAGTCCAAGATGATTAGTACTCTTAGATGTTACTAA
GTATCAGGATTTAGCGTACTAGATTAAACGTCTTTGAAAGTCATT
TTACCCTGCAAGGTGCATAAGCATATGCAGGGGAATACCCACCAC
TGGTTCGTTAGTCTAATTTATTCAAAGATGCCACCAACTGAGTGC
ACTGGGAGTCTGATTGAGCTGAATCAACCTAAAGCTCAAGCTTTC
CTGTGATGTAGCATGCAGTGTGCTACTGCTGCATAGAGCCAATCA
GGGGCGTCTTGAAGGCCTCCACCTTCCTCAGGCAACGATAAAGCA
GCTCACTGCTACACCCACCATGCAAATACAAGGATATGTAGGTCC
ATCACCCTGCTTGCTAATGTGGTCGGCTCCAGGACCATGGCCATA
GCGCTATCCGTCTAATTCAATCCGCCTCATCATATGCGTATACAA
TCCTCCACTGAGAAATGCACCTGCTCAATCCAATGACAAACTACA
ACAACACGAGATCATTATGGGGTCGATTCCCTTGAATCAGAAAGG
TAAAAACGGCAGCGGAGATGGAGAATACCAGACCTAATCGGCAAA
GAACAAAAGGTTGAAGACTGATTTAAAAATCATTATAACTTTTTG
TTTAGTGGGTCGGCATGGCATCTCCACCTCCTCGCGGTCCGGACC
TGGGCATCCGAAGGAGGACGCACGTCCACTCGGATGGCTAAGGGA
GAGCCTGCAGTAGCATAACCCCTTGGGGCCTCTAAACGGGTCTTG
AGGGGTTTTTTGGTGAAAGGAGGAACTATAGGCCGGCCCTGCAGC
AATGGCAACAACGTTGCGCAAACTATTAACTGGCGAACTACTTAC
TCTAGCTTCCCGGCAACAATTAATAGACTGGATGGAGGCGGATAA
AGTTGCAGGACCACTTCTGCGCTCGGCCCTTCCGGCTGGCTGGTT
TATTGCTGATAAATCTGGAGCCGGTGAGCGTGGGTCTCGCGGTAT
CATTGCAGCACTGGGGCCAGATGGTAAGCCCTCCCGTATCGTAGT
TATCTACACGACGGGGAGTCAGGCAACTATGGATGAACGAAATAG
ACAGATCGCTGAGATAGGTGCCTCACTGATTAAGCATTGGTAACT
GTCAGACCAAGTTTACTCATATATACTTTAGATTGATTTAAAACT
TCATTTTTAATTTAAAAGGATCTAGGTGAAGATCCTTTTTGATAA
TCTCATGACCAAAATCCCTTAACGTGAGTTTTCGTTCCACTGAGC
GTCAGACCCCGTAGAAAAGATCAAAGGATCTTCTTGAGATCCTTT
TTTTCTGCGCGTAATCTGCTGCTTGCAAACAAAAAAACCACCGCT
ACCAGCGGTGGTTTGTTTGCCGGATCAAGAGCTACCAACTCTTTT
TCCGAAGGTAACTGGCTTCAGCAGAGCGCAGATACCAAATACTGT
CCTTCTAGTGTAGCCGTAGTTAGGCCACCACTTCAAGAACTCTGT
AGCACCGCCTACATACCTCGCTCTGCTAATCCTGTTACCAGTGGC
TGCTGCCAGTGGCGATAAGTCGTGTCTTACCGGGTTGGACTCAAG
ACGATAGTTACCGGATAAGGCGCAGCGGTCGGGCTGAACGGGGGG
TTCGTGCACACAGCCCAGCTTGGAGCGAACGACCTACACCGAACT
GAGATACCTACAGCGTGAGCTATGAGAAAGCGCCACGCTTCCCGA
AGGGAGAAAGGCGGACAGGTATCCGGTAAGCGGCAGGGTCGGAAC
AGGAGAGCGCACGAGGGAGCTTCCAGGGGGAAACGCCTGGTATCT
TTATAGTCCTGTCGGGTTTCGCCACCTCTGACTTGAGCGTCGATT
TTTGTGATGCTCGTCAGGGGGGCGGAGCCTATGGAAAAACGCCAG
CAACGCGGCCTTTTTACGGTTCCTGGCCTTTTGCTGGCCTTTTGC
TCACATGTTCTTTCCTGCGTTATCCCCTGATTCTGTGGATAACCG
TATTACCGCCTTTGAGTGAGCTGATACCGCTCGCCGCAGCCGAAC
GACCGAGCGCAGCGAGTCAGTGAGCGAGGAAGCGGAAGAGCGCCT
GATGCGGTATTTTCTCCTTACGCATCTGTGCGGTATTTCACACCG
CATATGGTGCACTCTCAGTACAATCTGCTCTGATGCCGCATAGTT
AAGCCAGTATACACTCCGCTATCGCTACGTGACTGGGTCATGGCT
GCGCCCCGACACCCGCCAACACCCGCTGACGCGCCCTGACGGGCT
TGTCTGCTCCCGGCATCCGCTTACAGACAAGCTGTGACCGTCTCC
GGGAGCTGCATGTGTCAGAGGTTTTCACCGTCATCACCGAAACGC
GCGAGGCAGCTGCGGTAAAGCTCATCAGCGTGGTCGTGAAGCGAT
TCACAGATGTCTGCCTGTTCATCCGCGTCCAGCTCGTTGAGTTTC
TCCAGAAGCGTTAATGTCTGGCTTCTGATAAAGCGGGCCATGTTA
AGGGCGGTTTTTTCCTGTTTGGTCACTTGATGCCTCCGTGTAAGG
GGGAATTTCTGTTCATGGGGGTAATGATACCGATGAAACGAGAGA
GGATGCTCACGATACGGGTTACTGATGATGAACATGCCCGGTTAC
TGGAACGTTGTGAGGGTAAACAACTGGCGGTATGGATGCGGCGGG
ACCAGAGAAAAATCACTCAGGGTCAATGCCAGCGCTTCGTTAATA
CAGATGTAGGTGTTCCACAGGGTAGCCAGCAGCATCCTGCGATGC
AGATCCGGAACATAATGGTGCAGGGCGCTGACTTCCGCGTTTCCA
GACTTTACGAAACACGGAAACCGAAGACCATTCATGTTGTTGCTC
AGGTCGCAGACGTTTTGCAGCAGCAGTCGCTTCACGTTCGCTCGC
GTATCGGTGATTCATTCTGCTAACCAGTAAGGCAACCCCGCCAGC
CTAGCCGGGTCCTCAACGACAGGAGCACGATCATGCGCACCCGTG
GCCAGGACCCAACGCTGCCCGAGATGCGCCGCGTGCGGCTGCTGG
AGATGGCGGACGCGATGGATATGTTCTGCCAAGGGTTGGTTTGCG
CATTCACAGTTCTCCGCAAGAATTGATTGGCTCCAATTCTTGGAG
TGGTGAATCCGTTAGCGAGGTGCCGCCGGCTTCCATTCAGGTCGA
GGTGGCCCGGCTCCATGCACCGCGACGCAACGCGGGGAGGCAGAC
AAGGTATAGGGCGGCGCCTACAATCCATGCCAACCCGTTCCATGT
GCTCGCCGAGGCGGCATAAATCGCCGTGACGATCAGCGGTCCAGT
GATCGAAGTTAGGCTGGTAAGAGCCGCGAGCGATCCTTGAAGCTG
TCCCTGATGGTCGTCATCTACCTGCCTGGACAGCATGGCCTGCAA
CGCGGGCATCCCGATGCCGCCGGAAGCGAGAAGAATCATAATGGG
GAAGGCCATCCAGCCTCGCGTCGCGAACGCCAGCAAGACGTAGCC
CAGCGCGTCGGCCGCCATGCCGGCGATAATGGCCTGCTTCTCGCC
GAAACGTTTGGTGGCGGGACCAGTGACGAAGGCTTGAGCGAGGGC
GTGCAAGATTCCGAATACCGCAAGCGACAGGCCGATCATCGTCGC
GCTCCAGCGAAAGCGGTCCTCGCCGAAAATGACCCAGAGCGCTGC
CGGCACCTGTCCTACGAGTTGCATGATAAAGAAGACAGTCATAAG
TGCGGCGACGATAGTCATGCCCCGCGCCCACCGGAAGGAGCTGAC
TGGGTTGAAGGCTCTCAAGGGCATCGGTCGACGCTCTCCCTTATG
CGACTCCTGCATTAGGAAGCAGCCCAGTAGTAGGTTGAGGCCGTT
GAGCACCGCCGCCGCAAGGAATGGTGCATGCAAGGAGATGGCGCC
CAACAGTCCCCCGGCCACGGGGCCTGCCACCATACCCACGCCGAA
ACAAGCGCTCATGAGCCCGAAGTGGCGAGCCCGATCTTCCCCATC
GGTGATGTCGGCGATATAGGCGCCAGCAACCGCACCTGTGGCGCC
GGTGATGCCGGCCACGATGCGTCCGGCGTAGAGGATCCACAGGAC
GGGTGTGGTCGCCATGATCGCGTAGTCGATAGTGGCTCCAAGTAG
CGAAGCGAGCAGGACTGGGCGGCGGCCAAAGCGGTCGGACAGTGC
TCCGAGAACGGGTGCGCATAGAAATTGCATCAACGCATATAGCGC
TAGCAGCACGCCATAGTGACTGGCGATGCTGTCGGAATGGACGAT
ATCCCGCAAGAGGCCCGGCAGTACCGGCATAACCAAGCCTATGCC
TACAGCATCCAGGGTGACGGTGCCGAGGATGACGATGAGCGCATT
GTTAGATTTCATACACGGTGCCTGACTGCGTTAGCAATTTAACTG
TGATAAACTACCGCATTAAAGCTTATCGATGATAAGCTGTCAAAC
ATGAGAATTCGGCGCGCCTAATACGACTCACTATAGGG
pAPMV3_SARS_CoV-2-S_2P (“2P”
substitutions) SEQ ID NO: 3:
ACTAAACAGAAAATTAATACTTGTTAGTAGTCGCCGATTAATCCG
CTAATATATTAGGAGCGGAAGTCCTACACTCCGCCTCCGACCACG
AATTGAAATCATCATGGCTGGAATTTTTAATACTTATGAGCTCTT
TGTTAAGGATCAGACATGTATGCACAAGAGGGCTGCAAGCCTAAT
ATCGGGAGGTCAGCTTAAAAGCAATATACCAGTCTTCATCACCAC
AAAAGATGATCCAGCTGTCCGATGGGACCTTGTATGTTTTAATCT
CCGACTTGTTGTTAGTGAATCATCTACCTCGGTGATCCGGCAGGG
GGCTCTTATATCATTGCTTTCCATTACTGCTAGCAATATGAGGGC
TCTTGCAGCAATTGCTGGGCAGACTGATGAATCGATGATTAATAT
TATAGAAGTTGTGGACTTTGATGGACTTGAGCCCCAGTGTGACAC
TCGCAGTGGCTTAGACGCTCAGAAGCAGGAGATGTTTAAGGATAT
TGCCCGTGATATGCCGAGGGCACTATCCAGCGGGACCCCGTTCCA
GCACGGAAATGCGGAAGCGAATGGGCCTGAAGACACACACATGTT
CCTCCGATCCGCAATTAGCGTTCTGACACAGATCTGGATCCTGGT
TGCAAAAGCCATGACTAACATTGATGGCAGCAACGAGGCAAGCGA
TCGCCGCCTTGCCAAGTATATTCAACAGAATAGAATTGACAGGCG
ATTCATGCTTGCTCAAGCTACCAGGACTGTATGTCAGCAGGTCAT
AAAGGATTCTTTGACCTTGAGAAGGTTCCTTGTAACTGAGCTTCG
AAAGTCGCGGGGGGCATTGCATAGCGGGTCATCTTACTATGCTAT
GATTGGAGACATGCAGGCATACATATTTAATGCAGGCCTTACACC
ATTCCTCACTACCCTCAGGTACGGGATCGGGACTAAATACCATGC
CCTTGCAATTAGCTCACTTGCAGGTGACCTGAATAAGATTAAGGG
GTTACTGACTCTATACAAAGAGAAAGGGAGTGATGCGGGCTATAT
GGCACTTCTAGAGGATGCTGACTGCATGCAATTTGCGCCAGGTAA
TTACTCACTATTATATTCATATGCGATGGGAGTAGCAAGTGTACA
TGATGAAGGCATGAGGAATTACCAATACGCCCGGCGCTTCCTCCA
TAAGGGGATGTACCAATTTGGACGTGATATCGCAACACAACAGCA
ACATGCACTTGATGAATCCCTCGCCCAGGAGATGAGAATCACAGA
GGCTGACAGGGCAAATCTCAAGGTAATGATGGCAAACATTGGGGA
AGCATCAAGCTTCAGTGATATGCCTAATCCAGGAGCTAGCGGCAT
TCCTGCCTTCAATGACCGCCCTGATGAGATCTTCTCTGAGCCAGG
CTATCGCAAGCCCCCTCAAGATCAGCCGCTGATAAAGCTCCCAGA
TCTCGAGGAAGAAGAGCAGGACGAATTCGACATGTAGAGAGCCGC
CCACCACCCAGAGCCAGCGACAAACCCAGGCAAAACAACCCACCA
TCTCCGACCCGGAACACAACGCAACATCGAGGACCCCGGGCAGCC
GACACCGCAGCCGCAAGACCTCCGCATCATGAACGACCAGCATCC
AAACATCTGTCCTCCCTATTCTATCAGTTTAATAAAAAAGGCTTT
ACGTGCACAGACAGAGATCCTGACATAATTCAGCCGGGGGGATTC
AACTTAGGAGCGGAAGTATCCCTCCTAACCTGCACCTGCATTTCA
ATATGGATCTTGAGTTTAGTAGTGAGGAAGCAGTCGCAGCCCTCT
TGGATGTGAGTTCCTCAACCATAACCGAGGTTCTGAGCAAGCAAA
GTATTCCTGACCCTAGCTTTCTCACGTCCACGGAGGCATCCGGTG
AGCAAGTTCCAGATTCGAAGGTCGCCAATCACAAAGCCAGTGTCG
ATAAGACCCCAGATCAAGGCCAACCATCGGCAACGCCTTCGGCTC
CTCCTGAGACTGCCGAAAACATCAATACACCATCTTGCGAGGACG
GTTTACCCCCTAACTTCTTCATCCCAAGGGTAGAGAGTTACCACA
CTAACCTTTTTAAAGGGGGCCCCCAATTGGACTCAACGGAGCGGC
CGCCTGGTCACCAAATAGTTTGCGGAGATCAAGACGCGGACCTCT
CCCGAGCCGGGATAGCGAGAAAGAAGAAGAAGCAGAAGCATTGCA
AAGCGCTCAACTTTTCGGAATCCCCACTCACAGACAATCAGGCCA
TAGAAGGGAGTATTCAATCTCATGGAGCCCTGGATCAGGAACCTT
CCAGACAGAGACCTGGTGCAACCCAGCCTGCTCTCCAGTCACCGC
CTTCCCAAAACAATACAAGTGTGCATGCAGACAGTGCCCAAGATT
CTGCGATCTCTGTTTCAATCCCGCTGACTATGGTGGAATCATTGA
TCTCTCAAGTGTCAAAGTTGTCGGACCAAGTCTCGCAGATACAGA
AGTTGGTGAGTACCCTTCCTCAAATTAAGACTGATATTGCTTCGA
TTCGGAACATGCAAGCAGCTTTGGAAGGACAGCTCAGTATGATTC
GTATATTGGATCCAGGAAACTGTTCGGAATCATCACTCAATGCGC
TGAGAGGGTTCAGTGGTAAGGCACCTGTTATTGTGAGTGGTCCAG
GAAATCCCAATCAATGCATCAGCCAAGGCTCATCCACCACAATTT
GCCTGGATGAGTTAGCCCGGCCTGTGCCAAATCCTATACAGGTGA
AACTTCAAGATGATCAGAAGGACCTATCTGCACAGCGACATGCTG
TCATTGCATTACTCGAAACCAGAATCCCCCCGGGACCAAAACGTG
ACAAATTACTGTCATTGGTTGCAATTGCAAAGACTGCAAGTGATT
TAATCAAGATTAAGAGAATGGCAGTCCTCGGCCAGTGATATCACC
CTATGGCAACCAGGCTACAAATGTAAGCAGTTTCCGATATGTACC
GAATCAATAACACGACCCCTTCCGCAAAAGCAAGCGCGAGCATGA
CACACTCAGCTTCAGCCAATTCTAGCTTCACTAAGCAGGTGATCT
GACCATCTGGCTTTACTTCCGCTTTATAAAAAACTCCTAGATCTA
ACATGGAGGCAGACCGAGCTAATGCGGAGGGAGTACACAAGGGGA
ATCAAAGGAGCGGAAGGTACTAAGTCACTCCACCGCCTGCCCTCG
CCCATCCGCAGCCTCCCCCAACCCGCGGCGCCGCCACCATGTTCG
TGTTTCTGGTGCTGCTGCCTCTGGTGAGCTCCCAGTGCGTGAACC
TGACCACAAGGACCCAGCTGCCCCCTGCCTATACCAATTCCTTCA
CACGGGGCGTGTACTATCCCGACAAGGTGTTTAGATCTAGCGTGC
TGCACTCCACACAGGATCTGTTTCTGCCTTTCTTTTCTAACGTGA
CCTGGTTCCACGCCATCCACGTGAGCGGCACCAATGGCACAAAGC
GGTTCGACAATCCAGTGCTGCCCTTTAACGATGGCGTGTACTTCG
CCTCCACCGAGAAGTCTAACATCATCAGAGGCTGGATCTTTGGCA
CCACACTGGACAGCAAGACACAGTCCCTGCTGATCGTGAACAATG
CCACCAACGTGGTCATCAAGGTGTGCGAGTTCCAGTTTTGTAATG
ATCCATTCCTGGGCGTGTACTATCACAAGAACAATAAGTCTTGGA
TGGAGAGCGAGTTTCGCGTGTATTCCTCTGCCAACAATTGCACAT
TTGAGTACGTGTCCCAGCCCTTCCTGATGGACCTGGAGGGCAAGC
AGGGCAATTTCAAGAACCTGAGGGAGTTCGTGTTTAAGAATATCG
ATGGCTACTTCAAGATCTACTCCAAGCACACCCCAATCAACCTGG
TGCGCGACCTGCCACAGGGCTTCTCTGCCCTGGAGCCACTGGTGG
ATCTGCCCATCGGCATCAACATCACCCGGTTTCAGACACTGCTGG
CCCTGCACAGAAGCTACCTGACACCAGGCGACAGCTCCTCTGGAT
GGACCGCAGGAGCTGCCGCCTACTATGTGGGCTATCTGCAGCCCA
GGACCTTCCTGCTGAAGTACAACGAGAATGGCACCATCACAGACG
CCGTGGATTGCGCCCTGGATCCCCTGTCTGAGACCAAGTGTACAC
TGAAGAGCTTTACCGTGGAGAAGGGCATCTATCAGACAAGCAATT
TCAGGGTGCAGCCTACCGAGTCCATCGTGCGCTTTCCCAATATCA
CAAACCTGTGCCCTTTTGGCGAGGTGTTCAACGCAACCCGCTTCG
CCAGCGTGTACGCCTGGAATAGGAAGCGCATCTCCAACTGCGTGG
CCGACTATTCTGTGCTGTACAACAGCGCCTCCTTCTCTACCTTTA
AGTGCTATGGCGTGAGCCCCACAAAGCTGAATGACCTGTGCTTTA
CCAACGTGTACGCCGATTCCTTCGTGATCAGGGGCGACGAGGTGC
GCCAGATCGCCCCTGGCCAGACAGGCAAGATCGCCGACTACAATT
ATAAGCTGCCTGACGATTTCACCGGCTGCGTGATCGCCTGGAACT
CTAACAATCTGGATAGCAAAGTGGGCGGCAACTACAATTATCTGT
ACCGGCTGTTTAGAAAGTCTAATCTGAAGCCATTCGAGAGGGACA
TCTCCACAGAGATCTACCAGGCCGGCTCTACCCCCTGCAATGGCG
TGGAGGGCTTTAACTGTTATTTCCCTCTGCAGAGCTACGGCTTCC
AGCCAACAAACGGCGTGGGCTATCAGCCCTACCGCGTGGTGGTGC
TGTCTTTTGAGCTGCTGCACGCACCTGCAACAGTGTGCGGACCAA
AGAAGAGCACCAATCTGGTGAAGAACAAGTGCGTGAACTTCAACT
TCAACGGACTGACCGGCACAGGCGTGCTGACCGAGTCCAACAAGA
AGTTCCTGCCTTTTCAGCAGTTCGGCAGGGACATCGCAGATACCA
CAGACGCCGTGCGCGACCCTCAGACCCTGGAGATCCTGGATATCA
CACCATGCTCCTTCGGCGGCGTGTCTGTGATCACACCAGGCACCA
ATACAAGCAACCAGGTGGCCGTGCTGTATCAGGACGTGAATTGTA
CCGAGGTGCCCGTGGCAATCCACGCAGATCAGCTGACCCCTACAT
GGCGGGTGTACTCTACCGGCAGCAACGTGTTCCAGACAAGAGCCG
GATGCCTGATCGGAGCCGAGCACGTGAACAATAGCTATGAGTGCG
ACATCCCTATCGGCGCCGGCATCTGTGCCTCCTACCAGACCCAGA
CAAACTCCCCAGGGTCTGCCTCCTCTGTGGCCAGCCAGTCCATCA
TCGCCTATACCATGAGCCTGGGCGCCGAGAATTCCGTGGCCTACT
CCAACAATTCTATCGCCATCCCTACCAACTTCACAATCTCCGTGA
CCACAGAGATCCTGCCAGTGAGCATGACCAAGACATCCGTGGACT
GCACAATGTATATCTGTGGCGATTCCACCGAGTGCTCTAACCTGC
TGCTGCAGTACGGCTCTTTTTGTACCCAGCTGAATAGAGCCCTGA
CAGGCATCGCCGTGGAGCAGGACAAGAACACACAGGAGGTGTTCG
CCCAGGTGAAGCAGATCTACAAGACCCCACCCATCAAGGACTTTG
GCGGCTTCAACTTCAGCCAGATCCTGCCCGATCCTAGCAAGCCAT
CCAAGCGGTCTTTTATCGAGGACCTGCTGTTCAACAAGGTGACCC
TGGCCGATGCCGGCTTCATCAAGCAGTATGGCGATTGCCTGGGCG
ACATCGCCGCCAGAGACCTGATCTGTGCCCAGAAGTTTAATGGCC
TGACCGTGCTGCCTCCACTGCTGACAGATGAGATGATCGCCCAGT
ACACATCTGCCCTGCTGGCCGGCACCATCACAAGCGGATGGACCT
TCGGCGCAGGAGCCGCCCTGCAGATCCCCTTTGCCATGCAGATGG
CCTATCGGTTCAACGGCATCGGCGTGACCCAGAATGTGCTGTACG
AGAACCAGAAGCTGATCGCCAATCAGTTTAACTCCGCCATCGGCA
AGATCCAGGACTCTCTGAGCTCCACAGCCAGCGCCCTGGGCAAGC
TGCAGGATGTGGTGAATCAGAACGCCCAGGCCCTGAATACCCTGG
TGAAGCAGCTGTCTAGCAACTTCGGCGCCATCTCCTCTGTGCTGA
ATGATATCCTGAGCAGGCTGGACCCTCCAGAGGCAGAGGTGCAGA
TCGACCGGCTGATCACAGGCAGACTGCAGTCCCTGCAGACCTACG
TGACACAGCAGCTGATCAGGGCAGCAGAGATCAGGGCCTCTGCCA
ATCTGGCCGCCACCAAGATGAGCGAGTGCGTGCTGGGCCAGTCCA
AGAGAGTGGACTTTTGTGGCAAGGGCTATCACCTGATGAGCTTCC
CACAGTCCGCCCCTCACGGAGTGGTGTTTCTGCACGTGACCTACG
TGCCAGCCCAGGAGAAGAACTTCACCACAGCACCAGCAATCTGCC
ACGATGGCAAGGCACACTTTCCTAGGGAGGGCGTGTTCGTGAGCA
ACGGCACCCACTGGTTTGTGACACAGCGCAATTTCTACGAGCCAC
AGATCATCACCACAGACAATACATTCGTGTCCGGCAACTGTGACG
TGGTCATCGGCATCGTGAACAATACCGTGTATGATCCTCTGCAGC
CAGAGCTGGACTCTTTTAAGGAGGAGCTGGATAAGTACTTCAAGA
ATCACACCAGCCCCGACGTGGATCTGGGCGACATCTCTGGCATCA
ATGCCAGCGTGGTGAACATCCAGAAGGAGATCGACAGGCTGAACG
AGGTGGCCAAGAATCTGAACGAGTCCCTGATCGATCTGCAGGAGC
TGGGCAAGTATGAGCAGTACATCAAGTGGCCCTGGTATATCTGGC
TGGGCTTCATCGCCGGCCTGATCGCCATCGTGATGGTGACCATCA
TGCTGTGCTGTATGACAAGCTGCTGTTCCTGCCTGAAGGGCTGCT
GTTCTTGTGGCAGCTGCTGTAAGTTTGATGAGGACGATAGCGAGC
CTGTGCTGAAGGGCGTGAAGCTGCACTACACCTGATAGTTTATAA
AAAACATTGTGAGAATGCAATTTAAGGACGAAAGTAGGAGCGGAA
GCCGCGGCCCAGGCTCCATTCAAAAACCATGGCCGCTGCTCTCGG
GAACATGCACCCGTCATCGTCGATTACTCTTATGCATGATGACCC
GTCCATCCAGACGCAGTTGCTAGCCTTCCCTCTGATAAGTGAGAA
GACAGAGACAGGGACGACAAAGCTGCAGCCTCAAGTGAGAATGCA
ATCTTTCCTATCAACCGATAGCCAGAAGTATCATCTGGTCTTTAT
CAACACATATGGTTTCATTGCGGAGGATTTTAATTGTAGTCCAGC
AAACGGATTTGTGCCTGCACTATTTCAACCCAAGTCTAAAGTCTT
ATCGTCAGCGATGGTTACACTTGGGGCTGTCCCTGCTGACACTGT
TCTCCAGGACCTACAACGGGACTTAATCGCAATGAGATTTAAAGT
CCGAAAGAGTGCGTCAGCGAAGGAGCTAATATTATTCTCCACAGA
CAATGTCCCTGCAACATTAACAGGGTCATCAGTCTGGAAGAATAA
AGGAGTAATAGCTGATACAGCAACTGCAGTTAAAGCGCCCGGTCG
CATCTCATGCGATGCGGTATGCAGTTATTGCATTACATTCATCTC
TTTTTGCTTCTTCCACTCATCTGCTCTTTTTAAGGTGCCCAAGCC
TCTGCTGAATTTCGAGACGGCCATAGCATATTCCCTTGTACTCCA
GGTCGAGCTTGAGTTCCCAAATATCAAAGATACCTTACACGAGAA
GTATCTCAAAGTTAAAGACTCGAAGTGGTATTGTACAATTGACAT
CCATATTGGGAATCTTTTAAAGAGAACCGCCAAGCAACGAAGGAG
GACACCTTCAGAGATAATGCAGAAAGTCCGTAAGATGGGTTTCAG
GATTGGGTTATACGATTTGTGGGGTCCAACAATTGTGGTTGAATT
AACTGGGTCCTCAAGTAAGTCCCTACACGGATTCTTCTCGAGTGA
GCGGCTGGCCTGCCACCCGGTATCACAGTATAATCCTCATGTTGG
ACAGCTTATCTGGGCCCACGATGTATCCATCACGGGGTGCCACAT
GATTATATCTGAACTTGAGAAAAAGAAGGCACTTGCAATGGCTGA
CCTGACCGTGAGTGATGCAGTAGCTGTCAACACGAGCATCAAGAG
CTTATCGCCTTTCAGACTGTTCAAAAAATATTAGGGCTAGACAAC
TCCTAGGGTGTGTGCTTATAATTTGCATATTCTAGATTATAATCA
TTAACCGCGTCCAAATGATCAGACCGCATAATTAACAGCTCAGCC
AGGACTCGCAAATCCTGCATAGGCTCAAATAACAAGACGACATAT
ATACTCACACAACAAGGTGTAATATCTAGAAGGAATCAGAATTGA
GACAATATCTAGGTTATCAGCGCTACTGCCCTGCAACTCACTATG
TATGCTTGGCATTAATAAAAAACATTGTGAGAATGCAATTTAAGG
ACGAAAGTAGGAGCGGAACACGCTCAACTGAACCCAAGCGCACAC
CAGCCCCCCGGCACCGCACCGGAAGGACGCACAACAGCCGACCGC
CCCCACCAGGGCAGAGGCCATGCAACCAGGCTCGGCACTCCACCT
CCCGCACCTGTACATAATAATTGCCTTAGTCAGCGATGGCACTTT
AGGCCAAACAGCTAAAATTGATAGGCTGATACAGGCAGGGATTGT
CTTGGGTAGTGGAAAGGAGCTCCATATCTCACAAGATTCAGGGAC
ACTGGATTTATTCGTGAGGTTGTTACCAGTACTCCCTTCGAATTT
GTCCCATTGCCAGCTTGAAGCAATAACACAGTATAATAAGACGGT
GACTAGGTTATTGGCCCCAATAGGGAAGAACCTAGAACAGGTACT
ACAAGCGAGACCGCGTGGGAGGCTGTTCGGAGCAATAATAGGGTC
CATCGCGCTTGGTGTTGCCACATCAGCACAGATTACCGCTGCAAT
TGCACTCGTCCGTGCACAGCAAAATGCTAATGATATATTAGCACT
TAAAAACGCTCTCCAATCCAGCAATGAGGCAATCCGTCAACTCAC
GTATGGCCAGGACAAACAGTTACTTGCAATTTCTAAAATACAAAA
AGCTGTGAATGAGCAGATCCTCCCTGCACTCGATCAGCTGGACTG
TGCTGTCCTTGGAACCAAACTAGCTGTGCAGCTTAATCTGTATCT
CATTGAGATGACTACCATTTTCGGCGAGCAAATCAATAATCCAGT
CTTGGCGACCATCCCATTAAGTTACATTTTACGCCTGACCGGTGC
GGAGTTGAACAACGTCCTCATGAAGCAAGCCCGATCGTCCTTAAG
CCTTGTACAGTTAGTTTCAAAAGGCTTACTTAGTGGACAAGTAAT
TGGCTATGACCCATCTGTTCAAGGTCTGATAATAAGGGTGAATCT
GATGCGAACGCAGAAGATCGATAGAGCACTAGTTTATCAGCCGTA
TGTATTACCAATCACTCTTAACTCTAACATAGTAACACCAATCGC
CCCAGAATGCGTAATTCAGAAAGGGACGATCATAGAGGGTATGTC
ACGGAAGGATTGTACAGAGTTGGAGCAAGACATAATTTGTAGGAC
AGTTACGACATACACGCTTGCCAGAGACACCAGATTGTGTTTACA
AGGCAATATCTCTTCTTGTAGGTATCAACAGTCAGGCACCCAACT
ACACACCCCATTTATTACGTACAACGGGGCAGTTATCGCAAACTG
TGATTTAGTCTCTTGTCGGTGTCTCCGTCCTCCTATGATTATCAC
CCAAGTAAAAGGGTACCCTCTTACCATAATCACAAGGTCCGTATG
TCAAGAATTATCCGTTGACAACTTAGTCCTGAATATTGAGACACA
CCATAATTTCTCCCTTAATCCAACAATTATCGACCCTCTAACAAG
AGTAATAGCCACTACACCGTTAGAGATAGATTCCTTAATCCAAGA
GGCACAAGATCATGCGAATGCAGCGCTAGCTAAAGTGGAAGAGAG
TGATAAATATCTCAGGGCAGTTACTGGCGGTAATTATTCGAATTG
GTATATCGTTCTTGTCATCGTCTTACTATTTGGGAATTTGGGCTG
GTCGCTGTTGTTAACAGTACTTCTATGTAGGTCACGGAAACAACA
GCGACGTTATCAACAAGATGATTCCGTCGGGTCAGAAAGAGGTGT
TGGGGTTGGGACCATCCAGTATATGTCATAAGTGGTGTCAGACAT
CACAATCCAAGTCACTCAGTTATCTACTCCGCTGGCTCGGAGCCA
TCGACAACGGCGTGAGCCCTCATCCGGGGACAATCCACAACCTGA
TACTACCAGACAGTGGCTCCGCACGATGGCAGCAGCCCACAACGT
CCTGCATGTAAATATCATTGCGTTACTTTAATTAAAAAACCTCAA
GGTGACCCATCCTGGCCCATCCGATTGGAGGAGCGGAAGGTTTTA
AGGTAATCAGGCAAATTGTTTCCAACATGGAATCCCCTCCTTCTG
GCAAGGATGCGCCAGCCTTCCGCGAGCCTAAGCGAACTTGCAGAC
TCTGTTACAGGGCGACGACTCTCTCCCTTAATCTCACCATCGTTG
TATTATCAATTATAAGCATTTATGTATCAACTCAAACTGGGGCAA
ACAACTCTTGTGTCAATCCCACAATCGTAACTCCTGATTATTTAA
CTGGCAGCACGACAGGCTCAGTTGAAGATCTTGCTGACTTAGAGA
GTCAACTCCGGGAGATACGTAGAGACACAGGGATTAATCTCCCAG
TTCAGATCGATAATACAGAAAATCTAATACTGACAACTCTGGCGA
GTATCAACTCGAATTTACGCTTTCTGCAAAATGCAACAACTGAGA
GCCAGACCTGCCTATCTCCTGTCAATGACCCTCGCTTTGTAGCTG
GCATAAATAGGATCCCAGCAGGGTCGATGGCATATAATGACTTCA
GCAACCTCATTGAGCACGTAAATTTCATACCGTCGCCGACAACGT
TGTCAGGCTGCACCAGGATACCATCATTTTCACTCTCTAAGACGC
ACTGGTGTTATACCCACAATGTTATATCAAACGGGTGCCTTGACC
ATGCTGCAAGTTCACAATACATCTCAATAGGGATCGTTGATACCG
GCCTAAATAATGAGCCCTATTTTAGAACAATGTCTTCAAAGTCAC
TGAATGACGGATTAAATAGAAAGAGTTGCTCTGTGACCGCAGCCG
CTAATGCATGTTGGTTACTCTGTAGCGTAGTAACAGAATACGAAG
CTGCGGACTATCGATCACGAACTCCTACTGCTATGGTTCTGGGGC
GATTCGATTTTAATGGTGAATACACAGAAATAGCTGTCCCCTCCT
CACTATTCGATGGCCGGTTTGCATCTAACTATCCAGGTGTGGGAT
CGGGAACTCAAGTCAATGGAACATTATATTTCCCTTTATATGGAG
GGGTCCTTAATGGGTCAGATATAGAAACGGCAAACAAGGGGAAAT
CCTTCAGGCCTCAGAATCCTAAGAATAGGTGTCCAGACTCGGAGG
CGATCCAAAGCTTTAGAGCGCAAGATAGCTATTACCCGACGAGAT
TTGGGAAGGTGCTGATCCAACAGGCAATCATTGCATGTAGGATCA
GTAACAAAAGTTGTACTGATTTCTATCTCCTGTACTTCGACAACA
ACAGAGTGATGATGGGTGCAGAGGCACGGCTATATTATCTTAATA
ACCAATTATATCTATATCAACGCTCGTCGAGCTGGTGGCCGCATC
CTTTGTTCTATAGCATCTCATTACCGTCATGTCAAGCCCTTGCTG
TCTGTCAAATTACAGAGGCCCATCTGACACTGACTTACGCCACGT
CGAGGCCAGGAATGTCTATCTGTACAGGAGCATCAAGGTGTCCAA
ATAATTGCGTAGACGGTGTATATACTGATGTATGGCCATTGACAA
AGAACGATGCTCAGGACCCCAATCTCTTCTACACTGTATACTTAA
ATAATTCAACTCGGCGGATTAGCCCAACCATAAGTCTATACACTT
ATGACCGCAGGATCAAATCTAAGCTGGCAGTGGGTAGTGATATAG
GAGCAGCTTATACCACGAGTACCTGTTTCGGCCGATCAGATACAG
GGGCGGTTTATTGCTTAACTATAATGGAGACCGTTAACACAATAT
TTGGTCAGTACCGGATCGTGCCAATATTACTTAGAGTGACAAGCC
GGTAATGGATTGTTCCTAGTGAAGTGGGGCCTATGGTTACTCCGT
AACGGAGATGACCTGATTTATGCTATGTAACTAATTTTGCTCAAT
CGGGAGGGTGATCGGCCGGGTATGTCCACAATGACAATGACTCCT
ATTCCCTTGCTAGAGCTAGCTTCCTCTTTAATCCAATACAATCCC
TACCTGTAGTTTAATAAAAAAATCCTCTATATAAGATCCAGACGC
GTGCTATATAAGCTAATAGCTTAGGATGGAAACGACAGGAGCGGA
ACATATCCCTCCTACCATGTCTTCTCATAATATTATCCTCCCTGA
CCACCACTTAAATTCCCCTATTGTACTAAATAAATTGATGTACTA
TTGTAAACTCCTTAATATATTGCCTGACCCTAAGTCGCCATGGTA
CGAGAAGATTAAGTCTTGGACTAATTGCTGCATCCGTGTGTCAGA
CAGCAATCGCATGACTTTGTCACGCGCCTCCAAGCTACGAGAACT
ATTGGCAACGTACGGCGTATATTCGAAGAACCACCAGCAATGTTA
TACCACCATAATCCATCCCCAATCCCTATCACCCATCATGCAGAC
TGTCTCTCAACTGGGACGCTCAATCCCGACCTGGGCCAGGATCCG
AAAGGAGATCACACACAGTATCATAGCCCAAACGAATAAATTTGA
ATCCTTATTCCACAACATCTCAAAGGACCTGACAGGGAAAACCAA
CTTGTTTGGCGGATTCTGTGATTTACATGGGAGCATAAGCACTGC
TGCAAAGCGCTTAATGAATCAACCGGGTCTCTACTTGGATTCACC
TGATGCCCATGCATGTCAATTTTTGTTCCAGCTTAAAACCTGTCA
ACGGGAATTGATTCTACTTATGAGGCAGAATGCCACAACAGAGCT
GATTAGGGTTTTTGACTATCCTGAGCTACGGGTTCTTGTGACACC
TGAATATTCGGTCTGGGTTTTTACACGGACCAAACAAGTTACATT
ACTAACATTCGATTGCTTGTTAATGTACTGCGATCTTTCAGACGG
CCGGCACAACATCCTATTCACTTGTAAGCTTTTGTCTCACTTAAA
CCACCTCGGGTGTCGGATAAGGGACCTGCTGGTGCTAATTGATTC
ACTTGCCGAGAAACACCCCTTAATAGTTTATGATGTAGTTGCAAG
TCTGGAGTCGTTATCATATGGTGCTATACAGCTACATGACAAGGT
AGCTGGTTATGCTGGAACATTTTTCTCATTTATCCTTGCAGAAAT
ACAGGATTCCTTAGAAACAGTGTTGGATCAAGGTAATAGTGAATC
TATCATATCCCAAATAAGGAACATATACTCAGGTCTCACAGTAAA
CGAGGCTGCAGAATTGCTATGTGTTATGAGGCTTTGGGGCCACCC
TGCCCTAAACAGTGTAGACGCTGCAAGCAAGGTGAGACAGAGTAT
GTGCGCTGGCAAGCTGCTTAAATTTGATACGATCCAGTTAGTCCT
TGCATTCTTCAATACACTAATAATTAATGGATATAGGAGAAAGCA
CCATGGGCGATGGCCGAATGTTTGTAGTGACTCTATAATTGGGAA
TGAACTCAAGCGGATGTATTTTGATCAATGTGAGATCCCCCACGA
CTTCACATTAAAGAACTACCGGGAATTAAGTCTTCTTGAATTCGA
GTGCACATTTCCCATAGAATTATCAGATAAACTGAACATATTTTT
AAAAGATAAGGCAATAGCATTCCCGAAGTCCAGGTGGACATCTCC
TTTCAAAGCAGATATCACACCGCGCCATTTGCTGCAAGCGCCTGA
GTTTAAAACCCGTGCCAACAGACTACTGCTATCATTCTTACAACT
CGAGGAATTCTCCATCGAATCAGAACTAGAATATGTGACTACCCT
GGCTTACCTGGATGATGATGAGTTCAACGTATCTTACTCCCTCAA
AGAGAAGGAGGTAAAGACAGATGGTCGAATCTTCGCTAAGCTCAC
AAGGAAAATGCGGAGCTGTCAGGTGATATTCGAAGAATTGCTAGC
AGAGCATGTCTCTCCATTATTTAAAGACAACGGCGTTACTATGGC
CGAATTGTCACTTACTAAGAGCCTACTCGCTATTAGCAACCTAAG
CTCCACCTTATTTGAGACGCAAACAAGGCAAGGCGATCGGAATGC
AAGATTCACGCATGCGCACTTCATAACAACTGACCTGCAAAAGTA
CTGTTTAAACTGGAGGTACCAGAGTGTAAAACTGTTCGCACGTCA
ATTGAACCGGTTATTTGGTCTGCAGCATGGGTTTGAATGGATCCA
TTGCATACTAATGAAGTCCACTATGTATGTCGCAGACCCCTTTAA
CCCTCCACATAGCAATGCACGTGAGGCACTCGATGACAACTTAAA
TAGCGACATATTCATTGTATCACCTAGGGGGGCCATCGAGGGACT
ATGCCAAAAAATGTGGACAATCATCTCAATCTCGGCGATCCATGC
ATCCGCTGCTGTGGCTGGGCTACGGGTGGCATCTATGGTCCAAGG
TGATAACCAAGTTATTGGGGTAACACGCGAGTTCCTAGCTGGGCA
TGATCAAACTTTTGTAGACGATCAATTGACGGTTTCACTTGAGAA
CTTCACTCAGATATTCAAGGAAGTCAACTACGGGCTAGGTCATAA
TCTCAAGCTGAGGGAAACAATCAAGTCGAGCCACATGTTCATCTA
CTCAAAGAGGATCTTCTACGACGGCCGAGTGCTTCCACAACTTCT
AAAGAACATAAGTAAGTTAACCCTATCAGCTACAACTACAGGGGA
GAACTGTTTAACGAGTTGCGGGGACCTATCATCATGCATCACACG
ATGTATAGAGAATGGGTTCCCTAAAGATGCAGCATTCGTTTTGAA
TCAATTAGTGATTAGGATCCAGATACTTGCCGATCACTTTTATTC
CATCCTTGGAGGATGTTTCTCTGGGTTGAACCAGGCGGATATCCG
TCTCCTTCTATATGAGGGTGCAATTTTACCGGCACAATTAGGAGG
GTTCAACAACCTCAACATGTCACGGCTATTCTGTCGGAATATTGG
TGACCCATTAGTCGCGTCCATCGCAGACACCAAGAGATTTGTGAA
GTGCCAACTGCTCGTTCCTCACATTTTAGACTCTGTCGTTGCTAT
TACCGATAGGAAGGGGTCTTTTACGACACTGATGATGGATCCATA
TTCAATAAACTTGGATTATATCCAGCAACCAGAGACACGATTGAA
GCGACATGTGCAAAAGGTTTTACTACAAGAGTCAGTGAATCCGCT
ACTCCAAGGGGTCTTCCTAGAGTCTCAACAAGAGGAAGAGGAACA
ACTTGCAGCGTTCTTGCTGGACCGAGAAGTCGTAATGCCAAGGGT
TGCGCACGCTATATTTGAGTGTACCAGCTTAGGTAGGCGCCGACA
TATCCAGGGCCTCATCGACACGACAAAGACCATTATTGCACTTGC
GTTGGATACACAGCAGTTAAGTTATACTAAACGTGAACAGATTGT
GACGTACAATGCAACTTATATGAGGTCCTTAGCAAGCATGCTCAG
TTCAAGCCATAATCAGACACGTCGGTCTGTGATTGGTCACGCATC
CTTCAATATCACCGACTGCTCGGTCATCCTGGCTCAACAAGTGAG
ACGTGCAAGCTGGGCGCCCTTGCTGAATTGGCGGTCATTAGAAGG
CTTAGAAGTACCCGACCCTATTGAGTCAGTAGCCGGCTACCTGGG
TTTGGATTCTAACAACTGTTTCTTATGCTGCCACGAGCAGAACAG
CTATTCCTGGTTTTTCCTTCCCCGAATGTGCCATTTTGATGATTC
GAGACAATCTAACTCCATACAAAGGGTACCTTACATTGGTTCTAA
GACTGACGAGCGCCAGATGTCTACGATAAATCTACTTGAGAAAAC
TACCTGTCATGCTAGAGCAGCTACGAGGCTTGCATCATTATACAT
TTGGGCCTTTGGGGACTCAGACGATAGTTGGGATGCAGTCGAGAC
TCTATCTAACAGTCGATGCCAGATCACCCGAGAGCACTTGCAAGC
TCTGTGCCCTATGCCCTCTTCAGTGAATCTCCACCATCGATTGAA
CGACGGAATAACACAAGTGAAGTTTATGCCATCGACCAATAGCAG
GGTCTCTCGATTTGTTCATATTTCCAATGACCGGCAGAATTACGT
ATTGGATGACTCCGTTACTGACAGTAATCTGATTTACCAACAAGT
CATGTTGCTAGGACTTAGTATACTTGAAACATATTTCAGAGAACC
TACAGCGACGAACTTATCAAGCTTGGTGCTGCACCTTCACACTGA
TGTATCTTGTTGCTTACGAGAATGCCCGATGACACAATATGCGCC
CCCCCTGCGAGACATCCCAGAGTTAACTATAACTGCGTCAAATCC
TTTCTTATTTGATCAAGAACCTATTAGCGAAGCAGACCTGTGTAG
ACTCTCAAAGATAGCATTCCGTAGAGCAGGAGACAACTACGATGC
ATACGACCAGTTTCAACTAAGAGCAACACTGGCCGCAACTACAGG
TAAGGATGTGGCAGCAACAATCTTTGGTCCTCTAGCTGCAGTATC
AGCAAAAAATGATGCAATAGTCACAAATGACTATAGCGGGAATTG
GATTTCCGAGTTTAGGTATAGTGATCTCTATCTTTTGAGTGTTAG
CCTGGGCTACGAGATCCTTTTGATTTTCGCGTATCAACTTTACTA
CCTACGGATACGGTACCGGCAAAATATTGTATGCTATATGGAATC
TGTATTCCGACGTTGTCATTCTTTATGCTTGGGCGACTTAATCCA
GACTATTTCGCACTCTGAAATACTGGTTGGGTTAAATGCAGCGGG
CTTTAATCTTACCTTGGATCGAAGTGATTTGAAGGAGAATCAACT
CTCGCGGCTTGCTGTGAAATACCTCACATTGTGTGTCCAGACTGC
AATAAGTAATTTGGAGGTAGGATCAGAACCCCTTTGTATTATTGG
AGGTCAATTAGATGATGACATATCATTCCAAGTTGCCCACTTCCT
CTGTCGACGACTGTGCATCATCAGCTTGGTCCACTCAAATGTACA
GAACTTACCCCCGATTAGGGATAATGAAGTTGATGTAAAATCCAA
GTTGATCTACGACCATCTTAAGCTAGTTGCTACCACACTCAATAA
TAGGGACCAGTCGTATCTGCTAACACTACTGAATAAGCCTAACAT
CGAGCTTCATACTCCACAGGTTTACTTCATTATGAGGAAGTGTCT
GGGATTATTAAAAGTCTATGGACCTGTACCTCAAAAGCAACCACT
TCCAACCTCCCCAGTAGTATCACTACCAAATAAGTGCAAATCGAA
ATGGAAGCTTGAGGAGGTAATTGACAGCATTGAGTCACCTAAGTC
TTTTAACTGGGTTCCTGATACGACGATTCCATTGGATGCTGAGCA
AAATCCCCCAAATCCTAACAGGGTCATCGATAAGATAAACATTCT
AAGATCTCTGAGCCCGCGTCACTCAGTCTGGTACCGTAACCGGCA
ATACCGGTACGTGTTGAGCCAGTTAGGGCATGACCAGCTAGGTGG
TGCAACCCTATATCTTGGTGAAGGGAGCGGCTCCACAATCTTGAA
CATAGAGCCAAAAGTCAGGAGTGATAAGATATATTATCATACCTA
TTTCTCTGCAGACAAAAGCCCTGCACAGCGCAATTTCATCCCCCA
GCCAACTACTTTTCTGCGATCTAACTTTTATCACTACGAATTAGA
GCCGTCTGGATGTGAGTTCATCAATTGTTGGTCCGAGGATGTAAA
TGCAACGAATCTTACGGAACTGCGTTGCATAAATCACATATTAAC
TGTGATTCCAGTCGGATCTCTAACACGTATTATTTGTGACGTGGA
ATTGGCAAATGACACGTCAATACAATCAGTGGCCACCGCATACAT
GAACTTAAGCATTCTTGCACATGCTCTCCTCAACCAGGGAGGCGT
YTGCATCTGTAGGTGCCACCTGTTAGACACGTCCCATCTTGCGAT
TGTTTCGTTTGTACTTAAAACATTATCAAGCCAGCTGGCGATTTC
GTTTTCAGGATATTGTGGAACCAATGACCCATCTTGTGTCATAGG
AGTCACGAAAGAGTGCTCCATTAGCCTGGATGTCTTAAGTTCGAT
TGCAGCTGCATTTATAAATGAATTGCCCTCAAATGAGCTGCCGGT
CCCTCAATCATTATTAACGCTACTAGGGTGTTATCAAGACCAGTT
AGAGAGCCTTAGTGCACTCATTGAAAAAACATGGATCCAGGAGAT
CCGGAGGCCTCATTTAATGCAATGTGAGATGGAGTGGATAGGACT
TTTGGGAACAGATGCGCTGGGCGATGTCGACACATTTATCAGTTA
TCGCAATGATTCGTGTAATTCCATCCCTGATCTACTGACGCCAGC
TGTGAGTGCCCTCTTATTTGAATTAATCAGCCTGACACCTGAGCT
TCCTGGTACGGAGGACCAGCCGAGTAGGCGTGTAATCAGCATTGG
CCAAGCTTTTAACCTGACTATTTCGGGAAAAGTTAATACGATGAT
CAGATCATGCTGTGAGCAGTGCATTAAGTTATTAGCTGCCAATGT
CTCCATCCTTAGTGATGTAGATTTGTCGTATTTTGTGAGGGGGAT
TAGAGATGGTTCTTTTGCTTTGGGGTTGTTAATCACCCAGCGTCA
AATACTCAAGGCATCGAGAGCACCAAAGTACCTCAAAACGCATCA
GGTCCAGAACTGGATTGCGTTGTTACTGGAGGTCAAACTAGAGGA
AATCTTCTCTCGCCACTACCGGAAAGTGCTATTGAGAACTCTACG
GCTGTTATCCCTCTACGGGCTCCTGCAGCAGAAGGAAAGTTGATC
ATGATAGATGTCTCTCTGATTTCGCCTCAGCCCTATATACAGATA
CCTGCTACTGCTGTTGTAACTATGCAAATATAGGCTAACCTCGGT
CCGGAGTATTGAGGTAATGGGGAGACTCGTAATTGGAGAAAGAAG
AAGAACGGGGCAGATACCAGACCCCGGGATACACTACTATAAGCT
ACTTGGGTGACTCAGGACGTCACTAATAAGAGTACACTGGTTTTA
ACAATAACACTGATAGTGTAACACTGAATATTGTAAACAGTCTCA
GGGATTATTTAATTAAAAAGACATTGCGGATCATCATGTCAGGCC
TGAACCAATCAGAAACCCATCCTACAGGCATGAGTCGATTAATCC
AGTTTAATTTAGTCCAAGATGATTAGTACTCTTAGATGTTACTAA
GTATCAGGATTTAGCGTACTAGATTAAACGTCTTTGAAAGTCATT
TTACCCTGCAAGGTGCATAAGCATATGCAGGGGAATACCCACCAC
TGGTTCGTTAGTCTAATTTATTCAAAGATGCCACCAACTGAGTGC
ACTGGGAGTCTGATTGAGCTGAATCAACCTAAAGCTCAAGCTTTC
CTGTGATGTAGCATGCAGTGTGCTACTGCTGCATAGAGCCAATCA
GGGGCGTCTTGAAGGCCTCCACCTTCCTCAGGCAACGATAAAGCA
GCTCACTGCTACACCCACCATGCAAATACAAGGATATGTAGGTCC
ATCACCCTGCTTGCTAATGTGGTCGGCTCCAGGACCATGGCCATA
GCGCTATCCGTCTAATTCAATCCGCCTCATCATATGCGTATACAA
TCCTCCACTGAGAAATGCACCTGCTCAATCCAATGACAAACTACA
ACAACACGAGATCATTATGGGGTCGATTCCCTTGAATCAGAAAGG
TAAAAACGGCAGCGGAGATGGAGAATACCAGACCTAATCGGCAAA
GAACAAAAGGTTGAAGACTGATTTAAAAATCATTATAACTTTTTG
TTTAGTGGGTCGGCATGGCATCTCCACCTCCTCGCGGTCCGGACC
TGGGCATCCGAAGGAGGACGCACGTCCACTCGGATGGCTAAGGGA
GAGCCTGCAGTAGCATAACCCCTTGGGGCCTCTAAACGGGTCTTG
AGGGGTTTTTTGGTGAAAGGAGGAACTATAGGCCGGCCCTGCAGC
AATGGCAACAACGTTGCGCAAACTATTAACTGGCGAACTACTTAC
TCTAGCTTCCCGGCAACAATTAATAGACTGGATGGAGGCGGATAA
AGTTGCAGGACCACTTCTGCGCTCGGCCCTTCCGGCTGGCTGGTT
TATTGCTGATAAATCTGGAGCCGGTGAGCGTGGGTCTCGCGGTAT
CATTGCAGCACTGGGGCCAGATGGTAAGCCCTCCCGTATCGTAGT
TATCTACACGACGGGGAGTCAGGCAACTATGGATGAACGAAATAG
ACAGATCGCTGAGATAGGTGCCTCACTGATTAAGCATTGGTAACT
GTCAGACCAAGTTTACTCATATATACTTTAGATTGATTTAAAACT
TCATTTTTAATTTAAAAGGATCTAGGTGAAGATCCTTTTTGATAA
TCTCATGACCAAAATCCCTTAACGTGAGTTTTCGTTCCACTGAGC
GTCAGACCCCGTAGAAAAGATCAAAGGATCTTCTTGAGATCCTTT
TTTTCTGCGCGTAATCTGCTGCTTGCAAACAAAAAAACCACCGCT
ACCAGCGGTGGTTTGTTTGCCGGATCAAGAGCTACCAACTCTTTT
TCCGAAGGTAACTGGCTTCAGCAGAGCGCAGATACCAAATACTGT
CCTTCTAGTGTAGCCGTAGTTAGGCCACCACTTCAAGAACTCTGT
AGCACCGCCTACATACCTCGCTCTGCTAATCCTGTTACCAGTGGC
TGCTGCCAGTGGCGATAAGTCGTGTCTTACCGGGTTGGACTCAAG
ACGATAGTTACCGGATAAGGCGCAGCGGTCGGGCTGAACGGGGGG
TTCGTGCACACAGCCCAGCTTGGAGCGAACGACCTACACCGAACT
GAGATACCTACAGCGTGAGCTATGAGAAAGCGCCACGCTTCCCGA
AGGGAGAAAGGCGGACAGGTATCCGGTAAGCGGCAGGGTCGGAAC
AGGAGAGCGCACGAGGGAGCTTCCAGGGGGAAACGCCTGGTATCT
TTATAGTCCTGTCGGGTTTCGCCACCTCTGACTTGAGCGTCGATT
TTTGTGATGCTCGTCAGGGGGGCGGAGCCTATGGAAAAACGCCAG
CAACGCGGCCTTTTTACGGTTCCTGGCCTTTTGCTGGCCTTTTGC
TCACATGTTCTTTCCTGCGTTATCCCCTGATTCTGTGGATAACCG
TATTACCGCCTTTGAGTGAGCTGATACCGCTCGCCGCAGCCGAAC
GACCGAGCGCAGCGAGTCAGTGAGCGAGGAAGCGGAAGAGCGCCT
GATGCGGTATTTTCTCCTTACGCATCTGTGCGGTATTTCACACCG
CATATGGTGCACTCTCAGTACAATCTGCTCTGATGCCGCATAGTT
AAGCCAGTATACACTCCGCTATCGCTACGTGACTGGGTCATGGCT
GCGCCCCGACACCCGCCAACACCCGCTGACGCGCCCTGACGGGCT
TGTCTGCTCCCGGCATCCGCTTACAGACAAGCTGTGACCGTCTCC
GGGAGCTGCATGTGTCAGAGGTTTTCACCGTCATCACCGAAACGC
GCGAGGCAGCTGCGGTAAAGCTCATCAGCGTGGTCGTGAAGCGAT
TCACAGATGTCTGCCTGTTCATCCGCGTCCAGCTCGTTGAGTTTC
TCCAGAAGCGTTAATGTCTGGCTTCTGATAAAGCGGGCCATGTTA
AGGGCGGTTTTTTCCTGTTTGGTCACTTGATGCCTCCGTGTAAGG
GGGAATTTCTGTTCATGGGGGTAATGATACCGATGAAACGAGAGA
GGATGCTCACGATACGGGTTACTGATGATGAACATGCCCGGTTAC
TGGAACGTTGTGAGGGTAAACAACTGGCGGTATGGATGCGGCGGG
ACCAGAGAAAAATCACTCAGGGTCAATGCCAGCGCTTCGTTAATA
CAGATGTAGGTGTTCCACAGGGTAGCCAGCAGCATCCTGCGATGC
AGATCCGGAACATAATGGTGCAGGGCGCTGACTTCCGCGTTTCCA
GACTTTACGAAACACGGAAACCGAAGACCATTCATGTTGTTGCTC
AGGTCGCAGACGTTTTGCAGCAGCAGTCGCTTCACGTTCGCTCGC
GTATCGGTGATTCATTCTGCTAACCAGTAAGGCAACCCCGCCAGC
CTAGCCGGGTCCTCAACGACAGGAGCACGATCATGCGCACCCGTG
GCCAGGACCCAACGCTGCCCGAGATGCGCCGCGTGCGGCTGCTGG
AGATGGCGGACGCGATGGATATGTTCTGCCAAGGGTTGGTTTGCG
CATTCACAGTTCTCCGCAAGAATTGATTGGCTCCAATTCTTGGAG
TGGTGAATCCGTTAGCGAGGTGCCGCCGGCTTCCATTCAGGTCGA
GGTGGCCCGGCTCCATGCACCGCGACGCAACGCGGGGAGGCAGAC
AAGGTATAGGGCGGCGCCTACAATCCATGCCAACCCGTTCCATGT
GCTCGCCGAGGCGGCATAAATCGCCGTGACGATCAGCGGTCCAGT
GATCGAAGTTAGGCTGGTAAGAGCCGCGAGCGATCCTTGAAGCTG
TCCCTGATGGTCGTCATCTACCTGCCTGGACAGCATGGCCTGCAA
CGCGGGCATCCCGATGCCGCCGGAAGCGAGAAGAATCATAATGGG
GAAGGCCATCCAGCCTCGCGTCGCGAACGCCAGCAAGACGTAGCC
CAGCGCGTCGGCCGCCATGCCGGCGATAATGGCCTGCTTCTCGCC
GAAACGTTTGGTGGCGGGACCAGTGACGAAGGCTTGAGCGAGGGC
GTGCAAGATTCCGAATACCGCAAGCGACAGGCCGATCATCGTCGC
GCTCCAGCGAAAGCGGTCCTCGCCGAAAATGACCCAGAGCGCTGC
CGGCACCTGTCCTACGAGTTGCATGATAAAGAAGACAGTCATAAG
TGCGGCGACGATAGTCATGCCCCGCGCCCACCGGAAGGAGCTGAC
TGGGTTGAAGGCTCTCAAGGGCATCGGTCGACGCTCTCCCTTATG
CGACTCCTGCATTAGGAAGCAGCCCAGTAGTAGGTTGAGGCCGTT
GAGCACCGCCGCCGCAAGGAATGGTGCATGCAAGGAGATGGCGCC
CAACAGTCCCCCGGCCACGGGGCCTGCCACCATACCCACGCCGAA
ACAAGCGCTCATGAGCCCGAAGTGGCGAGCCCGATCTTCCCCATC
GGTGATGTCGGCGATATAGGCGCCAGCAACCGCACCTGTGGCGCC
GGTGATGCCGGCCACGATGCGTCCGGCGTAGAGGATCCACAGGAC
GGGTGTGGTCGCCATGATCGCGTAGTCGATAGTGGCTCCAAGTAG
CGAAGCGAGCAGGACTGGGCGGCGGCCAAAGCGGTCGGACAGTGC
TCCGAGAACGGGTGCGCATAGAAATTGCATCAACGCATATAGCGC
TAGCAGCACGCCATAGTGACTGGCGATGCTGTCGGAATGGACGAT
ATCCCGCAAGAGGCCCGGCAGTACCGGCATAACCAAGCCTATGCC
TACAGCATCCAGGGTGACGGTGCCGAGGATGACGATGAGCGCATT
GTTAGATTTCATACACGGTGCCTGACTGCGTTAGCAATTTAACTG
TGATAAACTACCGCATTAAAGCTTATCGATGATAAGCTGTCAAAC
ATGAGAATTCGGCGCGCCTAATACGACTCACTATAGGG
pAPMV3_SARS_CoV-2-S_6P
(4 additional P substitutions =
“6P” substitutions)
SEQ ID NO: 4:
ACTAAACAGAAAATTAATACTTGTTAGTAGTCGCCGATTAATCCG
CTAATATATTAGGAGCGGAAGTCCTACACTCCGCCTCCGACCACG
AATTGAAATCATCATGGCTGGAATTTTTAATACTTATGAGCTCTT
TGTTAAGGATCAGACATGTATGCACAAGAGGGCTGCAAGCCTAAT
ATCGGGAGGTCAGCTTAAAAGCAATATACCAGTCTTCATCACCAC
AAAAGATGATCCAGCTGTCCGATGGGACCTTGTATGTTTTAATCT
CCGACTTGTTGTTAGTGAATCATCTACCTCGGTGATCCGGCAGGG
GGCTCTTATATCATTGCTTTCCATTACTGCTAGCAATATGAGGGC
TCTTGCAGCAATTGCTGGGCAGACTGATGAATCGATGATTAATAT
TATAGAAGTTGTGGACTTTGATGGACTTGAGCCCCAGTGTGACAC
TCGCAGTGGCTTAGACGCTCAGAAGCAGGAGATGTTTAAGGATAT
TGCCCGTGATATGCCGAGGGCACTATCCAGCGGGACCCCGTTCCA
GCACGGAAATGCGGAAGCGAATGGGCCTGAAGACACACACATGTT
CCTCCGATCCGCAATTAGCGTTCTGACACAGATCTGGATCCTGGT
TGCAAAAGCCATGACTAACATTGATGGCAGCAACGAGGCAAGCGA
TCGCCGCCTTGCCAAGTATATTCAACAGAATAGAATTGACAGGCG
ATTCATGCTTGCTCAAGCTACCAGGACTGTATGTCAGCAGGTCAT
AAAGGATTCTTTGACCTTGAGAAGGTTCCTTGTAACTGAGCTTCG
AAAGTCGCGGGGGGCATTGCATAGCGGGTCATCTTACTATGCTAT
GATTGGAGACATGCAGGCATACATATTTAATGCAGGCCTTACACC
ATTCCTCACTACCCTCAGGTACGGGATCGGGACTAAATACCATGC
CCTTGCAATTAGCTCACTTGCAGGTGACCTGAATAAGATTAAGGG
GTTACTGACTCTATACAAAGAGAAAGGGAGTGATGCGGGCTATAT
GGCACTTCTAGAGGATGCTGACTGCATGCAATTTGCGCCAGGTAA
TTACTCACTATTATATTCATATGCGATGGGAGTAGCAAGTGTACA
TGATGAAGGCATGAGGAATTACCAATACGCCCGGCGCTTCCTCCA
TAAGGGGATGTACCAATTTGGACGTGATATCGCAACACAACAGCA
ACATGCACTTGATGAATCCCTCGCCCAGGAGATGAGAATCACAGA
GGCTGACAGGGCAAATCTCAAGGTAATGATGGCAAACATTGGGGA
AGCATCAAGCTTCAGTGATATGCCTAATCCAGGAGCTAGCGGCAT
TCCTGCCTTCAATGACCGCCCTGATGAGATCTTCTCTGAGCCAGG
CTATCGCAAGCCCCCTCAAGATCAGCCGCTGATAAAGCTCCCAGA
TCTCGAGGAAGAAGAGCAGGACGAATTCGACATGTAGAGAGCCGC
CCACCACCCAGAGCCAGCGACAAACCCAGGCAAAACAACCCACCA
TCTCCGACCCGGAACACAACGCAACATCGAGGACCCCGGGCAGCC
GACACCGCAGCCGCAAGACCTCCGCATCATGAACGACCAGCATCC
AAACATCTGTCCTCCCTATTCTATCAGTTTAATAAAAAAGGCTTT
ACGTGCACAGACAGAGATCCTGACATAATTCAGCCGGGGGGATTC
AACTTAGGAGCGGAAGTATCCCTCCTAACCTGCACCTGCATTTCA
ATATGGATCTTGAGTTTAGTAGTGAGGAAGCAGTCGCAGCCCTCT
TGGATGTGAGTTCCTCAACCATAACCGAGGTTCTGAGCAAGCAAA
GTATTCCTGACCCTAGCTTTCTCACGTCCACGGAGGCATCCGGTG
AGCAAGTTCCAGATTCGAAGGTCGCCAATCACAAAGCCAGTGTCG
ATAAGACCCCAGATCAAGGCCAACCATCGGCAACGCCTTCGGCTC
CTCCTGAGACTGCCGAAAACATCAATACACCATCTTGCGAGGACG
GTTTACCCCCTAACTTCTTCATCCCAAGGGTAGAGAGTTACCACA
CTAACCTTTTTAAAGGGGGCCCCCAATTGGACTCAACGGAGCGGC
CGCCTGGTCACCAAATAGTTTGCGGAGATCAAGACGCGGACCTCT
CCCGAGCCGGGATAGCGAGAAAGAAGAAGAAGCAGAAGCATTGCA
AAGCGCTCAACTTTTCGGAATCCCCACTCACAGACAATCAGGCCA
TAGAAGGGAGTATTCAATCTCATGGAGCCCTGGATCAGGAACCTT
CCAGACAGAGACCTGGTGCAACCCAGCCTGCTCTCCAGTCACCGC
CTTCCCAAAACAATACAAGTGTGCATGCAGACAGTGCCCAAGATT
CTGCGATCTCTGTTTCAATCCCGCTGACTATGGTGGAATCATTGA
TCTCTCAAGTGTCAAAGTTGTCGGACCAAGTCTCGCAGATACAGA
AGTTGGTGAGTACCCTTCCTCAAATTAAGACTGATATTGCTTCGA
TTCGGAACATGCAAGCAGCTTTGGAAGGACAGCTCAGTATGATTC
GTATATTGGATCCAGGAAACTGTTCGGAATCATCACTCAATGCGC
TGAGAGGGTTCAGTGGTAAGGCACCTGTTATTGTGAGTGGTCCAG
GAAATCCCAATCAATGCATCAGCCAAGGCTCATCCACCACAATTT
GCCTGGATGAGTTAGCCCGGCCTGTGCCAAATCCTATACAGGTGA
AACTTCAAGATGATCAGAAGGACCTATCTGCACAGCGACATGCTG
TCATTGCATTACTCGAAACCAGAATCCCCCCGGGACCAAAACGTG
ACAAATTACTGTCATTGGTTGCAATTGCAAAGACTGCAAGTGATT
TAATCAAGATTAAGAGAATGGCAGTCCTCGGCCAGTGATATCACC
CTATGGCAACCAGGCTACAAATGTAAGCAGTTTCCGATATGTACC
GAATCAATAACACGACCCCTTCCGCAAAAGCAAGCGCGAGCATGA
CACACTCAGCTTCAGCCAATTCTAGCTTCACTAAGCAGGTGATCT
GACCATCTGGCTTTACTTCCGCTTTATAAAAAACTCCTAGATCTA
ACATGGAGGCAGACCGAGCTAATGCGGAGGGAGTACACAAGGGGA
ATCAAAGGAGCGGAAGGTACTAAGTCACTCCACCGCCTGCCCTCG
CCCATCCGCAGCCTCCCCCAACCCGCGGCGCCGCCACCATGTTCG
TGTTTCTGGTGCTGCTGCCTCTGGTGAGCTCCCAGTGCGTGAACC
TGACCACAAGGACCCAGCTGCCCCCTGCCTATACCAATTCCTTCA
CACGGGGCGTGTACTATCCCGACAAGGTGTTTAGATCTAGCGTGC
TGCACTCCACACAGGATCTGTTTCTGCCTTTCTTTTCTAACGTGA
CCTGGTTCCACGCCATCCACGTGAGCGGCACCAATGGCACAAAGC
GGTTCGACAATCCAGTGCTGCCCTTTAACGATGGCGTGTACTTCG
CCTCCACCGAGAAGTCTAACATCATCAGAGGCTGGATCTTTGGCA
CCACACTGGACAGCAAGACACAGTCCCTGCTGATCGTGAACAATG
CCACCAACGTGGTCATCAAGGTGTGCGAGTTCCAGTTTTGTAATG
ATCCATTCCTGGGCGTGTACTATCACAAGAACAATAAGTCTTGGA
TGGAGAGCGAGTTTCGCGTGTATTCCTCTGCCAACAATTGCACAT
TTGAGTACGTGTCCCAGCCCTTCCTGATGGACCTGGAGGGCAAGC
AGGGCAATTTCAAGAACCTGAGGGAGTTCGTGTTTAAGAATATCG
ATGGCTACTTCAAGATCTACTCCAAGCACACCCCAATCAACCTGG
TGCGCGACCTGCCACAGGGCTTCTCTGCCCTGGAGCCACTGGTGG
ATCTGCCCATCGGCATCAACATCACCCGGTTTCAGACACTGCTGG
CCCTGCACAGAAGCTACCTGACACCAGGCGACAGCTCCTCTGGAT
GGACCGCAGGAGCTGCCGCCTACTATGTGGGCTATCTGCAGCCCA
GGACCTTCCTGCTGAAGTACAACGAGAATGGCACCATCACAGACG
CCGTGGATTGCGCCCTGGATCCCCTGTCTGAGACCAAGTGTACAC
TGAAGAGCTTTACCGTGGAGAAGGGCATCTATCAGACAAGCAATT
TCAGGGTGCAGCCTACCGAGTCCATCGTGCGCTTTCCCAATATCA
CAAACCTGTGCCCTTTTGGCGAGGTGTTCAACGCAACCCGCTTCG
CCAGCGTGTACGCCTGGAATAGGAAGCGCATCTCCAACTGCGTGG
CCGACTATTCTGTGCTGTACAACAGCGCCTCCTTCTCTACCTTTA
AGTGCTATGGCGTGAGCCCCACAAAGCTGAATGACCTGTGCTTTA
CCAACGTGTACGCCGATTCCTTCGTGATCAGGGGCGACGAGGTGC
GCCAGATCGCCCCTGGCCAGACAGGCAAGATCGCCGACTACAATT
ATAAGCTGCCTGACGATTTCACCGGCTGCGTGATCGCCTGGAACT
CTAACAATCTGGATAGCAAAGTGGGCGGCAACTACAATTATCTGT
ACCGGCTGTTTAGAAAGTCTAATCTGAAGCCATTCGAGAGGGACA
TCTCCACAGAGATCTACCAGGCCGGCTCTACCCCCTGCAATGGCG
TGGAGGGCTTTAACTGTTATTTCCCTCTGCAGAGCTACGGCTTCC
AGCCAACAAACGGCGTGGGCTATCAGCCCTACCGCGTGGTGGTGC
TGTCTTTTGAGCTGCTGCACGCACCTGCAACAGTGTGCGGACCAA
AGAAGAGCACCAATCTGGTGAAGAACAAGTGCGTGAACTTCAACT
TCAACGGACTGACCGGCACAGGCGTGCTGACCGAGTCCAACAAGA
AGTTCCTGCCTTTTCAGCAGTTCGGCAGGGACATCGCAGATACCA
CAGACGCCGTGCGCGACCCTCAGACCCTGGAGATCCTGGATATCA
CACCATGCTCCTTCGGCGGCGTGTCTGTGATCACACCAGGCACCA
ATACAAGCAACCAGGTGGCCGTGCTGTATCAGGACGTGAATTGTA
CCGAGGTGCCCGTGGCAATCCACGCAGATCAGCTGACCCCTACAT
GGCGGGTGTACTCTACCGGCAGCAACGTGTTCCAGACAAGAGCCG
GATGCCTGATCGGAGCCGAGCACGTGAACAATAGCTATGAGTGCG
ACATCCCTATCGGCGCCGGCATCTGTGCCTCCTACCAGACCCAGA
CAAACTCCCCAGGGTCTGCCTCCTCTGTGGCCAGCCAGTCCATCA
TCGCCTATACCATGAGCCTGGGCGCCGAGAATTCCGTGGCCTACT
CCAACAATTCTATCGCCATCCCTACCAACTTCACAATCTCCGTGA
CCACAGAGATCCTGCCAGTGAGCATGACCAAGACATCCGTGGACT
GCACAATGTATATCTGTGGCGATTCCACCGAGTGCTCTAACCTGC
TGCTGCAGTACGGCTCTTTTTGTACCCAGCTGAATAGAGCCCTGA
CAGGCATCGCCGTGGAGCAGGACAAGAACACACAGGAGGTGTTCG
CCCAGGTGAAGCAGATCTACAAGACCCCACCCATCAAGGACTTTG
GCGGCTTCAACTTCAGCCAGATCCTGCCCGATCCTAGCAAGCCAT
CCAAGCGGTCTCCTATCGAGGACCTGCTGTTCAACAAGGTGACCC
TGGCCGATGCCGGCTTCATCAAGCAGTATGGCGATTGCCTGGGCG
ACATCGCCGCCAGAGACCTGATCTGTGCCCAGAAGTTTAATGGCC
TGACCGTGCTGCCTCCACTGCTGACAGATGAGATGATCGCCCAGT
ACACATCTGCCCTGCTGGCCGGCACCATCACAAGCGGATGGACCT
TCGGCGCAGGACCCGCCCTGCAGATCCCCTTTCCCATGCAGATGG
CCTATCGGTTCAACGGCATCGGCGTGACCCAGAATGTGCTGTACG
AGAACCAGAAGCTGATCGCCAATCAGTTTAACTCCGCCATCGGCA
AGATCCAGGACTCTCTGAGCTCCACACCCAGCGCCCTGGGCAAGC
TGCAGGATGTGGTGAATCAGAACGCCCAGGCCCTGAATACCCTGG
TGAAGCAGCTGTCTAGCAACTTCGGCGCCATCTCCTCTGTGCTGA
ATGATATCCTGAGCAGGCTGGACCCTCCAGAGGCAGAGGTGCAGA
TCGACCGGCTGATCACAGGCAGACTGCAGTCCCTGCAGACCTACG
TGACACAGCAGCTGATCAGGGCAGCAGAGATCAGGGCCTCTGCCA
ATCTGGCCGCCACCAAGATGAGCGAGTGCGTGCTGGGCCAGTCCA
AGAGAGTGGACTTTTGTGGCAAGGGCTATCACCTGATGAGCTTCC
CACAGTCCGCCCCTCACGGAGTGGTGTTTCTGCACGTGACCTACG
TGCCAGCCCAGGAGAAGAACTTCACCACAGCACCAGCAATCTGCC
ACGATGGCAAGGCACACTTTCCTAGGGAGGGCGTGTTCGTGAGCA
ACGGCACCCACTGGTTTGTGACACAGCGCAATTTCTACGAGCCAC
AGATCATCACCACAGACAATACATTCGTGTCCGGCAACTGTGACG
TGGTCATCGGCATCGTGAACAATACCGTGTATGATCCTCTGCAGC
CAGAGCTGGACTCTTTTAAGGAGGAGCTGGATAAGTACTTCAAGA
ATCACACCAGCCCCGACGTGGATCTGGGCGACATCTCTGGCATCA
ATGCCAGCGTGGTGAACATCCAGAAGGAGATCGACAGGCTGAACG
AGGTGGCCAAGAATCTGAACGAGTCCCTGATCGATCTGCAGGAGC
TGGGCAAGTATGAGCAGTACATCAAGTGGCCCTGGTATATCTGGC
TGGGCTTCATCGCCGGCCTGATCGCCATCGTGATGGTGACCATCA
TGCTGTGCTGTATGACAAGCTGCTGTTCCTGCCTGAAGGGCTGCT
GTTCTTGTGGCAGCTGCTGTAAGTTTGATGAGGACGATAGCGAGC
CTGTGCTGAAGGGCGTGAAGCTGCACTACACCTGATAGTTTATAA
AAAACATTGTGAGAATGCAATTTAAGGACGAAAGTAGGAGCGGAA
GCCGCGGCCCAGGCTCCATTCAAAAACCATGGCCGCTGCTCTCGG
GAACATGCACCCGTCATCGTCGATTACTCTTATGCATGATGACCC
GTCCATCCAGACGCAGTTGCTAGCCTTCCCTCTGATAAGTGAGAA
GACAGAGACAGGGACGACAAAGCTGCAGCCTCAAGTGAGAATGCA
ATCTTTCCTATCAACCGATAGCCAGAAGTATCATCTGGTCTTTAT
CAACACATATGGTTTCATTGCGGAGGATTTTAATTGTAGTCCAGC
AAACGGATTTGTGCCTGCACTATTTCAACCCAAGTCTAAAGTCTT
ATCGTCAGCGATGGTTACACTTGGGGCTGTCCCTGCTGACACTGT
TCTCCAGGACCTACAACGGGACTTAATCGCAATGAGATTTAAAGT
CCGAAAGAGTGCGTCAGCGAAGGAGCTAATATTATTCTCCACAGA
CAATGTCCCTGCAACATTAACAGGGTCATCAGTCTGGAAGAATAA
AGGAGTAATAGCTGATACAGCAACTGCAGTTAAAGCGCCCGGTCG
CATCTCATGCGATGCGGTATGCAGTTATTGCATTACATTCATCTC
TTTTTGCTTCTTCCACTCATCTGCTCTTTTTAAGGTGCCCAAGCC
TCTGCTGAATTTCGAGACGGCCATAGCATATTCCCTTGTACTCCA
GGTCGAGCTTGAGTTCCCAAATATCAAAGATACCTTACACGAGAA
GTATCTCAAAGTTAAAGACTCGAAGTGGTATTGTACAATTGACAT
CCATATTGGGAATCTTTTAAAGAGAACCGCCAAGCAACGAAGGAG
GACACCTTCAGAGATAATGCAGAAAGTCCGTAAGATGGGTTTCAG
GATTGGGTTATACGATTTGTGGGGTCCAACAATTGTGGTTGAATT
AACTGGGTCCTCAAGTAAGTCCCTACACGGATTCTTCTCGAGTGA
GCGGCTGGCCTGCCACCCGGTATCACAGTATAATCCTCATGTTGG
ACAGCTTATCTGGGCCCACGATGTATCCATCACGGGGTGCCACAT
GATTATATCTGAACTTGAGAAAAAGAAGGCACTTGCAATGGCTGA
CCTGACCGTGAGTGATGCAGTAGCTGTCAACACGAGCATCAAGAG
CTTATCGCCTTTCAGACTGTTCAAAAAATATTAGGGCTAGACAAC
TCCTAGGGTGTGTGCTTATAATTTGCATATTCTAGATTATAATCA
TTAACCGCGTCCAAATGATCAGACCGCATAATTAACAGCTCAGCC
AGGACTCGCAAATCCTGCATAGGCTCAAATAACAAGACGACATAT
ATACTCACACAACAAGGTGTAATATCTAGAAGGAATCAGAATTGA
GACAATATCTAGGTTATCAGCGCTACTGCCCTGCAACTCACTATG
TATGCTTGGCATTAATAAAAAACATTGTGAGAATGCAATTTAAGG
ACGAAAGTAGGAGCGGAACACGCTCAACTGAACCCAAGCGCACAC
CAGCCCCCCGGCACCGCACCGGAAGGACGCACAACAGCCGACCGC
CCCCACCAGGGCAGAGGCCATGCAACCAGGCTCGGCACTCCACCT
CCCGCACCTGTACATAATAATTGCCTTAGTCAGCGATGGCACTTT
AGGCCAAACAGCTAAAATTGATAGGCTGATACAGGCAGGGATTGT
CTTGGGTAGTGGAAAGGAGCTCCATATCTCACAAGATTCAGGGAC
ACTGGATTTATTCGTGAGGTTGTTACCAGTACTCCCTTCGAATTT
GTCCCATTGCCAGCTTGAAGCAATAACACAGTATAATAAGACGGT
GACTAGGTTATTGGCCCCAATAGGGAAGAACCTAGAACAGGTACT
ACAAGCGAGACCGCGTGGGAGGCTGTTCGGAGCAATAATAGGGTC
CATCGCGCTTGGTGTTGCCACATCAGCACAGATTACCGCTGCAAT
TGCACTCGTCCGTGCACAGCAAAATGCTAATGATATATTAGCACT
TAAAAACGCTCTCCAATCCAGCAATGAGGCAATCCGTCAACTCAC
GTATGGCCAGGACAAACAGTTACTTGCAATTTCTAAAATACAAAA
AGCTGTGAATGAGCAGATCCTCCCTGCACTCGATCAGCTGGACTG
TGCTGTCCTTGGAACCAAACTAGCTGTGCAGCTTAATCTGTATCT
CATTGAGATGACTACCATTTTCGGCGAGCAAATCAATAATCCAGT
CTTGGCGACCATCCCATTAAGTTACATTTTACGCCTGACCGGTGC
GGAGTTGAACAACGTCCTCATGAAGCAAGCCCGATCGTCCTTAAG
CCTTGTACAGTTAGTTTCAAAAGGCTTACTTAGTGGACAAGTAAT
TGGCTATGACCCATCTGTTCAAGGTCTGATAATAAGGGTGAATCT
GATGCGAACGCAGAAGATCGATAGAGCACTAGTTTATCAGCCGTA
TGTATTACCAATCACTCTTAACTCTAACATAGTAACACCAATCGC
CCCAGAATGCGTAATTCAGAAAGGGACGATCATAGAGGGTATGTC
ACGGAAGGATTGTACAGAGTTGGAGCAAGACATAATTTGTAGGAC
AGTTACGACATACACGCTTGCCAGAGACACCAGATTGTGTTTACA
AGGCAATATCTCTTCTTGTAGGTATCAACAGTCAGGCACCCAACT
ACACACCCCATTTATTACGTACAACGGGGCAGTTATCGCAAACTG
TGATTTAGTCTCTTGTCGGTGTCTCCGTCCTCCTATGATTATCAC
CCAAGTAAAAGGGTACCCTCTTACCATAATCACAAGGTCCGTATG
TCAAGAATTATCCGTTGACAACTTAGTCCTGAATATTGAGACACA
CCATAATTTCTCCCTTAATCCAACAATTATCGACCCTCTAACAAG
AGTAATAGCCACTACACCGTTAGAGATAGATTCCTTAATCCAAGA
GGCACAAGATCATGCGAATGCAGCGCTAGCTAAAGTGGAAGAGAG
TGATAAATATCTCAGGGCAGTTACTGGCGGTAATTATTCGAATTG
GTATATCGTTCTTGTCATCGTCTTACTATTTGGGAATTTGGGCTG
GTCGCTGTTGTTAACAGTACTTCTATGTAGGTCACGGAAACAACA
GCGACGTTATCAACAAGATGATTCCGTCGGGTCAGAAAGAGGTGT
TGGGGTTGGGACCATCCAGTATATGTCATAAGTGGTGTCAGACAT
CACAATCCAAGTCACTCAGTTATCTACTCCGCTGGCTCGGAGCCA
TCGACAACGGCGTGAGCCCTCATCCGGGGACAATCCACAACCTGA
TACTACCAGACAGTGGCTCCGCACGATGGCAGCAGCCCACAACGT
CCTGCATGTAAATATCATTGCGTTACTTTAATTAAAAAACCTCAA
GGTGACCCATCCTGGCCCATCCGATTGGAGGAGCGGAAGGTTTTA
AGGTAATCAGGCAAATTGTTTCCAACATGGAATCCCCTCCTTCTG
GCAAGGATGCGCCAGCCTTCCGCGAGCCTAAGCGAACTTGCAGAC
TCTGTTACAGGGCGACGACTCTCTCCCTTAATCTCACCATCGTTG
TATTATCAATTATAAGCATTTATGTATCAACTCAAACTGGGGCAA
ACAACTCTTGTGTCAATCCCACAATCGTAACTCCTGATTATTTAA
CTGGCAGCACGACAGGCTCAGTTGAAGATCTTGCTGACTTAGAGA
GTCAACTCCGGGAGATACGTAGAGACACAGGGATTAATCTCCCAG
TTCAGATCGATAATACAGAAAATCTAATACTGACAACTCTGGCGA
GTATCAACTCGAATTTACGCTTTCTGCAAAATGCAACAACTGAGA
GCCAGACCTGCCTATCTCCTGTCAATGACCCTCGCTTTGTAGCTG
GCATAAATAGGATCCCAGCAGGGTCGATGGCATATAATGACTTCA
GCAACCTCATTGAGCACGTAAATTTCATACCGTCGCCGACAACGT
TGTCAGGCTGCACCAGGATACCATCATTTTCACTCTCTAAGACGC
ACTGGTGTTATACCCACAATGTTATATCAAACGGGTGCCTTGACC
ATGCTGCAAGTTCACAATACATCTCAATAGGGATCGTTGATACCG
GCCTAAATAATGAGCCCTATTTTAGAACAATGTCTTCAAAGTCAC
TGAATGACGGATTAAATAGAAAGAGTTGCTCTGTGACCGCAGCCG
CTAATGCATGTTGGTTACTCTGTAGCGTAGTAACAGAATACGAAG
CTGCGGACTATCGATCACGAACTCCTACTGCTATGGTTCTGGGGC
GATTCGATTTTAATGGTGAATACACAGAAATAGCTGTCCCCTCCT
CACTATTCGATGGCCGGTTTGCATCTAACTATCCAGGTGTGGGAT
CGGGAACTCAAGTCAATGGAACATTATATTTCCCTTTATATGGAG
GGGTCCTTAATGGGTCAGATATAGAAACGGCAAACAAGGGGAAAT
CCTTCAGGCCTCAGAATCCTAAGAATAGGTGTCCAGACTCGGAGG
CGATCCAAAGCTTTAGAGCGCAAGATAGCTATTACCCGACGAGAT
TTGGGAAGGTGCTGATCCAACAGGCAATCATTGCATGTAGGATCA
GTAACAAAAGTTGTACTGATTTCTATCTCCTGTACTTCGACAACA
ACAGAGTGATGATGGGTGCAGAGGCACGGCTATATTATCTTAATA
ACCAATTATATCTATATCAACGCTCGTCGAGCTGGTGGCCGCATC
CTTTGTTCTATAGCATCTCATTACCGTCATGTCAAGCCCTTGCTG
TCTGTCAAATTACAGAGGCCCATCTGACACTGACTTACGCCACGT
CGAGGCCAGGAATGTCTATCTGTACAGGAGCATCAAGGTGTCCAA
ATAATTGCGTAGACGGTGTATATACTGATGTATGGCCATTGACAA
AGAACGATGCTCAGGACCCCAATCTCTTCTACACTGTATACTTAA
ATAATTCAACTCGGCGGATTAGCCCAACCATAAGTCTATACACTT
ATGACCGCAGGATCAAATCTAAGCTGGCAGTGGGTAGTGATATAG
GAGCAGCTTATACCACGAGTACCTGTTTCGGCCGATCAGATACAG
GGGCGGTTTATTGCTTAACTATAATGGAGACCGTTAACACAATAT
TTGGTCAGTACCGGATCGTGCCAATATTACTTAGAGTGACAAGCC
GGTAATGGATTGTTCCTAGTGAAGTGGGGCCTATGGTTACTCCGT
AACGGAGATGACCTGATTTATGCTATGTAACTAATTTTGCTCAAT
CGGGAGGGTGATCGGCCGGGTATGTCCACAATGACAATGACTCCT
ATTCCCTTGCTAGAGCTAGCTTCCTCTTTAATCCAATACAATCCC
TACCTGTAGTTTAATAAAAAAATCCTCTATATAAGATCCAGACGC
GTGCTATATAAGCTAATAGCTTAGGATGGAAACGACAGGAGCGGA
ACATATCCCTCCTACCATGTCTTCTCATAATATTATCCTCCCTGA
CCACCACTTAAATTCCCCTATTGTACTAAATAAATTGATGTACTA
TTGTAAACTCCTTAATATATTGCCTGACCCTAAGTCGCCATGGTA
CGAGAAGATTAAGTCTTGGACTAATTGCTGCATCCGTGTGTCAGA
CAGCAATCGCATGACTTTGTCACGCGCCTCCAAGCTACGAGAACT
ATTGGCAACGTACGGCGTATATTCGAAGAACCACCAGCAATGTTA
TACCACCATAATCCATCCCCAATCCCTATCACCCATCATGCAGAC
TGTCTCTCAACTGGGACGCTCAATCCCGACCTGGGCCAGGATCCG
AAAGGAGATCACACACAGTATCATAGCCCAAACGAATAAATTTGA
ATCCTTATTCCACAACATCTCAAAGGACCTGACAGGGAAAACCAA
CTTGTTTGGCGGATTCTGTGATTTACATGGGAGCATAAGCACTGC
TGCAAAGCGCTTAATGAATCAACCGGGTCTCTACTTGGATTCACC
TGATGCCCATGCATGTCAATTTTTGTTCCAGCTTAAAACCTGTCA
ACGGGAATTGATTCTACTTATGAGGCAGAATGCCACAACAGAGCT
GATTAGGGTTTTTGACTATCCTGAGCTACGGGTTCTTGTGACACC
TGAATATTCGGTCTGGGTTTTTACACGGACCAAACAAGTTACATT
ACTAACATTCGATTGCTTGTTAATGTACTGCGATCTTTCAGACGG
CCGGCACAACATCCTATTCACTTGTAAGCTTTTGTCTCACTTAAA
CCACCTCGGGTGTCGGATAAGGGACCTGCTGGTGCTAATTGATTC
ACTTGCCGAGAAACACCCCTTAATAGTTTATGATGTAGTTGCAAG
TCTGGAGTCGTTATCATATGGTGCTATACAGCTACATGACAAGGT
AGCTGGTTATGCTGGAACATTTTTCTCATTTATCCTTGCAGAAAT
ACAGGATTCCTTAGAAACAGTGTTGGATCAAGGTAATAGTGAATC
TATCATATCCCAAATAAGGAACATATACTCAGGTCTCACAGTAAA
CGAGGCTGCAGAATTGCTATGTGTTATGAGGCTTTGGGGCCACCC
TGCCCTAAACAGTGTAGACGCTGCAAGCAAGGTGAGACAGAGTAT
GTGCGCTGGCAAGCTGCTTAAATTTGATACGATCCAGTTAGTCCT
TGCATTCTTCAATACACTAATAATTAATGGATATAGGAGAAAGCA
CCATGGGCGATGGCCGAATGTTTGTAGTGACTCTATAATTGGGAA
TGAACTCAAGCGGATGTATTTTGATCAATGTGAGATCCCCCACGA
CTTCACATTAAAGAACTACCGGGAATTAAGTCTTCTTGAATTCGA
GTGCACATTTCCCATAGAATTATCAGATAAACTGAACATATTTTT
AAAAGATAAGGCAATAGCATTCCCGAAGTCCAGGTGGACATCTCC
TTTCAAAGCAGATATCACACCGCGCCATTTGCTGCAAGCGCCTGA
GTTTAAAACCCGTGCCAACAGACTACTGCTATCATTCTTACAACT
CGAGGAATTCTCCATCGAATCAGAACTAGAATATGTGACTACCCT
GGCTTACCTGGATGATGATGAGTTCAACGTATCTTACTCCCTCAA
AGAGAAGGAGGTAAAGACAGATGGTCGAATCTTCGCTAAGCTCAC
AAGGAAAATGCGGAGCTGTCAGGTGATATTCGAAGAATTGCTAGC
AGAGCATGTCTCTCCATTATTTAAAGACAACGGCGTTACTATGGC
CGAATTGTCACTTACTAAGAGCCTACTCGCTATTAGCAACCTAAG
CTCCACCTTATTTGAGACGCAAACAAGGCAAGGCGATCGGAATGC
AAGATTCACGCATGCGCACTTCATAACAACTGACCTGCAAAAGTA
CTGTTTAAACTGGAGGTACCAGAGTGTAAAACTGTTCGCACGTCA
ATTGAACCGGTTATTTGGTCTGCAGCATGGGTTTGAATGGATCCA
TTGCATACTAATGAAGTCCACTATGTATGTCGCAGACCCCTTTAA
CCCTCCACATAGCAATGCACGTGAGGCACTCGATGACAACTTAAA
TAGCGACATATTCATTGTATCACCTAGGGGGGCCATCGAGGGACT
ATGCCAAAAAATGTGGACAATCATCTCAATCTCGGCGATCCATGC
ATCCGCTGCTGTGGCTGGGCTACGGGTGGCATCTATGGTCCAAGG
TGATAACCAAGTTATTGGGGTAACACGCGAGTTCCTAGCTGGGCA
TGATCAAACTTTTGTAGACGATCAATTGACGGTTTCACTTGAGAA
CTTCACTCAGATATTCAAGGAAGTCAACTACGGGCTAGGTCATAA
TCTCAAGCTGAGGGAAACAATCAAGTCGAGCCACATGTTCATCTA
CTCAAAGAGGATCTTCTACGACGGCCGAGTGCTTCCACAACTTCT
AAAGAACATAAGTAAGTTAACCCTATCAGCTACAACTACAGGGGA
GAACTGTTTAACGAGTTGCGGGGACCTATCATCATGCATCACACG
ATGTATAGAGAATGGGTTCCCTAAAGATGCAGCATTCGTTTTGAA
TCAATTAGTGATTAGGATCCAGATACTTGCCGATCACTTTTATTC
CATCCTTGGAGGATGTTTCTCTGGGTTGAACCAGGCGGATATCCG
TCTCCTTCTATATGAGGGTGCAATTTTACCGGCACAATTAGGAGG
GTTCAACAACCTCAACATGTCACGGCTATTCTGTCGGAATATTGG
TGACCCATTAGTCGCGTCCATCGCAGACACCAAGAGATTTGTGAA
GTGCCAACTGCTCGTTCCTCACATTTTAGACTCTGTCGTTGCTAT
TACCGATAGGAAGGGGTCTTTTACGACACTGATGATGGATCCATA
TTCAATAAACTTGGATTATATCCAGCAACCAGAGACACGATTGAA
GCGACATGTGCAAAAGGTTTTACTACAAGAGTCAGTGAATCCGCT
ACTCCAAGGGGTCTTCCTAGAGTCTCAACAAGAGGAAGAGGAACA
ACTTGCAGCGTTCTTGCTGGACCGAGAAGTCGTAATGCCAAGGGT
TGCGCACGCTATATTTGAGTGTACCAGCTTAGGTAGGCGCCGACA
TATCCAGGGCCTCATCGACACGACAAAGACCATTATTGCACTTGC
GTTGGATACACAGCAGTTAAGTTATACTAAACGTGAACAGATTGT
GACGTACAATGCAACTTATATGAGGTCCTTAGCAAGCATGCTCAG
TTCAAGCCATAATCAGACACGTCGGTCTGTGATTGGTCACGCATC
CTTCAATATCACCGACTGCTCGGTCATCCTGGCTCAACAAGTGAG
ACGTGCAAGCTGGGCGCCCTTGCTGAATTGGCGGTCATTAGAAGG
CTTAGAAGTACCCGACCCTATTGAGTCAGTAGCCGGCTACCTGGG
TTTGGATTCTAACAACTGTTTCTTATGCTGCCACGAGCAGAACAG
CTATTCCTGGTTTTTCCTTCCCCGAATGTGCCATTTTGATGATTC
GAGACAATCTAACTCCATACAAAGGGTACCTTACATTGGTTCTAA
GACTGACGAGCGCCAGATGTCTACGATAAATCTACTTGAGAAAAC
TACCTGTCATGCTAGAGCAGCTACGAGGCTTGCATCATTATACAT
TTGGGCCTTTGGGGACTCAGACGATAGTTGGGATGCAGTCGAGAC
TCTATCTAACAGTCGATGCCAGATCACCCGAGAGCACTTGCAAGC
TCTGTGCCCTATGCCCTCTTCAGTGAATCTCCACCATCGATTGAA
CGACGGAATAACACAAGTGAAGTTTATGCCATCGACCAATAGCAG
GGTCTCTCGATTTGTTCATATTTCCAATGACCGGCAGAATTACGT
ATTGGATGACTCCGTTACTGACAGTAATCTGATTTACCAACAAGT
CATGTTGCTAGGACTTAGTATACTTGAAACATATTTCAGAGAACC
TACAGCGACGAACTTATCAAGCTTGGTGCTGCACCTTCACACTGA
TGTATCTTGTTGCTTACGAGAATGCCCGATGACACAATATGCGCC
CCCCCTGCGAGACATCCCAGAGTTAACTATAACTGCGTCAAATCC
TTTCTTATTTGATCAAGAACCTATTAGCGAAGCAGACCTGTGTAG
ACTCTCAAAGATAGCATTCCGTAGAGCAGGAGACAACTACGATGC
ATACGACCAGTTTCAACTAAGAGCAACACTGGCCGCAACTACAGG
TAAGGATGTGGCAGCAACAATCTTTGGTCCTCTAGCTGCAGTATC
AGCAAAAAATGATGCAATAGTCACAAATGACTATAGCGGGAATTG
GATTTCCGAGTTTAGGTATAGTGATCTCTATCTTTTGAGTGTTAG
CCTGGGCTACGAGATCCTTTTGATTTTCGCGTATCAACTTTACTA
CCTACGGATACGGTACCGGCAAAATATTGTATGCTATATGGAATC
TGTATTCCGACGTTGTCATTCTTTATGCTTGGGCGACTTAATCCA
GACTATTTCGCACTCTGAAATACTGGTTGGGTTAAATGCAGCGGG
CTTTAATCTTACCTTGGATCGAAGTGATTTGAAGGAGAATCAACT
CTCGCGGCTTGCTGTGAAATACCTCACATTGTGTGTCCAGACTGC
AATAAGTAATTTGGAGGTAGGATCAGAACCCCTTTGTATTATTGG
AGGTCAATTAGATGATGACATATCATTCCAAGTTGCCCACTTCCT
CTGTCGACGACTGTGCATCATCAGCTTGGTCCACTCAAATGTACA
GAACTTACCCCCGATTAGGGATAATGAAGTTGATGTAAAATCCAA
GTTGATCTACGACCATCTTAAGCTAGTTGCTACCACACTCAATAA
TAGGGACCAGTCGTATCTGCTAACACTACTGAATAAGCCTAACAT
CGAGCTTCATACTCCACAGGTTTACTTCATTATGAGGAAGTGTCT
GGGATTATTAAAAGTCTATGGACCTGTACCTCAAAAGCAACCACT
TCCAACCTCCCCAGTAGTATCACTACCAAATAAGTGCAAATCGAA
ATGGAAGCTTGAGGAGGTAATTGACAGCATTGAGTCACCTAAGTC
TTTTAACTGGGTTCCTGATACGACGATTCCATTGGATGCTGAGCA
AAATCCCCCAAATCCTAACAGGGTCATCGATAAGATAAACATTCT
AAGATCTCTGAGCCCGCGTCACTCAGTCTGGTACCGTAACCGGCA
ATACCGGTACGTGTTGAGCCAGTTAGGGCATGACCAGCTAGGTGG
TGCAACCCTATATCTTGGTGAAGGGAGCGGCTCCACAATCTTGAA
CATAGAGCCAAAAGTCAGGAGTGATAAGATATATTATCATACCTA
TTTCTCTGCAGACAAAAGCCCTGCACAGCGCAATTTCATCCCCCA
GCCAACTACTTTTCTGCGATCTAACTTTTATCACTACGAATTAGA
GCCGTCTGGATGTGAGTTCATCAATTGTTGGTCCGAGGATGTAAA
TGCAACGAATCTTACGGAACTGCGTTGCATAAATCACATATTAAC
TGTGATTCCAGTCGGATCTCTAACACGTATTATTTGTGACGTGGA
ATTGGCAAATGACACGTCAATACAATCAGTGGCCACCGCATACAT
GAACTTAAGCATTCTTGCACATGCTCTCCTCAACCAGGGAGGCGT
YTGCATCTGTAGGTGCCACCTGTTAGACACGTCCCATCTTGCGAT
TGTTTCGTTTGTACTTAAAACATTATCAAGCCAGCTGGCGATTTC
GTTTTCAGGATATTGTGGAACCAATGACCCATCTTGTGTCATAGG
AGTCACGAAAGAGTGCTCCATTAGCCTGGATGTCTTAAGTTCGAT
TGCAGCTGCATTTATAAATGAATTGCCCTCAAATGAGCTGCCGGT
CCCTCAATCATTATTAACGCTACTAGGGTGTTATCAAGACCAGTT
AGAGAGCCTTAGTGCACTCATTGAAAAAACATGGATCCAGGAGAT
CCGGAGGCCTCATTTAATGCAATGTGAGATGGAGTGGATAGGACT
TTTGGGAACAGATGCGCTGGGCGATGTCGACACATTTATCAGTTA
TCGCAATGATTCGTGTAATTCCATCCCTGATCTACTGACGCCAGC
TGTGAGTGCCCTCTTATTTGAATTAATCAGCCTGACACCTGAGCT
TCCTGGTACGGAGGACCAGCCGAGTAGGCGTGTAATCAGCATTGG
CCAAGCTTTTAACCTGACTATTTCGGGAAAAGTTAATACGATGAT
CAGATCATGCTGTGAGCAGTGCATTAAGTTATTAGCTGCCAATGT
CTCCATCCTTAGTGATGTAGATTTGTCGTATTTTGTGAGGGGGAT
TAGAGATGGTTCTTTTGCTTTGGGGTTGTTAATCACCCAGCGTCA
AATACTCAAGGCATCGAGAGCACCAAAGTACCTCAAAACGCATCA
GGTCCAGAACTGGATTGCGTTGTTACTGGAGGTCAAACTAGAGGA
AATCTTCTCTCGCCACTACCGGAAAGTGCTATTGAGAACTCTACG
GCTGTTATCCCTCTACGGGCTCCTGCAGCAGAAGGAAAGTTGATC
ATGATAGATGTCTCTCTGATTTCGCCTCAGCCCTATATACAGATA
CCTGCTACTGCTGTTGTAACTATGCAAATATAGGCTAACCTCGGT
CCGGAGTATTGAGGTAATGGGGAGACTCGTAATTGGAGAAAGAAG
AAGAACGGGGCAGATACCAGACCCCGGGATACACTACTATAAGCT
ACTTGGGTGACTCAGGACGTCACTAATAAGAGTACACTGGTTTTA
ACAATAACACTGATAGTGTAACACTGAATATTGTAAACAGTCTCA
GGGATTATTTAATTAAAAAGACATTGCGGATCATCATGTCAGGCC
TGAACCAATCAGAAACCCATCCTACAGGCATGAGTCGATTAATCC
AGTTTAATTTAGTCCAAGATGATTAGTACTCTTAGATGTTACTAA
GTATCAGGATTTAGCGTACTAGATTAAACGTCTTTGAAAGTCATT
TTACCCTGCAAGGTGCATAAGCATATGCAGGGGAATACCCACCAC
TGGTTCGTTAGTCTAATTTATTCAAAGATGCCACCAACTGAGTGC
ACTGGGAGTCTGATTGAGCTGAATCAACCTAAAGCTCAAGCTTTC
CTGTGATGTAGCATGCAGTGTGCTACTGCTGCATAGAGCCAATCA
GGGGCGTCTTGAAGGCCTCCACCTTCCTCAGGCAACGATAAAGCA
GCTCACTGCTACACCCACCATGCAAATACAAGGATATGTAGGTCC
ATCACCCTGCTTGCTAATGTGGTCGGCTCCAGGACCATGGCCATA
GCGCTATCCGTCTAATTCAATCCGCCTCATCATATGCGTATACAA
TCCTCCACTGAGAAATGCACCTGCTCAATCCAATGACAAACTACA
ACAACACGAGATCATTATGGGGTCGATTCCCTTGAATCAGAAAGG
TAAAAACGGCAGCGGAGATGGAGAATACCAGACCTAATCGGCAAA
GAACAAAAGGTTGAAGACTGATTTAAAAATCATTATAACTTTTTG
TTTAGTGGGTCGGCATGGCATCTCCACCTCCTCGCGGTCCGGACC
TGGGCATCCGAAGGAGGACGCACGTCCACTCGGATGGCTAAGGGA
GAGCCTGCAGTAGCATAACCCCTTGGGGCCTCTAAACGGGTCTTG
AGGGGTTTTTTGGTGAAAGGAGGAACTATAGGCCGGCCCTGCAGC
AATGGCAACAACGTTGCGCAAACTATTAACTGGCGAACTACTTAC
TCTAGCTTCCCGGCAACAATTAATAGACTGGATGGAGGCGGATAA
AGTTGCAGGACCACTTCTGCGCTCGGCCCTTCCGGCTGGCTGGTT
TATTGCTGATAAATCTGGAGCCGGTGAGCGTGGGTCTCGCGGTAT
CATTGCAGCACTGGGGCCAGATGGTAAGCCCTCCCGTATCGTAGT
TATCTACACGACGGGGAGTCAGGCAACTATGGATGAACGAAATAG
ACAGATCGCTGAGATAGGTGCCTCACTGATTAAGCATTGGTAACT
GTCAGACCAAGTTTACTCATATATACTTTAGATTGATTTAAAACT
TCATTTTTAATTTAAAAGGATCTAGGTGAAGATCCTTTTTGATAA
TCTCATGACCAAAATCCCTTAACGTGAGTTTTCGTTCCACTGAGC
GTCAGACCCCGTAGAAAAGATCAAAGGATCTTCTTGAGATCCTTT
TTTTCTGCGCGTAATCTGCTGCTTGCAAACAAAAAAACCACCGCT
ACCAGCGGTGGTTTGTTTGCCGGATCAAGAGCTACCAACTCTTTT
TCCGAAGGTAACTGGCTTCAGCAGAGCGCAGATACCAAATACTGT
CCTTCTAGTGTAGCCGTAGTTAGGCCACCACTTCAAGAACTCTGT
AGCACCGCCTACATACCTCGCTCTGCTAATCCTGTTACCAGTGGC
TGCTGCCAGTGGCGATAAGTCGTGTCTTACCGGGTTGGACTCAAG
ACGATAGTTACCGGATAAGGCGCAGCGGTCGGGCTGAACGGGGGG
TTCGTGCACACAGCCCAGCTTGGAGCGAACGACCTACACCGAACT
GAGATACCTACAGCGTGAGCTATGAGAAAGCGCCACGCTTCCCGA
AGGGAGAAAGGCGGACAGGTATCCGGTAAGCGGCAGGGTCGGAAC
AGGAGAGCGCACGAGGGAGCTTCCAGGGGGAAACGCCTGGTATCT
TTATAGTCCTGTCGGGTTTCGCCACCTCTGACTTGAGCGTCGATT
TTTGTGATGCTCGTCAGGGGGGCGGAGCCTATGGAAAAACGCCAG
CAACGCGGCCTTTTTACGGTTCCTGGCCTTTTGCTGGCCTTTTGC
TCACATGTTCTTTCCTGCGTTATCCCCTGATTCTGTGGATAACCG
TATTACCGCCTTTGAGTGAGCTGATACCGCTCGCCGCAGCCGAAC
GACCGAGCGCAGCGAGTCAGTGAGCGAGGAAGCGGAAGAGCGCCT
GATGCGGTATTTTCTCCTTACGCATCTGTGCGGTATTTCACACCG
CATATGGTGCACTCTCAGTACAATCTGCTCTGATGCCGCATAGTT
AAGCCAGTATACACTCCGCTATCGCTACGTGACTGGGTCATGGCT
GCGCCCCGACACCCGCCAACACCCGCTGACGCGCCCTGACGGGCT
TGTCTGCTCCCGGCATCCGCTTACAGACAAGCTGTGACCGTCTCC
GGGAGCTGCATGTGTCAGAGGTTTTCACCGTCATCACCGAAACGC
GCGAGGCAGCTGCGGTAAAGCTCATCAGCGTGGTCGTGAAGCGAT
TCACAGATGTCTGCCTGTTCATCCGCGTCCAGCTCGTTGAGTTTC
TCCAGAAGCGTTAATGTCTGGCTTCTGATAAAGCGGGCCATGTTA
AGGGCGGTTTTTTCCTGTTTGGTCACTTGATGCCTCCGTGTAAGG
GGGAATTTCTGTTCATGGGGGTAATGATACCGATGAAACGAGAGA
GGATGCTCACGATACGGGTTACTGATGATGAACATGCCCGGTTAC
TGGAACGTTGTGAGGGTAAACAACTGGCGGTATGGATGCGGCGGG
ACCAGAGAAAAATCACTCAGGGTCAATGCCAGCGCTTCGTTAATA
CAGATGTAGGTGTTCCACAGGGTAGCCAGCAGCATCCTGCGATGC
AGATCCGGAACATAATGGTGCAGGGCGCTGACTTCCGCGTTTCCA
GACTTTACGAAACACGGAAACCGAAGACCATTCATGTTGTTGCTC
AGGTCGCAGACGTTTTGCAGCAGCAGTCGCTTCACGTTCGCTCGC
GTATCGGTGATTCATTCTGCTAACCAGTAAGGCAACCCCGCCAGC
CTAGCCGGGTCCTCAACGACAGGAGCACGATCATGCGCACCCGTG
GCCAGGACCCAACGCTGCCCGAGATGCGCCGCGTGCGGCTGCTGG
AGATGGCGGACGCGATGGATATGTTCTGCCAAGGGTTGGTTTGCG
CATTCACAGTTCTCCGCAAGAATTGATTGGCTCCAATTCTTGGAG
TGGTGAATCCGTTAGCGAGGTGCCGCCGGCTTCCATTCAGGTCGA
GGTGGCCCGGCTCCATGCACCGCGACGCAACGCGGGGAGGCAGAC
AAGGTATAGGGCGGCGCCTACAATCCATGCCAACCCGTTCCATGT
GCTCGCCGAGGCGGCATAAATCGCCGTGACGATCAGCGGTCCAGT
GATCGAAGTTAGGCTGGTAAGAGCCGCGAGCGATCCTTGAAGCTG
TCCCTGATGGTCGTCATCTACCTGCCTGGACAGCATGGCCTGCAA
CGCGGGCATCCCGATGCCGCCGGAAGCGAGAAGAATCATAATGGG
GAAGGCCATCCAGCCTCGCGTCGCGAACGCCAGCAAGACGTAGCC
CAGCGCGTCGGCCGCCATGCCGGCGATAATGGCCTGCTTCTCGCC
GAAACGTTTGGTGGCGGGACCAGTGACGAAGGCTTGAGCGAGGGC
GTGCAAGATTCCGAATACCGCAAGCGACAGGCCGATCATCGTCGC
GCTCCAGCGAAAGCGGTCCTCGCCGAAAATGACCCAGAGCGCTGC
CGGCACCTGTCCTACGAGTTGCATGATAAAGAAGACAGTCATAAG
TGCGGCGACGATAGTCATGCCCCGCGCCCACCGGAAGGAGCTGAC
TGGGTTGAAGGCTCTCAAGGGCATCGGTCGACGCTCTCCCTTATG
CGACTCCTGCATTAGGAAGCAGCCCAGTAGTAGGTTGAGGCCGTT
GAGCACCGCCGCCGCAAGGAATGGTGCATGCAAGGAGATGGCGCC
CAACAGTCCCCCGGCCACGGGGCCTGCCACCATACCCACGCCGAA
ACAAGCGCTCATGAGCCCGAAGTGGCGAGCCCGATCTTCCCCATC
GGTGATGTCGGCGATATAGGCGCCAGCAACCGCACCTGTGGCGCC
GGTGATGCCGGCCACGATGCGTCCGGCGTAGAGGATCCACAGGAC
GGGTGTGGTCGCCATGATCGCGTAGTCGATAGTGGCTCCAAGTAG
CGAAGCGAGCAGGACTGGGCGGCGGCCAAAGCGGTCGGACAGTGC
TCCGAGAACGGGTGCGCATAGAAATTGCATCAACGCATATAGCGC
TAGCAGCACGCCATAGTGACTGGCGATGCTGTCGGAATGGACGAT
ATCCCGCAAGAGGCCCGGCAGTACCGGCATAACCAAGCCTATGCC
TACAGCATCCAGGGTGACGGTGCCGAGGATGACGATGAGCGCATT
GTTAGATTTCATACACGGTGCCTGACTGCGTTAGCAATTTAACTG
TGATAAACTACCGCATTAAAGCTTATCGATGATAAGCTGTCAAAC
ATGAGAATTCGGCGCGCCTAATACGACTCACTATAGGG
pAPMV3_SARS_CoV-2-S_6P_B_1_617_2_delta
(S ORF), SEQ ID NO: 5:
ACTAAACAGAAAATTAATACTTGTTAGTAGTCGCCGATTAATCCG
CTAATATATTAGGAGCGGAAGTCCTACACTCCGCCTCCGACCACG
AATTGAAATCATCATGGCTGGAATTTTTAATACTTATGAGCTCTT
TGTTAAGGATCAGACATGTATGCACAAGAGGGCTGCAAGCCTAAT
ATCGGGAGGTCAGCTTAAAAGCAATATACCAGTCTTCATCACCAC
AAAAGATGATCCAGCTGTCCGATGGGACCTTGTATGTTTTAATCT
CCGACTTGTTGTTAGTGAATCATCTACCTCGGTGATCCGGCAGGG
GGCTCTTATATCATTGCTTTCCATTACTGCTAGCAATATGAGGGC
TCTTGCAGCAATTGCTGGGCAGACTGATGAATCGATGATTAATAT
TATAGAAGTTGTGGACTTTGATGGACTTGAGCCCCAGTGTGACAC
TCGCAGTGGCTTAGACGCTCAGAAGCAGGAGATGTTTAAGGATAT
TGCCCGTGATATGCCGAGGGCACTATCCAGCGGGACCCCGTTCCA
GCACGGAAATGCGGAAGCGAATGGGCCTGAAGACACACACATGTT
CCTCCGATCCGCAATTAGCGTTCTGACACAGATCTGGATCCTGGT
TGCAAAAGCCATGACTAACATTGATGGCAGCAACGAGGCAAGCGA
TCGCCGCCTTGCCAAGTATATTCAACAGAATAGAATTGACAGGCG
ATTCATGCTTGCTCAAGCTACCAGGACTGTATGTCAGCAGGTCAT
AAAGGATTCTTTGACCTTGAGAAGGTTCCTTGTAACTGAGCTTCG
AAAGTCGCGGGGGGCATTGCATAGCGGGTCATCTTACTATGCTAT
GATTGGAGACATGCAGGCATACATATTTAATGCAGGCCTTACACC
ATTCCTCACTACCCTCAGGTACGGGATCGGGACTAAATACCATGC
CCTTGCAATTAGCTCACTTGCAGGTGACCTGAATAAGATTAAGGG
GTTACTGACTCTATACAAAGAGAAAGGGAGTGATGCGGGCTATAT
GGCACTTCTAGAGGATGCTGACTGCATGCAATTTGCGCCAGGTAA
TTACTCACTATTATATTCATATGCGATGGGAGTAGCAAGTGTACA
TGATGAAGGCATGAGGAATTACCAATACGCCCGGCGCTTCCTCCA
TAAGGGGATGTACCAATTTGGACGTGATATCGCAACACAACAGCA
ACATGCACTTGATGAATCCCTCGCCCAGGAGATGAGAATCACAGA
GGCTGACAGGGCAAATCTCAAGGTAATGATGGCAAACATTGGGGA
AGCATCAAGCTTCAGTGATATGCCTAATCCAGGAGCTAGCGGCAT
TCCTGCCTTCAATGACCGCCCTGATGAGATCTTCTCTGAGCCAGG
CTATCGCAAGCCCCCTCAAGATCAGCCGCTGATAAAGCTCCCAGA
TCTCGAGGAAGAAGAGCAGGACGAATTCGACATGTAGAGAGCCGC
CCACCACCCAGAGCCAGCGACAAACCCAGGCAAAACAACCCACCA
TCTCCGACCCGGAACACAACGCAACATCGAGGACCCCGGGCAGCC
GACACCGCAGCCGCAAGACCTCCGCATCATGAACGACCAGCATCC
AAACATCTGTCCTCCCTATTCTATCAGTTTAATAAAAAAGGCTTT
ACGTGCACAGACAGAGATCCTGACATAATTCAGCCGGGGGGATTC
AACTTAGGAGCGGAAGTATCCCTCCTAACCTGCACCTGCATTTCA
ATATGGATCTTGAGTTTAGTAGTGAGGAAGCAGTCGCAGCCCTCT
TGGATGTGAGTTCCTCAACCATAACCGAGGTTCTGAGCAAGCAAA
GTATTCCTGACCCTAGCTTTCTCACGTCCACGGAGGCATCCGGTG
AGCAAGTTCCAGATTCGAAGGTCGCCAATCACAAAGCCAGTGTCG
ATAAGACCCCAGATCAAGGCCAACCATCGGCAACGCCTTCGGCTC
CTCCTGAGACTGCCGAAAACATCAATACACCATCTTGCGAGGACG
GTTTACCCCCTAACTTCTTCATCCCAAGGGTAGAGAGTTACCACA
CTAACCTTTTTAAAGGGGGCCCCCAATTGGACTCAACGGAGCGGC
CGCCTGGTCACCAAATAGTTTGCGGAGATCAAGACGCGGACCTCT
CCCGAGCCGGGATAGCGAGAAAGAAGAAGAAGCAGAAGCATTGCA
AAGCGCTCAACTTTTCGGAATCCCCACTCACAGACAATCAGGCCA
TAGAAGGGAGTATTCAATCTCATGGAGCCCTGGATCAGGAACCTT
CCAGACAGAGACCTGGTGCAACCCAGCCTGCTCTCCAGTCACCGC
CTTCCCAAAACAATACAAGTGTGCATGCAGACAGTGCCCAAGATT
CTGCGATCTCTGTTTCAATCCCGCTGACTATGGTGGAATCATTGA
TCTCTCAAGTGTCAAAGTTGTCGGACCAAGTCTCGCAGATACAGA
AGTTGGTGAGTACCCTTCCTCAAATTAAGACTGATATTGCTTCGA
TTCGGAACATGCAAGCAGCTTTGGAAGGACAGCTCAGTATGATTC
GTATATTGGATCCAGGAAACTGTTCGGAATCATCACTCAATGCGC
TGAGAGGGTTCAGTGGTAAGGCACCTGTTATTGTGAGTGGTCCAG
GAAATCCCAATCAATGCATCAGCCAAGGCTCATCCACCACAATTT
GCCTGGATGAGTTAGCCCGGCCTGTGCCAAATCCTATACAGGTGA
AACTTCAAGATGATCAGAAGGACCTATCTGCACAGCGACATGCTG
TCATTGCATTACTCGAAACCAGAATCCCCCCGGGACCAAAACGTG
ACAAATTACTGTCATTGGTTGCAATTGCAAAGACTGCAAGTGATT
TAATCAAGATTAAGAGAATGGCAGTCCTCGGCCAGTGATATCACC
CTATGGCAACCAGGCTACAAATGTAAGCAGTTTCCGATATGTACC
GAATCAATAACACGACCCCTTCCGCAAAAGCAAGCGCGAGCATGA
CACACTCAGCTTCAGCCAATTCTAGCTTCACTAAGCAGGTGATCT
GACCATCTGGCTTTACTTCCGCTTTATAAAAAACTCCTAGATCTA
ACATGGAGGCAGACCGAGCTAATGCGGAGGGAGTACACAAGGGGA
ATCAAAGGAGCGGAAGGTACTAAGTCACTCCACCGCCTGCCCTCG
CCCATCCGCAGCCTCCCCCAACCCGCGGCGCCGCCACCATGTTCG
TGTTTCTGGTGCTGCTGCCTCTGGTGAGCTCCCAGTGCGTGAACC
TGAGGACAAGGACCCAGCTGCCCCCTGCCTATACCAATTCCTTCA
CACGGGGCGTGTACTATCCCGACAAGGTGTTTAGATCTAGCGTGC
TGCACTCCACACAGGATCTGTTTCTGCCTTTCTTTTCTAACGTGA
CCTGGTTCCACGCCATCCACGTGAGCGGCACCAATGGCACAAAGC
GGTTCGACAATCCAGTGCTGCCCTTTAACGATGGCGTGTACTTCG
CCTCCACCGAGAAGTCTAACATCATCAGAGGCTGGATCTTTGGCA
CCACACTGGACAGCAAGACACAGTCCCTGCTGATCGTGAACAATG
CCACCAACGTGGTCATCAAGGTGTGCGAGTTCCAGTTTTGTAATG
ATCCATTCCTGGGCGTGTACTATCACAAGAACAATAAGTCTTGGA
TGGAGAGCGGCGTGTATTCCTCTGCCAACAATTGCACATTTGAGT
ACGTGTCCCAGCCCTTCCTGATGGACCTGGAGGGCAAGCAGGGCA
ATTTCAAGAACCTGAGGGAGTTCGTGTTTAAGAATATCGATGGCT
ACTTCAAGATCTACTCCAAGCACACCCCAATCAACCTGGTGCGCG
ACCTGCCACAGGGCTTCTCTGCCCTGGAGCCACTGGTGGATCTGC
CCATCGGCATCAACATCACCCGGTTTCAGACACTGCTGGCCCTGC
ACAGAAGCTACCTGACACCAGGCGACAGCTCCTCTGGATGGACCG
CAGGAGCTGCCGCCTACTATGTGGGCTATCTGCAGCCCAGGACCT
TCCTGCTGAAGTACAACGAGAATGGCACCATCACAGACGCCGTGG
ATTGCGCCCTGGATCCCCTGTCTGAGACCAAGTGTACACTGAAGA
GCTTTACCGTGGAGAAGGGCATCTATCAGACAAGCAATTTCAGGG
TGCAGCCTACCGAGTCCATCGTGCGCTTTCCCAATATCACAAACC
TGTGCCCTTTTGGCGAGGTGTTCAACGCAACCCGCTTCGCCAGCG
TGTACGCCTGGAATAGGAAGCGCATCTCCAACTGCGTGGCCGACT
ATTCTGTGCTGTACAACAGCGCCTCCTTCTCTACCTTTAAGTGCT
ATGGCGTGAGCCCCACAAAGCTGAATGACCTGTGCTTTACCAACG
TGTACGCCGATTCCTTCGTGATCAGGGGCGACGAGGTGCGCCAGA
TCGCCCCTGGCCAGACAGGCAAGATCGCCGACTACAATTATAAGC
TGCCTGACGATTTCACCGGCTGCGTGATCGCCTGGAACTCTAACA
ATCTGGATAGCAAAGTGGGCGGCAACTACAATTATCGGTACCGGC
TGTTTAGAAAGTCTAATCTGAAGCCATTCGAGAGGGACATCTCCA
CAGAGATCTACCAGGCCGGCTCTAAGCCCTGCAATGGCGTGGAGG
GCTTTAACTGTTATTTCCCTCTGCAGAGCTACGGCTTCCAGCCAA
CAAACGGCGTGGGCTATCAGCCCTACCGCGTGGTGGTGCTGTCTT
TTGAGCTGCTGCACGCACCTGCAACAGTGTGCGGACCAAAGAAGA
GCACCAATCTGGTGAAGAACAAGTGCGTGAACTTCAACTTCAACG
GACTGACCGGCACAGGCGTGCTGACCGAGTCCAACAAGAAGTTCC
TGCCTTTTCAGCAGTTCGGCAGGGACATCGCAGATACCACAGACG
CCGTGCGCGACCCTCAGACCCTGGAGATCCTGGATATCACACCAT
GCTCCTTCGGCGGCGTGTCTGTGATCACACCAGGCACCAATACAA
GCAACCAGGTGGCCGTGCTGTATCAGGGCGTGAATTGTACCGAGG
TGCCCGTGGCAATCCACGCAGATCAGCTGACCCCTACATGGCGGG
TGTACTCTACCGGCAGCAACGTGTTCCAGACAAGAGCCGGATGCC
TGATCGGAGCCGAGCACGTGAACAATAGCTATGAGTGCGACATCC
CTATCGGCGCCGGCATCTGTGCCTCCTACCAGACCCAGACAAACT
CCAGAGGGTCTGCCTCCTCTGTGGCCAGCCAGTCCATCATCGCCT
ATACCATGAGCCTGGGCGCCGAGAATTCCGTGGCCTACTCCAACA
ATTCTATCGCCATCCCTACCAACTTCACAATCTCCGTGACCACAG
AGATCCTGCCAGTGAGCATGACCAAGACATCCGTGGACTGCACAA
TGTATATCTGTGGCGATTCCACCGAGTGCTCTAACCTGCTGCTGC
AGTACGGCTCTTTTTGTACCCAGCTGAATAGAGCCCTGACAGGCA
TCGCCGTGGAGCAGGACAAGAACACACAGGAGGTGTTCGCCCAGG
TGAAGCAGATCTACAAGACCCCACCCATCAAGGACTTTGGCGGCT
TCAACTTCAGCCAGATCCTGCCCGATCCTAGCAAGCCATCCAAGC
GGTCTCCTATCGAGGACCTGCTGTTCAACAAGGTGACCCTGGCCG
ATGCCGGCTTCATCAAGCAGTATGGCGATTGCCTGGGCGACATCG
CCGCCAGAGACCTGATCTGTGCCCAGAAGTTTAATGGCCTGACCG
TGCTGCCTCCACTGCTGACAGATGAGATGATCGCCCAGTACACAT
CTGCCCTGCTGGCCGGCACCATCACAAGCGGATGGACCTTCGGCG
CAGGACCCGCCCTGCAGATCCCCTTTCCCATGCAGATGGCCTATC
GGTTCAACGGCATCGGCGTGACCCAGAATGTGCTGTACGAGAACC
AGAAGCTGATCGCCAATCAGTTTAACTCCGCCATCGGCAAGATCC
AGGACTCTCTGAGCTCCACACCCAGCGCCCTGGGCAAGCTGCAGA
ACGTGGTGAATCAGAACGCCCAGGCCCTGAATACCCTGGTGAAGC
AGCTGTCTAGCAACTTCGGCGCCATCTCCTCTGTGCTGAATGATA
TCCTGAGCAGGCTGGACCCTCCAGAGGCAGAGGTGCAGATCGACC
GGCTGATCACAGGCAGACTGCAGTCCCTGCAGACCTACGTGACAC
AGCAGCTGATCAGGGCAGCAGAGATCAGGGCCTCTGCCAATCTGG
CCGCCACCAAGATGAGCGAGTGCGTGCTGGGCCAGTCCAAGAGAG
TGGACTTTTGTGGCAAGGGCTATCACCTGATGAGCTTCCCACAGT
CCGCCCCTCACGGAGTGGTGTTTCTGCACGTGACCTACGTGCCAG
CCCAGGAGAAGAACTTCACCACAGCACCAGCAATCTGCCACGATG
GCAAGGCACACTTTCCTAGGGAGGGCGTGTTCGTGAGCAACGGCA
CCCACTGGTTTGTGACACAGCGCAATTTCTACGAGCCACAGATCA
TCACCACAGACAATACATTCGTGTCCGGCAACTGTGACGTGGTCA
TCGGCATCGTGAACAATACCGTGTATGATCCTCTGCAGCCAGAGC
TGGACTCTTTTAAGGAGGAGCTGGATAAGTACTTCAAGAATCACA
CCAGCCCCGACGTGGATCTGGGCGACATCTCTGGCATCAATGCCA
GCGTGGTGAACATCCAGAAGGAGATCGACAGGCTGAACGAGGTGG
CCAAGAATCTGAACGAGTCCCTGATCGATCTGCAGGAGCTGGGCA
AGTATGAGCAGTACATCAAGTGGCCCTGGTATATCTGGCTGGGCT
TCATCGCCGGCCTGATCGCCATCGTGATGGTGACCATCATGCTGT
GCTGTATGACAAGCTGCTGTTCCTGCCTGAAGGGCTGCTGTTCTT
GTGGCAGCTGCTGTAAGTTTGATGAGGACGATAGCGAGCCTGTGC
TGAAGGGCGTGAAGCTGCACTACACCTGATAGTTTATAAAAAACA
TTGTGAGAATGCAATTTAAGGACGAAAGTAGGAGCGGAAGCCGCG
GCCCAGGCTCCATTCAAAAACCATGGCCGCTGCTCTCGGGAACAT
GCACCCGTCATCGTCGATTACTCTTATGCATGATGACCCGTCCAT
CCAGACGCAGTTGCTAGCCTTCCCTCTGATAAGTGAGAAGACAGA
GACAGGGACGACAAAGCTGCAGCCTCAAGTGAGAATGCAATCTTT
CCTATCAACCGATAGCCAGAAGTATCATCTGGTCTTTATCAACAC
ATATGGTTTCATTGCGGAGGATTTTAATTGTAGTCCAGCAAACGG
ATTTGTGCCTGCACTATTTCAACCCAAGTCTAAAGTCTTATCGTC
AGCGATGGTTACACTTGGGGCTGTCCCTGCTGACACTGTTCTCCA
GGACCTACAACGGGACTTAATCGCAATGAGATTTAAAGTCCGAAA
GAGTGCGTCAGCGAAGGAGCTAATATTATTCTCCACAGACAATGT
CCCTGCAACATTAACAGGGTCATCAGTCTGGAAGAATAAAGGAGT
AATAGCTGATACAGCAACTGCAGTTAAAGCGCCCGGTCGCATCTC
ATGCGATGCGGTATGCAGTTATTGCATTACATTCATCTCTTTTTG
CTTCTTCCACTCATCTGCTCTTTTTAAGGTGCCCAAGCCTCTGCT
GAATTTCGAGACGGCCATAGCATATTCCCTTGTACTCCAGGTCGA
GCTTGAGTTCCCAAATATCAAAGATACCTTACACGAGAAGTATCT
CAAAGTTAAAGACTCGAAGTGGTATTGTACAATTGACATCCATAT
TGGGAATCTTTTAAAGAGAACCGCCAAGCAACGAAGGAGGACACC
TTCAGAGATAATGCAGAAAGTCCGTAAGATGGGTTTCAGGATTGG
GTTATACGATTTGTGGGGTCCAACAATTGTGGTTGAATTAACTGG
GTCCTCAAGTAAGTCCCTACACGGATTCTTCTCGAGTGAGCGGCT
GGCCTGCCACCCGGTATCACAGTATAATCCTCATGTTGGACAGCT
TATCTGGGCCCACGATGTATCCATCACGGGGTGCCACATGATTAT
ATCTGAACTTGAGAAAAAGAAGGCACTTGCAATGGCTGACCTGAC
CGTGAGTGATGCAGTAGCTGTCAACACGAGCATCAAGAGCTTATC
GCCTTTCAGACTGTTCAAAAAATATTAGGGCTAGACAACTCCTAG
GGTGTGTGCTTATAATTTGCATATTCTAGATTATAATCATTAACC
GCGTCCAAATGATCAGACCGCATAATTAACAGCTCAGCCAGGACT
CGCAAATCCTGCATAGGCTCAAATAACAAGACGACATATATACTC
ACACAACAAGGTGTAATATCTAGAAGGAATCAGAATTGAGACAAT
ATCTAGGTTATCAGCGCTACTGCCCTGCAACTCACTATGTATGCT
TGGCATTAATAAAAAACATTGTGAGAATGCAATTTAAGGACGAAA
GTAGGAGCGGAACACGCTCAACTGAACCCAAGCGCACACCAGCCC
CCCGGCACCGCACCGGAAGGACGCACAACAGCCGACCGCCCCCAC
CAGGGCAGAGGCCATGCAACCAGGCTCGGCACTCCACCTCCCGCA
CCTGTACATAATAATTGCCTTAGTCAGCGATGGCACTTTAGGCCA
AACAGCTAAAATTGATAGGCTGATACAGGCAGGGATTGTCTTGGG
TAGTGGAAAGGAGCTCCATATCTCACAAGATTCAGGGACACTGGA
TTTATTCGTGAGGTTGTTACCAGTACTCCCTTCGAATTTGTCCCA
TTGCCAGCTTGAAGCAATAACACAGTATAATAAGACGGTGACTAG
GTTATTGGCCCCAATAGGGAAGAACCTAGAACAGGTACTACAAGC
GAGACCGCGTGGGAGGCTGTTCGGAGCAATAATAGGGTCCATCGC
GCTTGGTGTTGCCACATCAGCACAGATTACCGCTGCAATTGCACT
CGTCCGTGCACAGCAAAATGCTAATGATATATTAGCACTTAAAAA
CGCTCTCCAATCCAGCAATGAGGCAATCCGTCAACTCACGTATGG
CCAGGACAAACAGTTACTTGCAATTTCTAAAATACAAAAAGCTGT
GAATGAGCAGATCCTCCCTGCACTCGATCAGCTGGACTGTGCTGT
CCTTGGAACCAAACTAGCTGTGCAGCTTAATCTGTATCTCATTGA
GATGACTACCATTTTCGGCGAGCAAATCAATAATCCAGTCTTGGC
GACCATCCCATTAAGTTACATTTTACGCCTGACCGGTGCGGAGTT
GAACAACGTCCTCATGAAGCAAGCCCGATCGTCCTTAAGCCTTGT
ACAGTTAGTTTCAAAAGGCTTACTTAGTGGACAAGTAATTGGCTA
TGACCCATCTGTTCAAGGTCTGATAATAAGGGTGAATCTGATGCG
AACGCAGAAGATCGATAGAGCACTAGTTTATCAGCCGTATGTATT
ACCAATCACTCTTAACTCTAACATAGTAACACCAATCGCCCCAGA
ATGCGTAATTCAGAAAGGGACGATCATAGAGGGTATGTCACGGAA
GGATTGTACAGAGTTGGAGCAAGACATAATTTGTAGGACAGTTAC
GACATACACGCTTGCCAGAGACACCAGATTGTGTTTACAAGGCAA
TATCTCTTCTTGTAGGTATCAACAGTCAGGCACCCAACTACACAC
CCCATTTATTACGTACAACGGGGCAGTTATCGCAAACTGTGATTT
AGTCTCTTGTCGGTGTCTCCGTCCTCCTATGATTATCACCCAAGT
AAAAGGGTACCCTCTTACCATAATCACAAGGTCCGTATGTCAAGA
ATTATCCGTTGACAACTTAGTCCTGAATATTGAGACACACCATAA
TTTCTCCCTTAATCCAACAATTATCGACCCTCTAACAAGAGTAAT
AGCCACTACACCGTTAGAGATAGATTCCTTAATCCAAGAGGCACA
AGATCATGCGAATGCAGCGCTAGCTAAAGTGGAAGAGAGTGATAA
ATATCTCAGGGCAGTTACTGGCGGTAATTATTCGAATTGGTATAT
CGTTCTTGTCATCGTCTTACTATTTGGGAATTTGGGCTGGTCGCT
GTTGTTAACAGTACTTCTATGTAGGTCACGGAAACAACAGCGACG
TTATCAACAAGATGATTCCGTCGGGTCAGAAAGAGGTGTTGGGGT
TGGGACCATCCAGTATATGTCATAAGTGGTGTCAGACATCACAAT
CCAAGTCACTCAGTTATCTACTCCGCTGGCTCGGAGCCATCGACA
ACGGCGTGAGCCCTCATCCGGGGACAATCCACAACCTGATACTAC
CAGACAGTGGCTCCGCACGATGGCAGCAGCCCACAACGTCCTGCA
TGTAAATATCATTGCGTTACTTTAATTAAAAAACCTCAAGGTGAC
CCATCCTGGCCCATCCGATTGGAGGAGCGGAAGGTTTTAAGGTAA
TCAGGCAAATTGTTTCCAACATGGAATCCCCTCCTTCTGGCAAGG
ATGCGCCAGCCTTCCGCGAGCCTAAGCGAACTTGCAGACTCTGTT
ACAGGGCGACGACTCTCTCCCTTAATCTCACCATCGTTGTATTAT
CAATTATAAGCATTTATGTATCAACTCAAACTGGGGCAAACAACT
CTTGTGTCAATCCCACAATCGTAACTCCTGATTATTTAACTGGCA
GCACGACAGGCTCAGTTGAAGATCTTGCTGACTTAGAGAGTCAAC
TCCGGGAGATACGTAGAGACACAGGGATTAATCTCCCAGTTCAGA
TCGATAATACAGAAAATCTAATACTGACAACTCTGGCGAGTATCA
ACTCGAATTTACGCTTTCTGCAAAATGCAACAACTGAGAGCCAGA
CCTGCCTATCTCCTGTCAATGACCCTCGCTTTGTAGCTGGCATAA
ATAGGATCCCAGCAGGGTCGATGGCATATAATGACTTCAGCAACC
TCATTGAGCACGTAAATTTCATACCGTCGCCGACAACGTTGTCAG
GCTGCACCAGGATACCATCATTTTCACTCTCTAAGACGCACTGGT
GTTATACCCACAATGTTATATCAAACGGGTGCCTTGACCATGCTG
CAAGTTCACAATACATCTCAATAGGGATCGTTGATACCGGCCTAA
ATAATGAGCCCTATTTTAGAACAATGTCTTCAAAGTCACTGAATG
ACGGATTAAATAGAAAGAGTTGCTCTGTGACCGCAGCCGCTAATG
CATGTTGGTTACTCTGTAGCGTAGTAACAGAATACGAAGCTGCGG
ACTATCGATCACGAACTCCTACTGCTATGGTTCTGGGGCGATTCG
ATTTTAATGGTGAATACACAGAAATAGCTGTCCCCTCCTCACTAT
TCGATGGCCGGTTTGCATCTAACTATCCAGGTGTGGGATCGGGAA
CTCAAGTCAATGGAACATTATATTTCCCTTTATATGGAGGGGTCC
TTAATGGGTCAGATATAGAAACGGCAAACAAGGGGAAATCCTTCA
GGCCTCAGAATCCTAAGAATAGGTGTCCAGACTCGGAGGCGATCC
AAAGCTTTAGAGCGCAAGATAGCTATTACCCGACGAGATTTGGGA
AGGTGCTGATCCAACAGGCAATCATTGCATGTAGGATCAGTAACA
AAAGTTGTACTGATTTCTATCTCCTGTACTTCGACAACAACAGAG
TGATGATGGGTGCAGAGGCACGGCTATATTATCTTAATAACCAAT
TATATCTATATCAACGCTCGTCGAGCTGGTGGCCGCATCCTTTGT
TCTATAGCATCTCATTACCGTCATGTCAAGCCCTTGCTGTCTGTC
AAATTACAGAGGCCCATCTGACACTGACTTACGCCACGTCGAGGC
CAGGAATGTCTATCTGTACAGGAGCATCAAGGTGTCCAAATAATT
GCGTAGACGGTGTATATACTGATGTATGGCCATTGACAAAGAACG
ATGCTCAGGACCCCAATCTCTTCTACACTGTATACTTAAATAATT
CAACTCGGCGGATTAGCCCAACCATAAGTCTATACACTTATGACC
GCAGGATCAAATCTAAGCTGGCAGTGGGTAGTGATATAGGAGCAG
CTTATACCACGAGTACCTGTTTCGGCCGATCAGATACAGGGGCGG
TTTATTGCTTAACTATAATGGAGACCGTTAACACAATATTTGGTC
AGTACCGGATCGTGCCAATATTACTTAGAGTGACAAGCCGGTAAT
GGATTGTTCCTAGTGAAGTGGGGCCTATGGTTACTCCGTAACGGA
GATGACCTGATTTATGCTATGTAACTAATTTTGCTCAATCGGGAG
GGTGATCGGCCGGGTATGTCCACAATGACAATGACTCCTATTCCC
TTGCTAGAGCTAGCTTCCTCTTTAATCCAATACAATCCCTACCTG
TAGTTTAATAAAAAAATCCTCTATATAAGATCCAGACGCGTGCTA
TATAAGCTAATAGCTTAGGATGGAAACGACAGGAGCGGAACATAT
CCCTCCTACCATGTCTTCTCATAATATTATCCTCCCTGACCACCA
CTTAAATTCCCCTATTGTACTAAATAAATTGATGTACTATTGTAA
ACTCCTTAATATATTGCCTGACCCTAAGTCGCCATGGTACGAGAA
GATTAAGTCTTGGACTAATTGCTGCATCCGTGTGTCAGACAGCAA
TCGCATGACTTTGTCACGCGCCTCCAAGCTACGAGAACTATTGGC
AACGTACGGCGTATATTCGAAGAACCACCAGCAATGTTATACCAC
CATAATCCATCCCCAATCCCTATCACCCATCATGCAGACTGTCTC
TCAACTGGGACGCTCAATCCCGACCTGGGCCAGGATCCGAAAGGA
GATCACACACAGTATCATAGCCCAAACGAATAAATTTGAATCCTT
ATTCCACAACATCTCAAAGGACCTGACAGGGAAAACCAACTTGTT
TGGCGGATTCTGTGATTTACATGGGAGCATAAGCACTGCTGCAAA
GCGCTTAATGAATCAACCGGGTCTCTACTTGGATTCACCTGATGC
CCATGCATGTCAATTTTTGTTCCAGCTTAAAACCTGTCAACGGGA
ATTGATTCTACTTATGAGGCAGAATGCCACAACAGAGCTGATTAG
GGTTTTTGACTATCCTGAGCTACGGGTTCTTGTGACACCTGAATA
TTCGGTCTGGGTTTTTACACGGACCAAACAAGTTACATTACTAAC
ATTCGATTGCTTGTTAATGTACTGCGATCTTTCAGACGGCCGGCA
CAACATCCTATTCACTTGTAAGCTTTTGTCTCACTTAAACCACCT
CGGGTGTCGGATAAGGGACCTGCTGGTGCTAATTGATTCACTTGC
CGAGAAACACCCCTTAATAGTTTATGATGTAGTTGCAAGTCTGGA
GTCGTTATCATATGGTGCTATACAGCTACATGACAAGGTAGCTGG
TTATGCTGGAACATTTTTCTCATTTATCCTTGCAGAAATACAGGA
TTCCTTAGAAACAGTGTTGGATCAAGGTAATAGTGAATCTATCAT
ATCCCAAATAAGGAACATATACTCAGGTCTCACAGTAAACGAGGC
TGCAGAATTGCTATGTGTTATGAGGCTTTGGGGCCACCCTGCCCT
AAACAGTGTAGACGCTGCAAGCAAGGTGAGACAGAGTATGTGCGC
TGGCAAGCTGCTTAAATTTGATACGATCCAGTTAGTCCTTGCATT
CTTCAATACACTAATAATTAATGGATATAGGAGAAAGCACCATGG
GCGATGGCCGAATGTTTGTAGTGACTCTATAATTGGGAATGAACT
CAAGCGGATGTATTTTGATCAATGTGAGATCCCCCACGACTTCAC
ATTAAAGAACTACCGGGAATTAAGTCTTCTTGAATTCGAGTGCAC
ATTTCCCATAGAATTATCAGATAAACTGAACATATTTTTAAAAGA
TAAGGCAATAGCATTCCCGAAGTCCAGGTGGACATCTCCTTTCAA
AGCAGATATCACACCGCGCCATTTGCTGCAAGCGCCTGAGTTTAA
AACCCGTGCCAACAGACTACTGCTATCATTCTTACAACTCGAGGA
ATTCTCCATCGAATCAGAACTAGAATATGTGACTACCCTGGCTTA
CCTGGATGATGATGAGTTCAACGTATCTTACTCCCTCAAAGAGAA
GGAGGTAAAGACAGATGGTCGAATCTTCGCTAAGCTCACAAGGAA
AATGCGGAGCTGTCAGGTGATATTCGAAGAATTGCTAGCAGAGCA
TGTCTCTCCATTATTTAAAGACAACGGCGTTACTATGGCCGAATT
GTCACTTACTAAGAGCCTACTCGCTATTAGCAACCTAAGCTCCAC
CTTATTTGAGACGCAAACAAGGCAAGGCGATCGGAATGCAAGATT
CACGCATGCGCACTTCATAACAACTGACCTGCAAAAGTACTGTTT
AAACTGGAGGTACCAGAGTGTAAAACTGTTCGCACGTCAATTGAA
CCGGTTATTTGGTCTGCAGCATGGGTTTGAATGGATCCATTGCAT
ACTAATGAAGTCCACTATGTATGTCGCAGACCCCTTTAACCCTCC
ACATAGCAATGCACGTGAGGCACTCGATGACAACTTAAATAGCGA
CATATTCATTGTATCACCTAGGGGGGCCATCGAGGGACTATGCCA
AAAAATGTGGACAATCATCTCAATCTCGGCGATCCATGCATCCGC
TGCTGTGGCTGGGCTACGGGTGGCATCTATGGTCCAAGGTGATAA
CCAAGTTATTGGGGTAACACGCGAGTTCCTAGCTGGGCATGATCA
AACTTTTGTAGACGATCAATTGACGGTTTCACTTGAGAACTTCAC
TCAGATATTCAAGGAAGTCAACTACGGGCTAGGTCATAATCTCAA
GCTGAGGGAAACAATCAAGTCGAGCCACATGTTCATCTACTCAAA
GAGGATCTTCTACGACGGCCGAGTGCTTCCACAACTTCTAAAGAA
CATAAGTAAGTTAACCCTATCAGCTACAACTACAGGGGAGAACTG
TTTAACGAGTTGCGGGGACCTATCATCATGCATCACACGATGTAT
AGAGAATGGGTTCCCTAAAGATGCAGCATTCGTTTTGAATCAATT
AGTGATTAGGATCCAGATACTTGCCGATCACTTTTATTCCATCCT
TGGAGGATGTTTCTCTGGGTTGAACCAGGCGGATATCCGTCTCCT
TCTATATGAGGGTGCAATTTTACCGGCACAATTAGGAGGGTTCAA
CAACCTCAACATGTCACGGCTATTCTGTCGGAATATTGGTGACCC
ATTAGTCGCGTCCATCGCAGACACCAAGAGATTTGTGAAGTGCCA
ACTGCTCGTTCCTCACATTTTAGACTCTGTCGTTGCTATTACCGA
TAGGAAGGGGTCTTTTACGACACTGATGATGGATCCATATTCAAT
AAACTTGGATTATATCCAGCAACCAGAGACACGATTGAAGCGACA
TGTGCAAAAGGTTTTACTACAAGAGTCAGTGAATCCGCTACTCCA
AGGGGTCTTCCTAGAGTCTCAACAAGAGGAAGAGGAACAACTTGC
AGCGTTCTTGCTGGACCGAGAAGTCGTAATGCCAAGGGTTGCGCA
CGCTATATTTGAGTGTACCAGCTTAGGTAGGCGCCGACATATCCA
GGGCCTCATCGACACGACAAAGACCATTATTGCACTTGCGTTGGA
TACACAGCAGTTAAGTTATACTAAACGTGAACAGATTGTGACGTA
CAATGCAACTTATATGAGGTCCTTAGCAAGCATGCTCAGTTCAAG
CCATAATCAGACACGTCGGTCTGTGATTGGTCACGCATCCTTCAA
TATCACCGACTGCTCGGTCATCCTGGCTCAACAAGTGAGACGTGC
AAGCTGGGCGCCCTTGCTGAATTGGCGGTCATTAGAAGGCTTAGA
AGTACCCGACCCTATTGAGTCAGTAGCCGGCTACCTGGGTTTGGA
TTCTAACAACTGTTTCTTATGCTGCCACGAGCAGAACAGCTATTC
CTGGTTTTTCCTTCCCCGAATGTGCCATTTTGATGATTCGAGACA
ATCTAACTCCATACAAAGGGTACCTTACATTGGTTCTAAGACTGA
CGAGCGCCAGATGTCTACGATAAATCTACTTGAGAAAACTACCTG
TCATGCTAGAGCAGCTACGAGGCTTGCATCATTATACATTTGGGC
CTTTGGGGACTCAGACGATAGTTGGGATGCAGTCGAGACTCTATC
TAACAGTCGATGCCAGATCACCCGAGAGCACTTGCAAGCTCTGTG
CCCTATGCCCTCTTCAGTGAATCTCCACCATCGATTGAACGACGG
AATAACACAAGTGAAGTTTATGCCATCGACCAATAGCAGGGTCTC
TCGATTTGTTCATATTTCCAATGACCGGCAGAATTACGTATTGGA
TGACTCCGTTACTGACAGTAATCTGATTTACCAACAAGTCATGTT
GCTAGGACTTAGTATACTTGAAACATATTTCAGAGAACCTACAGC
GACGAACTTATCAAGCTTGGTGCTGCACCTTCACACTGATGTATC
TTGTTGCTTACGAGAATGCCCGATGACACAATATGCGCCCCCCCT
GCGAGACATCCCAGAGTTAACTATAACTGCGTCAAATCCTTTCTT
ATTTGATCAAGAACCTATTAGCGAAGCAGACCTGTGTAGACTCTC
AAAGATAGCATTCCGTAGAGCAGGAGACAACTACGATGCATACGA
CCAGTTTCAACTAAGAGCAACACTGGCCGCAACTACAGGTAAGGA
TGTGGCAGCAACAATCTTTGGTCCTCTAGCTGCAGTATCAGCAAA
AAATGATGCAATAGTCACAAATGACTATAGCGGGAATTGGATTTC
CGAGTTTAGGTATAGTGATCTCTATCTTTTGAGTGTTAGCCTGGG
CTACGAGATCCTTTTGATTTTCGCGTATCAACTTTACTACCTACG
GATACGGTACCGGCAAAATATTGTATGCTATATGGAATCTGTATT
CCGACGTTGTCATTCTTTATGCTTGGGCGACTTAATCCAGACTAT
TTCGCACTCTGAAATACTGGTTGGGTTAAATGCAGCGGGCTTTAA
TCTTACCTTGGATCGAAGTGATTTGAAGGAGAATCAACTCTCGCG
GCTTGCTGTGAAATACCTCACATTGTGTGTCCAGACTGCAATAAG
TAATTTGGAGGTAGGATCAGAACCCCTTTGTATTATTGGAGGTCA
ATTAGATGATGACATATCATTCCAAGTTGCCCACTTCCTCTGTCG
ACGACTGTGCATCATCAGCTTGGTCCACTCAAATGTACAGAACTT
ACCCCCGATTAGGGATAATGAAGTTGATGTAAAATCCAAGTTGAT
CTACGACCATCTTAAGCTAGTTGCTACCACACTCAATAATAGGGA
CCAGTCGTATCTGCTAACACTACTGAATAAGCCTAACATCGAGCT
TCATACTCCACAGGTTTACTTCATTATGAGGAAGTGTCTGGGATT
ATTAAAAGTCTATGGACCTGTACCTCAAAAGCAACCACTTCCAAC
CTCCCCAGTAGTATCACTACCAAATAAGTGCAAATCGAAATGGAA
GCTTGAGGAGGTAATTGACAGCATTGAGTCACCTAAGTCTTTTAA
CTGGGTTCCTGATACGACGATTCCATTGGATGCTGAGCAAAATCC
CCCAAATCCTAACAGGGTCATCGATAAGATAAACATTCTAAGATC
TCTGAGCCCGCGTCACTCAGTCTGGTACCGTAACCGGCAATACCG
GTACGTGTTGAGCCAGTTAGGGCATGACCAGCTAGGTGGTGCAAC
CCTATATCTTGGTGAAGGGAGCGGCTCCACAATCTTGAACATAGA
GCCAAAAGTCAGGAGTGATAAGATATATTATCATACCTATTTCTC
TGCAGACAAAAGCCCTGCACAGCGCAATTTCATCCCCCAGCCAAC
TACTTTTCTGCGATCTAACTTTTATCACTACGAATTAGAGCCGTC
TGGATGTGAGTTCATCAATTGTTGGTCCGAGGATGTAAATGCAAC
GAATCTTACGGAACTGCGTTGCATAAATCACATATTAACTGTGAT
TCCAGTCGGATCTCTAACACGTATTATTTGTGACGTGGAATTGGC
AAATGACACGTCAATACAATCAGTGGCCACCGCATACATGAACTT
AAGCATTCTTGCACATGCTCTCCTCAACCAGGGAGGCGTYTGCAT
CTGTAGGTGCCACCTGTTAGACACGTCCCATCTTGCGATTGTTTC
GTTTGTACTTAAAACATTATCAAGCCAGCTGGCGATTTCGTTTTC
AGGATATTGTGGAACCAATGACCCATCTTGTGTCATAGGAGTCAC
GAAAGAGTGCTCCATTAGCCTGGATGTCTTAAGTTCGATTGCAGC
TGCATTTATAAATGAATTGCCCTCAAATGAGCTGCCGGTCCCTCA
ATCATTATTAACGCTACTAGGGTGTTATCAAGACCAGTTAGAGAG
CCTTAGTGCACTCATTGAAAAAACATGGATCCAGGAGATCCGGAG
GCCTCATTTAATGCAATGTGAGATGGAGTGGATAGGACTTTTGGG
AACAGATGCGCTGGGCGATGTCGACACATTTATCAGTTATCGCAA
TGATTCGTGTAATTCCATCCCTGATCTACTGACGCCAGCTGTGAG
TGCCCTCTTATTTGAATTAATCAGCCTGACACCTGAGCTTCCTGG
TACGGAGGACCAGCCGAGTAGGCGTGTAATCAGCATTGGCCAAGC
TTTTAACCTGACTATTTCGGGAAAAGTTAATACGATGATCAGATC
ATGCTGTGAGCAGTGCATTAAGTTATTAGCTGCCAATGTCTCCAT
CCTTAGTGATGTAGATTTGTCGTATTTTGTGAGGGGGATTAGAGA
TGGTTCTTTTGCTTTGGGGTTGTTAATCACCCAGCGTCAAATACT
CAAGGCATCGAGAGCACCAAAGTACCTCAAAACGCATCAGGTCCA
GAACTGGATTGCGTTGTTACTGGAGGTCAAACTAGAGGAAATCTT
CTCTCGCCACTACCGGAAAGTGCTATTGAGAACTCTACGGCTGTT
ATCCCTCTACGGGCTCCTGCAGCAGAAGGAAAGTTGATCATGATA
GATGTCTCTCTGATTTCGCCTCAGCCCTATATACAGATACCTGCT
ACTGCTGTTGTAACTATGCAAATATAGGCTAACCTCGGTCCGGAG
TATTGAGGTAATGGGGAGACTCGTAATTGGAGAAAGAAGAAGAAC
GGGGCAGATACCAGACCCCGGGATACACTACTATAAGCTACTTGG
GTGACTCAGGACGTCACTAATAAGAGTACACTGGTTTTAACAATA
ACACTGATAGTGTAACACTGAATATTGTAAACAGTCTCAGGGATT
ATTTAATTAAAAAGACATTGCGGATCATCATGTCAGGCCTGAACC
AATCAGAAACCCATCCTACAGGCATGAGTCGATTAATCCAGTTTA
ATTTAGTCCAAGATGATTAGTACTCTTAGATGTTACTAAGTATCA
GGATTTAGCGTACTAGATTAAACGTCTTTGAAAGTCATTTTACCC
TGCAAGGTGCATAAGCATATGCAGGGGAATACCCACCACTGGTTC
GTTAGTCTAATTTATTCAAAGATGCCACCAACTGAGTGCACTGGG
AGTCTGATTGAGCTGAATCAACCTAAAGCTCAAGCTTTCCTGTGA
TGTAGCATGCAGTGTGCTACTGCTGCATAGAGCCAATCAGGGGCG
TCTTGAAGGCCTCCACCTTCCTCAGGCAACGATAAAGCAGCTCAC
TGCTACACCCACCATGCAAATACAAGGATATGTAGGTCCATCACC
CTGCTTGCTAATGTGGTCGGCTCCAGGACCATGGCCATAGCGCTA
TCCGTCTAATTCAATCCGCCTCATCATATGCGTATACAATCCTCC
ACTGAGAAATGCACCTGCTCAATCCAATGACAAACTACAACAACA
CGAGATCATTATGGGGTCGATTCCCTTGAATCAGAAAGGTAAAAA
CGGCAGCGGAGATGGAGAATACCAGACCTAATCGGCAAAGAACAA
AAGGTTGAAGACTGATTTAAAAATCATTATAACTTTTTGTTTAGT
GGGTCGGCATGGCATCTCCACCTCCTCGCGGTCCGGACCTGGGCA
TCCGAAGGAGGACGCACGTCCACTCGGATGGCTAAGGGAGAGCCT
GCAGTAGCATAACCCCTTGGGGCCTCTAAACGGGTCTTGAGGGGT
TTTTTGGTGAAAGGAGGAACTATAGGCCGGCCCTGCAGCAATGGC
AACAACGTTGCGCAAACTATTAACTGGCGAACTACTTACTCTAGC
TTCCCGGCAACAATTAATAGACTGGATGGAGGCGGATAAAGTTGC
AGGACCACTTCTGCGCTCGGCCCTTCCGGCTGGCTGGTTTATTGC
TGATAAATCTGGAGCCGGTGAGCGTGGGTCTCGCGGTATCATTGC
AGCACTGGGGCCAGATGGTAAGCCCTCCCGTATCGTAGTTATCTA
CACGACGGGGAGTCAGGCAACTATGGATGAACGAAATAGACAGAT
CGCTGAGATAGGTGCCTCACTGATTAAGCATTGGTAACTGTCAGA
CCAAGTTTACTCATATATACTTTAGATTGATTTAAAACTTCATTT
TTAATTTAAAAGGATCTAGGTGAAGATCCTTTTTGATAATCTCAT
GACCAAAATCCCTTAACGTGAGTTTTCGTTCCACTGAGCGTCAGA
CCCCGTAGAAAAGATCAAAGGATCTTCTTGAGATCCTTTTTTTCT
GCGCGTAATCTGCTGCTTGCAAACAAAAAAACCACCGCTACCAGC
GGTGGTTTGTTTGCCGGATCAAGAGCTACCAACTCTTTTTCCGAA
GGTAACTGGCTTCAGCAGAGCGCAGATACCAAATACTGTCCTTCT
AGTGTAGCCGTAGTTAGGCCACCACTTCAAGAACTCTGTAGCACC
GCCTACATACCTCGCTCTGCTAATCCTGTTACCAGTGGCTGCTGC
CAGTGGCGATAAGTCGTGTCTTACCGGGTTGGACTCAAGACGATA
GTTACCGGATAAGGCGCAGCGGTCGGGCTGAACGGGGGGTTCGTG
CACACAGCCCAGCTTGGAGCGAACGACCTACACCGAACTGAGATA
CCTACAGCGTGAGCTATGAGAAAGCGCCACGCTTCCCGAAGGGAG
AAAGGCGGACAGGTATCCGGTAAGCGGCAGGGTCGGAACAGGAGA
GCGCACGAGGGAGCTTCCAGGGGGAAACGCCTGGTATCTTTATAG
TCCTGTCGGGTTTCGCCACCTCTGACTTGAGCGTCGATTTTTGTG
ATGCTCGTCAGGGGGGCGGAGCCTATGGAAAAACGCCAGCAACGC
GGCCTTTTTACGGTTCCTGGCCTTTTGCTGGCCTTTTGCTCACAT
GTTCTTTCCTGCGTTATCCCCTGATTCTGTGGATAACCGTATTAC
CGCCTTTGAGTGAGCTGATACCGCTCGCCGCAGCCGAACGACCGA
GCGCAGCGAGTCAGTGAGCGAGGAAGCGGAAGAGCGCCTGATGCG
GTATTTTCTCCTTACGCATCTGTGCGGTATTTCACACCGCATATG
GTGCACTCTCAGTACAATCTGCTCTGATGCCGCATAGTTAAGCCA
GTATACACTCCGCTATCGCTACGTGACTGGGTCATGGCTGCGCCC
CGACACCCGCCAACACCCGCTGACGCGCCCTGACGGGCTTGTCTG
CTCCCGGCATCCGCTTACAGACAAGCTGTGACCGTCTCCGGGAGC
TGCATGTGTCAGAGGTTTTCACCGTCATCACCGAAACGCGCGAGG
CAGCTGCGGTAAAGCTCATCAGCGTGGTCGTGAAGCGATTCACAG
ATGTCTGCCTGTTCATCCGCGTCCAGCTCGTTGAGTTTCTCCAGA
AGCGTTAATGTCTGGCTTCTGATAAAGCGGGCCATGTTAAGGGCG
GTTTTTTCCTGTTTGGTCACTTGATGCCTCCGTGTAAGGGGGAAT
TTCTGTTCATGGGGGTAATGATACCGATGAAACGAGAGAGGATGC
TCACGATACGGGTTACTGATGATGAACATGCCCGGTTACTGGAAC
GTTGTGAGGGTAAACAACTGGCGGTATGGATGCGGCGGGACCAGA
GAAAAATCACTCAGGGTCAATGCCAGCGCTTCGTTAATACAGATG
TAGGTGTTCCACAGGGTAGCCAGCAGCATCCTGCGATGCAGATCC
GGAACATAATGGTGCAGGGCGCTGACTTCCGCGTTTCCAGACTTT
ACGAAACACGGAAACCGAAGACCATTCATGTTGTTGCTCAGGTCG
CAGACGTTTTGCAGCAGCAGTCGCTTCACGTTCGCTCGCGTATCG
GTGATTCATTCTGCTAACCAGTAAGGCAACCCCGCCAGCCTAGCC
GGGTCCTCAACGACAGGAGCACGATCATGCGCACCCGTGGCCAGG
ACCCAACGCTGCCCGAGATGCGCCGCGTGCGGCTGCTGGAGATGG
CGGACGCGATGGATATGTTCTGCCAAGGGTTGGTTTGCGCATTCA
CAGTTCTCCGCAAGAATTGATTGGCTCCAATTCTTGGAGTGGTGA
ATCCGTTAGCGAGGTGCCGCCGGCTTCCATTCAGGTCGAGGTGGC
CCGGCTCCATGCACCGCGACGCAACGCGGGGAGGCAGACAAGGTA
TAGGGCGGCGCCTACAATCCATGCCAACCCGTTCCATGTGCTCGC
CGAGGCGGCATAAATCGCCGTGACGATCAGCGGTCCAGTGATCGA
AGTTAGGCTGGTAAGAGCCGCGAGCGATCCTTGAAGCTGTCCCTG
ATGGTCGTCATCTACCTGCCTGGACAGCATGGCCTGCAACGCGGG
CATCCCGATGCCGCCGGAAGCGAGAAGAATCATAATGGGGAAGGC
CATCCAGCCTCGCGTCGCGAACGCCAGCAAGACGTAGCCCAGCGC
GTCGGCCGCCATGCCGGCGATAATGGCCTGCTTCTCGCCGAAACG
TTTGGTGGCGGGACCAGTGACGAAGGCTTGAGCGAGGGCGTGCAA
GATTCCGAATACCGCAAGCGACAGGCCGATCATCGTCGCGCTCCA
GCGAAAGCGGTCCTCGCCGAAAATGACCCAGAGCGCTGCCGGCAC
CTGTCCTACGAGTTGCATGATAAAGAAGACAGTCATAAGTGCGGC
GACGATAGTCATGCCCCGCGCCCACCGGAAGGAGCTGACTGGGTT
GAAGGCTCTCAAGGGCATCGGTCGACGCTCTCCCTTATGCGACTC
CTGCATTAGGAAGCAGCCCAGTAGTAGGTTGAGGCCGTTGAGCAC
CGCCGCCGCAAGGAATGGTGCATGCAAGGAGATGGCGCCCAACAG
TCCCCCGGCCACGGGGCCTGCCACCATACCCACGCCGAAACAAGC
GCTCATGAGCCCGAAGTGGCGAGCCCGATCTTCCCCATCGGTGAT
GTCGGCGATATAGGCGCCAGCAACCGCACCTGTGGCGCCGGTGAT
GCCGGCCACGATGCGTCCGGCGTAGAGGATCCACAGGACGGGTGT
GGTCGCCATGATCGCGTAGTCGATAGTGGCTCCAAGTAGCGAAGC
GAGCAGGACTGGGCGGCGGCCAAAGCGGTCGGACAGTGCTCCGAG
AACGGGTGCGCATAGAAATTGCATCAACGCATATAGCGCTAGCAG
CACGCCATAGTGACTGGCGATGCTGTCGGAATGGACGATATCCCG
CAAGAGGCCCGGCAGTACCGGCATAACCAAGCCTATGCCTACAGC
ATCCAGGGTGACGGTGCCGAGGATGACGATGAGCGCATTGTTAGA
TTTCATACACGGTGCCTGACTGCGTTAGCAATTTAACTGTGATAA
ACTACCGCATTAAAGCTTATCGATGATAAGCTGTCAAACATGAGA
ATTCGGCGCGCCTAATACGACTCACTATAGGG

### Support Plasmids Used for Virus Recoveries:

pcDNA3_1 + APMV3_L (SEQ ID NO: 6):
GACGGATCGGGAGATCTCCCGATCCCCTATGGTGCACTCTCAGTACAATCTGCTCTG
ATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATG
AAGAATCTGCTTAGGGTTAGGCGTTTTGCGCTGCTTCGCGATGTACGGGCCAGATAT
ACGCGTTGACATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTT
CCTACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAA
AATGTCGTAACAACTCCGCCCCATTGACGCAAATGGGCGGTAGGCGTGTACGGTGG
GAGGTCTATATAAGCAGAGCTCTCTGGCTAACTAGAGAACCCACTGCTTACTGGCTT
ATCGAAATTAATACGACTCACTATAGGGAGACCCAAGCTGGCTAGCATGTCTTCTCA
TAATATTATCCTCCCTGACCACCACTTAAATTCCCCTATTGTACTAAATAAATTGATG
TACTATTGTAAACTCCTTAATATATTGCCTGACCCTAAGTCGCCATGGTACGAGAAG
ATTAAGTCTTGGACTAATTGCTGCATCCGTGTGTCAGACAGCAATCGCATGACTTTG
TCACGCGCCTCCAAGCTACGAGAACTATTGGCAACGTACGGCGTATATTCGAAGAA
CCACCAGCAATGTTATACCACCATAATCCATCCCCAATCCCTATCACCCATCATGCA
GACTGTCTCTCAACTGGGACGCTCAATCCCGACCTGGGCCAGGATCCGAAAGGAGA
TCACACACAGTATCATAGCCCAAACGAATAAATTTGAATCCTTATTCCACAACATCT
CAAAGGACCTGACAGGGAAAACCAACTTGTTTGGCGGATTCTGTGATTTACATGGG
AGCATAAGCACTGCTGCAAAGCGCTTAATGAATCAACCGGGTCTCTACTTGGATTCA
CCTGATGCCCATGCATGTCAATTTTTGTTCCAGCTTAAAACCTGTCAACGGGAATTG
ATTCTACTTATGAGGCAGAATGCCACAACAGAGCTGATTAGGGTTTTTGACTATCCT
GAGCTACGGGTTCTTGTGACACCTGAATATTCGGTCTGGGTTTTTACACGGACCAAA
CAAGTTACATTACTAACATTCGATTGCTTGTTAATGTACTGCGATCTTTCAGACGGCC
GGCACAACATCCTATTCACTTGTAAGCTTTTGTCTCACTTAAACCACCTCGGGTGTCG
GATAAGGGACCTGCTGGTGCTAATTGATTCACTTGCCGAGAAACACCCCTTAATAGT
TTATGATGTAGTTGCAAGTCTGGAGTCGTTATCATATGGTGCTATACAGCTACATGA
CAAGGTAGCTGGTTATGCTGGAACATTTTTCTCATTTATCCTTGCAGAAATACAGGA
TTCCTTAGAAACAGTGTTGGATCAAGGTAATAGTGAATCTATCATATCCCAAATAAG
GAACATATACTCAGGTCTCACAGTAAACGAGGCTGCAGAATTGCTATGTGTTATGAG
GCTTTGGGGCCACCCTGCCCTAAACAGTGTAGACGCTGCAAGCAAGGTGAGACAGA
GTATGTGCGCTGGCAAGCTGCTTAAATTTGATACGATCCAGTTAGTCCTTGCATTCTT
CAATACACTAATAATTAATGGATATAGGAGAAAGCACCATGGGCGATGGCCGAATG
TTTGTAGTGACTCTATAATTGGGAATGAACTCAAGCGGATGTATTTTGATCAATGTG
AGATCCCCCACGACTTCACATTAAAGAACTACCGGGAATTAAGTCTTCTTGAATTCG
AGTGCACATTTCCCATAGAATTATCAGATAAACTGAACATATTTTTAAAAGATAAGG
CAATAGCATTCCCGAAGTCCAGGTGGACATCTCCTTTCAAAGCAGATATCACACCGC
GCCATTTGCTGCAAGCGCCTGAGTTTAAAACCCGTGCCAACAGACTACTGCTATCAT
TCTTACAACTCGAGGAATTCTCCATCGAATCAGAACTAGAATATGTGACTACCCTGG
CTTACCTGGATGATGATGAGTTCAACGTATCTTACTCCCTCAAAGAGAAGGAGGTAA
AGACAGATGGTCGAATCTTCGCTAAGCTCACAAGGAAAATGCGGAGCTGTCAGGTG
ATATTCGAAGAATTGCTAGCAGAGCATGTCTCTCCATTATTTAAAGACAACGGCGTT
ACTATGGCCGAATTGTCACTTACTAAGAGCCTACTCGCTATTAGCAACCTAAGCTCC
ACCTTATTTGAGACGCAAACAAGGCAAGGCGATCGGAATGCAAGATTCACGCATGC
GCACTTCATAACAACTGACCTGCAAAAGTACTGTTTAAACTGGAGGTACCAGAGTGT
AAAACTGTTCGCACGTCAATTGAACCGGTTATTTGGTCTGCAGCATGGGTTTGAATG
GATCCATTGCATACTAATGAAGTCCACTATGTATGTCGCAGACCCCTTTAACCCTCC
ACATAGCAATGCACGTGAGGCACTCGATGACAACTTAAATAGCGACATATTCATTGT
ATCACCTAGGGGGGCCATCGAGGGACTATGCCAAAAAATGTGGACAATCATCTCAA
TCTCGGCGATCCATGCATCCGCTGCTGTGGCTGGGCTACGGGTGGCATCTATGGTCC
AAGGTGATAACCAAGTTATTGGGGTAACACGCGAGTTCCTAGCTGGGCATGATCAA
ACTTTTGTAGACGATCAATTGACGGTTTCACTTGAGAACTTCACTCAGATATTCAAG
GAAGTCAACTACGGGCTAGGTCATAATCTCAAGCTGAGGGAAACAATCAAGTCGAG
CCACATGTTCATCTACTCAAAGAGGATCTTCTACGACGGCCGAGTGCTTCCACAACT
TCTAAAGAACATAAGTAAGTTAACCCTATCAGCTACAACTACAGGGGAGAACTGTTT
AACGAGTTGCGGGGACCTATCATCATGCATCACACGATGTATAGAGAATGGGTTCCC
TAAAGATGCAGCATTCGTTTTGAATCAATTAGTGATTAGGATCCAGATACTTGCCGA
TCACTTTTATTCCATCCTTGGAGGATGTTTCTCTGGGTTGAACCAGGCGGATATCCGT
CTCCTTCTATATGAGGGTGCAATTTTACCGGCACAATTAGGAGGGTTCAACAACCTC
AACATGTCACGGCTATTCTGTCGGAATATTGGTGACCCATTAGTCGCGTCCATCGCA
GACACCAAGAGATTTGTGAAGTGCCAACTGCTCGTTCCTCACATTTTAGACTCTGTC
GTTGCTATTACCGATAGGAAGGGGTCTTTTACGACACTGATGATGGATCCATATTCA
ATAAACTTGGATTATATCCAGCAACCAGAGACACGATTGAAGCGACATGTGCAAAA
GGTTTTACTACAAGAGTCAGTGAATCCGCTACTCCAAGGGGTCTTCCTAGAGTCTCA
ACAAGAGGAAGAGGAACAACTTGCAGCGTTCTTGCTGGACCGAGAAGTCGTAATGC
CAAGGGTTGCGCACGCTATATTTGAGTGTACCAGCTTAGGTAGGCGCCGACATATCC
AGGGCCTCATCGACACGACAAAGACCATTATTGCACTTGCGTTGGATACACAGCAGT
TAAGTTATACTAAACGTGAACAGATTGTGACGTACAATGCAACTTATATGAGGTCCT
TAGCAAGCATGCTCAGTTCAAGCCATAATCAGACACGTCGGTCTGTGATTGGTCACG
CATCCTTCAATATCACCGACTGCTCGGTCATCCTGGCTCAACAAGTGAGACGTGCAA
GCTGGGCGCCCTTGCTGAATTGGCGGTCATTAGAAGGCTTAGAAGTACCCGACCCTA
TTGAGTCAGTAGCCGGCTACCTGGGTTTGGATTCTAACAACTGTTTCTTATGCTGCCA
CGAGCAGAACAGCTATTCCTGGTTTTTCCTTCCCCGAATGTGCCATTTTGATGATTCG
AGACAATCTAACTCCATACAAAGGGTACCTTACATTGGTTCTAAGACTGACGAGCGC
CAGATGTCTACGATAAATCTACTTGAGAAAACTACCTGTCATGCTAGAGCAGCTACG
AGGCTTGCATCATTATACATTTGGGCCTTTGGGGACTCAGACGATAGTTGGGATGCA
GTCGAGACTCTATCTAACAGTCGATGCCAGATCACCCGAGAGCACTTGCAAGCTCTG
TGCCCTATGCCCTCTTCAGTGAATCTCCACCATCGATTGAACGACGGAATAACACAA
GTGAAGTTTATGCCATCGACCAATAGCAGGGTCTCTCGATTTGTTCATATTTCCAATG
ACCGGCAGAATTACGTATTGGATGACTCCGTTACTGACAGTAATCTGATTTACCAAC
AAGTCATGTTGCTAGGACTTAGTATACTTGAAACATATTTCAGAGAACCTACAGCGA
CGAACTTATCAAGCTTGGTGCTGCACCTTCACACTGATGTATCTTGTTGCTTACGAGA
ATGCCCGATGACACAATATGCGCCCCCCCTGCGAGACATCCCAGAGTTAACTATAAC
TGCGTCAAATCCTTTCTTATTTGATCAAGAACCTATTAGCGAAGCAGACCTGTGTAG
ACTCTCAAAGATAGCATTCCGTAGAGCAGGAGACAACTACGATGCATACGACCAGT
TTCAACTAAGAGCAACACTGGCCGCAACTACAGGTAAGGATGTGGCAGCAACAATC
TTTGGTCCTCTAGCTGCAGTATCAGCAAAAAATGATGCAATAGTCACAAATGACTAT
AGCGGGAATTGGATTTCCGAGTTTAGGTATAGTGATCTCTATCTTTTGAGTGTTAGCC
TGGGCTACGAGATCCTTTTGATTTTCGCGTATCAACTTTACTACCTACGGATACGGTA
CCGGCAAAATATTGTATGCTATATGGAATCTGTATTCCGACGTTGTCATTCTTTATGC
TTGGGCGACTTAATCCAGACTATTTCGCACTCTGAAATACTGGTTGGGTTAAATGCA
GCGGGCTTTAATCTTACCTTGGATCGAAGTGATTTGAAGGAGAATCAACTCTCGCGG
CTTGCTGTGAAATACCTCACATTGTGTGTCCAGACTGCAATAAGTAATTTGGAGGTA
GGATCAGAACCCCTTTGTATTATTGGAGGTCAATTAGATGATGACATATCATTCCAA
GTTGCCCACTTCCTCTGTCGACGACTGTGCATCATCAGCTTGGTCCACTCAAATGTAC
AGAACTTACCCCCGATTAGGGATAATGAAGTTGATGTAAAATCCAAGTTGATCTACG
ACCATCTTAAGCTAGTTGCTACCACACTCAATAATAGGGACCAGTCGTATCTGCTAA
CACTACTGAATAAGCCTAACATCGAGCTTCATACTCCACAGGTTTACTTCATTATGA
GGAAGTGTCTGGGATTATTAAAAGTCTATGGACCTGTACCTCAAAAGCAACCACTTC
CAACCTCCCCAGTAGTATCACTACCAAATAAGTGCAAATCGAAATGGAAGCTTGAG
GAGGTAATTGACAGCATTGAGTCACCTAAGTCTTTTAACTGGGTTCCTGATACGACG
ATTCCATTGGATGCTGAGCAAAATCCCCCAAATCCTAACAGGGTCATCGATAAGATA
AACATTCTAAGATCTCTGAGCCCGCGTCACTCAGTCTGGTACCGTAACCGGCAATAC
CGGTACGTGTTGAGCCAGTTAGGGCATGACCAGCTAGGTGGTGCAACCCTATATCTT
GGTGAAGGGAGCGGCTCCACAATCTTGAACATAGAGCCAAAAGTCAGGAGTGATAA
GATATATTATCATACCTATTTCTCTGCAGACAAAAGCCCTGCACAGCGCAATTTCAT
CCCCCAGCCAACTACTTTTCTGCGATCTAACTTTTATCACTACGAATTAGAGCCGTCT
GGATGTGAGTTCATCAATTGTTGGTCCGAGGATGTAAATGCAACGAATCTTACGGAA
CTGCGTTGCATAAATCACATATTAACTGTGATTCCAGTCGGATCTCTAACACGTATT
ATTTGTGACGTGGAATTGGCAAATGACACGTCAATACAATCAGTGGCCACCGCATAC
ATGAACTTAAGCATTCTTGCACATGCTCTCCTCAACCAGGGAGGCGTYTGCATCTGT
AGGTGCCACCTGTTAGACACGTCCCATCTTGCGATTGTTTCGTTTGTACTTAAAACAT
TATCAAGCCAGCTGGCGATTTCGTTTTCAGGATATTGTGGAACCAATGACCCATCTT
GTGTCATAGGAGTCACGAAAGAGTGCTCCATTAGCCTGGATGTCTTAAGTTCGATTG
CAGCTGCATTTATAAATGAATTGCCCTCAAATGAGCTGCCGGTCCCTCAATCATTAT
TAACGCTACTAGGGTGTTATCAAGACCAGTTAGAGAGCCTTAGTGCACTCATTGAAA
AAACATGGATCCAGGAGATCCGGAGGCCTCATTTAATGCAATGTGAGATGGAGTGG
ATAGGACTTTTGGGAACAGATGCGCTGGGCGATGTCGACACATTTATCAGTTATCGC
AATGATTCGTGTAATTCCATCCCTGATCTACTGACGCCAGCTGTGAGTGCCCTCTTAT
TTGAATTAATCAGCCTGACACCTGAGCTTCCTGGTACGGAGGACCAGCCGAGTAGGC
GTGTAATCAGCATTGGCCAAGCTTTTAACCTGACTATTTCGGGAAAAGTTAATACGA
TGATCAGATCATGCTGTGAGCAGTGCATTAAGTTATTAGCTGCCAATGTCTCCATCC
TTAGTGATGTAGATTTGTCGTATTTTGTGAGGGGGATTAGAGATGGTTCTTTTGCTTT
GGGGTTGTTAATCACCCAGCGTCAAATACTCAAGGCATCGAGAGCACCAAAGTACC
TCAAAACGCATCAGGTCCAGAACTGGATTGCGTTGTTACTGGAGGTCAAACTAGAG
GAAATCTTCTCTCGCCACTACCGGAAAGTGCTATTGAGAACTCTACGGCTGTTATCC
CTCTACGGGCTCCTGCAGCAGAAGGAAAGTTGATCTAGAGGGCCCGTTTAAACCCG
CTGATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGGGGG
CAGGACAGCAAGGGGGAGGATTGGGAAGACAATAGCAGGCATGCTGGGGATGCGG
TGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTT
CTCGCCACGTTCGCCGGCTTTCCCCGTCAAGCTCTAAATCGGGGGCTCCCTTTAGGG
TTCCGATTTAGTGCTTTACGGCACCTCGACCCCAAAAAACTTGATTAGGGTGATGGT
TCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCG
GTCTATTCTTTTGATTTATAAGGGATTTTGCCGATTTCGGCCTATTGGTTAAAAAATG
AGCTGATTTAACAAAAATTTAACGCGAATTAATTCTGTGGAATGTGTGTCAGTTAGG
GTGTGGAAAGTCCCCAGGCTCCCCAGCAGGCAGAAGTATGCAAAGCATGCATCTCA
ATTAGTCAGCAACCAGGTGTGGAAAGTCCCCAGGCTCCCCAGCAGGCAGAAGTATG
CAAAGCATGCATCTCAATTAGTCAGCAACCATAGTCCCGCCCCTAACTCCGCCCATC
CCGCCCCTAACTCCGCCCAGTTCCGCCCATTCTCCGCCCCATGGCTGACTAATTTTTT
TTATTTATGCAGAGGCCGAGGCCGCCTCTGCCTCTGAGCTATTCCAGAAGTAGTGAG
GAGGCTTTTTTGGAGGCCTAGGCTTTTGCAAAAAGCTCCCGGGAGCTTGTATATCCA
TTTTCGGATCTGATCAAGAGACAGGATGAGGATCGTTTCGCATGATTGAACAAGATG
GATTGCACGCAGGTTCTCCGGCCGCTTGGGTGGAGAGGCTATTCGGCTATGACTGGG
CACAACAGACAATCGGCTGCTCTGATGCCGCCGTGTTCCGGCTGTCAGCGCAGGGG
CGCCCGGTTCTTTTTGTCAAGACCGACCTGTCCGGTGCCCTGAATGAACTGCAGGAC
GAGGCAGCGCGGCTATCGTGGCTGGCCACGACGGGCGTTCCTTGCGCAGCTGTGCTC
GACGTTGTCACTGAAGCGGGAAGGGACTGGCTGCTATTGGGCGAAGTGCCGGGGCA
GGATCTCCTGTCATCTCACCTTGCTCCTGCCGAGAAAGTATCCATCATGGCTGATGC
AATGCGGCGGCTGCATACGCTTGATCCGGCTACCTGCCCATTCGACCACCAAGCGAA
ACATCGCATCGAGCGAGCACGTACTCGGATGGAAGCCGGTCTTGTCGATCAGGATG
ATCTGGACGAAGAGCATCAGGGGCTCGCGCCAGCCGAACTGTTCGCCAGGCTCAAG
GCGCGCATGCCCGACGGCGAGGATCTCGTCGTGACCCATGGCGATGCCTGCTTGCCG
AATATCATGGTGGAAAATGGCCGCTTTTCTGGATTCATCGACTGTGGCCGGCTGGGT
GTGGCGGACCGCTATCAGGACATAGCGTTGGCTACCCGTGATATTGCTGAAGAGCTT
GGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTT
CGAAATGACCGACCAAGCGACGCCCAACCTGCCATCACGAGATTTCGATTCCACCG
CCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGC
AGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTATCATGTCT
GTATACCGTCGACCTCTAGCTAGAGCTTGGCGTAATCATGGTCATAGCTGTTTCCTGT
GTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGACT
CGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAG
CCACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAAC
CACCGCTGGTAGCGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACG
CTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTC
TCTTACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAG
CAAAAACAGGAAGGCAAAATGCCGCAAAAAAGGGAATAAGGGCGACACGGAAATG
TTGAATACTCATACTCTTCCTTTTTCAATATTATTGAAGCATTTATCAGGGTTATTGT
CTCATGAGCGGATACATATTTGAATGTATTTAGAAAAATAAACAAATAGGGGTTCCG
CGCACATTTCCCCGAAAAGTGCCACCTGACGTC
pTM 1_APMV3_L (SEQ ID NO: 7)
ATGGATCCCATTATTAATGGAAATTCTGCTAATGTTTATCTAACCGATAGTTATTTAA
AAGGTGTTATCTCTTTCTCAGAGTGTAATGCTTTAGGAAGTTACATATTCAATGGTCC
TTATCTCAAAAATGATTATACCAACTTAATTAGTAGACAAAATCCATTAATAGAACA
CATGAATCTAAAGAAACTAAATATAACACAGTCCTTAATATCTAAGTATCATAAAGG
TGAAATAAAATTAGAAGAACCTACTTATTTTCAGTCATTACTTATGACATACAAGAG
TATGACCTCGTCAGAACAGATTGCTACCACTAATTTACTTAAAAAGATAATAAGAAG
AGCTATAGAAATAAGTGATGTCAAAGTCTATGCTATATTGAATAAACTAGGGCTTAA
AGAAAAGGACAAGATTAAATCCAACAATGGACAAGATGAAGACAACTCAGTTATTA
CGACCATAATCAAAGATGATATACTTTCAGCTGTTAAAGATAATCAATCTCATCTTA
AAGCAGACAAAAATCACTCTACAAAACAAAAAGACACAATCAAAACAACACTCTTG
AAGAAATTGATGTGTTCAATGCAACATCCTCCATCATGGTTAATACATTGGTTTAAC
TTATACACAAAATTAAACAACATATTAACACAGTATCGATCAAATGAGGTAAAAAA
CCATGGGTTTACATTGATAGATAATCAAACTCTTAGTGGATTTCAATTTATTTTGAAC
CAATATGGTTGTATAGTTTATCATAAGGAACTCAAAAGAATTACTGTGACAACCTAT
AATCAATTCTTGACATGGAAAGATATTAGCCTTAGTAGATTAAATGTTTGTTTAATT
ACATGGATTAGTAACTGCTTGAACACATTAAATAAAAGCTTAGGCTTAAGATGCGG
ATTCAATAATGTTATCTTGACACAACTATTCCTTTATGGAGATTGTATACTAAAGCTA
TTTCACAATGAGGGGTTCTACATAATAAAAGAGGTAGAGGGATTTATTATGTCTCTA
ATTTTAAATATAACAGAAGAAGATCAATTCAGAAAACGATTTTATAATAGTATGCTC
AACAACATCACAGATGCTGCTAATAAAGCTCAGAAAAATCTGCTATCAAGAGTATG
TCATACATTATTAGATAAGACAGTGTCCGATAATATAATAAATGGCAGATGGATAAT
TCTATTAAGTAAGTTCCTTAAATTAATTAAGCTTGCAGGTGACAATAACCTTAACAA
TCTGAGTGAACTATATTTTTTGTTCAGAATATTTGGACACCCAATGGTAGATGAAAG
ACAAGCCATGGATGCTGTTAAAATTAATTGCAATGAGACCAAATTTTACTTGTTAAG
CAGTCTGAGTATGTTAAGAGGTGCCTTTATATATAGAATTATAAAAGGGTTTGTAAA
TAATTACAACAGATGGCCTACTTTAAGAAATGCTATTGTTTTACCCTTAAGATGGTT
AACTTACTATAAACTAAACACTTATCCTTCTTTGTTGGAACTTACAGAAAGAGATTT
GATTGTGTTATCAGGACTACGTTTCTATCGTGAGTTTCGGTTGCCTAAAAAAGTGGA
TCTTGAAATGATTATAAATGATAAAGCTATATCACCTCCTAAAAATTTGATATGGAC
TAGTTTCCCTAGAAATTACATGCCATCACACATACAAAACTATATAGAACATGAAAA
ATTAAAATTTTCCGAGAGTGATAAATCAAGAAGAGTATTAGAGTATTATTTAAGAGA
TAACAAATTCAATGAATGTGATTTATACAACTGTGTAGTTAATCAAAGTTATCTCAA
CAACCCTAATCATGTGGTATCATTGACAGGCAAAGAAAGAGAACTCAGTGTAGGTA
GAATGTTTGCAATGCAACCGGGAATGTTCAGACAGGTTCAAATATTGGCAGAGAAA
ATGATAGCTGAAAACATTTTACAATTCTTTCCTGAAAGTCTTACAAGATATGGTGAT
CTAGAACTACAAAAAATATTAGAATTGAAAGCAGGAATAAGTAACAAATCAAATCG
CTACAATGATAATTACAACAATTACATTAGTAAGTGCTCTATCATCACAGATCTCAG
CAAATTCAATCAAGCATTTCGATATGAAACGTCATGTATTTGTAGTGATGTGCTGGA
TGAACTGCATGGTGTACAATCTCTATTTTCCTGGTTACATTTAACTATTCCTCATGTC
ACAATAATATGCACATATAGGCATGCACCCCCCTATATAGGAGATCATATTGTAGAT
CTTAACAATGTAGATGAACAAAGTGGATTATATAGATATCACATGGGTGGCATCGA
AGGGTGGTGTCAAAAACTGTGGACCATAGAAGCTATATCACTATTGGATCTAATATC
TCTCAAAGGGAAATTCTCAATTACTGCTTTAATTAATGGTGACAATCAATCAATAGA
TATAAGCAAACCAATCAGACTCATGGAAGGTCAAACTCATGCTCAAGCAGATTATTT
GCTAGCATTAAATAGCCTTAAATTACTGTATAAAGAGTATGCAGGCATAGGCCACA
AATTAAAAGGAACTGAGACTTATATATCACGAGATATGCAATTTATGAGTAAAACA
ATTCAACATAACGGTGTATATTACCCAGCTAGTATAAAGAAAGTCCTAAGAGTGGG
ACCGTGGATAAACACTATACTTGATGATTTCAAAGTGAGTCTAGAATCTATAGGTAG
TTTGACACAAGAATTAGAATATAGAGGTGAAAGTCTATTATGCAGTTTAATATTTAG
AAATGTATGGTTATATAATCAGATTGCTCTACAATTAAAAAATCATGCATTATGTAA
CAATAAACTATATTTGGACATATTAAAGGTTCTGAAACACTTAAAAACCTTTTTTAA
TCTTGATAATATTGATACAGCATTAACATTGTATATGAATTTACCCATGTTATTTGGT
GGTGGTGATCCCAACTTGTTATATCGAAGTTTCTATAGAAGAACTCCTGACTTCCTC
ACAGAGGCTATAGTTCACTCTGTGTTCATACTTAGTTATTATACAAACCATGACTTA
AAAGATAAACTTCAAGATCTGTCAGATGATAGATTGAATAAGTTCTTAACATGCATA
ATCACGTTTGACAAAAACCCTAATGCTGAATTCGTAACATTGATGAGAGATCCTCAA
GCTTTAGGGTCTGAGAGACAAGCTAAAATTACTAGCGAAATCAATAGACTGGCAGT
TACAGAGGTTTTGAGTACAGCTCCAAACAAAATATTCTCCAAAAGTGCACAACATTA
TACTACTACAGAGATAGATCTAAATGATATTATGCAAAATATAGAACCTACATATCC
TCATGGGCTAAGAGTTGTTTATGAAAGTTTACCCTTTTATAAAGCAGAGAAAATAGT
AAATCTTATATCAGGTACAAAATCTATAACTAACATACTGGAAAAAACTTCTGCCAT
AGACTTAACAGATATTGATAGAGCCACTGAGATGATGAGGAAAAACATAACTTTGC
TTATAAGGATACTTCCATTGGATTGTAACAGAGATAAAAGAGAGATATTGAGTATG
GAAAACCTAAGTATTACTGAATTAAGCAAATATGTTAGGGAAAGATCTTGGTCTTTA
TCCAATATAGTTGGTGTTACATCACCCAGTATCATGTATACAATGGACATCAAATAT
ACTACAAGCACTATATCTAGTGGCATAATTATAGAGAAATATAATGTTAACAGTTTA
ACACGTGGTGAGAGAGGACCCACTAAACCATGGGTTGGTTCATCTACACAAGAGAA
AAAAACAATGCCAGTTTATAATAGACAAGTCTTAACCAAAAAACAGAGAGATCAAA
TAGATCTATTAGCAAAATTGGATTGGGTGTATGCATCTATAGATAACAAGGATGAAT
TCATGGAAGAACTCAGCATAGGAACCCTTGGGTTAACATATGAAAAGGCCAAGAAA
TTATTTCCACAATATTTAAGTGTCAATTATTTGCATCGCCTTACAGTCAGTAGTAGAC
CATGTGAATTCCCTGCATCAATACCAGCTTATAGAACAACAAATTATCACTTTGACA
CTAGCCCTATTAATCGCATATTAACAGAAAAGTATGGTGATGAAGATATTGACATAG
TATTCCAAAACTGTATAAGCTTTGGCCTTAGTTTAATGTCAGTAGTAGAACAATTTA
CTAATGTATGTCCTAACAGAATTATTCTCATACCTAAGCTTAATGAGATACATTTGAT
GAAACCTCCCATATTCACAGGTGATGTTGATATTCACAAGTTAAAACAAGTGATACA
AAAACAGCATATGTTTTTACCAGACAAAATAAGTTTGACTCAATATGTGGAATTATT
CTTAAGTAATAAAACACTCAAATCTGGATCTCATGTTAATTCTAATTTAATATTGGC
ACATAAAATATCTGACTATTTTCATAATACTTACATTTTAAGTACTAATTTAGCTGGA
CATTGGATTCTGATTATACAACTTATGAAAGATTCTAAAGGTATTTTTGAAAAAGAT
TGGGGAGAGGGATATATAACTGATCATATGTTTATTAATTTGAAAGTTTTCTTCAAT
GCTTATAAGACCTATCTCTTGTGTTTTCATAAAGGTTATGGCAAAGCAAAGCTGGAG
TGTGATATGAACACTTCAGATCTTCTATGTGTATTGGAATTAATAGACAGTAGTTATT
GGAAGTCTATGTCTAAGGTATTTTTAGAACAAAAAGTTATCAAATACATTCTTAGCC
AAGATGCAAGTTTACATAGAGTAAAAGGATGTCATAGCTTCAAATTATGGTTTCTTA
AACGTCTTAATGTAGCAGAATTCACAGTTTGCCCTTGGGTTGTTAACATAGATTATC
ATCCAACACATATGAAAGCAATATTAACTTATATAGATCTTGTTAGAATGGGATTGA
TAAATATAGATAGAATACACATTAAAAATAAACACAAATTCAATGATGAATTTTATA
CTTCTAATCTCTTCTACATTAATTATAACTTCTCAGATAATACTCATCTATTAACTAA
ACATATAAGGATTGCTAATTCTGAATTAGAAAATAATTACAACAAATTATATCATCC
TACACCAGAAACCCTAGAGAATATACTAGCCAATCCGATTAAAAGTAATGACAAAA
AGACACTGAATGACTATTGTATAGGTAAAAATGTTGACTCAATAATGTTACCATTGT
TATCTAATAAGAAGCTTATTAAATCGTCTGCAATGATTAGAACCAATTACAGCAAAC
AAGATTTGTATAATTTATTCCCTATGGTTGTGATTGATAGAATTATAGATCATTCAGG
CAATACAGCCAAATCCAACCAACTTTACACTACTACTTCCCACCAAATATCTTTAGT
GCACAATAGCACATCACTTTACTGCATGCTTCCTTGGCATCATATTAATAGATTCAAT
TTTGTATTTAGTTCTACAGGTTGTAAAATTAGTATAGAGTATATTTTAAAAGATCTTA
AAATTAAAGATCCCAATTGTATAGCATTCATAGGTGAAGGAGCAGGGAATTTATTAT
TGCGTACAGTAGTGGAACTTCATCCTGACATAAGATATATTTACAGAAGTCTGAAAG
ATTGCAATGATCATAGTTTACCTATTGAGTTTTTAAGGCTGTACAATGGACATATCA
ACATTGATTATGGTGAAAATTTGACCATTCCTGCTACAGATGCAACCAACAACATTC
ATTGGTCTTATTTACATATAAAGTTTGCTGAACCTATCAGTCTTTTTGTCTGTGATGC
CGAATTGTCTGTAACAGTCAACTGGAGTAAAATTATAATAGAATGGAGCAAGCATG
TAAGAAAGTGCAAGTACTGTTCCTCAGTTAATAAATGTATGTTAATAGTAAAATATC
ATGCTCAAGATGATATTGATTTCAAATTAGACAATATAACTATATTAAAAACTTATG
TATGCTTAGGCAGTAAGTTAAAGGGATCGGAGGTTTACTTAGTCCTTACAATAGGTC
CTGCGAATATATTCCCAGTATTTAATGTAGTACAAAATGCTAAATTGATACTATCAA
GAACCAAAAATTTCATCATGCCTAAGAAAGCTGATAAAGAGTCTATTGATGCAAAT
ATTAAAAGTTTGATACCCTTTCTTTGTTACCCTATAACAAAAAAAGGAATTAATACT
GCATTGTCAAAACTAAAGAGTGTTGTTAGTGGAGATATACTATCATATTCTATAGCT
GGACGTAATGAAGTTTTCAGCAATAAACTTATAAATCATAAGCATATGAACATCTTA
AAATGGTTCAATCATGTTTTAAATTTCAGATCAACAGAACTAAACTATAACCATTTA
TATATGGTAGAATCTACATATCCTTACCTAAGTGAATTGTTAAACAGCTTGACAACC
AATGAACTTAAAAAACTGATTAAAATCACAGGTAGTCTGTTATACAACTTTCATAAT
GAATAATGAATAAAGATCTTATAATAAAAATTCCCATAGCTGCAGCTCGAGAGGCC
TAATTAATTAAGTCGACGATCCGGCTGCTAACAAAGCCCGAAAGGAAGCTGAGTTG
GCTGCTGCCACCGCTGAGCAATAACTAGCATAACCCCTTGGGGCCTCTAAACGGGTC
TTGAGGGGTTTTTTGCTGAAAGGAGGAACTATATCCGGATCGAGATCAATTCTGTGA
GCGTATGGCAAACGAAGGAAAAATAGTTATAGTAGCCGCACTCGATGGGACATTTC
AACGTAAACCGTTTAATAATATTTTGAATCTTATTCCATTATCTGAAATGGTGGTAA
AACTAACTGCTGTGTGTATGAAATGCTTTAAGGAGGCTTCCTTTTCTAAACGATTGG
GTGAGGAAACCGAGATAGAAATAATAGGAGGTAATGATATGTATCAATCGGTGTGT
AGAAAGTGTTACATCGACTCATAATATTATATTTTTTATCTAAAAAACTAAAAATAA
ACATTGATTAAATTTTAATATAATACTTAAAAATGGATGTTGTGTCGTTAGATAAAC
CGTTTATGTATTTTGAGGAAATTGATAATGAGTTAGATTACGAACCAGAAAGTGCAA
ATGAGGTCGCAAAAAAACTGCCGTATCAAGGACAGTTAAAACTATTACTAGGAGAA
TTATTTTTTCTTAGTAAGTTACAGCGACACGGTATATTAGATGGTGCCACCGTAGTGT
ATATAGGATCTGCTCCCGGTACACATATACGTTATTTGAGAGATCATTTCTATAATTT
AGGAGTGATCATCAAATGGATGCTAATTGACGGCCGCCATCATGATCCTATTTTAAA
TGGATTGCGTGATGTGACTCTAGTGACTCGGTTCGTTGATGAGGAATATCTACGATC
CATCAAAAAACAACTGCATCCTTCTAAGATTATTTTAATTTCTGATGTGAGATCCAA
ACGAGGAGGAAATGAACCTAGTACGGCGGATTTACTAAGTAATTACGCTCTACAAA
ATGTCATGATTAGTATTTTAAACCCCGTGGCGTCTAGTCTTAAATGGAGATGCCCGT
TTCCAGATCAATGGATCAAGGACTTTTATATCCCACACGGTAATAAAATGTTACAAC
CTTTTGCTCCTTCATATTCAGGGCCGTCGTTTTACAACGTCGTGACTGGGAAAACCCT
GGCGTTACCCAACTTAATCGCCTTGCAGCACATCCCCCTTTCGCCAGCTGGCGTAAT
AGCGAAGAGGCCCGCACCGATCGCCCTTCCCAACAGTTGCGCAGCCTGAATGGCGA
ATGGCGCGACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCCC
TTCCTTTCTCGCCACGTTCGCCGGCTTTCCCCGTCAAGCTCTAAATCGGGGGCTCCCT
TTAGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGTTG
GAGTCCACGTTCTTTAATAGTGGACTCTTGTTCCAAACTGGAACAACACTCAACCCT
ATCTCGGTCTATTCTTTTGATTTATAAGGGATTTTGCCGATTTCGGCCTATTGGTTAA
AAAATGAGCTGATTTAACAAAAATTTAACGCGAATTTTAACAAAATATTAACGTTTA
CAATTTCCCAGGTGGCACTTTTCGGGGAAATGTGCGCGGAACCCCTATTTGTTTATTT
TTCTAAATACATTCAAATATGTATCCGCTCATGAGACAATAACCCTGATAAATGCTT
CAATAATATTGAAAAAGGAAGAGTATGAGTATTCAACATTTCCGTGTCGCCCTTATT
CCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTC
AACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGAG
CAACTCGGTCGCCGCATACACTATTCTCAGAATGACTTGGTTGAGTACTCACCAGTC
ACAGAAAAGCATCTTACGGATGGCATGACAGTAAGAGAATTATGCAGTGCTGCCAT
AACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTT
GGGAACCGGAGCTGAATGAAGCCATACCAAACGACGAGCGTGACACCACGATGCCT
GTAGCAATGGCAACAACGTTGCGCAAACTATTAACTGGCGAACTACTTACTCTAGCT
TCCCGGCAACAATTAATAGACTGGATGGAGGCGGATAAAGTTGCAGGACCACTTCT
GCGCTCGGCCCTTCCGGCTGGCTGGTTTATTGCTGATAAATCTGGAGCCGGTGAGCG
TGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCG
CTGAGATAGGTGCCTCACTGATTAAGCATTGGTAACTGTCAGACCAAGTTTACTCAT
ATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCG
TCAGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACCA
AATACTGTCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCTTTGAGTGA
GCTGATACCGCTCGCCGCAGCCGAACGACCGAGCGCAGCGAGTCAGTGAGCGAGGA
AGCGGAAGAGCGCCCAATACGCAAACCGCCTCTCCCCGCGCGTTGGCCGATTCATT
AATGCAGCTGGCACGACAGGTTTCCCGACTGGAAAGCGGGCAGTGAGCGCAACGCA
ATTAATGTGAGTTAGCTCACTCATTAGGCACCCCAGGCTTTACACTTTATGCTTCCGG
CTCGTATGTTGTGTGGAATTGTGAGCGGATAACAATTTCACACAGGAAACAGCTATG
ACCATGATTACGCCAAGCTTTTGCGATCAATAAATGGATCACAACCAGTATCTCTTA
ACGATGTTCTTCGCAGATGATGATTCATTTTTTAAGTATTTGGCTAGTCAAGATGATG
AATCTTCATTATCTGATATATTGCAAATCACTCAATATCTAGACTTTCTGTTATTATT
ATTGATCCAATCAAAAAATAAATTAGAAGCCGTGGGTCATTGTTATGAATCTCTTTC
AGAGGAATACAGACAATTGACAAAATTCACAGACTTTCAAGATTTTAAAAAACTGT
TTAACAAGGTCCCTATTGTTACAGATGGAAGGGTCAAACTTAATAAAGGATATTTGT
TCGACTTTGTGATTAGTTTGATGCGATTCAAAAAAGAATCCTCTCTAGCTACCACCG
CAATAGATCCTGTTAGATACATAGATCCTCGTCGCAATATCGCATTTTCTAACGTGA
TGGATATATTAAAGTCGAATAAAGTGAACAATAATTAATTCTTTATTGTCATCATGA
ACGGCGGACATATTCAGTTGATAATCGGCCCCATGTTTTCAGGTAAAAGTACAGAAT
TAATTAGACGAGTTAGACGTTATCAAATAGCTCAATATAAATGCGTGACTATAAAAT
ATTCTAACGATAATAGATACGGAACGGGACTATGGACGCATGATAAGAATAATTTT
GAAGCATTGGAAGCAACTAAACTATGTGATGTCTTGGAATCAATTACAGATTTCTCC
GTGATAGGTATCGATGAAGGACAGTTCTTTCCAGACATTGTTGAATTGATCTCGATC
CCGCGAAATTAATACGACTCACTATAGGGAGACCACAACGGTTTCCCTCTAGCGGG
ATCAATTCCGCCCCTCTCCCTCCCCCCCCCCTAACGTTACTGGCCGAAGCCGCTTGG
AATAAGGCCGGTGTGCGTTTGTCTATATGTTATTTTCCACCATATTGCCGTCTTTTGG
CAATGTGAGGGCCCGGAAACCTGGCCCTGTCTTCTTGACGAGCATTCCTAGGGGTCT
TTCCCCTCTCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGGCAGCGGA
ACCCCCCACCTGGCGACAGGTGCCTCTGCGGCCAAAAGCCACGTGTATAAGATACA
CCTGCAAAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCCAGAAGG
TACCCCATTGTATGGGATCTGATCTGGGGCCTCGGTGCACATGCTTTACATGTGTTTA
GTCGAGGTTAAAAAACGTCTAGGCCCCCCGAACCACGGGGACGTGGTTTTCCTTTGA
AAAACACGATAATACC
pTM 1_APMV3_N (SEQ ID NO: 8):
CCATGGCTGGAATTTTTAATACTTATGAGCTCTTTGTTAAGGATCAGACATGTATGC
ACAAGAGGGCTGCAAGCCTAATATCGGGAGGTCAGCTTAAAAGCAATATACCAGTC
TTCATCACCACAAAAGATGATCCAGCTGTCCGATGGGACCTTGTATGTTTTAATCTC
CGACTTGTTGTTAGTGAATCATCTACCTCGGTGATCCGGCAGGGGGCTCTTATATCA
TTGCTTTCCATTACTGCTAGCAATATGAGGGCTCTTGCAGCAATTGCTGGGCAGACT
GATGAATCGATGATTAATATTATAGAAGTTGTGGACTTTGATGGACTTGAGCCCCAG
TGTGACACTCGCAGTGGCTTAGACGCTCAGAAGCAGGAGATGTTTAAGGATATTGCC
CGTGATATGCCGAGGGCACTATCCAGCGGGACCCCGTTCCAGCACGGAAATGCGGA
AGCGAATGGGCCTGAAGACACACACATGTTCCTCCGATCCGCAATTAGCGTTCTGAC
ACAGATCTGGATCCTGGTTGCAAAAGCCATGACTAACATTGATGGCAGCAACGAGG
CAAGCGATCGCCGCCTTGCCAAGTATATTCAACAGAATAGAATTGACAGGCGATTC
ATGCTTGCTCAAGCTACCAGGACTGTATGTCAGCAGGTCATAAAGGATTCTTTGACC
TTGAGAAGGTTCCTTGTAACTGAGCTTCGAAAGTCGCGGGGGGCATTGCATAGCGG
GTCATCTTACTATGCTATGATTGGAGACATGCAGGCATACATATTTAATGCAGGCCT
TACACCATTCCTCACTACCCTCAGGTACGGGATCGGGACTAAATACCATGCCCTTGC
AATTAGCTCACTTGCAGGTGACCTGAATAAGATTAAGGGGTTACTGACTCTATACAA
AGAGAAAGGGAGTGATGCGGGCTATATGGCACTTCTAGAGGATGCTGACTGCATGC
AATTTGCGCCAGGTAATTACTCACTATTATATTCATATGCGATGGGAGTAGCAAGTG
TACATGATGAAGGCATGAGGAATTACCAATACGCCCGGCGCTTCCTCCATAAGGGG
ATGTACCAATTTGGACGTGATATCGCAACACAACAGCAACATGCACTTGATGAATCC
CTCGCCCAGGAGATGAGAATCACAGAGGCTGACAGGGCAAATCTCAAGGTAATGAT
GGCAAACATTGGGGAAGCATCAAGCTTCAGTGATATGCCTAATCCAGGAGCTAGCG
GCATTCCTGCCTTCAATGACCGCCCTGATGAGATCTTCTCTGAGCCAGGCTATCGCA
AGCCCCCTCAAGATCAGCCGCTGATAAAGCTCCCAGATCTCGAGGAAGAAGAGCAG
GACGAATTCGACATGTAGACTAGTGGATCCCTGCAGCTCGAGAGGCCTAATTAATTA
AGTCGACGATCCGGCTGCTAACAAAGCCCGAAAGGAAGCTGAGTTGGCTGCTGCCA
CCGCTGAGCAATAACTAGCATAACCCCTTGGGGCCTCTAAACGGGTCTTGAGGGGTT
TTTTGCTGAAAGGAGGAACTATATCCGGATCGAGATCAATTCTGTGAGCGTATGGCA
AACGAAGGAAAAATAGTTATAGTAGCCGCACTCGATGGGACATTTCAACGTAAACC
GTTTAATAATATTTTGAATCTTATTCCATTATCTGAAATGGTGGTAAAACTAACTGCT
GTGTGTATGAAATGCTTTAAGGAGGCTTCCTTTTCTAAACGATTGGGTGAGGAAACC
GAGATAGAAATAATAGGAGGTAATGATATGTATCAATCGGTGTGTAGAAAGTGTTA
CATCGACTCATAATATTATATTTTTTATCTAAAAAACTAAAAATAAACATTGATTAA
ATTTTAATATAATACTTAAAAATGGATGTTGTGTCGTTAGATAAACCGTTTATGTATT
TTGAGGAAATTGATAATGAGTTAGATTACGAACCAGAAAGTGCAAATGAGGTCGCA
AAAAAACTGCCGTATCAAGGACAGTTAAAACTATTACTAGGAGAATTATTTTTTCTT
AGTAAGTTACAGCGACACGGTATATTAGATGGTGCCACCGTAGTGTATATAGGATCT
GCTCCCGGTACACATATACGTTATTTGAGAGATCATTTCTATAATTTAGGAGTGATC
ATCAAATGGATGCTAATTGACGGCCGCCATCATGATCCTATTTTAAATGGATTGCGT
GATGTGACTCTAGTGACTCGGTTCGTTGATGAGGAATATCTACGATCCATCAAAAAA
CAACTGCATCCTTCTAAGATTATTTTAATTTCTGATGTGAGATCCAAACGAGGAGGA
AATGAACCTAGTACGGCGGATTTACTAAGTAATTACGCTCTACAAAATGTCATGATT
AGTATTTTAAACCCCGTGGCGTCTAGTCTTAAATGGAGATGCCCGTTTCCAGATCAA
TGGATCAAGGACTTTTATATCCCACACGGTAATAAAATGTTACAACCTTTTGCTCCTT
CATATTCAGGGCCGTCGTTTTACAACGTCGTGACTGGGAAAACCCTGGCGTTACCCA
ACTTAATCGCCTTGCAGCACATCCCCCTTTCGCCAGCTGGCGTAATAGCGAAGAGGC
CCGCACCGATCGCCCTTCCCAACAGTTGCGCAGCCTGAATGGCGAATGGCGCGACG
CGCCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCC
GATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGT
TCTTTAATAGTGGACTCTTGTTCCAAACTGGAACAACACTCAACCCTATCTCGGTCTA
TTCTTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTCCCAG
GTGGCACTTTTCGGGGAAATGTGCGCGGAACCCCTATTTGTTTATTTTTCTAAATACA
TTCAAATATGTATCCGCTCATGAGACAATAACCCTGATAAATGCTTCAATAATATTG
AAAAAGGAAGAGTATGAGTATTCAACATTTCCGTGTCGCCCTTATTCCCTTTTTTGCG
GCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAA
GATCCTTGAGAGTTTTCGCCCCGAAGAACGTTTTCCAATGATGAGCACTTTTAAAGT
TCTGCTATGTGGCGCGGTATTATCCCGTATTGACGCCGGGCAAGAGCAACTCGGTCG
CCGCATACACTATTCTCAGAATGACTTGGTTGAGTACTCACCAGTCACAGAAAAGCA
TCTTACGGATGGCATGACAGTAAGAGAATTATGCAGTGCTGCCATAACCATGAGTG
ATAACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGGAG
CTGAATGAAGCCATACCAAACGACGAGCGTGACACCACGATGCCTGTAGCAATGGC
AACAACGTTGCGCAAACTATTAACTGGCGAACTACTTACTCTAGCTTCCCGGCAACA
ATTAATAGACTGGATGGAGGCGGATAAAGTTGCAGGACCACTTCTGCGCTCGGCCCT
TCCGGCTGGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTATCTACAC
GACGGGGAGTCAGGCAACTATGGATGAACGAAATAGACAGATCGCTGAGATAGGTG
CCTCACTGATTAAGCATTGGTAACTGTCAGACCAAGTTTACTCATATATACTTTAGAT
TGATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTA
GAAAAGATCAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTT
CTAGTGTAGCCGTAGTTAGGCCACCACTTCAAGAACTCTGTAGCACCGCCTACATAC
CTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTCTTTCCTGCG
TTATCCCCTGATTCTGTGGATAACCGTATTACCGCCTTTGAGTGAGCTGATACCGCTC
GCCGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATGCAGCTGGCA
CGACAGGTTTCCCGACTGGAAAGCGGGCAGTGAGCGCAACGCAATTAATGTGAGTT
AGCTCACTCATTAGGCACCCCAGGCTTTACACTTTATGCTTCCGGCTCGTATGTTGTG
TGGAATTGTGAGCGGATAACAATTTCACACAGGAAACAGCTATGACCATGATTACG
CCAAGCTTTTGCGATCAATAAATGGATCACAACCAGTATCTCTTAACGATGTTCTTC
GCAGATGATGATTCATTTTTTAAGTATTTGGCTAGTCAAGATGATGAATCTTCATTAT
CTGATATATTGCAAATCACTCAATATCTAGACTTTCTGTTATTATTATTGATCCAATC
AAAAAATAAATTAGAAGCCGTGGGTCATTGTTATGAATCTCTTTCAGAGGAATACAG
ACAATTGACAAAATTCACAGACTTTCAAGATTTTAAAAAACTGTTTAACAAGGTCCC
TATTGTTACAGATGGAAGGGTCAAACTTAATAAAGGATATTTGTTCGACTTTGTGAT
TAGTTTGATGCGATTCAAAAAAGAATCCTCTCTAGCTACCACCGCAATAGATCCTGT
TAGATACATAGATCCTCGTCGCAATATCGCATTTTCTAACGTGATGGATATATTAAA
GTCGAATAAAGTGAACAATAATTAATTCTTTATTGTCATCATGAACGGCGGACATAT
TCAGTTGATAATCGGCCCCATGTTTTCAGGTAAAAGTACAGAATTAATTAGACGAGT
TAGACGTTATCAAATAGCTCAATATAAATGCGTGACTATAAAATATTCTAACGATAA
TAGATACGGAACGGGACTATGGACGCATGATAAGAATAATTTTGAAGCATTGGAAG
CAACTAAACTATGTGATGTCTTGGAATCAATTACAGATTTCTCCGTGATAGGTATCG
ATGAAGGACAGTTCTTTCCAGACATTGTTGAATTGATCTCGATCCCGCGAAATTAAT
ACGACTCACTATAGGGAGACCACAACGGTTTCCCTCTAGCGGGATCAATTCCGCCCC
TCTCCCTCCCCCCCCCCTAACGTTACTGGCCGAAGCCGCTTGGAATAAGGCCGGTGT
GCGTTTGTCTATATGTTATTTTCCACCATATTGCCGTCTTTTGGCAATGTGAGGGCCC
GGAAACCTGGCCCTGTCTTCTTGACGAGCATTCCTAGGGGTCTTTCCCCTCTCGCCA
AAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGC
GACAGGTGCCTCTGCGGCCAAAAGCCACGTGTATAAGATACACCTGCAAAGGCGGC
ACAACCCCAGTGCCACGTTGTGAGTTGGATAGTTGTGGAAAGAGTCAAATGGCTCTC
CTCAAGCGTATTCAACAAGGGGCTGAAGGATGCCCAGAAGGTACCCCATTGTATGG
GATCTGATCTGGGGCCTCGGTGCACATGCTTTACATGTGTTTAGTCGAGGTTAAAAA
ACGTCTAGGCCCCCCGAACCACGGGGACGTGGTTTTCCTTTGAAAAACACGATAATA
pTM 1_APMV3_P (SEQ ID NO: 9):
CCATGGATCTTGAGTTTAGTAGTGAGGAAGCAGTCGCAGCCCTCTTGGATGTGAGTT
CCTCAACCATAACCGAGGTTCTGAGCAAGCAAAGTATTCCTGACCCCAGCTTTCTCA
CGTCCACGGAGGCATCCGGTGAGCAAGTTCCAGATTCGAAGGTCGCCAATCACAAA
GCCAGTGTCGATAAGACCCCAGATCAAGGCCAACCATCGGCAACGCCTTCGGCTCC
TCCTGAGACTGCCGAAAACATCAATACACCATCTTGCGAGGACGGTTTACCCCCTAA
CTTCTTCATCCCAAGGGTAGAGAGTTACCACACTAACCTTTTTAAAGGGGGCCCCCA
ATTGGACTCAACGGAGCGGCCGCCTGGTCACCAAATAGTTTGCGGAGATCAAGACG
CGGACCTCTCCCGAGCCGGGATAGCGAGAAAGAAGAAGAAGCAGAAGCATTGCAA
AGCGCTCAACTTTTCGGAATCCCCACTCACAGACAATCAGGCCATAGAAGGGAGTA
TTCAATCTCATGGAGCCCTGGATCAGGAACCTTCCAGACAGAGACCTGGTGCAACCC
AGCCTGCTCTCCAGTCACCGCCTTCCCAAAACAATACAAGTGTGCATGCAGACAGTG
CCCAAGATTCTGCGATCTCTGTTTCAATCCCGCTGACTATGGTGGAATCATTGATCTC
TCAAGTGTCAAAGTTGTCGGACCAAGTCTCGCAGATACAGAAGTTGGTGAGTACCCT
TCCTCAAATTAAGACTGATATTGCTTCGATTCGGAACATGCAAGCAGCTTTAGAAGG
ACAGCTCAGTATGATTCGTATATTGGATCCAGGAAACTGTTCGGAATCATCACTCAA
TGCGCTGAGAGGGTTCAGTGGTAAGGCACCTGTTATTGTGAGTGGTCCAGGAAATCC
CAATCAATGCATCAGCCAAGGCTCATCCACCACAATTTGCCTGGATGAGTTAGCCCG
GCCTGTGCCAAATCCTATACAGGTGAAACTTCAAGATGATCAGAAGGACCTATCTGC
ACAGCGACATGCTGTCATTGCATTACTCGAAACCAGAATCCCCCCGGGACCAAAAC
GTGACAAATTACTGTCATTGGTTGCAATTGCAAAGACTGCAAGTGATTTAATCAAGA
TTAAGAGAATGGCAGTCCTCGGCCAGTGACTCGAGAGGCCTAATTAATTAAGTCGA
CGATCCGGCTGCTAACAAAGCCCGAAAGGAAGCTGAGTTGGCTGCTGCCACCGCTG
AGCAATAACTAGCATAACCCCTTGGGGCCTCTAAACGGGTCTTGAGGGGTTTTTTGC
TGAAAGGAGGAACTATATCCGGATCGAGATCAATTCTGTGAGCGTATGGCAAACGA
AGGAAAAATAGTTATAGTAGCCGCACTCGATGGGACATTTCAACGTAAACCGTTTA
ATAATATTTTGAATCTTATTCCATTATCTGAAATGGTGGTAAAACTAACTGCTGTGTG
TATGAAATGCTTTAAGGAGGCTTCCTTTTCTAAACGATTGGGTGAGGAAACCGAGAT
AGAAATAATAGGAGGTAATGATATGTATCAATCGGTGTGTAGAAAGTGTTACATCG
ACTCATAATATTATATTTTTTATCTAAAAAACTAAAAATAAACATTGATTAAATTTTA
ATATAATACTTAAAAATGGATGTTGTGTCGTTAGATAAACCGTTTATGTATTTTGAG
GAAATTGATAATGAGTTAGATTACGAACCAGAAAGTGCAAATGAGGTCGCAAAAAA
ACTGCCGTATCAAGGACAGTTAAAACTATTACTAGGAGAATTATTTTTTCTTAGTAA
GTTACAGCGACACGGTATATTAGATGGTGCCACCGTAGTGTATATAGGATCTGCTCC
CGGTACACATATACGTTATTTGAGAGATCATTTCTATAATTTAGGAGTGATCATCAA
ATGGATGCTAATTGACGGCCGCCATCATGATCCTATTTTAAATGGATTGCGTGATGT
GACTCTAGTGACTCGGTTCGTTGATGAGGAATATCTACGATCCATCAAAAAACAACT
GCATCCTTCTAAGATTATTTTAATTTCTGATGTGAGATCCAAACGAGGAGGAAATGA
ACCTAGTACGGCGGATTTACTAAGTAATTACGCTCTACAAAATGTCATGATTAGTAT
TTTAAACCCCGTGGCGTCTAGTCTTAAATGGAGATGCCCGTTTCCAGATCAATGGAT
CAAGGACTTTTATATCCCACACGGTAATAAAATGTTACAACCTTTTGCTCCTTCATAT
TCAGGGCCGTCGTTTTACAACGTCGTGACTGGGAAAACCCTGGCGTTACCCAACTTA
ATCGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGCGCGACGCGCCCT
GTAGCGGCGCATTAAGCGCGGCGGGTGTGGTGGTTACGCGCAGCGTGACCGCTACA
CTTGCCAGCGCCCTAGCGCCCGCTCCTTTCGCTTTCTTCCCTTCCTTTCTCGCCACGTT
CGCCGGCTTTCCCCGTCAAGCTCTAAATCGGGGGCTCCCTTTAGGGTTCCGATTTAGT
GCTTTACGGCACCTCGACCCCAAAAAACTTGATTAGGGTGATGGTTCACGTAGTGGG
CCATCGCCCTGATAGACGGTTTTTCGCCCTTTGACGTTGGAGTCCACGTTCTTTAATA
GTGGACTCTTGTTCCAAACTGGAACAACACTCAACCCTATCTCGGTCTATTCTTTTGA
TTTATAAGGGATTTTGCCGATTTCGGCCTATTGGTTAAAAAATGAGCTGATTTAACA
AAAATTTAACGCGAATTTTAACAAAATATTAACGTTTACAATTTCCCAGGTGGCACT
TTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTG
CCTTCCTGTTTTTGCTCACCCAGAAACGCTGGTGAAAGTAAAAGATGCTGAAGATCA
GTTGGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTATGT
GGCGCGGTATTATCCCGTATTGACGCCGGGCAAGAGCAACTCGGTCGCCGCATACA
CTATTCTCAGAATGACTTGGTTGAGTACTCACCAGTCACAGAAAAGCATCTTACGGA
TGGCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGAA
GCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTATCATTGC
AGCACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCACTG
ATTAAGCATTGGTAACTGTCAGACCAAGTTTACTCATATATACTTTAGATTGATTTAA
AACTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGC
CGAACGACCGAGCGCAGCGAGTCAGTGAGCGAGGAAGCGGAAGAGCGCCCAATAC
GCAAACCGCCTCTCCCCGCGCGTTGGCCGATTCATTAATGCAGCTGGCACGACAGGT
TTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTG
TGAGCGGATAACAATTTCACACAGGAAACAGCTATGACCATGATTACGCCAAGCTTT
TGCGATCAATAAATGGATCACAACCAGTATCTCTTAACGATGTTCTTCGCAGATGAT
GATTCATTTTTTAAGTATTTGGCTAGTCAAGATGATGAATCTTCATTATCTGATATAT
TGCAAATCACTCAATATCTAGACTTTCTGTTATTATTATTGATCCAATCAAAAAATAA
ATTAGAAGCCGTGGGTCATTGTTATGAATCTCTTTCAGAGGAATACAGACAATTGAC
AAAATTCACAGACTTTCAAGATTTTAAAAAACTGTTTAACAAGGTCCCTATTGTTAC
AGATGGAAGGGTCAAACTTAATAAAGGATATTTGTTCGACTTTGTGATTAGTTTGAT
GCGATTCAAAAAAGAATCCTCTCTAGCTACCACCGCAATAGATCCTGTTAGATACAT
AGATCCTCGTCGCAATATCGCATTTTCTAACGTGATGGATATATTAAAGTCGAATAA
AGTGAACAATAATTAATTCTTTATTGTCATCATGAACGGCGGACATATTCAGTTGAT
AATCGGCCCCATGTTTTCAGGTAAAAGTACAGAATTAATTAGACGAGTTAGACGTTA
TCAAATAGCTCAATATAAATGCGTGACTATAAAATATTCTAACGATAATAGATACGG
AACGGGACTATGGACGCATGATAAGAATAATTTTGAAGCATTGGAAGCAACTAAAC
TATGTGATGTCTTGGAATCAATTACAGATTTCTCCGTGATAGGTATCGATGAAGGAC
AGTTCTTTCCAGACATTGTTGAATTGATCTCGATCCCGCGAAATTAATACGACTCACT
ATAGGGAGACCACAACGGTTTCCCTCTAGCGGGATCAATTCCGCCCCTCTCCCTCCC
CCCCCCCTAACGTTACTGGCCGAAGCCGCTTGGAATAAGGCCGGTGTGCGTTTGTCT
ATATGTTATTTTCCACCATATTGCCGTCTTTTGGCAATGTGAGGGCCCGGAAACCTG
GCCCTGTCTTCTTGACGAGCATTCCTAGGGGTCTTTCCCCTCTCGCCAAAGGAATGC
AAGGTCTGTTGAATGTCGTGAAGGAAGCAGTTCCTCTGGAAGCTTCTTGAAGACAAA
CAACGTCTGTAGCGACCCTTTGCAGGCAGCGGAACCCCCCACCTGGCGACAGGTGC
CTCTGCGGCCAAAAGCCACGTGTATAAGATACACCTGCAAAGGCGGCACAACCCCA
GTGCCACGTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGATCTGATC
TGGGGCCTCGGTGCACATGCTTTACATGTGTTTAGTCGAGGTTAAAAAACGTCTAGG
CCCCCCGAACCACGGGGACGTGGTTTTCCTTTGAAAAACACGATAATA
pTM 1_APMV3_CO_N (APMV3 N, P, L ORFs optimized for human codon
usage; CO), SEQ ID NO: 10:
ATGGCCGGCATCTTCAACACCTACGAGCTGTTTGTGAAGGACCAGACATGCATGCAC
AAGAGGGCAGCCTCCCTGATCTCTGGAGGACAGCTGAAGAGCAACATCCCCGTGTT
CATCACCACAAAGGACGATCCAGCCGTGAGGTGGGACCTGGTGTGCTTTAATCTGCG
CCTGGTGGTGTCTGAGAGCTCCACCAGCGTGATCAGGCAGGGCGCCCTGATCAGCCT
GCTGTCCATCACCGCCTCTAACATGAGAGCCCTGGCCGCAATCGCAGGACAGACAG
ACGAGAGCATGATCAATATCATCGAGGTGGTGGACTTCGATGGACTGGAGCCACAG
TGCGACACACGGTCCGGCCTGGATGCCCAGAAGCAGGAGATGTTTAAGGACATCGC
CAGGGATATGCCACGCGCCCTGTCTAGCGGCACCCCATTCCAGCACGGAAACGCAG
AGGCAAATGGACCTGAGGATACACACATGTTTCTGAGATCTGCCATCAGCGTGCTGA
CCCAGATCTGGATCCTGGTGGCCAAGGCCATGACAAACATCGACGGCTCCAATGAG
GCCAGCGATCGGAGACTGGCCAAGTACATCCAGCAGAATAGGATCGACAGGCGCTT
CATGCTGGCCCAGGCAACCAGGACAGTGTGCCAGCAGGTCATCAAGGATAGCCTGA
CCCTGCGGAGATTTCTGGTGACAGAGCTGCGGAAGTCCAGAGGCGCCCTGCACTCT
GGCTCCTCTTACTATGCCATGATCGGCGACATGCAGGCTTACATCTTCAACGCCGGC
CTGACCCCATTTCTGACCACACTGCGGTACGGCATCGGCACAAAGTATCACGCCCTG
GCCATCAGCTCCCTGGCCGGCGACCTGAATAAGATCAAGGGCCTGCTGACCCTGTAC
AAGGAGAAGGGCTCCGATGCCGGCTATATGGCCCTGCTGGAGGACGCCGATTGTAT
GCAGTTCGCCCCCGGCAACTACAGCCTGCTGTACTCCTATGCTATGGGCGTGGCCTC
TGTGCACGACGAGGGCATGAGGAATTACCAGTATGCCAGGCGCTTCCTGCACAAGG
GCATGTATCAGTTTGGAAGGGACATCGCAACCCAGCAGCAGCACGCCCTGGATGAG
AGCCTGGCCCAGGAGATGCGGATCACAGAGGCCGACAGAGCCAACCTGAAAGTGAT
GATGGCCAATATCGGCGAGGCCAGCAGCTTCAGCGATATGCCAAACCCAGGAGCCT
CCGGCATCCCCGCCTTCAATGACCGGCCTGATGAGATCTTTTCCGAGCCTGGCTACA
GAAAGCCCCCTCAGGACCAGCCTCTGATCAAGCTGCCAGATCTGGAGGAGGAGGAG
CAGGACGAGTTTGATATGTAATGAATAAAGATCTTATAATAAAAATTCCCATAGCTG
CAGCTCGAGAGGCCTAATTAATTAAGTCGACGATCCGGCTGCTAACAAAGCCCGAA
AGGAAGCTGAGTTGGCTGCTGCCACCGCTGAGCAATAACTAGCATAACCCCTTGGG
GCCTCTAAACGGGTCTTGAGGGGTTTTTTGCTGAAAGGAGGAACTATATCCGGATCG
AGATCAATTCTGTGAGCGTATGGCAAACGAAGGAAAAATAGTTATAGTAGCCGCAC
TCGATGGGACATTTCAACGTAAACCGTTTAATAATATTTTGAATCTTATTCCATTATC
TGAAATGGTGGTAAAACTAACTGCTGTGTGTATGAAATGCTTTAAGGAGGCTTCCTT
TTCTAAACGATTGGGTGAGGAAACCGAGATAGAAATAATAGGAGGTAATGATATGT
ATCAATCGGTGTGTAGAAAGTGTTACATCGACTCATAATATTATATTTTTTATCTAAA
AAACTAAAAATAAACATTGATTAAATTTTAATATAATACTTAAAAATGGATGTTGTG
TCGTTAGATAAACCGTTTATGTATTTTGAGGAAATTGATAATGAGTTAGATTACGAA
CCAGAAAGTGCAAATGAGGTCGCAAAAAAACTGCCGTATCAAGGACAGTTAAAACT
ATTACTAGGAGAATTATTTTTTCTTAGTAAGTTACAGCGACACGGTATATTAGATGG
TGCCACCGTAGTGTATATAGGATCTGCTCCCGGTACACATATACGTTATTTGAGAGA
TCATTTCTATAATTTAGGAGTGATCATCAAATGGATGCTAATTGACGGCCGCCATCA
TGATCCTATTTTAAATGGATTGCGTGATGTGACTCTAGTGACTCGGTTCGTTGATGAG
GAATATCTACGATCCATCAAAAAACAACTGCATCCTTCTAAGATTATTTTAATTTCTG
ATGTGAGATCCAAACGAGGAGGAAATGAACCTAGTACGGCGGATTTACTAAGTAAT
TACGCTCTACAAAATGTCATGATTAGTATTTTAAACCCCGTGGCGTCTAGTCTTAAAT
GGAGATGCCCGTTTCCAGATCAATGGATCAAGGACTTTTATATCCCACACGGTAATA
AAATGTTACAACCTTTTGCTCCTTCATATTCAGGGCCGTCGTTTTACAACGTCGTGAC
TGGGAAAACCCTGGCGTTACCCAACTTAATCGCCTTGCAGCACATCCCCCTTTCGCC
AGCTGGCGTAATAGCGAAGAGGCCCGCACCGATCGCCCTTCCCAACAGTTGCGCAG
CCTGAATGGCGAATGGCGCGACGCGCCCTGTAGCGGCGCATTAAGCGCGGCGGGTG
TGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAACA
ACACTCAACCCTATCTCGGTCTATTCTTTTGATTTATAAGGGATTTTGCCGATTTCGG
CCTATTGGTTAAAAAATGAGCTGATTTAACAAAAATTTAACGCGAATTTTAACAAAA
TATTAACGTTTACAATTTCCCAGGTGGCACTTTTCGGGGAAATGTGCGCGGAACCCC
TATTTGTTTATTTTTCTAAATACATTCAAATATGTATCCGCTCATGAGACAATAACCC
TGATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAAAC
GCTGGTGAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAACGTTTTC
CAATGATGAGCACTTTTAAAGTTCTGCTATGTGGCGCGGTATTATCCCGTATTGACG
CCGGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTATGC
AGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAACTA
CTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGA
GCCGGTGAGCGTGGGTCTCGCGGTATCATTGCAGCACTGGGGCCAGATGGTAAGCC
CTCCCGTATCGTAGTTATCTACACGACGGGGAGTCAGGCAACTATGGATGAACGAA
ATAGACAGATCGCTGAGATAGGTGCCTCACTGATTAAGCATTGGTAACTGTCAGACC
AAGTTTACTCATATATACTTTAGATTGATTTAAAACTTCATTTTTAATTTAAAAGGAT
CTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGA
GCGCAGATACCAAATACTGTCCTTCTAGTGTAGCCGTAGTTAGGCCACCACTTCAAG
AACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTTGCTGGC
CTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTC
AGTGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCAGT
GAGCGCAACGCAATTAATGTGAGTTAGCTCACTCATTAGGCACCCCAGGCTTTACAC
TTTATGCTTCCGGCTCGTATGTTGTGTGGAATTGTGAGCGGATAACAATTTCACACA
GGAAACAGCTATGACCATGATTACGCCAAGCTTTTGCGATCAATAAATGGATCACA
ACCAGTATCTCTTAACGATGTTCTTCGCAGATGATGATTCATTTTTTAAGTATTTGGC
TAGTCAAGATGATGAATCTTCATTATCTGATATATTGCAAATCACTCAATATCTAGA
CTTTCTGTTATTATTATTGATCCAATCAAAAAATAAATTAGAAGCCGTGGGTCATTGT
TATGAATCTCTTTCAGAGGAATACAGACAATTGACAAAATTCACAGACTTTCAAGAT
TTTAAAAAACTGTTTAACAAGGTCCCTATTGTTACAGATGGAAGGGTCAAACTTAAT
AAAGGATATTTGTTCGACTTTGTGATTAGTTTGATGCGATTCAAAAAAGAATCCTCT
CTAGCTACCACCGCAATAGATCCTGTTAGATACATAGATCCTCGTCGCAATATCGCA
TTTTCTAACGTGATGGATATATTAAAGTCGAATAAAGTGAACAATAATTAATTCTTT
ATTGTCATCATGAACGGCGGACATATTCAGTTGATAATCGGCCCCATGTTTTCAGGT
AAAAGTACAGAATTAATTAGACGAGTTAGACGTTATCAAATAGCTCAATATAAATG
CGTGACTATAAAATATTCTAACGATAATAGATACGGAACGGGACTATGGACGCATG
ATAAGAATAATTTTGAAGCATTGGAAGCAACTAAACTATGTGATGTCTTGGAATCAA
TTACAGATTTCTCCGTGATAGGTATCGATGAAGGACAGTTCTTTCCAGACATTGTTG
AATTGATCTCGATCCCGCGAAATTAATACGACTCACTATAGGGAGACCACAACGGTT
TCCCTCTAGCGGGATCAATTCCGCCCCTCTCCCTCCCCCCCCCCTAACGTTACTGGCC
GAAGCCGCTTGGAATAAGGCCGGTGTGCGTTTGTCTATATGTTATTTTCCACCATATT
GCCGTCTTTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCGACCCTTTG
CAGGCAGCGGAACCCCCCACCTGGCGACAGGTGCCTCTGCGGCCAAAAGCCACGTG
TATAAGATACACCTGCAAAGGCGGCACAACCCCAGTGCCACGTTGTGAGTTGGATA
GTTGTGGAAAGAGTCAAATGGCTCTCCTCAAGCGTATTCAACAAGGGGCTGAAGGA
TGCCCAGAAGGTACCCCATTGTATGGGATCTGATCTGGGGCCTCGGTGCACATGCTT
TACATGTGTTTAGTCGAGGTTAAAAAACGTCTAGGCCCCCCGAACCACGGGGACGT
GGTTTTCCTTTGAAAAACACGATAATACC
pTM 1_APMV3_CO_P (SEQ ID NO: 11):
ATGGATCTGGAGTTCAGCTCCGAGGAGGCAGTGGCCGCCCTGCTGGACGTGTCTAGC
TCCACCATCACAGAGGTGCTGTCCAAGCAGTCTATCCCTGATCCATCTTTTCTGACC
AGCACAGAGGCCAGCGGAGAGCAGGTGCCCGACTCTAAGGTGGCCAACCACAAGG
CCAGCGTGGATAAGACCCCTGACCAGGGACAGCCATCTGCCACACCAAGCGCCCCA
CCTGAGACCGCCGAGAACATCAATACACCTAGCTGCGAGGATGGCCTGCCACCCAA
CTTCTTTATCCCACGGGTGGAGTCCTACCACACCAATCTGTTCAAGGGCGGCCCCCA
GCTGGACTCTACAGAGAGACCTCCAGGCCACCAGATCGTGTGCGGCGACCAGGATG
CAGACCTGTCCAGGGCCGGAATCGCACGGAAGAAGAAGAAGCAGAAGCACTGTAA
GGCCCTGAACTTTAGCGAGTCCCCTCTGACCGATAATCAGGCCATCGAGGGAAGCA
TCCAGTCCCACGGCGCCCTGGACCAGGAGCCTAGCCGGCAGAGACCAGGAGCAACC
CAGCCCGCCCTCCAGTCTCCCCCTAGCCAGAACAATACATCCGTGCACGCCGATTCC
GCCCAGGACTCTGCCATCTCTGTGAGCATCCCTCTGACAATGGTGGAGTCCCTGATC
TCTCAGGTGAGCAAGCTGTCCGATCAGGTGAGCCAGATCCAGAAGCTGGTGTCCAC
CCTGCCACAGATCAAGACAGACATCGCCAGCATCAGGAACATGCAGGCCGCCCTGG
AGGGACAGCTGTCTATGATCCGCATCCTGGATCCAGGCAACTGTAGCGAGTCTAGCC
TGAATGCACTGAGGGGCTTCTCCGGCAAGGCACCCGTGATCGTGTCTGGACCCGGC
AACCCTAATCAGTGCATCAGCCAGGGCTCCTCTACCACAATCTGTCTGGATGAGCTG
GCCAGGCCCGTGCCTAATCCAATCCAGGTGAAGCTCCAGGACGATCAGAAGGACCT
GTCCGCCCAGAGGCACGCCGTGATCGCCCTGCTGGAGACCCGGATCCCACCAGGAC
CAAAGAGAGATAAGCTGCTGAGCCTGGTGGCCATCGCCAAGACAGCCTCCGACCTG
ATCAAGATCAAGAGAATGGCCGTGCTGGGCCAGTAATGAATAAAGATCTTATAATA
AAAATTCCCATAGCTGCAGCTCGAGAGGCCTAATTAATTAAGTCGACGATCCGGCTG
CTAACAAAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAAAGGAGGA
ACTATATCCGGATCGAGATCAATTCTGTGAGCGTATGGCAAACGAAGGAAAAATAG
TTATAGTAGCCGCACTCGATGGGACATTTCAACGTAAACCGTTTAATAATATTTTGA
ATCTTATTCCATTATCTGAAATGGTGGTAAAACTAACTGCTGTGTGTATGAAATGCTT
TAAGGAGGCTTCCTTTTCTAAACGATTGGGTGAGGAAACCGAGATAGAAATAATAG
GAGGTAATGATATGTATCAATCGGTGTGTAGAAAGTGTTACATCGACTCATAATATT
ATATTTTTTATCTAAAAAACTAAAAATAAACATTGATTAAATTTTAATATAATACTTA
AAAATGGATGTTGTGTCGTTAGATAAACCGTTTATGTATTTTGAGGAAATTGATAAT
GAGTTAGATTACGAACCAGAAAGTGCAAATGAGGTCGCAAAAAAACTGCCGTATCA
AGGACAGTTAAAACTATTACTAGGAGAATTATTTTTTCTTAGTAAGTTACAGCGACA
CGGTATATTAGATGGTGCCACCGTAGTGTATATAGGATCTGCTCCCGGTACACATAT
ACGTTATTTGAGAGATCATTTCTATAATTTAGGAGTGATCATCAAATGGATGCTAAT
TGACGGCCGCCATCATGATCCTATTTTAAATGGATTGCGTGATGTGACTCTAGTGAC
TCGGTTCGTTGATGAGGAATATCTACGATCCATCAAAAAACAACTGCATCCTTCTAA
GATTATTTTAATTTCTGATGTGAGATCCAAACGAGGAGGAAATGAACCTAGTACGGC
GGATTTACTAAGTAATTACGCTCTACAAAATGTCATGATTAGTATTTTAAACCCCGT
GGCGTCTAGTCTTAAATGGAGATGCCCGTTTCCAGATCAATGGATCAAGGACTTTTA
TATCCCACACGGTAATAAAATGTTACAACCTTTTGCTCCTTCATATTCAGGGCCGTCG
TTTTACAACGTCGTGACTGGGAAAACCCTGGCGTTACCCAACTTAATCGCCTTGCAG
CACATCCCCCTTTCGCCAGCTGGCGTAATAGCGAAGAGGCCCGCACCGATCGCCCTT
CCCAACAGTTGCGCAGCCTGAATGGCGAATGGCGCGACGCGCCCTGTAGCGGCGCA
TTAAGCGCGGCGGGTGTGGTGGTTACGCGCAGCGTGACCGCTACACTTGCCAGCGC
CCTAGCGCCCGCTCCTTTCGCTTTCTTCCCTTCCTTTCTCGCCACGTTCGCCGGCTTTC
CCCGTCAAGCTCTAAATCGGGGGCTCCCTTTAGGGTTCCGATTTAGTGCTTTACGGC
ACCTCGACCCCAAAAAACTTGATTAGGGTGATGGTTCACGTAGTGGGCCATCGCCCT
GATAGACGGTTTTTCGCCCTTTGACGTTGGAGTCCACGTTCTTTAATAGTGGACTCTT
GTTCCAAACTGGAACAACACTCAACCCTATCTCGGTCTATTCTTTTGATTTATAAGGG
ATTTTGCCGATTTCGGCCTATTGGTTAAAAAATGAGCTGATTTAACAAAAATTTAAC
GCGAATTTTAACAAAATATTAACGTTTACAATTTCCCAGGTGGCACTTTTCGGGGAA
ATGTGCGCGGAACCCCTATTTGTTTATTTTTCTAAATACATTCAAATATGTATCCGCT
CATGAGACAATAACCCTGATAAATGCTTCAATAATATTGAAAAAGGAAGAGTATGA
GTATTCAACATTTCCGTGTCGCCCTTATTCCCTTTTTTGCGGCATTTTGCCTTCCTGTT
TTTGCTCACCCAGAAACGCTGGTGAAAGTAAAAGATGCTGAAGATCAGTTGGGTGC
ACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGT
ATTATCCCGTATTGACGCCGGGCAAGAGCAACTCGGTCGCCGCATACACTATTCTCA
GAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAAC
TTACTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATACC
AAACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGATGG
AGGCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACTGG
GGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCA
TTGGTAACTGTCAGACCAAGTTTACTCATATATACTTTAGATTGATTTAAAACTTCAT
TTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCAC
CGCTACCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCCGTAGT
TAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAA
GACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATG
CTCGTCAGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCCCCTGATTCT
GTGGATAACCGTATTACCGCCTTTGAGTGAGCTGATACCGCTCGCCGCAGCCGAACG
ACCGAGCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGGTTTCCCG
ACTGGAAAGCGGGCAGTGAGCGCAACGCAATTAATGTGAGTTAGCTCACTCATTAG
GCACCCCAGGCTTTACACTTTATGCTTCCGGCTCGTATGTTGTGTGGAATTGTGAGCG
GATAACAATTTCACACAGGAAACAGCTATGACCATGATTACGCCAAGCTTTTGCGAT
CAATAAATGGATCACAACCAGTATCTCTTAACGATGTTCTTCGCAGATGATGATTCA
TTTTTTAAGTATTTGGCTAGTCAAGATGATGAATCTTCATTATCTGATATATTGCAAA
TCACTCAATATCTAGACTTTCTGTTATTATTATTGATCCAATCAAAAAATAAATTAGA
AGCCGTGGGTCATTGTTATGAATCTCTTTCAGAGGAATACAGACAATTGACAAAATT
CACAGACTTTCAAGATTTTAAAAAACTGTTTAACAAGGTCCCTATTGTTACAGATGG
AAGGGTCAAACTTAATAAAGGATATTTGTTCGACTTTGTGATTAGTTTGATGCGATT
CAAAAAAGAATCCTCTCTAGCTACCACCGCAATAGATCCTGTTAGATACATAGATCC
TCGTCGCAATATCGCATTTTCTAACGTGATGGATATATTAAAGTCGAATAAAGTGAA
CAATAATTAATTCTTTATTGTCATCATGAACGGCGGACATATTCAGTTGATAATCGG
CCCCATGTTTTCAGGTAAAAGTACAGAATTAATTAGACGAGTTAGACGTTATCAAAT
AGCTCAATATAAATGCGTGACTATAAAATATTCTAACGATAATAGATACGGAACGG
GACTATGGACGCATGATAAGAATAATTTTGAAGCATTGGAAGCAACTAAACTATGT
GATGTCTTGGAATCAATTACAGATTTCTCCGTGATAGGTATCGATGAAGGACAGTTC
TTTCCAGACATTGTTGAATTGATCTCGATCCCGCGAAATTAATACGACTCACTATAG
GGAGACCACAACGGTTTCCCTCTAGCGGGATCAATTCCGCCCCTCTCCCTCCCCCCC
CCCTAACGTTACTGGCCGAAGCCGCTTGGAATAAGGCCGGTGTGCGTTTGTCTATAT
GTTATTTTCCACCATATTGCCGTCTTTTGGCAATGTGAGGGCCCGGAAACCTGGCCCT
GTCTTCTTGACGAGCATTCCTAGGGGTCTTTCCCCTCTCGCCAAAGGAATGCAAGGT
CTGTTGAATGTCGTGAAGGAAGCAGTTCCTCTGGAAGCTTCTTGAAGACAAACAACG
TCTGTAGCGACCCTTTGCAGGCAGCGGAACCCCCCACCTGGCGACAGGTGCCTCTGC
GGCCAAAAGCCACGTGTATAAGATACACCTGCAAAGGCGGCACAACCCCAGTGCCA
CGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGC
CTCGGTGCACATGCTTTACATGTGTTTAGTCGAGGTTAAAAAACGTCTAGGCCCCCC
GAACCACGGGGACGTGGTTTTCCTTTGAAAAACACGATAATACC
pTM 1_APMV3_CO_L (SEQ ID NO: 12):
ATGAGCTCCCACAACATCATCCTGCCAGACCACCACCTGAACAGCCCCATCGTGCTG
AATAAGCTGATGTACTATTGTAAGCTGCTGAATATCCTGCCAGATCCCAAGTCCCCT
TGGTACGAGAAGATCAAGTCTTGGACCAACTGCTGTATCCGCGTGTCCGACTCTAAT
AGGATGACACTGTCCCGCGCCTCTAAGCTGAGGGAGCTGCTGGCCACCTACGGCGT
GTATAGCAAGAACCACCAGCAGTGCTATACCACAATCATCCACCCCCAGAGCCTGT
CCCCTATCATGCAGACCGTGAGCCAGCTGGGCCGGTCCATCCCAACATGGGCACGC
ATCCGGAAGGAGATCACACACTCCATCATCGCCCAGACCAACAAGTTCGAGAGCCT
GTTTCACAATATCTCCAAGGATCTGACAGGCAAGACCAACCTGTTCGGCGGCTTTTG
CGACCTGCACGGCTCTATCAGCACAGCCGCCAAGCGCCTGATGAATCAGCCTGGCCT
GTACCTGGACAGCCCAGATGCCCACGCCTGTCAGTTCCTGTTTCAGCTGAAGACCTG
CCAGAGAGAGCTGATCCTGCTGATGAGGCAGAACGCCACCACAGAGCTGATCCGCG
TGTTCGATTACCCAGAGCTGCGGGTGCTGGTGACCCCAGAGTATTCCGTGTGGGTGT
TCACAAGAACCAAGCAGGTGACACTGCTGACCTTTGACTGTCTGCTGATGTATTGCG
ACCTGTCCGATGGCAGGCACAACATCCTGTTTACCTGTAAGCTGCTGTCTCACCTGA
ATCACCTGGGCTGCCGGATCCGGGATCTGCTGGTGCTGATCGACTCCCTGGCCGAGA
AGCACCCCCTGATCGTGTACGATGTGGTGGCCTCTCTGGAGTCCCTGTCTTATGGCG
CCATCCAGCTGCACGACAAGGTGGCAGGATACGCAGGCACATTCTTTTCCTTCATCC
TGGCCGAGATCCAGGATTCTCTGGAGACCGTGCTGGACCAGGGCAACAGCGAGTCC
ATCATCTCCCAGATCAGAAACATCTATTCTGGCCTGACCGTGAATGAGGCCGCCGAG
CTGCTGTGCGTGATGAGACTGTGGGGACACCCCGCCCTGAACAGCGTGGATGCAGC
CAGCAAGGTGAGGCAGTCCATGTGCGCCGGCAAGCTGCTGAAGTTTGACACCATCC
AGCTGGTGCTGGCCTTCTTTAACACACTGATCATCAATGGCTACCGGAGAAAGCACC
ACGGCCGCTGGCCAAACGTGTGCAGCGATAGCATCATCGGCAATGAGCTGAAGCGG
ATGTACTTCGATCAGTGCGAGATCCCTCACGACTTTACACTGAAGAACTATAGGGAG
CTGTCCCTGCTGGAGTTCGAGTGTACCTTTCCAATCGAGCTGTCTGATAAGCTGAAT
ATCTTCCTGAAGGACAAGGCCATCGCCTTTCCCAAGAGCAGATGGACCTCCCCTTTC
AAGGCCGACATCACACCAAGGCACCTGCTCCAGGCCCCCGAGTTTAAGACCCGGGC
CAACAGACTGCTGCTGAGCTTCCTCCAGCTGGAGGAGTTTAGCATCGAGTCCGAGCT
GGAGTACGTGACCACACTGGCCTATCTGGACGATGACGAGTTCAACGTGAGCTACTC
TCTGAAGGAGAAGGAGGTGAAGACCGATGGCCGGATCTTTGCCAAGCTGACAAGGA
AGATGCGCAGCTGCCAGGTCATCTTCGAGGAGCTGCTGGCCGAGCACGTGTCCCCCC
TGTTTAAGGACAACGGCGTGACAATGGCCGAGCTGTCTCTGACCAAGAGCCTGCTG
GCCATCAGCAACCTGTCTAGCACACTGTTCGAGACACAGACCAGACAGGGCGATCG
GAATGCCAGATTCACCCACGCCCACTTTATCACCACAGACCTCCAGAAGTACTGTCT
GAACTGGAGGTATCAGTCCGTGAAGCTGTTTGCCCGGCAGCTGAATAGACTGTTCGG
CCTCCAGCACGGCTTTGAGTGGATCCACTGCATCCTGATGAAGAGCACCATGTACGT
GGCCGATCCCTTCAACCCACCTCACTCCAATGCCCGCGAGGCCCTGGATGACAACCT
GAACAGCGACATCTTTATCGTGAGCCCTCGGGGCGCAATCGAGGGCCTGTGCCAGA
AGATGTGGACCATCATCAGCATCTCCGCCATCCACGCCTCTGCCGCCGTGGCCGGCC
TGAGAGTGGCCAGCATGGTGCAGGGCGATAACCAGGTCATCGGAGTGACCAGGGAG
TTCCTGGCAGGACACGACCAGACATTTGTGGATGACCAGCTGACCGTGTCCCTGGAG
AATTTCACACAGATCTTTAAGGAGGTGAACTACGGCCTGGGCCACAATCTGAAGCTG
CGCGAGACAATCAAGTCCTCTCACATGTTCATCTACAGCAAGAGAATCTTTTATGAC
GGCAGGGTGCTGCCACAGCTGCTGAAGAACATCTCTAAGCTGACCCTGAGCGCCAC
CACAACCGGCGAGAATTGTCTGACCTCCTGCGGCGATCTGAGCTCCTGTATCACAAG
ATGCATCGAGAACGGCTTCCCCAAGGACGCCGCCTTTGTGCTGAATCAGCTGGTCAT
CAGGATCCAGATCCTGGCCGATCACTTCTACAGCATCCTGGGCGGCTGCTTTTCCGG
CCTGAACCAGGCAGACATCAGGCTGCTGCTGTATGAGGGAGCAATCCTGCCAGCAC
AGCTGGGAGGCTTCAACAATCTGAACATGTCTAGGCTGTTTTGTCGCAATATCGGCG
ATCCCCTGGTGGCCAGCATCGCAGACACAAAGCGGTTCGTGAAGTGCCAGCTGCTG
GTGCCTCACATCCTGGATTCCGTGGTGGCCATCACCGACCGGAAGGGCTCTTTTACA
ACCCTGATGATGGATCCTTACAGCATCAACCTGGACTATATCCAGCAGCCAGAGACC
AGGCTGAAGCGCCACGTGCAGAAGGTGCTGCTCCAGGAGTCTGTGAATCCACTGCT
CCAGGGCGTGTTCCTGGAGAGCCAGCAGGAGGAGGAGGAGCAGCTGGCCGCCTTTC
TGCTGGACAGGGAGGTGGTCATGCCCCGGGTGGCACACGCAATCTTCGAGTGTACA
TCCCTGGGCAGGCGCCGGCACATCCAGGGCCTGATCGATACAACCAAGACCATCAT
CGCCCTGGCCCTGGACACACAGCAGCTGAGCTACACCAAGCGCGAGCAGATCGTGA
CATACAACGCCACCTATATGCGGTCTCTGGCCAGCATGCTGTCTAGCTCCCACAACC
AGACAAGAAGGTCCGTGATCGGCCACGCCTCTTTTAATATCACCGACTGCTCCGTGA
TCCTGGCACAGCAGGTGCGCAGGGCCAGCTGGGCCCCTCTGCTGAATTGGAGATCC
CTGGAGGGCCTGGAGGTGCCTGATCCAATCGAGTCTGTGGCCGGCTACCTGGGCCTG
GACAGCAACAATTGTTTCCTGTGCTGTCACGAGCAGAACTCTTATAGCTGGTTCTTTC
TGCCTCGCATGTGCCACTTTGATGACAGCCGCCAGTCCAATTCTATCCAGCGGGTGC
CATACATCGGCAGCAAGACCGATGAGCGGCAGATGTCCACAATCAACCTGCTGGAG
AAGACAACCTGCCACGCCAGAGCCGCAACCAGACTGGCCTCCCTGTATATCTGGGC
CTTCGGCGATTCCGATGACTCTTGGGACGCCGTGGAGACCCTGAGCAATTCCAGATG
TCAGATCACAAGGGAGCACCTCCAGGCCCTGTGCCCAATGCCTTCTAGCGTGAACCT
GCACCACAGACTGAATGACGGCATCACCCAGGTGAAGTTCATGCCCAGCACAAACT
CCCGGGTGTCTAGATTTGTGCACATCAGCAACGATAGGCAGAATTACGTGCTGGATG
ACTCCGTGACAGACTCTAACCTGATCTATCAGCAGGTCATGCTGCTGGGCCTGTCTA
TCCTGGAGACCTACTTCAGAGAGCCTACAGCCACCAATCTGTCCTCTCTGGTGCTGC
ACCTGCACACCGATGTGAGCTGCTGTCTGAGAGAGTGTCCCATGACACAGTATGCCC
CACCCCTGAGGGACATCCCTGAGCTGACAATCACCGCCTCCAACCCTTTCCTGTTTG
ATCAGGAGCCAATCTCTGAGGCCGACCTGTGCCGCCTGAGCAAGATCGCCTTCAGA
AGGGCCGGCGACAATTACGATGCCTATGACCAGTTTCAGCTGAGGGCCACCCTGGC
CGCAACAACCGGCAAGGATGTGGCAGCAACAATCTTCGGACCCCTGGCCGCCGTGA
GCGCCAAGAACGATGCCATCGTGACCAACGACTACTCCGGCAATTGGATCTCTGAG
TTCAGGTACAGCGACCTGTATCTGCTGTCTGTGAGCCTGGGCTACGAGATCCTGCTG
ATCTTTGCCTATCAGCTGTACTATCTGCGCATCCGGTACAGACAGAATATCGTGTGC
TATATGGAGAGCGTGTTCCGGCGGTGCCACAGCCTGTGCCTGGGCGACCTGATCCAG
ACAATCTCCCACTCTGAGATCCTGGTGGGCCTGAACGCCGCCGGCTTTAATCTGACC
CTGGATAGATCCGACCTGAAGGAGAACCAGCTGTCTAGGCTGGCCGTGAAGTACCT
GACCCTGTGCGTGCAGACCGCCATCAGCAATCTGGAGGTCGGCTCCGAGCCTCTGTG
CATCATCGGCGGCCAGCTGGATGACGATATCAGCTTCCAGGTGGCACACTTCCTGTG
CCGGCGGCTGTGCATCATCAGCCTGGTGCACTCCAACGTGCAGAATCTGCCTCCAAT
CAGAGATAACGAGGTGGACGTGAAGAGCAAGCTGATCTACGATCACCTGAAGCTGG
TGGCCACAACCCTGAACAATAGGGACCAGTCCTATCTGCTGACCCTGCTGAACAAG
CCCAATATCGAGCTGCACACACCTCAGGTGTACTTCATCATGCGGAAGTGTCTGGGC
CTGCTGAAGGTGTATGGCCCAGTGCCCCAGAAGCAGCCTCTGCCAACCTCTCCAGTG
GTGAGCCTGCCCAACAAGTGCAAGTCCAAGTGGAAGCTGGAGGAAGTGATCGACAG
CATCGAGTCCCCCAAGTCTTTTAATTGGGTGCCTGATACAACCATCCCACTGGACGC
CGAGCAGAACCCCCCTAACCCAAATCGCGTGATCGATAAGATCAATATCCTGCGCA
GCCTGTCCCCAAGGCACAGCGTGTGGTACAGAAACCGCCAGTACAGATATGTGCTG
AGCCAGCTGGGACACGACCAGCTGGGAGGAGCCACCCTGTATCTGGGAGAGGGCTC
TGGCAGCACAATCCTGAATATCGAGCCAAAGGTGCGCTCCGATAAGATCTACTATCA
CACCTACTTCTCCGCCGACAAGTCTCCCGCCCAGCGCAACTTTATCCCCCAGCCTAC
AACCTTCCTGCGGAGCAACTTCTACCACTATGAGCTGGAGCCCTCCGGCTGTGAGTT
CATCAACTGCTGGTCTGAGGATGTGAACGCCACAAATCTGACCGAGCTGCGCTGTAT
CAATCACATCCTGACCGTGATCCCTGTGGGCTCTCTGACACGGATCATCTGCGATGT
GGAGCTGGCCAACGACACCAGCATCCAGTCCGTGGCCACAGCCTACATGAACCTGA
GCATCCTGGCACACGCACTGCTGAATCAGGGAGGCGTGTGCATCTGTCGGTGCCACC
TGCTGGACACCTCTCACCTGGCCATCGTGAGCTTCGTGCTGAAGACACTGAGCTCCC
AGCTGGCCATCTCCTTTTCTGGCTATTGTGGCACCAACGATCCTAGCTGCGTGATCG
GCGTGACAAAGGAGTGTAGCATCTCCCTGGACGTGCTGTCTAGCATCGCCGCCGCCT
TCATCAACGAGCTGCCAAGCAATGAGCTGCCAGTGCCCCAGTCCCTGCTGACACTGC
TGGGCTGCTACCAGGATCAGCTGGAGTCTCTGAGCGCCCTGATCGAGAAGACCTGG
ATCCAGGAGATCCGCCGGCCACACCTGATGCAGTGTGAGATGGAGTGGATCGGCCT
GCTGGGCACCGACGCACTGGGCGACGTGGATACATTCATCTCCTATAGAAACGATTC
TTGCAATAGCATCCCAGACCTGCTGACCCCTGCCGTGTCTGCCCTGCTGTTTGAGCT
GATCAGCCTGACACCTGAGCTGCCAGGCACCGAGGATCAGCCCTCCAGAAGAGTGA
TCTCTATCGGCCAGGCTTTCAACCTGACCATCAGCGGCAAGGTCAATACAATGATCA
GGTCCTGCTGTGAGCAGTGCATCAAGCTGCTGGCCGCCAACGTGTCCATCCTGTCTG
ACGTGGATCTGTCCTACTTCGTGAGGGGCATCCGCGACGGCTCTTTTGCCCTGGGCC
TGCTGATCACACAGAGACAGATCCTGAAGGCCAGCCGGGCCCCCAAGTATCTGAAG
ACCCACCAGGTGCAGAATTGGATCGCCCTGCTGCTGGAGGTGAAGCTGGAGGAGAT
CTTCAGCCGGCACTACAGAAAGGTGCTGCTGAGGACCCTGCGCCTGCTGAGCCTGTA
TGGCCTGCTCCAGCAGAAGGAGTCCTAATGAATAAAGATCTTATAATAAAAATTCCC
ATAGCTGCAGCTCGAGAGGCCTAATTAATTAAGTCGACGATCCGGCTGCTAACAAA
GCCCGAAAGGAAGCTGAGTTGGCTGCTGCCACCGCTGAGCAATAACTAGCATAACC
CCTTGGGGCCTCTAAACGGGTCTTGAGGGGTTTTTTGCTGAAAGGAGGAACTATATC
CGGATCGAGATCAATTCTGTGAGCGTATGGCAAACGAAGGAAAAATAGTTATAGTA
GCCGCACTCGATGGGACATTTCAACGTAAACCGTTTAATAATATTTTGAATCTTATTC
CATTATCTGAAATGGTGGTAAAACTAACTGCTGTGTGTATGAAATGCTTTAAGGAGG
CTTCCTTTTCTAAACGATTGGGTGAGGAAACCGAGATAGAAATAATAGGAGGTAAT
GATATGTATCAATCGGTGTGTAGAAAGTGTTACATCGACTCATAATATTATATTTTTT
ATCTAAAAAACTAAAAATAAACATTGATTAAATTTTAATATAATACTTAAAAATGGA
TGTTGTGTCGTTAGATAAACCGTTTATGTATTTTGAGGAAATTGATAATGAGTTAGA
TTACGAACCAGAAAGTGCAAATGAGGTCGCAAAAAAACTGCCGTATCAAGGACAGT
TAAAACTATTACTAGGAGAATTATTTTTTCTTAGTAAGTTACAGCGACACGGTATAT
TAGATGGTGCCACCGTAGTGTATATAGGATCTGCTCCCGGTACACATATACGTTATT
TGAGAGATCATTTCTATAATTTAGGAGTGATCATCAAATGGATGCTAATTGACGGCC
GCCATCATGATCCTATTTTAAATGGATTGCGTGATGTGACTCTAGTGACTCGGTTCGT
TGATGAGGAATATCTACGATCCATCAAAAAACAACTGCATCCTTCTAAGATTATTTT
AATTTCTGATGTGAGATCCAAACGAGGAGGAAATGAACCTAGTACGGCGGATTTAC
TAAGTAATTACGCTCTACAAAATGTCATGATTAGTATTTTAAACCCCGTGGCGTCTA
GTCTTAAATGGAGATGCCCGTTTCCAGATCAATGGATCAAGGACTTTTATATCCCAC
ACGGTAATAAAATGTTACAACCTTTTGCTCCTTCATATTCAGGGCCGTCGTTTTACAA
CGTCGTGACTGGGAAAACCCTGGCGTTACCCAACTTAATCGCCTTGCAGCACATCCC
CCTTTCGCCAGCTGGCGTAATAGCGAAGAGGCCCGCACCGATCGCCCTTCCCAACAG
TTGCGCAGCCTGAATGGCGAATGGCGCGACGCGCCCTGTAGCGGCGCATTAAGCGC
GGCGGGTGTGGTGGTTACGCGCAGCGTGACCGCTACACTTGCCAGCGCCCTAGCGC
CCGCTCCTTTCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTT
TAACAAAATATTAACGTTTACAATTTCCCAGGTGGCACTTTTCGGGGAAATGTGCGC
GGAACCCCTATTTGTTTATTTTTCTAAATACATTCAAATATGTATCCGCTCATGAGAC
AATAACCCTGATAAATGCTTCAATAATATTGAAAAAGGAAGAGTATGAGTATTCAA
CATTTCCGTGTCGCCCTTATTCCCTTTTTTGCGGCATTTTGCCTTCCTGTTTTTGCTCA
CCCAGAAACGCTGGTGAAAGTAAAAGATGCTGAAGATCAGTTGGGTGCACGAGTGG
GTTACATCGAACTGGATCTCAACAGCGGTAAGATCCTTGAGAGTTTTCGCCCCGAAG
AACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTT
GGTTGAGTACTCACCAGTCACAGAAAAGCATCTTACGGATGGCATGACAGTAAGAG
AATTATGCAGTGCTGCCATAACCATGAGTGATAACACTGCGGCCAACTTACTTCTGA
CAACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGA
GCGTGACACCACGATGCCTGTAGCAATGGCAACAACGTTGCGCAAACTATTAACTG
GCGAACTACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTGCTGATA
AATCTGGAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAACTATGGAT
GAACGAAATAGACAGATCGCTGAGATAGGTGCCTCACTGATTAAGCATTGGTAACT
GTCAGACCAAGTTTACTCATATATACTTTAGATTGATTTAAAACTTCATTTTTAATTT
AAAAGGATCTAGGTGAAGATCCTTTTTGATAATCTCATGACCAAAATCCCTTAACGT
GAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCA
GTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAG
CTTGGAGCGAACGACCTACACCGAACTGAGATACCTACAGCGTGAGCTATGAGAAA
GCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCCTTT
TGCTGGCCTTTTGCTCACATGTTCTTTCCTGCGTTATCCCCTGATTCTGTGGATAACC
GTATTACCGCCTTTGAGTGAGCTGATACCGCTCGCCGCAGCCGAACGACCGAGCGC
AGCGAGTCAGTGAGCGAGGAAGCGGAAGAGCGCCCAATACGCAAACCGCCTCTCCC
CGCGCGTTGGCCGATTCATTAATGCAGCTGGCACGACAGGTTTCCCGACTGGAAAGC
GGGCAGTGAGCGCAACGCAATTAATGTGAGTTAGCTCACTCATTAGGCACCCCAGG
CTTTACACTTTATGCTTCCGGCTCGTATGTTGTGTGGAATTGTGAGCGGATAACAATT
TCACACAGGAAACAGCTATGACCATGATTACGCCAAGCTTTTGCGATCAATAAATGG
ATCACAACCAGTATCTCTTAACGATGTTCTTCGCAGATGATGATTCATTTTTTAAGTA
TTTGGCTAGTCAAGATGATGAATCTTCATTATCTGATATATTGCAAATCACTCAATAT
CTAGACTTTCTGTTATTATTATTGATCCAATCAAAAAATAAATTAGAAGCCGTGGGT
CATTGTTATGAATCTCTTTCAGAGGAATACAGACAATTGACAAAATTCACAGACTTT
CAAGATTTTAAAAAACTGTTTAACAAGGTCCCTATTGTTACAGATGGAAGGGTCAAA
CTTAATAAAGGATATTTGTTCGACTTTGTGATTAGTTTGATGCGATTCAAAAAAGAA
TCCTCTCTAGCTACCACCGCAATAGATCCTGTTAGATACATAGATCCTCGTCGCAAT
ATCGCATTTTCTAACGTGATGGATATATTAAAGTCGAATAAAGTGAACAATAATTAA
TTCTTTATTGTCATCATGAACGGCGGACATATTCAGTTGATAATCGGCCCCATGTTTT
CAGGTAAAAGTACAGAATTAATTAGACGAGTTAGACGTTATCAAATAGCTCAATAT
AAATGCGTGACTATAAAATATTCTAACGATAATAGATACGGAACGGGACTATGGAC
GCATGATAAGAATAATTTTGAAGCATTGGAAGCAACTAAACTATGTGATGTCTTGGA
ATCAATTACAGATTTCTCCGTGATAGGTATCGATGAAGGACAGTTCTTTCCAGACAT
TGTTGAATTGATCTCGATCCCGCGAAATTAATACGACTCACTATAGGGAGACCACAA
CGGTTTCCCTCTAGCGGGATCAATTCCGCCCCTCTCCCTCCCCCCCCCCTAACGTTAC
TGGCCGAAGCCGCTTGGAATAAGGCCGGTGTGCGTTTGTCTATATGTTATTTTCCAC
CATATTGCCGTCTTTTGGCAATGTGAGGGCCCGGAAACCTGGCCCTGTCTTCTTGAC
GAGCATTCCTAGGGGTCTTTCCCCTCTCGCCAAAGGAATGCAAGGTCTGTTGAATGT
CGTGAAGGAAGCAGTTCCTCTGGAAGCTTCTTGAAGACAAACAACGTCTGTAGCGA
CCCTTTGCAGGCAGCGGAACCCCCCACCTGGCGACAGGTGCCTCTGCGGCCAAAAG
CCACGTGTATAAGATACACCTGCAAAGGCGGCACAACCCCAGTGCCACGTTGTGAG
TTGGATAGTTGTGGAAAGAGTCAAATGGCTCTCCTCAAGCGTATTCAACAAGGGGCT
GAAGGATGCCCAGAAGGTACCCCATTGTATGGGATCTGATCTGGGGCCTCGGTGCA
CATGCTTTACATGTGTTTAGTCGAGGTTAAAAAACGTCTAGGCCCCCCGAACCACGG
GGACGTGGTTTTCCTTTGAAAAACACGATAATACC

S-6P ORF matching a representative of a Delta variant; with description of changes compared to S-6P:

hCoV-19/USA/CT-JAX-JAX001420/2021. The S ORF sequence of this seq ID was selected as a B.1.617.2 representative (Nextstrain.org: nextstrain.org/ncov/gisaid/global?gmin=15). Six proline substitutions were included in amino acid locations corresponding to those of the original SARS-CoV-2 S sequence (GenBank accession number NC 045512.2, in which the sequence of nucleic acids 21563 to 25384 is a coding sequence for Spike protein (S)).

The open reading frame encoding S-6P was modified to match the changes present in the S ORF of the sequence of hCoV-19/USA/CT-JAX-JAX001420/2021. Specifically, codons 157 (encoding a phenyl alanine residue) and 158 (encoding an arginine residue) were deleted, and 13 nucleotide substitutions encoding seven amino acid substitutions were introduced in the S-6P ORF [T19R (ACC to Agg), E156G (GAG to Ggc), L452R (CAG to CgG), T478K (ACC to Aag), D614G (GAC to GgC), P681R (CCA to agA), D950N (GAT to aac)].

SARS-COV2-S-6P_B_1_617_2 delta (SEQ ID NO: 13):
ATGTTCGTGTTTCTGGTGCTGCTGCCTCTGGTGAGCTCCCAGTGCGTGAACCTGAGG
ACAAGGACCCAGCTGCCCCCTGCCTATACCAATTCCTTCACACGGGGCGTGTACTAT
CCCGACAAGGTGTTTAGATCTAGCGTGCTGCACTCCACACAGGATCTGTTTCTGCCT
TTCTTTTCTAACGTGACCTGGTTCCACGCCATCCACGTGAGCGGCACCAATGGCACA
AAGCGGTTCGACAATCCAGTGCTGCCCTTTAACGATGGCGTGTACTTCGCCTCCACC
GAGAAGTCTAACATCATCAGAGGCTGGATCTTTGGCACCACACTGGACAGCAAGAC
ACAGTCCCTGCTGATCGTGAACAATGCCACCAACGTGGTCATCAAGGTGTGCGAGTT
CCAGTTTTGTAATGATCCATTCCTGGGCGTGTACTATCACAAGAACAATAAGTCTTG
GATGGAGAGCGGCGTGTATTCCTCTGCCAACAATTGCACATTTGAGTACGTGTCCCA
GCCCTTCCTGATGGACCTGGAGGGCAAGCAGGGCAATTTCAAGAACCTGAGGGAGT
TCGTGTTTAAGAATATCGATGGCTACTTCAAGATCTACTCCAAGCACACCCCAATCA
ACCTGGTGCGCGACCTGCCACAGGGCTTCTCTGCCCTGGAGCCACTGGTGGATCTGC
CCATCGGCATCAACATCACCCGGTTTCAGACACTGCTGGCCCTGCACAGAAGCTACC
TGACACCAGGCGACAGCTCCTCTGGATGGACCGCAGGAGCTGCCGCCTACTATGTG
GGCTATCTGCAGCCCAGGACCTTCCTGCTGAAGTACAACGAGAATGGCACCATCAC
AGACGCCGTGGATTGCGCCCTGGATCCCCTGTCTGAGACCAAGTGTACACTGAAGA
GCTTTACCGTGGAGAAGGGCATCTATCAGACAAGCAATTTCAGGGTGCAGCCTACC
GAGTCCATCGTGCGCTTTCCCAATATCACAAACCTGTGCCCTTTTGGCGAGGTGTTC
AACGCAACCCGCTTCGCCAGCGTGTACGCCTGGAATAGGAAGCGCATCTCCAACTG
CGTGGCCGACTATTCTGTGCTGTACAACAGCGCCTCCTTCTCTACCTTTAAGTGCTAT
GGCGTGAGCCCCACAAAGCTGAATGACCTGTGCTTTACCAACGTGTACGCCGATTCC
TTCGTGATCAGGGGCGACGAGGTGCGCCAGATCGCCCCTGGCCAGACAGGCAAGAT
CGCCGACTACAATTATAAGCTGCCTGACGATTTCACCGGCTGCGTGATCGCCTGGAA
CTCTAACAATCTGGATAGCAAAGTGGGCGGCAACTACAATTATCGGTACCGGCTGTT
TAGAAAGTCTAATCTGAAGCCATTCGAGAGGGACATCTCCACAGAGATCTACCAGG
CCGGCTCTAAGCCCTGCAATGGCGTGGAGGGCTTTAACTGTTATTTCCCTCTGCAGA
GCTACGGCTTCCAGCCAACAAACGGCGTGGGCTATCAGCCCTACCGCGTGGTGGTG
CTGTCTTTTGAGCTGCTGCACGCACCTGCAACAGTGTGCGGACCAAAGAAGAGCAC
CAATCTGGTGAAGAACAAGTGCGTGAACTTCAACTTCAACGGACTGACCGGCACAG
GCGTGCTGACCGAGTCCAACAAGAAGTTCCTGCCTTTTCAGCAGTTCGGCAGGGACA
TCGCAGATACCACAGACGCCGTGCGCGACCCTCAGACCCTGGAGATCCTGGATATC
ACACCATGCTCCTTCGGCGGCGTGTCTGTGATCACACCAGGCACCAATACAAGCAAC
CAGGTGGCCGTGCTGTATCAGGGCGTGAATTGTACCGAGGTGCCCGTGGCAATCCAC
GCAGATCAGCTGACCCCTACATGGCGGGTGTACTCTACCGGCAGCAACGTGTTCCAG
ACAAGAGCCGGATGCCTGATCGGAGCCGAGCACGTGAACAATAGCTATGAGTGCGA
CATCCCTATCGGCGCCGGCATCTGTGCCTCCTACCAGACCCAGACAAACTCCAGAGG
GTCTGCCTCCTCTGTGGCCAGCCAGTCCATCATCGCCTATACCATGAGCCTGGGCGC
CGAGAATTCCGTGGCCTACTCCAACAATTCTATCGCCATCCCTACCAACTTCACAAT
CTCCGTGACCACAGAGATCCTGCCAGTGAGCATGACCAAGACATCCGTGGACTGCA
CAATGTATATCTGTGGCGATTCCACCGAGTGCTCTAACCTGCTGCTGCAGTACGGCT
CTTTTTGTACCCAGCTGAATAGAGCCCTGACAGGCATCGCCGTGGAGCAGGACAAG
AACACACAGGAGGTGTTCGCCCAGGTGAAGCAGATCTACAAGACCCCACCCATCAA
GGACTTTGGCGGCTTCAACTTCAGCCAGATCCTGCCCGATCCTAGCAAGCCATCCAA
GCGGTCTCCTATCGAGGACCTGCTGTTCAACAAGGTGACCCTGGCCGATGCCGGCTT
CATCAAGCAGTATGGCGATTGCCTGGGCGACATCGCCGCCAGAGACCTGATCTGTG
CCCAGAAGTTTAATGGCCTGACCGTGCTGCCTCCACTGCTGACAGATGAGATGATCG
CCCAGTACACATCTGCCCTGCTGGCCGGCACCATCACAAGCGGATGGACCTTCGGCG
CAGGACCCGCCCTGCAGATCCCCTTTCCCATGCAGATGGCCTATCGGTTCAACGGCA
TCGGCGTGACCCAGAATGTGCTGTACGAGAACCAGAAGCTGATCGCCAATCAGTTT
AACTCCGCCATCGGCAAGATCCAGGACTCTCTGAGCTCCACACCCAGCGCCCTGGGC
AAGCTGCAGAACGTGGTGAATCAGAACGCCCAGGCCCTGAATACCCTGGTGAAGCA
GCTGTCTAGCAACTTCGGCGCCATCTCCTCTGTGCTGAATGATATCCTGAGCAGGCT
GGACCCTCCAGAGGCAGAGGTGCAGATCGACCGGCTGATCACAGGCAGACTGCAGT
CCCTGCAGACCTACGTGACACAGCAGCTGATCAGGGCAGCAGAGATCAGGGCCTCT
GCCAATCTGGCCGCCACCAAGATGAGCGAGTGCGTGCTGGGCCAGTCCAAGAGAGT
GGACTTTTGTGGCAAGGGCTATCACCTGATGAGCTTCCCACAGTCCGCCCCTCACGG
AGTGGTGTTTCTGCACGTGACCTACGTGCCAGCCCAGGAGAAGAACTTCACCACAG
CACCAGCAATCTGCCACGATGGCAAGGCACACTTTCCTAGGGAGGGCGTGTTCGTG
AGCAACGGCACCCACTGGTTTGTGACACAGCGCAATTTCTACGAGCCACAGATCATC
ACCACAGACAATACATTCGTGTCCGGCAACTGTGACGTGGTCATCGGCATCGTGAAC
AATACCGTGTATGATCCTCTGCAGCCAGAGCTGGACTCTTTTAAGGAGGAGCTGGAT
AAGTACTTCAAGAATCACACCAGCCCCGACGTGGATCTGGGCGACATCTCTGGCATC
AATGCCAGCGTGGTGAACATCCAGAAGGAGATCGACAGGCTGAACGAGGTGGCCAA
GAATCTGAACGAGTCCCTGATCGATCTGCAGGAGCTGGGCAAGTATGAGCAGTACA
TCAAGTGGCCCTGGTATATCTGGCTGGGCTTCATCGCCGGCCTGATCGCCATCGTGA
TGGTGACCATCATGCTGTGCTGTATGACAAGCTGCTGTTCCTGCCTGAAGGGCTGCT
GTTCTTGTGGCAGCTGCTGTAAGTTTGATGAGGACGATAGCGAGCCTGTGCTGAAGG
GCGTGAAGCTGCACTACACC
APMV3_SARS_CoV2_6P (SEQ ID NO: 14):
ACTAAACAGAAAATTAATACTTGTTAGTAGTCGCCGATTAATCCGCTAATATATTAG
GAGCGGAAGTCCTACACTCCGCCTCCGACCACGAATTGAAATCATCATGGCTGGAA
TTTTTAATACTTATGAGCTCTTTGTTAAGGATCAGACATGTATGCACAAGAGGGCTG
CAAGCCTAATATCGGGAGGTCAGCTTAAAAGCAATATACCAGTCTTCATCACCACAA
AAGATGATCCAGCTGTCCGATGGGACCTTGTATGTTTTAATCTCCGACTTGTTGTTAG
TGAATCATCTACCTCGGTGATCCGGCAGGGGGCTCTTATATCATTGCTTTCCATTACT
GCTAGCAATATGAGGGCTCTTGCAGCAATTGCTGGGCAGACTGATGAATCGATGATT
AATATTATAGAAGTTGTGGACTTTGATGGACTTGAGCCCCAGTGTGACACTCGCAGT
GGCTTAGACGCTCAGAAGCAGGAGATGTTTAAGGATATTGCCCGTGATATGCCGAG
GGCACTATCCAGCGGGACCCCGTTCCAGCACGGAAATGCGGAAGCGAATGGGCCTG
AAGACACACACATGTTCCTCCGATCCGCAATTAGCGTTCTGACACAGATCTGGATCC
TGGTTGCAAAAGCCATGACTAACATTGATGGCAGCAACGAGGCAAGCGATCGCCGC
CTTGCCAAGTATATTCAACAGAATAGAATTGACAGGCGATTCATGCTTGCTCAAGCT
ACCAGGACTGTATGTCAGCAGGTCATAAAGGATTCTTTGACCTTGAGAAGGTTCCTT
GTAACTGAGCTTCGAAAGTCGCGGGGGGCATTGCATAGCGGGTCATCTTACTATGCT
ATGATTGGAGACATGCAGGCATACATATTTAATGCAGGCCTTACACCATTCCTCACT
ACCCTCAGGTACGGGATCGGGACTAAATACCATGCCCTTGCAATTAGCTCACTTGCA
GGTGACCTGAATAAGATTAAGGGGTTACTGACTCTATACAAAGAGAAAGGGAGTGA
TGCGGGCTATATGGCACTTCTAGAGGATGCTGACTGCATGCAATTTGCGCCAGGTAA
TTACTCACTATTATATTCATATGCGATGGGAGTAGCAAGTGTACATGATGAAGGCAT
GAGGAATTACCAATACGCCCGGCGCTTCCTCCATAAGGGGATGTACCAATTTGGACG
TGATATCGCAACACAACAGCAACATGCACTTGATGAATCCCTCGCCCAGGAGATGA
GAATCACAGAGGCTGACAGGGCAAATCTCAAGGTAATGATGGCAAACATTGGGGAA
GCATCAAGCTTCAGTGATATGCCTAATCCAGGAGCTAGCGGCATTCCTGCCTTCAAT
GACCGCCCTGATGAGATCTTCTCTGAGCCAGGCTATCGCAAGCCCCCTCAAGATCAG
CCGCTGATAAAGCTCCCAGATCTCGAGGAAGAAGAGCAGGACGAATTCGACATGTA
GAGAGCCGCCCACCACCCAGAGCCAGCGACAAACCCAGGCAAAACAACCCACCATC
TCCGACCCGGAACACAACGCAACATCGAGGACCCCGGGCAGCCGACACCGCAGCCG
CAAGACCTCCGCATCATGAACGACCAGCATCCAAACATCTGTCCTCCCTATTCTATC
AGTTTAATAAAAAAGGCTTTACGTGCACAGACAGAGATCCTGACATAATTCAGCCG
GGGGGATTCAACTTAGGAGCGGAAGTATCCCTCCTAACCTGCACCTGCATTTCAATA
TGGATCTTGAGTTTAGTAGTGAGGAAGCAGTCGCAGCCCTCTTGGATGTGAGTTCCT
CAACCATAACCGAGGTTCTGAGCAAGCAAAGTATTCCTGACCCTAGCTTTCTCACGT
CCACGGAGGCATCCGGTGAGCAAGTTCCAGATTCGAAGGTCGCCAATCACAAAGCC
AGTGTCGATAAGACCCCAGATCAAGGCCAACCATCGGCAACGCCTTCGGCTCCTCCT
GAGACTGCCGAAAACATCAATACACCATCTTGCGAGGACGGTTTACCCCCTAACTTC
TTCATCCCAAGGGTAGAGAGTTACCACACTAACCTTTTTAAAGGGGGCCCCCAATTG
GACTCAACGGAGCGGCCGCCTGGTCACCAAATAGTTTGCGGAGATCAAGACGCGGA
CCTCTCCCGAGCCGGGATAGCGAGAAAGAAGAAGAAGCAGAAGCATTGCAAAGCG
CTCAACTTTTCGGAATCCCCACTCACAGACAATCAGGCCATAGAAGGGAGTATTCAA
TCTCATGGAGCCCTGGATCAGGAACCTTCCAGACAGAGACCTGGTGCAACCCAGCC
TGCTCTCCAGTCACCGCCTTCCCAAAACAATACAAGTGTGCATGCAGACAGTGCCCA
AGATTCTGCGATCTCTGTTTCAATCCCGCTGACTATGGTGGAATCATTGATCTCTCAA
GTGTCAAAGTTGTCGGACCAAGTCTCGCAGATACAGAAGTTGGTGAGTACCCTTCCT
CAAATTAAGACTGATATTGCTTCGATTCGGAACATGCAAGCAGCTTTGGAAGGACA
GCTCAGTATGATTCGTATATTGGATCCAGGAAACTGTTCGGAATCATCACTCAATGC
GCTGAGAGGGTTCAGTGGTAAGGCACCTGTTATTGTGAGTGGTCCAGGAAATCCCA
ATCAATGCATCAGCCAAGGCTCATCCACCACAATTTGCCTGGATGAGTTAGCCCGGC
CTGTGCCAAATCCTATACAGGTGAAACTTCAAGATGATCAGAAGGACCTATCTGCAC
AGCGACATGCTGTCATTGCATTACTCGAAACCAGAATCCCCCCGGGACCAAAACGT
GACAAATTACTGTCATTGGTTGCAATTGCAAAGACTGCAAGTGATTTAATCAAGATT
AAGAGAATGGCAGTCCTCGGCCAGTGATATCACCCTATGGCAACCAGGCTACAAAT
GTAAGCAGTTTCCGATATGTACCGAATCAATAACACGACCCCTTCCGCAAAAGCAA
GCGCGAGCATGACACACTCAGCTTCAGCCAATTCTAGCTTCACTAAGCAGGTGATCT
GACCATCTGGCTTTACTTCCGCTTTATAAAAAACTCCTAGATCTAACATGGAGGCAG
ACCGAGCTAATGCGGAGGGAGTACACAAGGGGAATCAAAGGAGCGGAAGGTACTA
AGTCACTCCACCGCCTGCCCTCGCCCATCCGCAGCCTCCCCCAACCCGCGGCGCCGC
CACCATGTTCGTGTTTCTGGTGCTGCTGCCTCTGGTGAGCTCCCAGTGCGTGAACCTG
ACCACAAGGACCCAGCTGCCCCCTGCCTATACCAATTCCTTCACACGGGGCGTGTAC
TATCCCGACAAGGTGTTTAGATCTAGCGTGCTGCACTCCACACAGGATCTGTTTCTG
CCTTTCTTTTCTAACGTGACCTGGTTCCACGCCATCCACGTGAGCGGCACCAATGGC
ACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAA
GACACAGTCCCTGCTGATCGTGAACAATGCCACCAACGTGGTCATCAAGGTGTGCG
AGTTCCAGTTTTGTAATGATCCATTCCTGGGCGTGTACTATCACAAGAACAATAAGT
CTTGGATGGAGAGCGAGTTTCGCGTGTATTCCTCTGCCAACAATTGCACATTTGAGT
ACGTGTCCCAGCCCTTCCTGATGGACCTGGAGGGCAAGCAGGGCAATTTCAAGAAC
CTGAGGGAGTTCGTGTTTAAGAATATCGATGGCTACTTCAAGATCTACTCCAAGCAC
ACCCCAATCAACCTGGTGCGCGACCTGCCACAGGGCTTCTCTGCCCTGGAGCCACTG
GTGGATCTGCCCATCGGCATCAACATCACCCGGTTTCAGACACTGCTGGCCCTGCAC
AGAAGCTACCTGACACCAGGCGACAGCTCCTCTGGATGGACCGCAGGAGCTGCCGC
CTACTATGTGGGCTATCTGCAGCCCAGGACCTTCCTGCTGAAGTACAACGAGAATGG
CACCATCACAGACGCCGTGGATTGCGCCCTGGATCCCCTGTCTGAGACCAAGTGTAC
ACTGAAGAGCTTTACCGTGGAGAAGGGCATCTATCAGACAAGCAATTTCAGGGTGC
AGCCTACCGAGTCCATCGTGCGCTTTCCCAATATCACAAACCTGTGCCCTTTTGGCG
AGGTGTTCAACGCAACCCGCTTCGCCAGCGTGTACGCCTGGAATAGGAAGCGCATC
TCCAACTGCGTGGCCGACTATTCTGTGCTGTACAACAGCGCCTCCTTCTCTACCTTTA
AGTGCTATGGCGTGAGCCCCACAAAGCTGAATGACCTGTGCTTTACCAACGTGTACG
CCGATTCCTTCGTGATCAGGGGCGACGAGGTGCGCCAGATCGCCCCTGGCCAGACA
GGCAAGATCGCCGACTACAATTATAAGCTGCCTGACGATTTCACCGGCTGCGTGATC
GCCTGGAACTCTAACAATCTGGATAGCAAAGTGGGCGGCAACTACAATTATCTGTAC
CGGCTGTTTAGAAAGTCTAATCTGAAGCCATTCGAGAGGGACATCTCCACAGAGATC
TACCAGGCCGGCTCTACCCCCTGCAATGGCGTGGAGGGCTTTAACTGTTATTTCCCT
CTGCAGAGCTACGGCTTCCAGCCAACAAACGGCGTGGGCTATCAGCCCTACCGCGT
GGTGGTGCTGTCTTTTGAGCTGCTGCACGCACCTGCAACAGTGTGCGGACCAAAGAA
GAGCACCAATCTGGTGAAGAACAAGTGCGTGAACTTCAACTTCAACGGACTGACCG
GCACAGGCGTGCTGACCGAGTCCAACAAGAAGTTCCTGCCTTTTCAGCAGTTCGGCA
GGGACATCGCAGATACCACAGACGCCGTGCGCGACCCTCAGACCCTGGAGATCCTG
GATATCACACCATGCTCCTTCGGCGGCGTGTCTGTGATCACACCAGGCACCAATACA
AGCAACCAGGTGGCCGTGCTGTATCAGGACGTGAATTGTACCGAGGTGCCCGTGGC
AATCCACGCAGATCAGCTGACCCCTACATGGCGGGTGTACTCTACCGGCAGCAACG
TGTTCCAGACAAGAGCCGGATGCCTGATCGGAGCCGAGCACGTGAACAATAGCTAT
GAGTGCGACATCCCTATCGGCGCCGGCATCTGTGCCTCCTACCAGACCCAGACAAAC
TCCCCAGGGTCTGCCTCCTCTGTGGCCAGCCAGTCCATCATCGCCTATACCATGAGC
CTGGGCGCCGAGAATTCCGTGGCCTACTCCAACAATTCTATCGCCATCCCTACCAAC
TTCACAATCTCCGTGACCACAGAGATCCTGCCAGTGAGCATGACCAAGACATCCGTG
GACTGCACAATGTATATCTGTGGCGATTCCACCGAGTGCTCTAACCTGCTGCTGCAG
TACGGCTCTTTTTGTACCCAGCTGAATAGAGCCCTGACAGGCATCGCCGTGGAGCAG
GACAAGAACACACAGGAGGTGTTCGCCCAGGTGAAGCAGATCTACAAGACCCCACC
CATCAAGGACTTTGGCGGCTTCAACTTCAGCCAGATCCTGCCCGATCCTAGCAAGCC
ATCCAAGCGGTCTCCTATCGAGGACCTGCTGTTCAACAAGGTGACCCTGGCCGATGC
CGGCTTCATCAAGCAGTATGGCGATTGCCTGGGCGACATCGCCGCCAGAGACCTGA
TCTGTGCCCAGAAGTTTAATGGCCTGACCGTGCTGCCTCCACTGCTGACAGATGAGA
TGATCGCCCAGTACACATCTGCCCTGCTGGCCGGCACCATCACAAGCGGATGGACCT
TCGGCGCAGGACCCGCCCTGCAGATCCCCTTTCCCATGCAGATGGCCTATCGGTTCA
ACGGCATCGGCGTGACCCAGAATGTGCTGTACGAGAACCAGAAGCTGATCGCCAAT
CAGTTTAACTCCGCCATCGGCAAGATCCAGGACTCTCTGAGCTCCACACCCAGCGCC
CTGGGCAAGCTGCAGGATGTGGTGAATCAGAACGCCCAGGCCCTGAATACCCTGGT
GAAGCAGCTGTCTAGCAACTTCGGCGCCATCTCCTCTGTGCTGAATGATATCCTGAG
CAGGCTGGACCCTCCAGAGGCAGAGGTGCAGATCGACCGGCTGATCACAGGCAGAC
TGCAGTCCCTGCAGACCTACGTGACACAGCAGCTGATCAGGGCAGCAGAGATCAGG
GCCTCTGCCAATCTGGCCGCCACCAAGATGAGCGAGTGCGTGCTGGGCCAGTCCAA
GAGAGTGGACTTTTGTGGCAAGGGCTATCACCTGATGAGCTTCCCACAGTCCGCCCC
TCACGGAGTGGTGTTTCTGCACGTGACCTACGTGCCAGCCCAGGAGAAGAACTTCAC
CACAGCACCAGCAATCTGCCACGATGGCAAGGCACACTTTCCTAGGGAGGGCGTGT
TCGTGAGCAACGGCACCCACTGGTTTGTGACACAGCGCAATTTCTACGAGCCACAG
ATCATCACCACAGACAATACATTCGTGTCCGGCAACTGTGACGTGGTCATCGGCATC
GTGAACAATACCGTGTATGATCCTCTGCAGCCAGAGCTGGACTCTTTTAAGGAGGAG
CTGGATAAGTACTTCAAGAATCACACCAGCCCCGACGTGGATCTGGGCGACATCTCT
GGCATCAATGCCAGCGTGGTGAACATCCAGAAGGAGATCGACAGGCTGAACGAGGT
GGCCAAGAATCTGAACGAGTCCCTGATCGATCTGCAGGAGCTGGGCAAGTATGAGC
AGTACATCAAGTGGCCCTGGTATATCTGGCTGGGCTTCATCGCCGGCCTGATCGCCA
TCGTGATGGTGACCATCATGCTGTGCTGTATGACAAGCTGCTGTTCCTGCCTGAAGG
GCTGCTGTTCTTGTGGCAGCTGCTGTAAGTTTGATGAGGACGATAGCGAGCCTGTGC
TGAAGGGCGTGAAGCTGCACTACACCTGATAGTTTATAAAAAACATTGTGAGAATG
CAATTTAAGGACGAAAGTAGGAGCGGAAGCCGCGGCCCAGGCTCCATTCAAAAACC
ATGGCCGCTGCTCTCGGGAACATGCACCCGTCATCGTCGATTACTCTTATGCATGAT
GACCCGTCCATCCAGACGCAGTTGCTAGCCTTCCCTCTGATAAGTGAGAAGACAGA
GACAGGGACGACAAAGCTGCAGCCTCAAGTGAGAATGCAATCTTTCCTATCAACCG
ATAGCCAGAAGTATCATCTGGTCTTTATCAACACATATGGTTTCATTGCGGAGGATT
TTAATTGTAGTCCAGCAAACGGATTTGTGCCTGCACTATTTCAACCCAAGTCTAAAG
TCTTATCGTCAGCGATGGTTACACTTGGGGCTGTCCCTGCTGACACTGTTCTCCAGGA
CCTACAACGGGACTTAATCGCAATGAGATTTAAAGTCCGAAAGAGTGCGTCAGCGA
AGGAGCTAATATTATTCTCCACAGACAATGTCCCTGCAACATTAACAGGGTCATCAG
TCTGGAAGAATAAAGGAGTAATAGCTGATACAGCAACTGCAGTTAAAGCGCCCGGT
CGCATCTCATGCGATGCGGTATGCAGTTATTGCATTACATTCATCTCTTTTTGCTTCT
TCCACTCATCTGCTCTTTTTAAGGTGCCCAAGCCTCTGCTGAATTTCGAGACGGCCAT
AGCATATTCCCTTGTACTCCAGGTCGAGCTTGAGTTCCCAAATATCAAAGATACCTT
ACACGAGAAGTATCTCAAAGTTAAAGACTCGAAGTGGTATTGTACAATTGACATCC
ATATTGGGAATCTTTTAAAGAGAACCGCCAAGCAACGAAGGAGGACACCTTCAGAG
ATAATGCAGAAAGTCCGTAAGATGGGTTTCAGGATTGGGTTATACGATTTGTGGGGT
CCAACAATTGTGGTTGAATTAACTGGGTCCTCAAGTAAGTCCCTACACGGATTCTTC
TCGAGTGAGCGGCTGGCCTGCCACCCGGTATCACAGTATAATCCTCATGTTGGACAG
CTTATCTGGGCCCACGATGTATCCATCACGGGGTGCCACATGATTATATCTGAACTT
GAGAAAAAGAAGGCACTTGCAATGGCTGACCTGACCGTGAGTGATGCAGTAGCTGT
CAACACGAGCATCAAGAGCTTATCGCCTTTCAGACTGTTCAAAAAATATTAGGGCTA
GACAACTCCTAGGGTGTGTGCTTATAATTTGCATATTCTAGATTATAATCATTAACCG
CGTCCAAATGATCAGACCGCATAATTAACAGCTCAGCCAGGACTCGCAAATCCTGC
ATAGGCTCAAATAACAAGACGACATATATACTCACACAACAAGGTGTAATATCTAG
AAGGAATCAGAATTGAGACAATATCTAGGTTATCAGCGCTACTGCCCTGCAACTCAC
TATGTATGCTTGGCATTAATAAAAAACATTGTGAGAATGCAATTTAAGGACGAAAGT
AGGAGCGGAACACGCTCAACTGAACCCAAGCGCACACCAGCCCCCCGGCACCGCAC
CGGAAGGACGCACAACAGCCGACCGCCCCCACCAGGGCAGAGGCCATGCAACCAG
GCTCGGCACTCCACCTCCCGCACCTGTACATAATAATTGCCTTAGTCAGCGATGGCA
CTTTAGGCCAAACAGCTAAAATTGATAGGCTGATACAGGCAGGGATTGTCTTGGGTA
GTGGAAAGGAGCTCCATATCTCACAAGATTCAGGGACACTGGATTTATTCGTGAGGT
TGTTACCAGTACTCCCTTCGAATTTGTCCCATTGCCAGCTTGAAGCAATAACACAGT
ATAATAAGACGGTGACTAGGTTATTGGCCCCAATAGGGAAGAACCTAGAACAGGTA
CTACAAGCGAGACCGCGTGGGAGGCTGTTCGGAGCAATAATAGGGTCCATCGCGCT
TGGTGTTGCCACATCAGCACAGATTACCGCTGCAATTGCACTCGTCCGTGCACAGCA
AAATGCTAATGATATATTAGCACTTAAAAACGCTCTCCAATCCAGCAATGAGGCAAT
CCGTCAACTCACGTATGGCCAGGACAAACAGTTACTTGCAATTTCTAAAATACAAAA
AGCTGTGAATGAGCAGATCCTCCCTGCACTCGATCAGCTGGACTGTGCTGTCCTTGG
AACCAAACTAGCTGTGCAGCTTAATCTGTATCTCATTGAGATGACTACCATTTTCGG
CGAGCAAATCAATAATCCAGTCTTGGCGACCATCCCATTAAGTTACATTTTACGCCT
GACCGGTGCGGAGTTGAACAACGTCCTCATGAAGCAAGCCCGATCGTCCTTAAGCC
TTGTACAGTTAGTTTCAAAAGGCTTACTTAGTGGACAAGTAATTGGCTATGACCCAT
CTGTTCAAGGTCTGATAATAAGGGTGAATCTGATGCGAACGCAGAAGATCGATAGA
GCACTAGTTTATCAGCCGTATGTATTACCAATCACTCTTAACTCTAACATAGTAACA
CCAATCGCCCCAGAATGCGTAATTCAGAAAGGGACGATCATAGAGGGTATGTCACG
GAAGGATTGTACAGAGTTGGAGCAAGACATAATTTGTAGGACAGTTACGACATACA
CGCTTGCCAGAGACACCAGATTGTGTTTACAAGGCAATATCTCTTCTTGTAGGTATC
AACAGTCAGGCACCCAACTACACACCCCATTTATTACGTACAACGGGGCAGTTATCG
CAAACTGTGATTTAGTCTCTTGTCGGTGTCTCCGTCCTCCTATGATTATCACCCAAGT
AAAAGGGTACCCTCTTACCATAATCACAAGGTCCGTATGTCAAGAATTATCCGTTGA
CAACTTAGTCCTGAATATTGAGACACACCATAATTTCTCCCTTAATCCAACAATTATC
GACCCTCTAACAAGAGTAATAGCCACTACACCGTTAGAGATAGATTCCTTAATCCAA
GAGGCACAAGATCATGCGAATGCAGCGCTAGCTAAAGTGGAAGAGAGTGATAAATA
TCTCAGGGCAGTTACTGGCGGTAATTATTCGAATTGGTATATCGTTCTTGTCATCGTC
TTACTATTTGGGAATTTGGGCTGGTCGCTGTTGTTAACAGTACTTCTATGTAGGTCAC
GGAAACAACAGCGACGTTATCAACAAGATGATTCCGTCGGGTCAGAAAGAGGTGTT
GGGGTTGGGACCATCCAGTATATGTCATAAGTGGTGTCAGACATCACAATCCAAGTC
ACTCAGTTATCTACTCCGCTGGCTCGGAGCCATCGACAACGGCGTGAGCCCTCATCC
GGGGACAATCCACAACCTGATACTACCAGACAGTGGCTCCGCACGATGGCAGCAGC
CCACAACGTCCTGCATGTAAATATCATTGCGTTACTTTAATTAAAAAACCTCAAGGT
GACCCATCCTGGCCCATCCGATTGGAGGAGCGGAAGGTTTTAAGGTAATCAGGCAA
ATTGTTTCCAACATGGAATCCCCTCCTTCTGGCAAGGATGCGCCAGCCTTCCGCGAG
CCTAAGCGAACTTGCAGACTCTGTTACAGGGCGACGACTCTCTCCCTTAATCTCACC
ATCGTTGTATTATCAATTATAAGCATTTATGTATCAACTCAAACTGGGGCAAACAAC
TCTTGTGTCAATCCCACAATCGTAACTCCTGATTATTTAACTGGCAGCACGACAGGC
TCAGTTGAAGATCTTGCTGACTTAGAGAGTCAACTCCGGGAGATACGTAGAGACAC
AGGGATTAATCTCCCAGTTCAGATCGATAATACAGAAAATCTAATACTGACAACTCT
GGCGAGTATCAACTCGAATTTACGCTTTCTGCAAAATGCAACAACTGAGAGCCAGA
CCTGCCTATCTCCTGTCAATGACCCTCGCTTTGTAGCTGGCATAAATAGGATCCCAG
CAGGGTCGATGGCATATAATGACTTCAGCAACCTCATTGAGCACGTAAATTTCATAC
CGTCGCCGACAACGTTGTCAGGCTGCACCAGGATACCATCATTTTCACTCTCTAAGA
CGCACTGGTGTTATACCCACAATGTTATATCAAACGGGTGCCTTGACCATGCTGCAA
GTTCACAATACATCTCAATAGGGATCGTTGATACCGGCCTAAATAATGAGCCCTATT
TTAGAACAATGTCTTCAAAGTCACTGAATGACGGATTAAATAGAAAGAGTTGCTCTG
TGACCGCAGCCGCTAATGCATGTTGGTTACTCTGTAGCGTAGTAACAGAATACGAAG
CTGCGGACTATCGATCACGAACTCCTACTGCTATGGTTCTGGGGCGATTCGATTTTA
ATGGTGAATACACAGAAATAGCTGTCCCCTCCTCACTATTCGATGGCCGGTTTGCAT
CTAACTATCCAGGTGTGGGATCGGGAACTCAAGTCAATGGAACATTATATTTCCCTT
TATATGGAGGGGTCCTTAATGGGTCAGATATAGAAACGGCAAACAAGGGGAAATCC
TTCAGGCCTCAGAATCCTAAGAATAGGTGTCCAGACTCGGAGGCGATCCAAAGCTTT
AGAGCGCAAGATAGCTATTACCCGACGAGATTTGGGAAGGTGCTGATCCAACAGGC
AATCATTGCATGTAGGATCAGTAACAAAAGTTGTACTGATTTCTATCTCCTGTACTTC
GACAACAACAGAGTGATGATGGGTGCAGAGGCACGGCTATATTATCTTAATAACCA
ATTATATCTATATCAACGCTCGTCGAGCTGGTGGCCGCATCCTTTGTTCTATAGCATC
TCATTACCGTCATGTCAAGCCCTTGCTGTCTGTCAAATTACAGAGGCCCATCTGACA
CTGACTTACGCCACGTCGAGGCCAGGAATGTCTATCTGTACAGGAGCATCAAGGTGT
CCAAATAATTGCGTAGACGGTGTATATACTGATGTATGGCCATTGACAAAGAACGAT
GCTCAGGACCCCAATCTCTTCTACACTGTATACTTAAATAATTCAACTCGGCGGATT
AGCCCAACCATAAGTCTATACACTTATGACCGCAGGATCAAATCTAAGCTGGCAGTG
GGTAGTGATATAGGAGCAGCTTATACCACGAGTACCTGTTTCGGCCGATCAGATACA
GGGGCGGTTTATTGCTTAACTATAATGGAGACCGTTAACACAATATTTGGTCAGTAC
CGGATCGTGCCAATATTACTTAGAGTGACAAGCCGGTAATGGATTGTTCCTAGTGAA
GTGGGGCCTATGGTTACTCCGTAACGGAGATGACCTGATTTATGCTATGTAACTAAT
TTTGCTCAATCGGGAGGGTGATCGGCCGGGTATGTCCACAATGACAATGACTCCTAT
TCCCTTGCTAGAGCTAGCTTCCTCTTTAATCCAATACAATCCCTACCTGTAGTTTAAT
AAAAAAATCCTCTATATAAGATCCAGACGCGTGCTATATAAGCTAATAGCTTAGGAT
GGAAACGACAGGAGCGGAACATATCCCTCCTACCATGTCTTCTCATAATATTATCCT
CCCTGACCACCACTTAAATTCCCCTATTGTACTAAATAAATTGATGTACTATTGTAAA
CTCCTTAATATATTGCCTGACCCTAAGTCGCCATGGTACGAGAAGATTAAGTCTTGG
ACTAATTGCTGCATCCGTGTGTCAGACAGCAATCGCATGACTTTGTCACGCGCCTCC
AAGCTACGAGAACTATTGGCAACGTACGGCGTATATTCGAAGAACCACCAGCAATG
TTATACCACCATAATCCATCCCCAATCCCTATCACCCATCATGCAGACTGTCTCTCAA
CTGGGACGCTCAATCCCGACCTGGGCCAGGATCCGAAAGGAGATCACACACAGTAT
CATAGCCCAAACGAATAAATTTGAATCCTTATTCCACAACATCTCAAAGGACCTGAC
AGGGAAAACCAACTTGTTTGGCGGATTCTGTGATTTACATGGGAGCATAAGCACTGC
TGCAAAGCGCTTAATGAATCAACCGGGTCTCTACTTGGATTCACCTGATGCCCATGC
ATGTCAATTTTTGTTCCAGCTTAAAACCTGTCAACGGGAATTGATTCTACTTATGAGG
CAGAATGCCACAACAGAGCTGATTAGGGTTTTTGACTATCCTGAGCTACGGGTTCTT
GTGACACCTGAATATTCGGTCTGGGTTTTTACACGGACCAAACAAGTTACATTACTA
ACATTCGATTGCTTGTTAATGTACTGCGATCTTTCAGACGGCCGGCACAACATCCTA
TTCACTTGTAAGCTTTTGTCTCACTTAAACCACCTCGGGTGTCGGATAAGGGACCTG
CTGGTGCTAATTGATTCACTTGCCGAGAAACACCCCTTAATAGTTTATGATGTAGTT
GCAAGTCTGGAGTCGTTATCATATGGTGCTATACAGCTACATGACAAGGTAGCTGGT
TATGCTGGAACATTTTTCTCATTTATCCTTGCAGAAATACAGGATTCCTTAGAAACA
GTGTTGGATCAAGGTAATAGTGAATCTATCATATCCCAAATAAGGAACATATACTCA
GGTCTCACAGTAAACGAGGCTGCAGAATTGCTATGTGTTATGAGGCTTTGGGGCCAC
CCTGCCCTAAACAGTGTAGACGCTGCAAGCAAGGTGAGACAGAGTATGTGCGCTGG
CAAGCTGCTTAAATTTGATACGATCCAGTTAGTCCTTGCATTCTTCAATACACTAATA
ATTAATGGATATAGGAGAAAGCACCATGGGCGATGGCCGAATGTTTGTAGTGACTC
TATAATTGGGAATGAACTCAAGCGGATGTATTTTGATCAATGTGAGATCCCCCACGA
CTTCACATTAAAGAACTACCGGGAATTAAGTCTTCTTGAATTCGAGTGCACATTTCC
CATAGAATTATCAGATAAACTGAACATATTTTTAAAAGATAAGGCAATAGCATTCCC
GAAGTCCAGGTGGACATCTCCTTTCAAAGCAGATATCACACCGCGCCATTTGCTGCA
AGCGCCTGAGTTTAAAACCCGTGCCAACAGACTACTGCTATCATTCTTACAACTCGA
GGAATTCTCCATCGAATCAGAACTAGAATATGTGACTACCCTGGCTTACCTGGATGA
TGATGAGTTCAACGTATCTTACTCCCTCAAAGAGAAGGAGGTAAAGACAGATGGTC
GAATCTTCGCTAAGCTCACAAGGAAAATGCGGAGCTGTCAGGTGATATTCGAAGAA
TTGCTAGCAGAGCATGTCTCTCCATTATTTAAAGACAACGGCGTTACTATGGCCGAA
TTGTCACTTACTAAGAGCCTACTCGCTATTAGCAACCTAAGCTCCACCTTATTTGAGA
CGCAAACAAGGCAAGGCGATCGGAATGCAAGATTCACGCATGCGCACTTCATAACA
ACTGACCTGCAAAAGTACTGTTTAAACTGGAGGTACCAGAGTGTAAAACTGTTCGCA
CGTCAATTGAACCGGTTATTTGGTCTGCAGCATGGGTTTGAATGGATCCATTGCATA
CTAATGAAGTCCACTATGTATGTCGCAGACCCCTTTAACCCTCCACATAGCAATGCA
CGTGAGGCACTCGATGACAACTTAAATAGCGACATATTCATTGTATCACCTAGGGGG
GCCATCGAGGGACTATGCCAAAAAATGTGGACAATCATCTCAATCTCGGCGATCCAT
GCATCCGCTGCTGTGGCTGGGCTACGGGTGGCATCTATGGTCCAAGGTGATAACCAA
GTTATTGGGGTAACACGCGAGTTCCTAGCTGGGCATGATCAAACTTTTGTAGACGAT
CAATTGACGGTTTCACTTGAGAACTTCACTCAGATATTCAAGGAAGTCAACTACGGG
CTAGGTCATAATCTCAAGCTGAGGGAAACAATCAAGTCGAGCCACATGTTCATCTAC
TCAAAGAGGATCTTCTACGACGGCCGAGTGCTTCCACAACTTCTAAAGAACATAAGT
AAGTTAACCCTATCAGCTACAACTACAGGGGAGAACTGTTTAACGAGTTGCGGGGA
CCTATCATCATGCATCACACGATGTATAGAGAATGGGTTCCCTAAAGATGCAGCATT
CGTTTTGAATCAATTAGTGATTAGGATCCAGATACTTGCCGATCACTTTTATTCCATC
CTTGGAGGATGTTTCTCTGGGTTGAACCAGGCGGATATCCGTCTCCTTCTATATGAG
GGTGCAATTTTACCGGCACAATTAGGAGGGTTCAACAACCTCAACATGTCACGGCTA
TTCTGTCGGAATATTGGTGACCCATTAGTCGCGTCCATCGCAGACACCAAGAGATTT
GTGAAGTGCCAACTGCTCGTTCCTCACATTTTAGACTCTGTCGTTGCTATTACCGATA
GGAAGGGGTCTTTTACGACACTGATGATGGATCCATATTCAATAAACTTGGATTATA
TCCAGCAACCAGAGACACGATTGAAGCGACATGTGCAAAAGGTTTTACTACAAGAG
TCAGTGAATCCGCTACTCCAAGGGGTCTTCCTAGAGTCTCAACAAGAGGAAGAGGA
ACAACTTGCAGCGTTCTTGCTGGACCGAGAAGTCGTAATGCCAAGGGTTGCGCACGC
TATATTTGAGTGTACCAGCTTAGGTAGGCGCCGACATATCCAGGGCCTCATCGACAC
GACAAAGACCATTATTGCACTTGCGTTGGATACACAGCAGTTAAGTTATACTAAACG
TGAACAGATTGTGACGTACAATGCAACTTATATGAGGTCCTTAGCAAGCATGCTCAG
TTCAAGCCATAATCAGACACGTCGGTCTGTGATTGGTCACGCATCCTTCAATATCAC
CGACTGCTCGGTCATCCTGGCTCAACAAGTGAGACGTGCAAGCTGGGCGCCCTTGCT
GAATTGGCGGTCATTAGAAGGCTTAGAAGTACCCGACCCTATTGAGTCAGTAGCCG
GCTACCTGGGTTTGGATTCTAACAACTGTTTCTTATGCTGCCACGAGCAGAACAGCT
ATTCCTGGTTTTTCCTTCCCCGAATGTGCCATTTTGATGATTCGAGACAATCTAACTC
CATACAAAGGGTACCTTACATTGGTTCTAAGACTGACGAGCGCCAGATGTCTACGAT
AAATCTACTTGAGAAAACTACCTGTCATGCTAGAGCAGCTACGAGGCTTGCATCATT
ATACATTTGGGCCTTTGGGGACTCAGACGATAGTTGGGATGCAGTCGAGACTCTATC
TAACAGTCGATGCCAGATCACCCGAGAGCACTTGCAAGCTCTGTGCCCTATGCCCTC
TTCAGTGAATCTCCACCATCGATTGAACGACGGAATAACACAAGTGAAGTTTATGCC
ATCGACCAATAGCAGGGTCTCTCGATTTGTTCATATTTCCAATGACCGGCAGAATTA
CGTATTGGATGACTCCGTTACTGACAGTAATCTGATTTACCAACAAGTCATGTTGCT
AGGACTTAGTATACTTGAAACATATTTCAGAGAACCTACAGCGACGAACTTATCAAG
CTTGGTGCTGCACCTTCACACTGATGTATCTTGTTGCTTACGAGAATGCCCGATGAC
ACAATATGCGCCCCCCCTGCGAGACATCCCAGAGTTAACTATAACTGCGTCAAATCC
TTTCTTATTTGATCAAGAACCTATTAGCGAAGCAGACCTGTGTAGACTCTCAAAGAT
AGCATTCCGTAGAGCAGGAGACAACTACGATGCATACGACCAGTTTCAACTAAGAG
CAACACTGGCCGCAACTACAGGTAAGGATGTGGCAGCAACAATCTTTGGTCCTCTAG
CTGCAGTATCAGCAAAAAATGATGCAATAGTCACAAATGACTATAGCGGGAATTGG
ATTTCCGAGTTTAGGTATAGTGATCTCTATCTTTTGAGTGTTAGCCTGGGCTACGAGA
TCCTTTTGATTTTCGCGTATCAACTTTACTACCTACGGATACGGTACCGGCAAAATAT
TGTATGCTATATGGAATCTGTATTCCGACGTTGTCATTCTTTATGCTTGGGCGACTTA
ATCCAGACTATTTCGCACTCTGAAATACTGGTTGGGTTAAATGCAGCGGGCTTTAAT
CTTACCTTGGATCGAAGTGATTTGAAGGAGAATCAACTCTCGCGGCTTGCTGTGAAA
TACCTCACATTGTGTGTCCAGACTGCAATAAGTAATTTGGAGGTAGGATCAGAACCC
CTTTGTATTATTGGAGGTCAATTAGATGATGACATATCATTCCAAGTTGCCCACTTCC
TCTGTCGACGACTGTGCATCATCAGCTTGGTCCACTCAAATGTACAGAACTTACCCC
CGATTAGGGATAATGAAGTTGATGTAAAATCCAAGTTGATCTACGACCATCTTAAGC
TAGTTGCTACCACACTCAATAATAGGGACCAGTCGTATCTGCTAACACTACTGAATA
AGCCTAACATCGAGCTTCATACTCCACAGGTTTACTTCATTATGAGGAAGTGTCTGG
GATTATTAAAAGTCTATGGACCTGTACCTCAAAAGCAACCACTTCCAACCTCCCCAG
TAGTATCACTACCAAATAAGTGCAAATCGAAATGGAAGCTTGAGGAGGTAATTGAC
AGCATTGAGTCACCTAAGTCTTTTAACTGGGTTCCTGATACGACGATTCCATTGGAT
GCTGAGCAAAATCCCCCAAATCCTAACAGGGTCATCGATAAGATAAACATTCTAAG
ATCTCTGAGCCCGCGTCACTCAGTCTGGTACCGTAACCGGCAATACCGGTACGTGTT
GAGCCAGTTAGGGCATGACCAGCTAGGTGGTGCAACCCTATATCTTGGTGAAGGGA
GCGGCTCCACAATCTTGAACATAGAGCCAAAAGTCAGGAGTGATAAGATATATTAT
CATACCTATTTCTCTGCAGACAAAAGCCCTGCACAGCGCAATTTCATCCCCCAGCCA
ACTACTTTTCTGCGATCTAACTTTTATCACTACGAATTAGAGCCGTCTGGATGTGAGT
TCATCAATTGTTGGTCCGAGGATGTAAATGCAACGAATCTTACGGAACTGCGTTGCA
TAAATCACATATTAACTGTGATTCCAGTCGGATCTCTAACACGTATTATTTGTGACGT
GGAATTGGCAAATGACACGTCAATACAATCAGTGGCCACCGCATACATGAACTTAA
GCATTCTTGCACATGCTCTCCTCAACCAGGGAGGCGTYTGCATCTGTAGGTGCCACC
TGTTAGACACGTCCCATCTTGCGATTGTTTCGTTTGTACTTAAAACATTATCAAGCCA
GCTGGCGATTTCGTTTTCAGGATATTGTGGAACCAATGACCCATCTTGTGTCATAGG
AGTCACGAAAGAGTGCTCCATTAGCCTGGATGTCTTAAGTTCGATTGCAGCTGCATT
TATAAATGAATTGCCCTCAAATGAGCTGCCGGTCCCTCAATCATTATTAACGCTACT
AGGGTGTTATCAAGACCAGTTAGAGAGCCTTAGTGCACTCATTGAAAAAACATGGA
TCCAGGAGATCCGGAGGCCTCATTTAATGCAATGTGAGATGGAGTGGATAGGACTTT
TGGGAACAGATGCGCTGGGCGATGTCGACACATTTATCAGTTATCGCAATGATTCGT
GTAATTCCATCCCTGATCTACTGACGCCAGCTGTGAGTGCCCTCTTATTTGAATTAAT
CAGCCTGACACCTGAGCTTCCTGGTACGGAGGACCAGCCGAGTAGGCGTGTAATCA
GCATTGGCCAAGCTTTTAACCTGACTATTTCGGGAAAAGTTAATACGATGATCAGAT
CATGCTGTGAGCAGTGCATTAAGTTATTAGCTGCCAATGTCTCCATCCTTAGTGATGT
AGATTTGTCGTATTTTGTGAGGGGGATTAGAGATGGTTCTTTTGCTTTGGGGTTGTTA
ATCACCCAGCGTCAAATACTCAAGGCATCGAGAGCACCAAAGTACCTCAAAACGCA
TCAGGTCCAGAACTGGATTGCGTTGTTACTGGAGGTCAAACTAGAGGAAATCTTCTC
TCGCCACTACCGGAAAGTGCTATTGAGAACTCTACGGCTGTTATCCCTCTACGGGCT
CCTGCAGCAGAAGGAAAGTTGATCATGATAGATGTCTCTCTGATTTCGCCTCAGCCC
TATATACAGATACCTGCTACTGCTGTTGTAACTATGCAAATATAGGCTAACCTCGGT
CCGGAGTATTGAGGTAATGGGGAGACTCGTAATTGGAGAAAGAAGAAGAACGGGG
CAGATACCAGACCCCGGGATACACTACTATAAGCTACTTGGGTGACTCAGGACGTC
ACTAATAAGAGTACACTGGTTTTAACAATAACACTGATAGTGTAACACTGAATATTG
TAAACAGTCTCAGGGATTATTTAATTAAAAAGACATTGCGGATCATCATGTCAGGCC
TGAACCAATCAGAAACCCATCCTACAGGCATGAGTCGATTAATCCAGTTTAATTTAG
TCCAAGATGATTAGTACTCTTAGATGTTACTAAGTATCAGGATTTAGCGTACTAGAT
TAAACGTCTTTGAAAGTCATTTTACCCTGCAAGGTGCATAAGCATATGCAGGGGAAT
ACCCACCACTGGTTCGTTAGTCTAATTTATTCAAAGATGCCACCAACTGAGTGCACT
GGGAGTCTGATTGAGCTGAATCAACCTAAAGCTCAAGCTTTCCTGTGATGTAGCATG
CAGTGTGCTACTGCTGCATAGAGCCAATCAGGGGCGTCTTGAAGGCCTCCACCTTCC
TCAGGCAACGATAAAGCAGCTCACTGCTACACCCACCATGCAAATACAAGGATATG
TAGGTCCATCACCCTGCTTGCTAATGTGGTCGGCTCCAGGACCATGGCCATAGCGCT
ATCCGTCTAATTCAATCCGCCTCATCATATGCGTATACAATCCTCCACTGAGAAATG
CACCTGCTCAATCCAATGACAAACTACAACAACACGAGATCATTATGGGGTCGATTC
CCTTGAATCAGAAAGGTAAAAACGGCAGCGGAGATGGAGAATACCAGACCTAATCG
GCAAAGAACAAAAGGTTGAAGACTGATTTAAAAATCATTATAACTTTTTGTTTAGT
AMPV3_SARS_COV2-S-6P_B_1_617_2_delta (SEQ ID NO: 15):
ACTAAACAGAAAATTAATACTTGTTAGTAGTCGCCGATTAATCCGCTAATATATTAG
GAGCGGAAGTCCTACACTCCGCCTCCGACCACGAATTGAAATCATCATGGCTGGAA
TTTTTAATACTTATGAGCTCTTTGTTAAGGATCAGACATGTATGCACAAGAGGGCTG
CAAGCCTAATATCGGGAGGTCAGCTTAAAAGCAATATACCAGTCTTCATCACCACAA
AAGATGATCCAGCTGTCCGATGGGACCTTGTATGTTTTAATCTCCGACTTGTTGTTAG
TGAATCATCTACCTCGGTGATCCGGCAGGGGGCTCTTATATCATTGCTTTCCATTACT
GCTAGCAATATGAGGGCTCTTGCAGCAATTGCTGGGCAGACTGATGAATCGATGATT
AATATTATAGAAGTTGTGGACTTTGATGGACTTGAGCCCCAGTGTGACACTCGCAGT
GGCTTAGACGCTCAGAAGCAGGAGATGTTTAAGGATATTGCCCGTGATATGCCGAG
GGCACTATCCAGCGGGACCCCGTTCCAGCACGGAAATGCGGAAGCGAATGGGCCTG
AAGACACACACATGTTCCTCCGATCCGCAATTAGCGTTCTGACACAGATCTGGATCC
TGGTTGCAAAAGCCATGACTAACATTGATGGCAGCAACGAGGCAAGCGATCGCCGC
CTTGCCAAGTATATTCAACAGAATAGAATTGACAGGCGATTCATGCTTGCTCAAGCT
ACCAGGACTGTATGTCAGCAGGTCATAAAGGATTCTTTGACCTTGAGAAGGTTCCTT
GTAACTGAGCTTCGAAAGTCGCGGGGGGCATTGCATAGCGGGTCATCTTACTATGCT
ATGATTGGAGACATGCAGGCATACATATTTAATGCAGGCCTTACACCATTCCTCACT
ACCCTCAGGTACGGGATCGGGACTAAATACCATGCCCTTGCAATTAGCTCACTTGCA
GGTGACCTGAATAAGATTAAGGGGTTACTGACTCTATACAAAGAGAAAGGGAGTGA
TGCGGGCTATATGGCACTTCTAGAGGATGCTGACTGCATGCAATTTGCGCCAGGTAA
TTACTCACTATTATATTCATATGCGATGGGAGTAGCAAGTGTACATGATGAAGGCAT
GAGGAATTACCAATACGCCCGGCGCTTCCTCCATAAGGGGATGTACCAATTTGGACG
TGATATCGCAACACAACAGCAACATGCACTTGATGAATCCCTCGCCCAGGAGATGA
GAATCACAGAGGCTGACAGGGCAAATCTCAAGGTAATGATGGCAAACATTGGGGAA
GCATCAAGCTTCAGTGATATGCCTAATCCAGGAGCTAGCGGCATTCCTGCCTTCAAT
GACCGCCCTGATGAGATCTTCTCTGAGCCAGGCTATCGCAAGCCCCCTCAAGATCAG
CCGCTGATAAAGCTCCCAGATCTCGAGGAAGAAGAGCAGGACGAATTCGACATGTA
GAGAGCCGCCCACCACCCAGAGCCAGCGACAAACCCAGGCAAAACAACCCACCATC
TCCGACCCGGAACACAACGCAACATCGAGGACCCCGGGCAGCCGACACCGCAGCCG
CAAGACCTCCGCATCATGAACGACCAGCATCCAAACATCTGTCCTCCCTATTCTATC
AGTTTAATAAAAAAGGCTTTACGTGCACAGACAGAGATCCTGACATAATTCAGCCG
GGGGGATTCAACTTAGGAGCGGAAGTATCCCTCCTAACCTGCACCTGCATTTCAATA
TGGATCTTGAGTTTAGTAGTGAGGAAGCAGTCGCAGCCCTCTTGGATGTGAGTTCCT
CAACCATAACCGAGGTTCTGAGCAAGCAAAGTATTCCTGACCCTAGCTTTCTCACGT
CCACGGAGGCATCCGGTGAGCAAGTTCCAGATTCGAAGGTCGCCAATCACAAAGCC
AGTGTCGATAAGACCCCAGATCAAGGCCAACCATCGGCAACGCCTTCGGCTCCTCCT
GAGACTGCCGAAAACATCAATACACCATCTTGCGAGGACGGTTTACCCCCTAACTTC
TTCATCCCAAGGGTAGAGAGTTACCACACTAACCTTTTTAAAGGGGGCCCCCAATTG
GACTCAACGGAGCGGCCGCCTGGTCACCAAATAGTTTGCGGAGATCAAGACGCGGA
CCTCTCCCGAGCCGGGATAGCGAGAAAGAAGAAGAAGCAGAAGCATTGCAAAGCG
CTCAACTTTTCGGAATCCCCACTCACAGACAATCAGGCCATAGAAGGGAGTATTCAA
TCTCATGGAGCCCTGGATCAGGAACCTTCCAGACAGAGACCTGGTGCAACCCAGCC
TGCTCTCCAGTCACCGCCTTCCCAAAACAATACAAGTGTGCATGCAGACAGTGCCCA
AGATTCTGCGATCTCTGTTTCAATCCCGCTGACTATGGTGGAATCATTGATCTCTCAA
GTGTCAAAGTTGTCGGACCAAGTCTCGCAGATACAGAAGTTGGTGAGTACCCTTCCT
CAAATTAAGACTGATATTGCTTCGATTCGGAACATGCAAGCAGCTTTGGAAGGACA
GCTCAGTATGATTCGTATATTGGATCCAGGAAACTGTTCGGAATCATCACTCAATGC
GCTGAGAGGGTTCAGTGGTAAGGCACCTGTTATTGTGAGTGGTCCAGGAAATCCCA
ATCAATGCATCAGCCAAGGCTCATCCACCACAATTTGCCTGGATGAGTTAGCCCGGC
CTGTGCCAAATCCTATACAGGTGAAACTTCAAGATGATCAGAAGGACCTATCTGCAC
AGCGACATGCTGTCATTGCATTACTCGAAACCAGAATCCCCCCGGGACCAAAACGT
GACAAATTACTGTCATTGGTTGCAATTGCAAAGACTGCAAGTGATTTAATCAAGATT
AAGAGAATGGCAGTCCTCGGCCAGTGATATCACCCTATGGCAACCAGGCTACAAAT
GTAAGCAGTTTCCGATATGTACCGAATCAATAACACGACCCCTTCCGCAAAAGCAA
GCGCGAGCATGACACACTCAGCTTCAGCCAATTCTAGCTTCACTAAGCAGGTGATCT
GACCATCTGGCTTTACTTCCGCTTTATAAAAAACTCCTAGATCTAACATGGAGGCAG
ACCGAGCTAATGCGGAGGGAGTACACAAGGGGAATCAAAGGAGCGGAAGGTACTA
AGTCACTCCACCGCCTGCCCTCGCCCATCCGCAGCCTCCCCCAACCCGCGGCGCCGC
CACCATGTTCGTGTTTCTGGTGCTGCTGCCTCTGGTGAGCTCCCAGTGCGTGAACCTG
AGGACAAGGACCCAGCTGCCCCCTGCCTATACCAATTCCTTCACACGGGGCGTGTAC
TATCCCGACAAGGTGTTTAGATCTAGCGTGCTGCACTCCACACAGGATCTGTTTCTG
CCTTTCTTTTCTAACGTGACCTGGTTCCACGCCATCCACGTGAGCGGCACCAATGGC
ACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAA
GACACAGTCCCTGCTGATCGTGAACAATGCCACCAACGTGGTCATCAAGGTGTGCG
AGTTCCAGTTTTGTAATGATCCATTCCTGGGCGTGTACTATCACAAGAACAATAAGT
CTTGGATGGAGAGCGGCGTGTATTCCTCTGCCAACAATTGCACATTTGAGTACGTGT
CCCAGCCCTTCCTGATGGACCTGGAGGGCAAGCAGGGCAATTTCAAGAACCTGAGG
GAGTTCGTGTTTAAGAATATCGATGGCTACTTCAAGATCTACTCCAAGCACACCCCA
ATCAACCTGGTGCGCGACCTGCCACAGGGCTTCTCTGCCCTGGAGCCACTGGTGGAT
CTGCCCATCGGCATCAACATCACCCGGTTTCAGACACTGCTGGCCCTGCACAGAAGC
TACCTGACACCAGGCGACAGCTCCTCTGGATGGACCGCAGGAGCTGCCGCCTACTAT
GTGGGCTATCTGCAGCCCAGGACCTTCCTGCTGAAGTACAACGAGAATGGCACCAT
CACAGACGCCGTGGATTGCGCCCTGGATCCCCTGTCTGAGACCAAGTGTACACTGAA
GAGCTTTACCGTGGAGAAGGGCATCTATCAGACAAGCAATTTCAGGGTGCAGCCTA
CCGAGTCCATCGTGCGCTTTCCCAATATCACAAACCTGTGCCCTTTTGGCGAGGTGTT
CAACGCAACCCGCTTCGCCAGCGTGTACGCCTGGAATAGGAAGCGCATCTCCAACT
GCGTGGCCGACTATTCTGTGCTGTACAACAGCGCCTCCTTCTCTACCTTTAAGTGCTA
TGGCGTGAGCCCCACAAAGCTGAATGACCTGTGCTTTACCAACGTGTACGCCGATTC
CTTCGTGATCAGGGGCGACGAGGTGCGCCAGATCGCCCCTGGCCAGACAGGCAAGA
TCGCCGACTACAATTATAAGCTGCCTGACGATTTCACCGGCTGCGTGATCGCCTGGA
ACTCTAACAATCTGGATAGCAAAGTGGGCGGCAACTACAATTATCGGTACCGGCTGT
TTAGAAAGTCTAATCTGAAGCCATTCGAGAGGGACATCTCCACAGAGATCTACCAG
GCCGGCTCTAAGCCCTGCAATGGCGTGGAGGGCTTTAACTGTTATTTCCCTCTGCAG
AGCTACGGCTTCCAGCCAACAAACGGCGTGGGCTATCAGCCCTACCGCGTGGTGGT
GCTGTCTTTTGAGCTGCTGCACGCACCTGCAACAGTGTGCGGACCAAAGAAGAGCA
CCAATCTGGTGAAGAACAAGTGCGTGAACTTCAACTTCAACGGACTGACCGGCACA
GGCGTGCTGACCGAGTCCAACAAGAAGTTCCTGCCTTTTCAGCAGTTCGGCAGGGAC
ATCGCAGATACCACAGACGCCGTGCGCGACCCTCAGACCCTGGAGATCCTGGATAT
CACACCATGCTCCTTCGGCGGCGTGTCTGTGATCACACCAGGCACCAATACAAGCAA
CCAGGTGGCCGTGCTGTATCAGGGCGTGAATTGTACCGAGGTGCCCGTGGCAATCCA
CGCAGATCAGCTGACCCCTACATGGCGGGTGTACTCTACCGGCAGCAACGTGTTCCA
GACAAGAGCCGGATGCCTGATCGGAGCCGAGCACGTGAACAATAGCTATGAGTGCG
ACATCCCTATCGGCGCCGGCATCTGTGCCTCCTACCAGACCCAGACAAACTCCAGAG
GGTCTGCCTCCTCTGTGGCCAGCCAGTCCATCATCGCCTATACCATGAGCCTGGGCG
CCGAGAATTCCGTGGCCTACTCCAACAATTCTATCGCCATCCCTACCAACTTCACAA
TCTCCGTGACCACAGAGATCCTGCCAGTGAGCATGACCAAGACATCCGTGGACTGC
ACAATGTATATCTGTGGCGATTCCACCGAGTGCTCTAACCTGCTGCTGCAGTACGGC
TCTTTTTGTACCCAGCTGAATAGAGCCCTGACAGGCATCGCCGTGGAGCAGGACAAG
AACACACAGGAGGTGTTCGCCCAGGTGAAGCAGATCTACAAGACCCCACCCATCAA
GGACTTTGGCGGCTTCAACTTCAGCCAGATCCTGCCCGATCCTAGCAAGCCATCCAA
GCGGTCTCCTATCGAGGACCTGCTGTTCAACAAGGTGACCCTGGCCGATGCCGGCTT
CATCAAGCAGTATGGCGATTGCCTGGGCGACATCGCCGCCAGAGACCTGATCTGTG
CCCAGAAGTTTAATGGCCTGACCGTGCTGCCTCCACTGCTGACAGATGAGATGATCG
CCCAGTACACATCTGCCCTGCTGGCCGGCACCATCACAAGCGGATGGACCTTCGGCG
CAGGACCCGCCCTGCAGATCCCCTTTCCCATGCAGATGGCCTATCGGTTCAACGGCA
TCGGCGTGACCCAGAATGTGCTGTACGAGAACCAGAAGCTGATCGCCAATCAGTTT
AACTCCGCCATCGGCAAGATCCAGGACTCTCTGAGCTCCACACCCAGCGCCCTGGGC
AAGCTGCAGAACGTGGTGAATCAGAACGCCCAGGCCCTGAATACCCTGGTGAAGCA
GCTGTCTAGCAACTTCGGCGCCATCTCCTCTGTGCTGAATGATATCCTGAGCAGGCT
GGACCCTCCAGAGGCAGAGGTGCAGATCGACCGGCTGATCACAGGCAGACTGCAGT
CCCTGCAGACCTACGTGACACAGCAGCTGATCAGGGCAGCAGAGATCAGGGCCTCT
GCCAATCTGGCCGCCACCAAGATGAGCGAGTGCGTGCTGGGCCAGTCCAAGAGAGT
GGACTTTTGTGGCAAGGGCTATCACCTGATGAGCTTCCCACAGTCCGCCCCTCACGG
AGTGGTGTTTCTGCACGTGACCTACGTGCCAGCCCAGGAGAAGAACTTCACCACAG
CACCAGCAATCTGCCACGATGGCAAGGCACACTTTCCTAGGGAGGGCGTGTTCGTG
AGCAACGGCACCCACTGGTTTGTGACACAGCGCAATTTCTACGAGCCACAGATCATC
ACCACAGACAATACATTCGTGTCCGGCAACTGTGACGTGGTCATCGGCATCGTGAAC
AATACCGTGTATGATCCTCTGCAGCCAGAGCTGGACTCTTTTAAGGAGGAGCTGGAT
AAGTACTTCAAGAATCACACCAGCCCCGACGTGGATCTGGGCGACATCTCTGGCATC
AATGCCAGCGTGGTGAACATCCAGAAGGAGATCGACAGGCTGAACGAGGTGGCCAA
GAATCTGAACGAGTCCCTGATCGATCTGCAGGAGCTGGGCAAGTATGAGCAGTACA
TCAAGTGGCCCTGGTATATCTGGCTGGGCTTCATCGCCGGCCTGATCGCCATCGTGA
TGGTGACCATCATGCTGTGCTGTATGACAAGCTGCTGTTCCTGCCTGAAGGGCTGCT
GTTCTTGTGGCAGCTGCTGTAAGTTTGATGAGGACGATAGCGAGCCTGTGCTGAAGG
GCGTGAAGCTGCACTACACCTGATAGTTTATAAAAAACATTGTGAGAATGCAATTTA
AGGACGAAAGTAGGAGCGGAAGCCGCGGCCCAGGCTCCATTCAAAAACCATGGCC
GCTGCTCTCGGGAACATGCACCCGTCATCGTCGATTACTCTTATGCATGATGACCCG
TCCATCCAGACGCAGTTGCTAGCCTTCCCTCTGATAAGTGAGAAGACAGAGACAGG
GACGACAAAGCTGCAGCCTCAAGTGAGAATGCAATCTTTCCTATCAACCGATAGCC
AGAAGTATCATCTGGTCTTTATCAACACATATGGTTTCATTGCGGAGGATTTTAATTG
TAGTCCAGCAAACGGATTTGTGCCTGCACTATTTCAACCCAAGTCTAAAGTCTTATC
GTCAGCGATGGTTACACTTGGGGCTGTCCCTGCTGACACTGTTCTCCAGGACCTACA
ACGGGACTTAATCGCAATGAGATTTAAAGTCCGAAAGAGTGCGTCAGCGAAGGAGC
TAATATTATTCTCCACAGACAATGTCCCTGCAACATTAACAGGGTCATCAGTCTGGA
AGAATAAAGGAGTAATAGCTGATACAGCAACTGCAGTTAAAGCGCCCGGTCGCATC
TCATGCGATGCGGTATGCAGTTATTGCATTACATTCATCTCTTTTTGCTTCTTCCACTC
ATCTGCTCTTTTTAAGGTGCCCAAGCCTCTGCTGAATTTCGAGACGGCCATAGCATA
TTCCCTTGTACTCCAGGTCGAGCTTGAGTTCCCAAATATCAAAGATACCTTACACGA
GAAGTATCTCAAAGTTAAAGACTCGAAGTGGTATTGTACAATTGACATCCATATTGG
GAATCTTTTAAAGAGAACCGCCAAGCAACGAAGGAGGACACCTTCAGAGATAATGC
AGAAAGTCCGTAAGATGGGTTTCAGGATTGGGTTATACGATTTGTGGGGTCCAACAA
TTGTGGTTGAATTAACTGGGTCCTCAAGTAAGTCCCTACACGGATTCTTCTCGAGTG
AGCGGCTGGCCTGCCACCCGGTATCACAGTATAATCCTCATGTTGGACAGCTTATCT
GGGCCCACGATGTATCCATCACGGGGTGCCACATGATTATATCTGAACTTGAGAAAA
AGAAGGCACTTGCAATGGCTGACCTGACCGTGAGTGATGCAGTAGCTGTCAACACG
AGCATCAAGAGCTTATCGCCTTTCAGACTGTTCAAAAAATATTAGGGCTAGACAACT
CCTAGGGTGTGTGCTTATAATTTGCATATTCTAGATTATAATCATTAACCGCGTCCAA
ATGATCAGACCGCATAATTAACAGCTCAGCCAGGACTCGCAAATCCTGCATAGGCT
CAAATAACAAGACGACATATATACTCACACAACAAGGTGTAATATCTAGAAGGAAT
CAGAATTGAGACAATATCTAGGTTATCAGCGCTACTGCCCTGCAACTCACTATGTAT
GCTTGGCATTAATAAAAAACATTGTGAGAATGCAATTTAAGGACGAAAGTAGGAGC
GGAACACGCTCAACTGAACCCAAGCGCACACCAGCCCCCCGGCACCGCACCGGAAG
GACGCACAACAGCCGACCGCCCCCACCAGGGCAGAGGCCATGCAACCAGGCTCGGC
ACTCCACCTCCCGCACCTGTACATAATAATTGCCTTAGTCAGCGATGGCACTTTAGG
CCAAACAGCTAAAATTGATAGGCTGATACAGGCAGGGATTGTCTTGGGTAGTGGAA
AGGAGCTCCATATCTCACAAGATTCAGGGACACTGGATTTATTCGTGAGGTTGTTAC
CAGTACTCCCTTCGAATTTGTCCCATTGCCAGCTTGAAGCAATAACACAGTATAATA
AGACGGTGACTAGGTTATTGGCCCCAATAGGGAAGAACCTAGAACAGGTACTACAA
GCGAGACCGCGTGGGAGGCTGTTCGGAGCAATAATAGGGTCCATCGCGCTTGGTGT
TGCCACATCAGCACAGATTACCGCTGCAATTGCACTCGTCCGTGCACAGCAAAATGC
TAATGATATATTAGCACTTAAAAACGCTCTCCAATCCAGCAATGAGGCAATCCGTCA
ACTCACGTATGGCCAGGACAAACAGTTACTTGCAATTTCTAAAATACAAAAAGCTGT
GAATGAGCAGATCCTCCCTGCACTCGATCAGCTGGACTGTGCTGTCCTTGGAACCAA
ACTAGCTGTGCAGCTTAATCTGTATCTCATTGAGATGACTACCATTTTCGGCGAGCA
AATCAATAATCCAGTCTTGGCGACCATCCCATTAAGTTACATTTTACGCCTGACCGG
TGCGGAGTTGAACAACGTCCTCATGAAGCAAGCCCGATCGTCCTTAAGCCTTGTACA
GTTAGTTTCAAAAGGCTTACTTAGTGGACAAGTAATTGGCTATGACCCATCTGTTCA
AGGTCTGATAATAAGGGTGAATCTGATGCGAACGCAGAAGATCGATAGAGCACTAG
TTTATCAGCCGTATGTATTACCAATCACTCTTAACTCTAACATAGTAACACCAATCGC
CCCAGAATGCGTAATTCAGAAAGGGACGATCATAGAGGGTATGTCACGGAAGGATT
GTACAGAGTTGGAGCAAGACATAATTTGTAGGACAGTTACGACATACACGCTTGCC
AGAGACACCAGATTGTGTTTACAAGGCAATATCTCTTCTTGTAGGTATCAACAGTCA
GGCACCCAACTACACACCCCATTTATTACGTACAACGGGGCAGTTATCGCAAACTGT
GATTTAGTCTCTTGTCGGTGTCTCCGTCCTCCTATGATTATCACCCAAGTAAAAGGGT
ACCCTCTTACCATAATCACAAGGTCCGTATGTCAAGAATTATCCGTTGACAACTTAG
TCCTGAATATTGAGACACACCATAATTTCTCCCTTAATCCAACAATTATCGACCCTCT
AACAAGAGTAATAGCCACTACACCGTTAGAGATAGATTCCTTAATCCAAGAGGCAC
AAGATCATGCGAATGCAGCGCTAGCTAAAGTGGAAGAGAGTGATAAATATCTCAGG
GCAGTTACTGGCGGTAATTATTCGAATTGGTATATCGTTCTTGTCATCGTCTTACTAT
TTGGGAATTTGGGCTGGTCGCTGTTGTTAACAGTACTTCTATGTAGGTCACGGAAAC
AACAGCGACGTTATCAACAAGATGATTCCGTCGGGTCAGAAAGAGGTGTTGGGGTT
GGGACCATCCAGTATATGTCATAAGTGGTGTCAGACATCACAATCCAAGTCACTCAG
TTATCTACTCCGCTGGCTCGGAGCCATCGACAACGGCGTGAGCCCTCATCCGGGGAC
AATCCACAACCTGATACTACCAGACAGTGGCTCCGCACGATGGCAGCAGCCCACAA
CGTCCTGCATGTAAATATCATTGCGTTACTTTAATTAAAAAACCTCAAGGTGACCCA
TCCTGGCCCATCCGATTGGAGGAGCGGAAGGTTTTAAGGTAATCAGGCAAATTGTTT
CCAACATGGAATCCCCTCCTTCTGGCAAGGATGCGCCAGCCTTCCGCGAGCCTAAGC
GAACTTGCAGACTCTGTTACAGGGCGACGACTCTCTCCCTTAATCTCACCATCGTTGT
ATTATCAATTATAAGCATTTATGTATCAACTCAAACTGGGGCAAACAACTCTTGTGT
CAATCCCACAATCGTAACTCCTGATTATTTAACTGGCAGCACGACAGGCTCAGTTGA
AGATCTTGCTGACTTAGAGAGTCAACTCCGGGAGATACGTAGAGACACAGGGATTA
ATCTCCCAGTTCAGATCGATAATACAGAAAATCTAATACTGACAACTCTGGCGAGTA
TCAACTCGAATTTACGCTTTCTGCAAAATGCAACAACTGAGAGCCAGACCTGCCTAT
CTCCTGTCAATGACCCTCGCTTTGTAGCTGGCATAAATAGGATCCCAGCAGGGTCGA
TGGCATATAATGACTTCAGCAACCTCATTGAGCACGTAAATTTCATACCGTCGCCGA
CAACGTTGTCAGGCTGCACCAGGATACCATCATTTTCACTCTCTAAGACGCACTGGT
GTTATACCCACAATGTTATATCAAACGGGTGCCTTGACCATGCTGCAAGTTCACAAT
ACATCTCAATAGGGATCGTTGATACCGGCCTAAATAATGAGCCCTATTTTAGAACAA
TGTCTTCAAAGTCACTGAATGACGGATTAAATAGAAAGAGTTGCTCTGTGACCGCAG
CCGCTAATGCATGTTGGTTACTCTGTAGCGTAGTAACAGAATACGAAGCTGCGGACT
ATCGATCACGAACTCCTACTGCTATGGTTCTGGGGCGATTCGATTTTAATGGTGAAT
ACACAGAAATAGCTGTCCCCTCCTCACTATTCGATGGCCGGTTTGCATCTAACTATC
CAGGTGTGGGATCGGGAACTCAAGTCAATGGAACATTATATTTCCCTTTATATGGAG
GGGTCCTTAATGGGTCAGATATAGAAACGGCAAACAAGGGGAAATCCTTCAGGCCT
CAGAATCCTAAGAATAGGTGTCCAGACTCGGAGGCGATCCAAAGCTTTAGAGCGCA
AGATAGCTATTACCCGACGAGATTTGGGAAGGTGCTGATCCAACAGGCAATCATTG
CATGTAGGATCAGTAACAAAAGTTGTACTGATTTCTATCTCCTGTACTTCGACAACA
ACAGAGTGATGATGGGTGCAGAGGCACGGCTATATTATCTTAATAACCAATTATATC
TATATCAACGCTCGTCGAGCTGGTGGCCGCATCCTTTGTTCTATAGCATCTCATTACC
GTCATGTCAAGCCCTTGCTGTCTGTCAAATTACAGAGGCCCATCTGACACTGACTTA
CGCCACGTCGAGGCCAGGAATGTCTATCTGTACAGGAGCATCAAGGTGTCCAAATA
ATTGCGTAGACGGTGTATATACTGATGTATGGCCATTGACAAAGAACGATGCTCAGG
ACCCCAATCTCTTCTACACTGTATACTTAAATAATTCAACTCGGCGGATTAGCCCAA
CCATAAGTCTATACACTTATGACCGCAGGATCAAATCTAAGCTGGCAGTGGGTAGTG
ATATAGGAGCAGCTTATACCACGAGTACCTGTTTCGGCCGATCAGATACAGGGGCG
GTTTATTGCTTAACTATAATGGAGACCGTTAACACAATATTTGGTCAGTACCGGATC
GTGCCAATATTACTTAGAGTGACAAGCCGGTAATGGATTGTTCCTAGTGAAGTGGGG
CCTATGGTTACTCCGTAACGGAGATGACCTGATTTATGCTATGTAACTAATTTTGCTC
AATCGGGAGGGTGATCGGCCGGGTATGTCCACAATGACAATGACTCCTATTCCCTTG
CTAGAGCTAGCTTCCTCTTTAATCCAATACAATCCCTACCTGTAGTTTAATAAAAAA
ATCCTCTATATAAGATCCAGACGCGTGCTATATAAGCTAATAGCTTAGGATGGAAAC
GACAGGAGCGGAACATATCCCTCCTACCATGTCTTCTCATAATATTATCCTCCCTGA
CCACCACTTAAATTCCCCTATTGTACTAAATAAATTGATGTACTATTGTAAACTCCTT
AATATATTGCCTGACCCTAAGTCGCCATGGTACGAGAAGATTAAGTCTTGGACTAAT
TGCTGCATCCGTGTGTCAGACAGCAATCGCATGACTTTGTCACGCGCCTCCAAGCTA
CGAGAACTATTGGCAACGTACGGCGTATATTCGAAGAACCACCAGCAATGTTATAC
CACCATAATCCATCCCCAATCCCTATCACCCATCATGCAGACTGTCTCTCAACTGGG
ACGCTCAATCCCGACCTGGGCCAGGATCCGAAAGGAGATCACACACAGTATCATAG
CCCAAACGAATAAATTTGAATCCTTATTCCACAACATCTCAAAGGACCTGACAGGGA
AAACCAACTTGTTTGGCGGATTCTGTGATTTACATGGGAGCATAAGCACTGCTGCAA
AGCGCTTAATGAATCAACCGGGTCTCTACTTGGATTCACCTGATGCCCATGCATGTC
AATTTTTGTTCCAGCTTAAAACCTGTCAACGGGAATTGATTCTACTTATGAGGCAGA
ATGCCACAACAGAGCTGATTAGGGTTTTTGACTATCCTGAGCTACGGGTTCTTGTGA
CACCTGAATATTCGGTCTGGGTTTTTACACGGACCAAACAAGTTACATTACTAACAT
TCGATTGCTTGTTAATGTACTGCGATCTTTCAGACGGCCGGCACAACATCCTATTCAC
TTGTAAGCTTTTGTCTCACTTAAACCACCTCGGGTGTCGGATAAGGGACCTGCTGGT
GCTAATTGATTCACTTGCCGAGAAACACCCCTTAATAGTTTATGATGTAGTTGCAAG
TCTGGAGTCGTTATCATATGGTGCTATACAGCTACATGACAAGGTAGCTGGTTATGC
TGGAACATTTTTCTCATTTATCCTTGCAGAAATACAGGATTCCTTAGAAACAGTGTTG
GATCAAGGTAATAGTGAATCTATCATATCCCAAATAAGGAACATATACTCAGGTCTC
ACAGTAAACGAGGCTGCAGAATTGCTATGTGTTATGAGGCTTTGGGGCCACCCTGCC
CTAAACAGTGTAGACGCTGCAAGCAAGGTGAGACAGAGTATGTGCGCTGGCAAGCT
GCTTAAATTTGATACGATCCAGTTAGTCCTTGCATTCTTCAATACACTAATAATTAAT
GGATATAGGAGAAAGCACCATGGGCGATGGCCGAATGTTTGTAGTGACTCTATAAT
TGGGAATGAACTCAAGCGGATGTATTTTGATCAATGTGAGATCCCCCACGACTTCAC
ATTAAAGAACTACCGGGAATTAAGTCTTCTTGAATTCGAGTGCACATTTCCCATAGA
ATTATCAGATAAACTGAACATATTTTTAAAAGATAAGGCAATAGCATTCCCGAAGTC
CAGGTGGACATCTCCTTTCAAAGCAGATATCACACCGCGCCATTTGCTGCAAGCGCC
TGAGTTTAAAACCCGTGCCAACAGACTACTGCTATCATTCTTACAACTCGAGGAATT
CTCCATCGAATCAGAACTAGAATATGTGACTACCCTGGCTTACCTGGATGATGATGA
GTTCAACGTATCTTACTCCCTCAAAGAGAAGGAGGTAAAGACAGATGGTCGAATCTT
CGCTAAGCTCACAAGGAAAATGCGGAGCTGTCAGGTGATATTCGAAGAATTGCTAG
CAGAGCATGTCTCTCCATTATTTAAAGACAACGGCGTTACTATGGCCGAATTGTCAC
TTACTAAGAGCCTACTCGCTATTAGCAACCTAAGCTCCACCTTATTTGAGACGCAAA
CAAGGCAAGGCGATCGGAATGCAAGATTCACGCATGCGCACTTCATAACAACTGAC
CTGCAAAAGTACTGTTTAAACTGGAGGTACCAGAGTGTAAAACTGTTCGCACGTCAA
TTGAACCGGTTATTTGGTCTGCAGCATGGGTTTGAATGGATCCATTGCATACTAATG
AAGTCCACTATGTATGTCGCAGACCCCTTTAACCCTCCACATAGCAATGCACGTGAG
GCACTCGATGACAACTTAAATAGCGACATATTCATTGTATCACCTAGGGGGGCCATC
GAGGGACTATGCCAAAAAATGTGGACAATCATCTCAATCTCGGCGATCCATGCATCC
GCTGCTGTGGCTGGGCTACGGGTGGCATCTATGGTCCAAGGTGATAACCAAGTTATT
GGGGTAACACGCGAGTTCCTAGCTGGGCATGATCAAACTTTTGTAGACGATCAATTG
ACGGTTTCACTTGAGAACTTCACTCAGATATTCAAGGAAGTCAACTACGGGCTAGGT
CATAATCTCAAGCTGAGGGAAACAATCAAGTCGAGCCACATGTTCATCTACTCAAA
GAGGATCTTCTACGACGGCCGAGTGCTTCCACAACTTCTAAAGAACATAAGTAAGTT
AACCCTATCAGCTACAACTACAGGGGAGAACTGTTTAACGAGTTGCGGGGACCTAT
CATCATGCATCACACGATGTATAGAGAATGGGTTCCCTAAAGATGCAGCATTCGTTT
TGAATCAATTAGTGATTAGGATCCAGATACTTGCCGATCACTTTTATTCCATCCTTGG
AGGATGTTTCTCTGGGTTGAACCAGGCGGATATCCGTCTCCTTCTATATGAGGGTGC
AATTTTACCGGCACAATTAGGAGGGTTCAACAACCTCAACATGTCACGGCTATTCTG
TCGGAATATTGGTGACCCATTAGTCGCGTCCATCGCAGACACCAAGAGATTTGTGAA
GTGCCAACTGCTCGTTCCTCACATTTTAGACTCTGTCGTTGCTATTACCGATAGGAAG
GGGTCTTTTACGACACTGATGATGGATCCATATTCAATAAACTTGGATTATATCCAG
CAACCAGAGACACGATTGAAGCGACATGTGCAAAAGGTTTTACTACAAGAGTCAGT
GAATCCGCTACTCCAAGGGGTCTTCCTAGAGTCTCAACAAGAGGAAGAGGAACAAC
TTGCAGCGTTCTTGCTGGACCGAGAAGTCGTAATGCCAAGGGTTGCGCACGCTATAT
TTGAGTGTACCAGCTTAGGTAGGCGCCGACATATCCAGGGCCTCATCGACACGACA
AAGACCATTATTGCACTTGCGTTGGATACACAGCAGTTAAGTTATACTAAACGTGAA
CAGATTGTGACGTACAATGCAACTTATATGAGGTCCTTAGCAAGCATGCTCAGTTCA
AGCCATAATCAGACACGTCGGTCTGTGATTGGTCACGCATCCTTCAATATCACCGAC
TGCTCGGTCATCCTGGCTCAACAAGTGAGACGTGCAAGCTGGGCGCCCTTGCTGAAT
TGGCGGTCATTAGAAGGCTTAGAAGTACCCGACCCTATTGAGTCAGTAGCCGGCTAC
CTGGGTTTGGATTCTAACAACTGTTTCTTATGCTGCCACGAGCAGAACAGCTATTCCT
GGTTTTTCCTTCCCCGAATGTGCCATTTTGATGATTCGAGACAATCTAACTCCATACA
AAGGGTACCTTACATTGGTTCTAAGACTGACGAGCGCCAGATGTCTACGATAAATCT
ACTTGAGAAAACTACCTGTCATGCTAGAGCAGCTACGAGGCTTGCATCATTATACAT
TTGGGCCTTTGGGGACTCAGACGATAGTTGGGATGCAGTCGAGACTCTATCTAACAG
TCGATGCCAGATCACCCGAGAGCACTTGCAAGCTCTGTGCCCTATGCCCTCTTCAGT
GAATCTCCACCATCGATTGAACGACGGAATAACACAAGTGAAGTTTATGCCATCGA
CCAATAGCAGGGTCTCTCGATTTGTTCATATTTCCAATGACCGGCAGAATTACGTAT
TGGATGACTCCGTTACTGACAGTAATCTGATTTACCAACAAGTCATGTTGCTAGGAC
TTAGTATACTTGAAACATATTTCAGAGAACCTACAGCGACGAACTTATCAAGCTTGG
TGCTGCACCTTCACACTGATGTATCTTGTTGCTTACGAGAATGCCCGATGACACAAT
ATGCGCCCCCCCTGCGAGACATCCCAGAGTTAACTATAACTGCGTCAAATCCTTTCT
TATTTGATCAAGAACCTATTAGCGAAGCAGACCTGTGTAGACTCTCAAAGATAGCAT
TCCGTAGAGCAGGAGACAACTACGATGCATACGACCAGTTTCAACTAAGAGCAACA
CTGGCCGCAACTACAGGTAAGGATGTGGCAGCAACAATCTTTGGTCCTCTAGCTGCA
GTATCAGCAAAAAATGATGCAATAGTCACAAATGACTATAGCGGGAATTGGATTTC
CGAGTTTAGGTATAGTGATCTCTATCTTTTGAGTGTTAGCCTGGGCTACGAGATCCTT
TTGATTTTCGCGTATCAACTTTACTACCTACGGATACGGTACCGGCAAAATATTGTAT
GCTATATGGAATCTGTATTCCGACGTTGTCATTCTTTATGCTTGGGCGACTTAATCCA
GACTATTTCGCACTCTGAAATACTGGTTGGGTTAAATGCAGCGGGCTTTAATCTTAC
CTTGGATCGAAGTGATTTGAAGGAGAATCAACTCTCGCGGCTTGCTGTGAAATACCT
CACATTGTGTGTCCAGACTGCAATAAGTAATTTGGAGGTAGGATCAGAACCCCTTTG
TATTATTGGAGGTCAATTAGATGATGACATATCATTCCAAGTTGCCCACTTCCTCTGT
CGACGACTGTGCATCATCAGCTTGGTCCACTCAAATGTACAGAACTTACCCCCGATT
AGGGATAATGAAGTTGATGTAAAATCCAAGTTGATCTACGACCATCTTAAGCTAGTT
GCTACCACACTCAATAATAGGGACCAGTCGTATCTGCTAACACTACTGAATAAGCCT
AACATCGAGCTTCATACTCCACAGGTTTACTTCATTATGAGGAAGTGTCTGGGATTA
TTAAAAGTCTATGGACCTGTACCTCAAAAGCAACCACTTCCAACCTCCCCAGTAGTA
TCACTACCAAATAAGTGCAAATCGAAATGGAAGCTTGAGGAGGTAATTGACAGCAT
TGAGTCACCTAAGTCTTTTAACTGGGTTCCTGATACGACGATTCCATTGGATGCTGA
GCAAAATCCCCCAAATCCTAACAGGGTCATCGATAAGATAAACATTCTAAGATCTCT
GAGCCCGCGTCACTCAGTCTGGTACCGTAACCGGCAATACCGGTACGTGTTGAGCCA
GTTAGGGCATGACCAGCTAGGTGGTGCAACCCTATATCTTGGTGAAGGGAGCGGCT
CCACAATCTTGAACATAGAGCCAAAAGTCAGGAGTGATAAGATATATTATCATACCT
ATTTCTCTGCAGACAAAAGCCCTGCACAGCGCAATTTCATCCCCCAGCCAACTACTT
TTCTGCGATCTAACTTTTATCACTACGAATTAGAGCCGTCTGGATGTGAGTTCATCAA
TTGTTGGTCCGAGGATGTAAATGCAACGAATCTTACGGAACTGCGTTGCATAAATCA
CATATTAACTGTGATTCCAGTCGGATCTCTAACACGTATTATTTGTGACGTGGAATTG
GCAAATGACACGTCAATACAATCAGTGGCCACCGCATACATGAACTTAAGCATTCTT
GCACATGCTCTCCTCAACCAGGGAGGCGTYTGCATCTGTAGGTGCCACCTGTTAGAC
ACGTCCCATCTTGCGATTGTTTCGTTTGTACTTAAAACATTATCAAGCCAGCTGGCGA
TTTCGTTTTCAGGATATTGTGGAACCAATGACCCATCTTGTGTCATAGGAGTCACGA
AAGAGTGCTCCATTAGCCTGGATGTCTTAAGTTCGATTGCAGCTGCATTTATAAATG
AATTGCCCTCAAATGAGCTGCCGGTCCCTCAATCATTATTAACGCTACTAGGGTGTT
ATCAAGACCAGTTAGAGAGCCTTAGTGCACTCATTGAAAAAACATGGATCCAGGAG
ATCCGGAGGCCTCATTTAATGCAATGTGAGATGGAGTGGATAGGACTTTTGGGAAC
AGATGCGCTGGGCGATGTCGACACATTTATCAGTTATCGCAATGATTCGTGTAATTC
CATCCCTGATCTACTGACGCCAGCTGTGAGTGCCCTCTTATTTGAATTAATCAGCCTG
ACACCTGAGCTTCCTGGTACGGAGGACCAGCCGAGTAGGCGTGTAATCAGCATTGG
CCAAGCTTTTAACCTGACTATTTCGGGAAAAGTTAATACGATGATCAGATCATGCTG
TGAGCAGTGCATTAAGTTATTAGCTGCCAATGTCTCCATCCTTAGTGATGTAGATTTG
TCGTATTTTGTGAGGGGGATTAGAGATGGTTCTTTTGCTTTGGGGTTGTTAATCACCC
AGCGTCAAATACTCAAGGCATCGAGAGCACCAAAGTACCTCAAAACGCATCAGGTC
CAGAACTGGATTGCGTTGTTACTGGAGGTCAAACTAGAGGAAATCTTCTCTCGCCAC
TACCGGAAAGTGCTATTGAGAACTCTACGGCTGTTATCCCTCTACGGGCTCCTGCAG
CAGAAGGAAAGTTGATCATGATAGATGTCTCTCTGATTTCGCCTCAGCCCTATATAC
AGATACCTGCTACTGCTGTTGTAACTATGCAAATATAGGCTAACCTCGGTCCGGAGT
ATTGAGGTAATGGGGAGACTCGTAATTGGAGAAAGAAGAAGAACGGGGCAGATAC
CAGACCCCGGGATACACTACTATAAGCTACTTGGGTGACTCAGGACGTCACTAATAA
GAGTACACTGGTTTTAACAATAACACTGATAGTGTAACACTGAATATTGTAAACAGT
CTCAGGGATTATTTAATTAAAAAGACATTGCGGATCATCATGTCAGGCCTGAACCAA
TCAGAAACCCATCCTACAGGCATGAGTCGATTAATCCAGTTTAATTTAGTCCAAGAT
GATTAGTACTCTTAGATGTTACTAAGTATCAGGATTTAGCGTACTAGATTAAACGTC
TTTGAAAGTCATTTTACCCTGCAAGGTGCATAAGCATATGCAGGGGAATACCCACCA
CTGGTTCGTTAGTCTAATTTATTCAAAGATGCCACCAACTGAGTGCACTGGGAGTCT
GATTGAGCTGAATCAACCTAAAGCTCAAGCTTTCCTGTGATGTAGCATGCAGTGTGC
TACTGCTGCATAGAGCCAATCAGGGGCGTCTTGAAGGCCTCCACCTTCCTCAGGCAA
CGATAAAGCAGCTCACTGCTACACCCACCATGCAAATACAAGGATATGTAGGTCCA
TCACCCTGCTTGCTAATGTGGTCGGCTCCAGGACCATGGCCATAGCGCTATCCGTCT
AATTCAATCCGCCTCATCATATGCGTATACAATCCTCCACTGAGAAATGCACCTGCT
CAATCCAATGACAAACTACAACAACACGAGATCATTATGGGGTCGATTCCCTTGAAT
CAGAAAGGTAAAAACGGCAGCGGAGATGGAGAATACCAGACCTAATCGGCAAAGA
ACAAAAGGTTGAAGACTGATTTAAAAATCATTATAACTTTTTGTTTAGT
AMPV3_SARS-COV2-S_wt (SEQ ID NO: 16):
ACTAAACAGAAAATTAATACTTGTTAGTAGTCGCCGATTAATCCGCTAATATATTAG
GAGCGGAAGTCCTACACTCCGCCTCCGACCACGAATTGAAATCATCATGGCTGGAA
TTTTTAATACTTATGAGCTCTTTGTTAAGGATCAGACATGTATGCACAAGAGGGCTG
CAAGCCTAATATCGGGAGGTCAGCTTAAAAGCAATATACCAGTCTTCATCACCACAA
AAGATGATCCAGCTGTCCGATGGGACCTTGTATGTTTTAATCTCCGACTTGTTGTTAG
TGAATCATCTACCTCGGTGATCCGGCAGGGGGCTCTTATATCATTGCTTTCCATTACT
GCTAGCAATATGAGGGCTCTTGCAGCAATTGCTGGGCAGACTGATGAATCGATGATT
AATATTATAGAAGTTGTGGACTTTGATGGACTTGAGCCCCAGTGTGACACTCGCAGT
GGCTTAGACGCTCAGAAGCAGGAGATGTTTAAGGATATTGCCCGTGATATGCCGAG
GGCACTATCCAGCGGGACCCCGTTCCAGCACGGAAATGCGGAAGCGAATGGGCCTG
AAGACACACACATGTTCCTCCGATCCGCAATTAGCGTTCTGACACAGATCTGGATCC
TGGTTGCAAAAGCCATGACTAACATTGATGGCAGCAACGAGGCAAGCGATCGCCGC
CTTGCCAAGTATATTCAACAGAATAGAATTGACAGGCGATTCATGCTTGCTCAAGCT
ACCAGGACTGTATGTCAGCAGGTCATAAAGGATTCTTTGACCTTGAGAAGGTTCCTT
GTAACTGAGCTTCGAAAGTCGCGGGGGGCATTGCATAGCGGGTCATCTTACTATGCT
ATGATTGGAGACATGCAGGCATACATATTTAATGCAGGCCTTACACCATTCCTCACT
ACCCTCAGGTACGGGATCGGGACTAAATACCATGCCCTTGCAATTAGCTCACTTGCA
GGTGACCTGAATAAGATTAAGGGGTTACTGACTCTATACAAAGAGAAAGGGAGTGA
TGCGGGCTATATGGCACTTCTAGAGGATGCTGACTGCATGCAATTTGCGCCAGGTAA
TTACTCACTATTATATTCATATGCGATGGGAGTAGCAAGTGTACATGATGAAGGCAT
GAGGAATTACCAATACGCCCGGCGCTTCCTCCATAAGGGGATGTACCAATTTGGACG
TGATATCGCAACACAACAGCAACATGCACTTGATGAATCCCTCGCCCAGGAGATGA
GAATCACAGAGGCTGACAGGGCAAATCTCAAGGTAATGATGGCAAACATTGGGGAA
GCATCAAGCTTCAGTGATATGCCTAATCCAGGAGCTAGCGGCATTCCTGCCTTCAAT
GACCGCCCTGATGAGATCTTCTCTGAGCCAGGCTATCGCAAGCCCCCTCAAGATCAG
CCGCTGATAAAGCTCCCAGATCTCGAGGAAGAAGAGCAGGACGAATTCGACATGTA
GAGAGCCGCCCACCACCCAGAGCCAGCGACAAACCCAGGCAAAACAACCCACCATC
TCCGACCCGGAACACAACGCAACATCGAGGACCCCGGGCAGCCGACACCGCAGCCG
CAAGACCTCCGCATCATGAACGACCAGCATCCAAACATCTGTCCTCCCTATTCTATC
AGTTTAATAAAAAAGGCTTTACGTGCACAGACAGAGATCCTGACATAATTCAGCCG
GGGGGATTCAACTTAGGAGCGGAAGTATCCCTCCTAACCTGCACCTGCATTTCAATA
TGGATCTTGAGTTTAGTAGTGAGGAAGCAGTCGCAGCCCTCTTGGATGTGAGTTCCT
CAACCATAACCGAGGTTCTGAGCAAGCAAAGTATTCCTGACCCTAGCTTTCTCACGT
CCACGGAGGCATCCGGTGAGCAAGTTCCAGATTCGAAGGTCGCCAATCACAAAGCC
AGTGTCGATAAGACCCCAGATCAAGGCCAACCATCGGCAACGCCTTCGGCTCCTCCT
GAGACTGCCGAAAACATCAATACACCATCTTGCGAGGACGGTTTACCCCCTAACTTC
TTCATCCCAAGGGTAGAGAGTTACCACACTAACCTTTTTAAAGGGGGCCCCCAATTG
GACTCAACGGAGCGGCCGCCTGGTCACCAAATAGTTTGCGGAGATCAAGACGCGGA
CCTCTCCCGAGCCGGGATAGCGAGAAAGAAGAAGAAGCAGAAGCATTGCAAAGCG
CTCAACTTTTCGGAATCCCCACTCACAGACAATCAGGCCATAGAAGGGAGTATTCAA
TCTCATGGAGCCCTGGATCAGGAACCTTCCAGACAGAGACCTGGTGCAACCCAGCC
TGCTCTCCAGTCACCGCCTTCCCAAAACAATACAAGTGTGCATGCAGACAGTGCCCA
AGATTCTGCGATCTCTGTTTCAATCCCGCTGACTATGGTGGAATCATTGATCTCTCAA
GTGTCAAAGTTGTCGGACCAAGTCTCGCAGATACAGAAGTTGGTGAGTACCCTTCCT
CAAATTAAGACTGATATTGCTTCGATTCGGAACATGCAAGCAGCTTTGGAAGGACA
GCTCAGTATGATTCGTATATTGGATCCAGGAAACTGTTCGGAATCATCACTCAATGC
GCTGAGAGGGTTCAGTGGTAAGGCACCTGTTATTGTGAGTGGTCCAGGAAATCCCA
ATCAATGCATCAGCCAAGGCTCATCCACCACAATTTGCCTGGATGAGTTAGCCCGGC
CTGTGCCAAATCCTATACAGGTGAAACTTCAAGATGATCAGAAGGACCTATCTGCAC
AGCGACATGCTGTCATTGCATTACTCGAAACCAGAATCCCCCCGGGACCAAAACGT
GACAAATTACTGTCATTGGTTGCAATTGCAAAGACTGCAAGTGATTTAATCAAGATT
AAGAGAATGGCAGTCCTCGGCCAGTGATATCACCCTATGGCAACCAGGCTACAAAT
GTAAGCAGTTTCCGATATGTACCGAATCAATAACACGACCCCTTCCGCAAAAGCAA
GCGCGAGCATGACACACTCAGCTTCAGCCAATTCTAGCTTCACTAAGCAGGTGATCT
GACCATCTGGCTTTACTTCCGCTTTATAAAAAACTCCTAGATCTAACATGGAGGCAG
ACCGAGCTAATGCGGAGGGAGTACACAAGGGGAATCAAAGGAGCGGAAGGTACTA
AGTCACTCCACCGCCTGCCCTCGCCCATCCGCAGCCTCCCCCAACCCGCGGCGCCGC
CACCATGTTTGTTTTTCTTGTTTTATTGCCACTAGTCTCTAGTCAGTGTGTTAATCTTA
CAACCAGAACTCAATTACCCCCTGCATACACTAATTCTTTCACACGTGGTGTTTATTA
CCCTGACAAAGTTTTCAGATCCTCAGTTTTACATTCAACTCAGGACTTGTTCTTACCT
TTCTTTTCCAATGTTACTTGGTTCCATGCTATACATGTCTCTGGGACCAATGGTACTA
AGAGGTTTGATAACCCTGTCCTACCATTTAATGATGGTGTTTATTTTGCTTCCACTGA
GAAGTCTAACATAATAAGAGGCTGGATTTTTGGTACTACTTTAGATTCGAAGACCCA
GTCCCTACTTATTGTTAATAACGCTACTAATGTTGTTATTAAAGTCTGTGAATTTCAA
TTTTGTAATGATCCATTTTTGGGTGTTTATTACCACAAAAACAACAAAAGTTGGATG
GAAAGTGAGTTCAGAGTTTATTCTAGTGCGAATAATTGCACTTTTGAATATGTCTCTC
AGCCTTTTCTTATGGACCTTGAAGGAAAACAGGGTAATTTCAAAAATCTTAGGGAAT
TTGTGTTTAAGAATATTGATGGTTATTTTAAAATATATTCTAAGCACACGCCTATTAA
TTTAGTGCGTGATCTCCCTCAGGGTTTTTCGGCTTTAGAACCATTGGTAGATTTGCCA
ATAGGTATTAACATCACTAGGTTTCAAACTTTACTTGCTTTACATAGAAGTTATTTGA
CTCCTGGTGATTCTTCTTCAGGTTGGACAGCTGGTGCTGCAGCTTATTATGTGGGTTA
TCTTCAACCTAGGACTTTTCTATTAAAATATAATGAAAATGGAACCATTACAGATGC
TGTAGACTGTGCACTTGACCCTCTCTCAGAAACAAAGTGTACGTTGAAATCCTTCAC
TGTAGAAAAAGGAATCTATCAAACTTCTAACTTTAGAGTCCAACCAACAGAATCTAT
TGTTAGATTTCCTAATATTACAAACTTGTGCCCTTTTGGTGAAGTTTTTAACGCCACC
AGATTTGCATCTGTTTATGCTTGGAACAGGAAGAGAATCAGCAACTGTGTTGCTGAT
TATTCTGTCCTATATAATTCCGCATCATTTTCCACTTTTAAGTGTTATGGAGTGTCTCC
TACTAAATTAAATGATCTCTGCTTTACTAATGTCTATGCAGATTCATTTGTAATTAGA
GGTGATGAAGTCAGACAAATCGCTCCAGGGCAAACTGGAAAGATTGCTGATTATAA
TTATAAATTACCAGATGATTTTACAGGCTGCGTTATAGCTTGGAATTCTAACAATCTT
GATTCTAAGGTTGGTGGTAATTATAATTACCTGTATAGATTGTTTAGGAAGTCTAAT
CTCAAACCTTTTGAGAGAGATATTTCAACTGAAATCTATCAGGCCGGTAGCACACCT
TGTAATGGTGTTGAAGGTTTTAATTGTTACTTTCCTTTACAATCATATGGTTTCCAAC
CCACTAATGGTGTTGGTTACCAACCATACAGAGTAGTAGTACTTTCTTTTGAACTTCT
ACATGCACCAGCAACTGTTTGTGGACCTAAAAAGTCTACTAATTTGGTTAAAAACAA
ATGTGTCAATTTCAACTTCAATGGTTTAACAGGCACAGGTGTTCTTACTGAGTCTAA
CAAAAAGTTTCTGCCTTTCCAACAATTTGGCAGAGACATTGCTGACACTACTGATGC
TGTCCGTGATCCACAGACACTTGAGATTCTTGACATTACACCATGTTCTTTTGGTGGT
GTCAGTGTTATAACACCAGGAACAAATACTTCTAACCAGGTTGCTGTTCTTTATCAG
GATGTTAACTGCACAGAAGTCCCTGTTGCTATTCATGCAGATCAACTTACTCCTACTT
GGCGTGTTTATTCTACAGGTTCTAATGTTTTTCAAACACGTGCAGGCTGTTTAATAGG
GGCTGAACATGTCAACAACTCATATGAGTGTGACATACCCATTGGTGCAGGTATATG
CGCTAGTTATCAGACTCAGACTAATTCTCCTCGGCGGGCACGTAGTGTAGCTAGTCA
ATCCATCATTGCCTACACTATGTCACTTGGTGCAGAAAATTCAGTTGCTTACTCTAAT
AACTCTATTGCCATACCCACAAATTTTACTATTAGTGTTACCACAGAAATTCTACCA
GTGTCTATGACCAAGACATCAGTAGATTGTACAATGTACATTTGTGGTGATTCAACT
GAATGCAGCAATCTTTTGTTGCAATATGGCAGTTTTTGTACACAATTAAACCGTGCTT
TAACTGGAATAGCTGTTGAACAAGACAAAAACACCCAAGAAGTTTTTGCACAAGTC
AAACAAATTTACAAAACACCACCAATTAAAGATTTTGGTGGTTTTAATTTTTCACAA
ATATTACCAGATCCATCAAAACCAAGCAAGAGGTCATTTATTGAAGATCTACTTTTC
AACAAAGTGACACTTGCAGATGCTGGCTTCATCAAACAATATGGTGATTGCCTTGGT
GATATTGCTGCTAGAGACCTCATTTGTGCACAAAAGTTTAACGGCCTTACTGTTTTGC
CACCTTTGCTCACAGATGAAATGATTGCTCAATACACTTCTGCACTGTTAGCGGGTA
CAATCACTTCTGGTTGGACCTTTGGTGCAGGTGCTGCATTACAAATACCATTTGCTAT
GCAAATGGCTTATAGGTTTAATGGTATTGGAGTTACACAGAATGTTCTCTATGAGAA
CCAAAAATTGATTGCCAACCAATTTAATAGTGCTATTGGCAAAATTCAAGACTCACT
TTCTTCCACAGCAAGTGCACTTGGAAAACTTCAAGATGTGGTCAACCAAAATGCACA
AGCTTTAAACACGCTTGTTAAACAACTTAGCTCCAATTTTGGTGCAATTTCAAGTGTT
TTAAATGATATCCTTTCACGTCTTGACAAAGTTGAGGCTGAAGTGCAAATTGATAGG
TTGATCACAGGCAGACTTCAAAGTTTGCAGACATATGTGACTCAACAATTAATTAGA
GCTGCAGAAATCAGAGCTTCTGCTAATCTTGCTGCTACTAAAATGTCAGAGTGTGTA
CTTGGACAATCAAAAAGAGTTGATTTTTGTGGAAAGGGCTATCATCTTATGTCCTTC
CCTCAGTCAGCACCTCATGGTGTAGTCTTCTTGCATGTGACTTATGTCCCTGCACAAG
AAAAGAACTTCACAACTGCTCCTGCCATTTGTCATGATGGAAAAGCACACTTTCCTC
GTGAAGGTGTCTTTGTTTCAAATGGCACACACTGGTTTGTAACACAAAGGAATTTTT
ATGAACCACAAATCATTACTACAGACAACACATTTGTGTCTGGTAACTGTGATGTTG
TAATAGGAATTGTCAACAACACAGTTTATGATCCTTTGCAACCTGAATTAGACTCAT
TCAAGGAGGAGTTAGATAAATATTTTAAGAATCATACATCACCAGATGTTGATTTAG
GTGACATCTCTGGCATTAATGCTTCAGTTGTAAACATTCAAAAAGAAATTGACCGCC
TCAATGAGGTTGCCAAGAATTTAAATGAATCTCTCATCGATCTCCAAGAACTTGGAA
AGTATGAGCAGTACATCAAGTGGCCATGGTACATTTGGCTAGGTTTTATAGCTGGCT
TGATTGCCATAGTAATGGTGACAATTATGCTTTGCTGTATGACCAGTTGCTGTAGTTG
TCTCAAGGGCTGTTGTTCTTGTGGATCCTGCTGCAAATTTGATGAAGACGACTCTGA
GCCAGTGCTCAAAGGAGTCAAATTACATTACACATAATGATTTATAAAAAACATTGT
GAGAATGCAATTTAAGGACGAAAGTAGGAGCGGAAGCCGCGGCCCAGGCTCCATTC
AAAAACCATGGCCGCTGCTCTCGGGAACATGCACCCGTCATCGTCGATTACTCTTAT
GCATGATGACCCGTCCATCCAGACGCAGTTGCTAGCCTTCCCTCTGATAAGTGAGAA
GACAGAGACAGGGACGACAAAGCTGCAGCCTCAAGTGAGAATGCAATCTTTCCTAT
CAACCGATAGCCAGAAGTATCATCTGGTCTTTATCAACACATATGGTTTCATTGCGG
AGGATTTTAATTGTAGTCCAGCAAACGGATTTGTGCCTGCACTATTTCAACCCAAGT
CTAAAGTCTTATCGTCAGCGATGGTTACACTTGGGGCTGTCCCTGCTGACACTGTTCT
CCAGGACCTACAACGGGACTTAATCGCAATGAGATTTAAAGTCCGAAAGAGTGCGT
CAGCGAAGGAGCTAATATTATTCTCCACAGACAATGTCCCTGCAACATTAACAGGGT
CATCAGTCTGGAAGAATAAAGGAGTAATAGCTGATACAGCAACTGCAGTTAAAGCG
CCCGGTCGCATCTCATGCGATGCGGTATGCAGTTATTGCATTACATTCATCTCTTTTT
GCTTCTTCCACTCATCTGCTCTTTTTAAGGTGCCCAAGCCTCTGCTGAATTTCGAGAC
GGCCATAGCATATTCCCTTGTACTCCAGGTCGAGCTTGAGTTCCCAAATATCAAAGA
TACCTTACACGAGAAGTATCTCAAAGTTAAAGACTCGAAGTGGTATTGTACAATTGA
CATCCATATTGGGAATCTTTTAAAGAGAACCGCCAAGCAACGAAGGAGGACACCTT
CAGAGATAATGCAGAAAGTCCGTAAGATGGGTTTCAGGATTGGGTTATACGATTTGT
GGGGTCCAACAATTGTGGTTGAATTAACTGGGTCCTCAAGTAAGTCCCTACACGGAT
TCTTCTCGAGTGAGCGGCTGGCCTGCCACCCGGTATCACAGTATAATCCTCATGTTG
GACAGCTTATCTGGGCCCACGATGTATCCATCACGGGGTGCCACATGATTATATCTG
AACTTGAGAAAAAGAAGGCACTTGCAATGGCTGACCTGACCGTGAGTGATGCAGTA
GCTGTCAACACGAGCATCAAGAGCTTATCGCCTTTCAGACTGTTCAAAAAATATTAG
GGCTAGACAACTCCTAGGGTGTGTGCTTATAATTTGCATATTCTAGATTATAATCATT
AACCGCGTCCAAATGATCAGACCGCATAATTAACAGCTCAGCCAGGACTCGCAAAT
CCTGCATAGGCTCAAATAACAAGACGACATATATACTCACACAACAAGGTGTAATA
TCTAGAAGGAATCAGAATTGAGACAATATCTAGGTTATCAGCGCTACTGCCCTGCAA
CTCACTATGTATGCTTGGCATTAATAAAAAACATTGTGAGAATGCAATTTAAGGACG
AAAGTAGGAGCGGAACACGCTCAACTGAACCCAAGCGCACACCAGCCCCCCGGCAC
CGCACCGGAAGGACGCACAACAGCCGACCGCCCCCACCAGGGCAGAGGCCATGCA
ACCAGGCTCGGCACTCCACCTCCCGCACCTGTACATAATAATTGCCTTAGTCAGCGA
TGGCACTTTAGGCCAAACAGCTAAAATTGATAGGCTGATACAGGCAGGGATTGTCTT
GGGTAGTGGAAAGGAGCTCCATATCTCACAAGATTCAGGGACACTGGATTTATTCGT
GAGGTTGTTACCAGTACTCCCTTCGAATTTGTCCCATTGCCAGCTTGAAGCAATAAC
ACAGTATAATAAGACGGTGACTAGGTTATTGGCCCCAATAGGGAAGAACCTAGAAC
AGGTACTACAAGCGAGACCGCGTGGGAGGCTGTTCGGAGCAATAATAGGGTCCATC
GCGCTTGGTGTTGCCACATCAGCACAGATTACCGCTGCAATTGCACTCGTCCGTGCA
CAGCAAAATGCTAATGATATATTAGCACTTAAAAACGCTCTCCAATCCAGCAATGAG
GCAATCCGTCAACTCACGTATGGCCAGGACAAACAGTTACTTGCAATTTCTAAAATA
CAAAAAGCTGTGAATGAGCAGATCCTCCCTGCACTCGATCAGCTGGACTGTGCTGTC
CTTGGAACCAAACTAGCTGTGCAGCTTAATCTGTATCTCATTGAGATGACTACCATT
TTCGGCGAGCAAATCAATAATCCAGTCTTGGCGACCATCCCATTAAGTTACATTTTA
CGCCTGACCGGTGCGGAGTTGAACAACGTCCTCATGAAGCAAGCCCGATCGTCCTTA
AGCCTTGTACAGTTAGTTTCAAAAGGCTTACTTAGTGGACAAGTAATTGGCTATGAC
CCATCTGTTCAAGGTCTGATAATAAGGGTGAATCTGATGCGAACGCAGAAGATCGA
TAGAGCACTAGTTTATCAGCCGTATGTATTACCAATCACTCTTAACTCTAACATAGT
AACACCAATCGCCCCAGAATGCGTAATTCAGAAAGGGACGATCATAGAGGGTATGT
CACGGAAGGATTGTACAGAGTTGGAGCAAGACATAATTTGTAGGACAGTTACGACA
TACACGCTTGCCAGAGACACCAGATTGTGTTTACAAGGCAATATCTCTTCTTGTAGG
TATCAACAGTCAGGCACCCAACTACACACCCCATTTATTACGTACAACGGGGCAGTT
ATCGCAAACTGTGATTTAGTCTCTTGTCGGTGTCTCCGTCCTCCTATGATTATCACCC
AAGTAAAAGGGTACCCTCTTACCATAATCACAAGGTCCGTATGTCAAGAATTATCCG
TTGACAACTTAGTCCTGAATATTGAGACACACCATAATTTCTCCCTTAATCCAACAA
TTATCGACCCTCTAACAAGAGTAATAGCCACTACACCGTTAGAGATAGATTCCTTAA
TCCAAGAGGCACAAGATCATGCGAATGCAGCGCTAGCTAAAGTGGAAGAGAGTGAT
AAATATCTCAGGGCAGTTACTGGCGGTAATTATTCGAATTGGTATATCGTTCTTGTC
ATCGTCTTACTATTTGGGAATTTGGGCTGGTCGCTGTTGTTAACAGTACTTCTATGTA
GGTCACGGAAACAACAGCGACGTTATCAACAAGATGATTCCGTCGGGTCAGAAAGA
GGTGTTGGGGTTGGGACCATCCAGTATATGTCATAAGTGGTGTCAGACATCACAATC
CAAGTCACTCAGTTATCTACTCCGCTGGCTCGGAGCCATCGACAACGGCGTGAGCCC
TCATCCGGGGACAATCCACAACCTGATACTACCAGACAGTGGCTCCGCACGATGGC
AGCAGCCCACAACGTCCTGCATGTAAATATCATTGCGTTACTTTAATTAAAAAACCT
CAAGGTGACCCATCCTGGCCCATCCGATTGGAGGAGCGGAAGGTTTTAAGGTAATC
AGGCAAATTGTTTCCAACATGGAATCCCCTCCTTCTGGCAAGGATGCGCCAGCCTTC
CGCGAGCCTAAGCGAACTTGCAGACTCTGTTACAGGGCGACGACTCTCTCCCTTAAT
CTCACCATCGTTGTATTATCAATTATAAGCATTTATGTATCAACTCAAACTGGGGCA
AACAACTCTTGTGTCAATCCCACAATCGTAACTCCTGATTATTTAACTGGCAGCACG
ACAGGCTCAGTTGAAGATCTTGCTGACTTAGAGAGTCAACTCCGGGAGATACGTAG
AGACACAGGGATTAATCTCCCAGTTCAGATCGATAATACAGAAAATCTAATACTGA
CAACTCTGGCGAGTATCAACTCGAATTTACGCTTTCTGCAAAATGCAACAACTGAGA
GCCAGACCTGCCTATCTCCTGTCAATGACCCTCGCTTTGTAGCTGGCATAAATAGGA
TCCCAGCAGGGTCGATGGCATATAATGACTTCAGCAACCTCATTGAGCACGTAAATT
TCATACCGTCGCCGACAACGTTGTCAGGCTGCACCAGGATACCATCATTTTCACTCT
CTAAGACGCACTGGTGTTATACCCACAATGTTATATCAAACGGGTGCCTTGACCATG
CTGCAAGTTCACAATACATCTCAATAGGGATCGTTGATACCGGCCTAAATAATGAGC
CCTATTTTAGAACAATGTCTTCAAAGTCACTGAATGACGGATTAAATAGAAAGAGTT
GCTCTGTGACCGCAGCCGCTAATGCATGTTGGTTACTCTGTAGCGTAGTAACAGAAT
ACGAAGCTGCGGACTATCGATCACGAACTCCTACTGCTATGGTTCTGGGGCGATTCG
ATTTTAATGGTGAATACACAGAAATAGCTGTCCCCTCCTCACTATTCGATGGCCGGT
TTGCATCTAACTATCCAGGTGTGGGATCGGGAACTCAAGTCAATGGAACATTATATT
TCCCTTTATATGGAGGGGTCCTTAATGGGTCAGATATAGAAACGGCAAACAAGGGG
AAATCCTTCAGGCCTCAGAATCCTAAGAATAGGTGTCCAGACTCGGAGGCGATCCA
AAGCTTTAGAGCGCAAGATAGCTATTACCCGACGAGATTTGGGAAGGTGCTGATCC
AACAGGCAATCATTGCATGTAGGATCAGTAACAAAAGTTGTACTGATTTCTATCTCC
TGTACTTCGACAACAACAGAGTGATGATGGGTGCAGAGGCACGGCTATATTATCTTA
ATAACCAATTATATCTATATCAACGCTCGTCGAGCTGGTGGCCGCATCCTTTGTTCTA
TAGCATCTCATTACCGTCATGTCAAGCCCTTGCTGTCTGTCAAATTACAGAGGCCCA
TCTGACACTGACTTACGCCACGTCGAGGCCAGGAATGTCTATCTGTACAGGAGCATC
AAGGTGTCCAAATAATTGCGTAGACGGTGTATATACTGATGTATGGCCATTGACAAA
GAACGATGCTCAGGACCCCAATCTCTTCTACACTGTATACTTAAATAATTCAACTCG
GCGGATTAGCCCAACCATAAGTCTATACACTTATGACCGCAGGATCAAATCTAAGCT
GGCAGTGGGTAGTGATATAGGAGCAGCTTATACCACGAGTACCTGTTTCGGCCGATC
AGATACAGGGGCGGTTTATTGCTTAACTATAATGGAGACCGTTAACACAATATTTGG
TCAGTACCGGATCGTGCCAATATTACTTAGAGTGACAAGCCGGTAATGGATTGTTCC
TAGTGAAGTGGGGCCTATGGTTACTCCGTAACGGAGATGACCTGATTTATGCTATGT
AACTAATTTTGCTCAATCGGGAGGGTGATCGGCCGGGTATGTCCACAATGACAATGA
CTCCTATTCCCTTGCTAGAGCTAGCTTCCTCTTTAATCCAATACAATCCCTACCTGTA
GTTTAATAAAAAAATCCTCTATATAAGATCCAGACGCGTGCTATATAAGCTAATAGC
TTAGGATGGAAACGACAGGAGCGGAACATATCCCTCCTACCATGTCTTCTCATAATA
TTATCCTCCCTGACCACCACTTAAATTCCCCTATTGTACTAAATAAATTGATGTACTA
TTGTAAACTCCTTAATATATTGCCTGACCCTAAGTCGCCATGGTACGAGAAGATTAA
GTCTTGGACTAATTGCTGCATCCGTGTGTCAGACAGCAATCGCATGACTTTGTCACG
CGCCTCCAAGCTACGAGAACTATTGGCAACGTACGGCGTATATTCGAAGAACCACC
AGCAATGTTATACCACCATAATCCATCCCCAATCCCTATCACCCATCATGCAGACTG
TCTCTCAACTGGGACGCTCAATCCCGACCTGGGCCAGGATCCGAAAGGAGATCACA
CACAGTATCATAGCCCAAACGAATAAATTTGAATCCTTATTCCACAACATCTCAAAG
GACCTGACAGGGAAAACCAACTTGTTTGGCGGATTCTGTGATTTACATGGGAGCATA
AGCACTGCTGCAAAGCGCTTAATGAATCAACCGGGTCTCTACTTGGATTCACCTGAT
GCCCATGCATGTCAATTTTTGTTCCAGCTTAAAACCTGTCAACGGGAATTGATTCTAC
TTATGAGGCAGAATGCCACAACAGAGCTGATTAGGGTTTTTGACTATCCTGAGCTAC
GGGTTCTTGTGACACCTGAATATTCGGTCTGGGTTTTTACACGGACCAAACAAGTTA
CATTACTAACATTCGATTGCTTGTTAATGTACTGCGATCTTTCAGACGGCCGGCACA
ACATCCTATTCACTTGTAAGCTTTTGTCTCACTTAAACCACCTCGGGTGTCGGATAAG
GGACCTGCTGGTGCTAATTGATTCACTTGCCGAGAAACACCCCTTAATAGTTTATGA
TGTAGTTGCAAGTCTGGAGTCGTTATCATATGGTGCTATACAGCTACATGACAAGGT
AGCTGGTTATGCTGGAACATTTTTCTCATTTATCCTTGCAGAAATACAGGATTCCTTA
GAAACAGTGTTGGATCAAGGTAATAGTGAATCTATCATATCCCAAATAAGGAACAT
ATACTCAGGTCTCACAGTAAACGAGGCTGCAGAATTGCTATGTGTTATGAGGCTTTG
GGGCCACCCTGCCCTAAACAGTGTAGACGCTGCAAGCAAGGTGAGACAGAGTATGT
GCGCTGGCAAGCTGCTTAAATTTGATACGATCCAGTTAGTCCTTGCATTCTTCAATAC
ACTAATAATTAATGGATATAGGAGAAAGCACCATGGGCGATGGCCGAATGTTTGTA
GTGACTCTATAATTGGGAATGAACTCAAGCGGATGTATTTTGATCAATGTGAGATCC
CCCACGACTTCACATTAAAGAACTACCGGGAATTAAGTCTTCTTGAATTCGAGTGCA
CATTTCCCATAGAATTATCAGATAAACTGAACATATTTTTAAAAGATAAGGCAATAG
CATTCCCGAAGTCCAGGTGGACATCTCCTTTCAAAGCAGATATCACACCGCGCCATT
TGCTGCAAGCGCCTGAGTTTAAAACCCGTGCCAACAGACTACTGCTATCATTCTTAC
AACTCGAGGAATTCTCCATCGAATCAGAACTAGAATATGTGACTACCCTGGCTTACC
TGGATGATGATGAGTTCAACGTATCTTACTCCCTCAAAGAGAAGGAGGTAAAGACA
GATGGTCGAATCTTCGCTAAGCTCACAAGGAAAATGCGGAGCTGTCAGGTGATATTC
GAAGAATTGCTAGCAGAGCATGTCTCTCCATTATTTAAAGACAACGGCGTTACTATG
GCCGAATTGTCACTTACTAAGAGCCTACTCGCTATTAGCAACCTAAGCTCCACCTTA
TTTGAGACGCAAACAAGGCAAGGCGATCGGAATGCAAGATTCACGCATGCGCACTT
CATAACAACTGACCTGCAAAAGTACTGTTTAAACTGGAGGTACCAGAGTGTAAAAC
TGTTCGCACGTCAATTGAACCGGTTATTTGGTCTGCAGCATGGGTTTGAATGGATCC
ATTGCATACTAATGAAGTCCACTATGTATGTCGCAGACCCCTTTAACCCTCCACATA
GCAATGCACGTGAGGCACTCGATGACAACTTAAATAGCGACATATTCATTGTATCAC
CTAGGGGGGCCATCGAGGGACTATGCCAAAAAATGTGGACAATCATCTCAATCTCG
GCGATCCATGCATCCGCTGCTGTGGCTGGGCTACGGGTGGCATCTATGGTCCAAGGT
GATAACCAAGTTATTGGGGTAACACGCGAGTTCCTAGCTGGGCATGATCAAACTTTT
GTAGACGATCAATTGACGGTTTCACTTGAGAACTTCACTCAGATATTCAAGGAAGTC
AACTACGGGCTAGGTCATAATCTCAAGCTGAGGGAAACAATCAAGTCGAGCCACAT
GTTCATCTACTCAAAGAGGATCTTCTACGACGGCCGAGTGCTTCCACAACTTCTAAA
GAACATAAGTAAGTTAACCCTATCAGCTACAACTACAGGGGAGAACTGTTTAACGA
GTTGCGGGGACCTATCATCATGCATCACACGATGTATAGAGAATGGGTTCCCTAAAG
ATGCAGCATTCGTTTTGAATCAATTAGTGATTAGGATCCAGATACTTGCCGATCACT
TTTATTCCATCCTTGGAGGATGTTTCTCTGGGTTGAACCAGGCGGATATCCGTCTCCT
TCTATATGAGGGTGCAATTTTACCGGCACAATTAGGAGGGTTCAACAACCTCAACAT
GTCACGGCTATTCTGTCGGAATATTGGTGACCCATTAGTCGCGTCCATCGCAGACAC
CAAGAGATTTGTGAAGTGCCAACTGCTCGTTCCTCACATTTTAGACTCTGTCGTTGCT
ATTACCGATAGGAAGGGGTCTTTTACGACACTGATGATGGATCCATATTCAATAAAC
TTGGATTATATCCAGCAACCAGAGACACGATTGAAGCGACATGTGCAAAAGGTTTT
ACTACAAGAGTCAGTGAATCCGCTACTCCAAGGGGTCTTCCTAGAGTCTCAACAAGA
GGAAGAGGAACAACTTGCAGCGTTCTTGCTGGACCGAGAAGTCGTAATGCCAAGGG
TTGCGCACGCTATATTTGAGTGTACCAGCTTAGGTAGGCGCCGACATATCCAGGGCC
TCATCGACACGACAAAGACCATTATTGCACTTGCGTTGGATACACAGCAGTTAAGTT
ATACTAAACGTGAACAGATTGTGACGTACAATGCAACTTATATGAGGTCCTTAGCAA
GCATGCTCAGTTCAAGCCATAATCAGACACGTCGGTCTGTGATTGGTCACGCATCCT
TCAATATCACCGACTGCTCGGTCATCCTGGCTCAACAAGTGAGACGTGCAAGCTGGG
CGCCCTTGCTGAATTGGCGGTCATTAGAAGGCTTAGAAGTACCCGACCCTATTGAGT
CAGTAGCCGGCTACCTGGGTTTGGATTCTAACAACTGTTTCTTATGCTGCCACGAGC
AGAACAGCTATTCCTGGTTTTTCCTTCCCCGAATGTGCCATTTTGATGATTCGAGACA
ATCTAACTCCATACAAAGGGTACCTTACATTGGTTCTAAGACTGACGAGCGCCAGAT
GTCTACGATAAATCTACTTGAGAAAACTACCTGTCATGCTAGAGCAGCTACGAGGCT
TGCATCATTATACATTTGGGCCTTTGGGGACTCAGACGATAGTTGGGATGCAGTCGA
GACTCTATCTAACAGTCGATGCCAGATCACCCGAGAGCACTTGCAAGCTCTGTGCCC
TATGCCCTCTTCAGTGAATCTCCACCATCGATTGAACGACGGAATAACACAAGTGAA
GTTTATGCCATCGACCAATAGCAGGGTCTCTCGATTTGTTCATATTTCCAATGACCGG
CAGAATTACGTATTGGATGACTCCGTTACTGACAGTAATCTGATTTACCAACAAGTC
ATGTTGCTAGGACTTAGTATACTTGAAACATATTTCAGAGAACCTACAGCGACGAAC
TTATCAAGCTTGGTGCTGCACCTTCACACTGATGTATCTTGTTGCTTACGAGAATGCC
CGATGACACAATATGCGCCCCCCCTGCGAGACATCCCAGAGTTAACTATAACTGCGT
CAAATCCTTTCTTATTTGATCAAGAACCTATTAGCGAAGCAGACCTGTGTAGACTCT
CAAAGATAGCATTCCGTAGAGCAGGAGACAACTACGATGCATACGACCAGTTTCAA
CTAAGAGCAACACTGGCCGCAACTACAGGTAAGGATGTGGCAGCAACAATCTTTGG
TCCTCTAGCTGCAGTATCAGCAAAAAATGATGCAATAGTCACAAATGACTATAGCG
GGAATTGGATTTCCGAGTTTAGGTATAGTGATCTCTATCTTTTGAGTGTTAGCCTGGG
CTACGAGATCCTTTTGATTTTCGCGTATCAACTTTACTACCTACGGATACGGTACCGG
CAAAATATTGTATGCTATATGGAATCTGTATTCCGACGTTGTCATTCTTTATGCTTGG
GCGACTTAATCCAGACTATTTCGCACTCTGAAATACTGGTTGGGTTAAATGCAGCGG
GCTTTAATCTTACCTTGGATCGAAGTGATTTGAAGGAGAATCAACTCTCGCGGCTTG
CTGTGAAATACCTCACATTGTGTGTCCAGACTGCAATAAGTAATTTGGAGGTAGGAT
CAGAACCCCTTTGTATTATTGGAGGTCAATTAGATGATGACATATCATTCCAAGTTG
CCCACTTCCTCTGTCGACGACTGTGCATCATCAGCTTGGTCCACTCAAATGTACAGA
ACTTACCCCCGATTAGGGATAATGAAGTTGATGTAAAATCCAAGTTGATCTACGACC
ATCTTAAGCTAGTTGCTACCACACTCAATAATAGGGACCAGTCGTATCTGCTAACAC
TACTGAATAAGCCTAACATCGAGCTTCATACTCCACAGGTTTACTTCATTATGAGGA
AGTGTCTGGGATTATTAAAAGTCTATGGACCTGTACCTCAAAAGCAACCACTTCCAA
CCTCCCCAGTAGTATCACTACCAAATAAGTGCAAATCGAAATGGAAGCTTGAGGAG
GTAATTGACAGCATTGAGTCACCTAAGTCTTTTAACTGGGTTCCTGATACGACGATT
CCATTGGATGCTGAGCAAAATCCCCCAAATCCTAACAGGGTCATCGATAAGATAAA
CATTCTAAGATCTCTGAGCCCGCGTCACTCAGTCTGGTACCGTAACCGGCAATACCG
GTACGTGTTGAGCCAGTTAGGGCATGACCAGCTAGGTGGTGCAACCCTATATCTTGG
TGAAGGGAGCGGCTCCACAATCTTGAACATAGAGCCAAAAGTCAGGAGTGATAAGA
TATATTATCATACCTATTTCTCTGCAGACAAAAGCCCTGCACAGCGCAATTTCATCCC
CCAGCCAACTACTTTTCTGCGATCTAACTTTTATCACTACGAATTAGAGCCGTCTGGA
TGTGAGTTCATCAATTGTTGGTCCGAGGATGTAAATGCAACGAATCTTACGGAACTG
CGTTGCATAAATCACATATTAACTGTGATTCCAGTCGGATCTCTAACACGTATTATTT
GTGACGTGGAATTGGCAAATGACACGTCAATACAATCAGTGGCCACCGCATACATG
AACTTAAGCATTCTTGCACATGCTCTCCTCAACCAGGGAGGCGTYTGCATCTGTAGG
TGCCACCTGTTAGACACGTCCCATCTTGCGATTGTTTCGTTTGTACTTAAAACATTAT
CAAGCCAGCTGGCGATTTCGTTTTCAGGATATTGTGGAACCAATGACCCATCTTGTG
TCATAGGAGTCACGAAAGAGTGCTCCATTAGCCTGGATGTCTTAAGTTCGATTGCAG
CTGCATTTATAAATGAATTGCCCTCAAATGAGCTGCCGGTCCCTCAATCATTATTAA
CGCTACTAGGGTGTTATCAAGACCAGTTAGAGAGCCTTAGTGCACTCATTGAAAAAA
CATGGATCCAGGAGATCCGGAGGCCTCATTTAATGCAATGTGAGATGGAGTGGATA
GGACTTTTGGGAACAGATGCGCTGGGCGATGTCGACACATTTATCAGTTATCGCAAT
GATTCGTGTAATTCCATCCCTGATCTACTGACGCCAGCTGTGAGTGCCCTCTTATTTG
AATTAATCAGCCTGACACCTGAGCTTCCTGGTACGGAGGACCAGCCGAGTAGGCGT
GTAATCAGCATTGGCCAAGCTTTTAACCTGACTATTTCGGGAAAAGTTAATACGATG
ATCAGATCATGCTGTGAGCAGTGCATTAAGTTATTAGCTGCCAATGTCTCCATCCTT
AGTGATGTAGATTTGTCGTATTTTGTGAGGGGGATTAGAGATGGTTCTTTTGCTTTGG
GGTTGTTAATCACCCAGCGTCAAATACTCAAGGCATCGAGAGCACCAAAGTACCTC
AAAACGCATCAGGTCCAGAACTGGATTGCGTTGTTACTGGAGGTCAAACTAGAGGA
AATCTTCTCTCGCCACTACCGGAAAGTGCTATTGAGAACTCTACGGCTGTTATCCCTC
TACGGGCTCCTGCAGCAGAAGGAAAGTTGATCATGATAGATGTCTCTCTGATTTCGC
CTCAGCCCTATATACAGATACCTGCTACTGCTGTTGTAACTATGCAAATATAGGCTA
ACCTCGGTCCGGAGTATTGAGGTAATGGGGAGACTCGTAATTGGAGAAAGAAGAAG
AACGGGGCAGATACCAGACCCCGGGATACACTACTATAAGCTACTTGGGTGACTCA
GGACGTCACTAATAAGAGTACACTGGTTTTAACAATAACACTGATAGTGTAACACTG
AATATTGTAAACAGTCTCAGGGATTATTTAATTAAAAAGACATTGCGGATCATCATG
TCAGGCCTGAACCAATCAGAAACCCATCCTACAGGCATGAGTCGATTAATCCAGTTT
AATTTAGTCCAAGATGATTAGTACTCTTAGATGTTACTAAGTATCAGGATTTAGCGT
ACTAGATTAAACGTCTTTGAAAGTCATTTTACCCTGCAAGGTGCATAAGCATATGCA
GGGGAATACCCACCACTGGTTCGTTAGTCTAATTTATTCAAAGATGCCACCAACTGA
GTGCACTGGGAGTCTGATTGAGCTGAATCAACCTAAAGCTCAAGCTTTCCTGTGATG
TAGCATGCAGTGTGCTACTGCTGCATAGAGCCAATCAGGGGCGTCTTGAAGGCCTCC
ACCTTCCTCAGGCAACGATAAAGCAGCTCACTGCTACACCCACCATGCAAATACAA
GGATATGTAGGTCCATCACCCTGCTTGCTAATGTGGTCGGCTCCAGGACCATGGCCA
TAGCGCTATCCGTCTAATTCAATCCGCCTCATCATATGCGTATACAATCCTCCACTGA
GAAATGCACCTGCTCAATCCAATGACAAACTACAACAACACGAGATCATTATGGGG
TCGATTCCCTTGAATCAGAAAGGTAAAAACGGCAGCGGAGATGGAGAATACCAGAC
CTAATCGGCAAAGAACAAAAGGTTGAAGACTGATTTAAAAATCATTATAACTTTTTG
TTTAGT
APMV3_SARS-COV2-S_2P (SEQ ID NO: 17):
ACTAAACAGAAAATTAATACTTGTTAGTAGTCGCCGATTAATCCGCTAATATATTAG
GAGCGGAAGTCCTACACTCCGCCTCCGACCACGAATTGAAATCATCATGGCTGGAA
TTTTTAATACTTATGAGCTCTTTGTTAAGGATCAGACATGTATGCACAAGAGGGCTG
CAAGCCTAATATCGGGAGGTCAGCTTAAAAGCAATATACCAGTCTTCATCACCACAA
AAGATGATCCAGCTGTCCGATGGGACCTTGTATGTTTTAATCTCCGACTTGTTGTTAG
TGAATCATCTACCTCGGTGATCCGGCAGGGGGCTCTTATATCATTGCTTTCCATTACT
GCTAGCAATATGAGGGCTCTTGCAGCAATTGCTGGGCAGACTGATGAATCGATGATT
AATATTATAGAAGTTGTGGACTTTGATGGACTTGAGCCCCAGTGTGACACTCGCAGT
GGCTTAGACGCTCAGAAGCAGGAGATGTTTAAGGATATTGCCCGTGATATGCCGAG
GGCACTATCCAGCGGGACCCCGTTCCAGCACGGAAATGCGGAAGCGAATGGGCCTG
AAGACACACACATGTTCCTCCGATCCGCAATTAGCGTTCTGACACAGATCTGGATCC
TGGTTGCAAAAGCCATGACTAACATTGATGGCAGCAACGAGGCAAGCGATCGCCGC
CTTGCCAAGTATATTCAACAGAATAGAATTGACAGGCGATTCATGCTTGCTCAAGCT
ACCAGGACTGTATGTCAGCAGGTCATAAAGGATTCTTTGACCTTGAGAAGGTTCCTT
GTAACTGAGCTTCGAAAGTCGCGGGGGGCATTGCATAGCGGGTCATCTTACTATGCT
ATGATTGGAGACATGCAGGCATACATATTTAATGCAGGCCTTACACCATTCCTCACT
ACCCTCAGGTACGGGATCGGGACTAAATACCATGCCCTTGCAATTAGCTCACTTGCA
GGTGACCTGAATAAGATTAAGGGGTTACTGACTCTATACAAAGAGAAAGGGAGTGA
TGCGGGCTATATGGCACTTCTAGAGGATGCTGACTGCATGCAATTTGCGCCAGGTAA
TTACTCACTATTATATTCATATGCGATGGGAGTAGCAAGTGTACATGATGAAGGCAT
GAGGAATTACCAATACGCCCGGCGCTTCCTCCATAAGGGGATGTACCAATTTGGACG
TGATATCGCAACACAACAGCAACATGCACTTGATGAATCCCTCGCCCAGGAGATGA
GAATCACAGAGGCTGACAGGGCAAATCTCAAGGTAATGATGGCAAACATTGGGGAA
GCATCAAGCTTCAGTGATATGCCTAATCCAGGAGCTAGCGGCATTCCTGCCTTCAAT
GACCGCCCTGATGAGATCTTCTCTGAGCCAGGCTATCGCAAGCCCCCTCAAGATCAG
CCGCTGATAAAGCTCCCAGATCTCGAGGAAGAAGAGCAGGACGAATTCGACATGTA
GAGAGCCGCCCACCACCCAGAGCCAGCGACAAACCCAGGCAAAACAACCCACCATC
TCCGACCCGGAACACAACGCAACATCGAGGACCCCGGGCAGCCGACACCGCAGCCG
CAAGACCTCCGCATCATGAACGACCAGCATCCAAACATCTGTCCTCCCTATTCTATC
AGTTTAATAAAAAAGGCTTTACGTGCACAGACAGAGATCCTGACATAATTCAGCCG
GGGGGATTCAACTTAGGAGCGGAAGTATCCCTCCTAACCTGCACCTGCATTTCAATA
TGGATCTTGAGTTTAGTAGTGAGGAAGCAGTCGCAGCCCTCTTGGATGTGAGTTCCT
CAACCATAACCGAGGTTCTGAGCAAGCAAAGTATTCCTGACCCTAGCTTTCTCACGT
CCACGGAGGCATCCGGTGAGCAAGTTCCAGATTCGAAGGTCGCCAATCACAAAGCC
AGTGTCGATAAGACCCCAGATCAAGGCCAACCATCGGCAACGCCTTCGGCTCCTCCT
GAGACTGCCGAAAACATCAATACACCATCTTGCGAGGACGGTTTACCCCCTAACTTC
TTCATCCCAAGGGTAGAGAGTTACCACACTAACCTTTTTAAAGGGGGCCCCCAATTG
GACTCAACGGAGCGGCCGCCTGGTCACCAAATAGTTTGCGGAGATCAAGACGCGGA
CCTCTCCCGAGCCGGGATAGCGAGAAAGAAGAAGAAGCAGAAGCATTGCAAAGCG
CTCAACTTTTCGGAATCCCCACTCACAGACAATCAGGCCATAGAAGGGAGTATTCAA
TCTCATGGAGCCCTGGATCAGGAACCTTCCAGACAGAGACCTGGTGCAACCCAGCC
TGCTCTCCAGTCACCGCCTTCCCAAAACAATACAAGTGTGCATGCAGACAGTGCCCA
AGATTCTGCGATCTCTGTTTCAATCCCGCTGACTATGGTGGAATCATTGATCTCTCAA
GTGTCAAAGTTGTCGGACCAAGTCTCGCAGATACAGAAGTTGGTGAGTACCCTTCCT
CAAATTAAGACTGATATTGCTTCGATTCGGAACATGCAAGCAGCTTTGGAAGGACA
GCTCAGTATGATTCGTATATTGGATCCAGGAAACTGTTCGGAATCATCACTCAATGC
GCTGAGAGGGTTCAGTGGTAAGGCACCTGTTATTGTGAGTGGTCCAGGAAATCCCA
ATCAATGCATCAGCCAAGGCTCATCCACCACAATTTGCCTGGATGAGTTAGCCCGGC
CTGTGCCAAATCCTATACAGGTGAAACTTCAAGATGATCAGAAGGACCTATCTGCAC
AGCGACATGCTGTCATTGCATTACTCGAAACCAGAATCCCCCCGGGACCAAAACGT
GACAAATTACTGTCATTGGTTGCAATTGCAAAGACTGCAAGTGATTTAATCAAGATT
AAGAGAATGGCAGTCCTCGGCCAGTGATATCACCCTATGGCAACCAGGCTACAAAT
GTAAGCAGTTTCCGATATGTACCGAATCAATAACACGACCCCTTCCGCAAAAGCAA
GCGCGAGCATGACACACTCAGCTTCAGCCAATTCTAGCTTCACTAAGCAGGTGATCT
GACCATCTGGCTTTACTTCCGCTTTATAAAAAACTCCTAGATCTAACATGGAGGCAG
ACCGAGCTAATGCGGAGGGAGTACACAAGGGGAATCAAAGGAGCGGAAGGTACTA
AGTCACTCCACCGCCTGCCCTCGCCCATCCGCAGCCTCCCCCAACCCGCGGCGCCGC
CACCATGTTCGTGTTTCTGGTGCTGCTGCCTCTGGTGAGCTCCCAGTGCGTGAACCTG
ACCACAAGGACCCAGCTGCCCCCTGCCTATACCAATTCCTTCACACGGGGCGTGTAC
TATCCCGACAAGGTGTTTAGATCTAGCGTGCTGCACTCCACACAGGATCTGTTTCTG
CCTTTCTTTTCTAACGTGACCTGGTTCCACGCCATCCACGTGAGCGGCACCAATGGC
ACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAA
GACACAGTCCCTGCTGATCGTGAACAATGCCACCAACGTGGTCATCAAGGTGTGCG
AGTTCCAGTTTTGTAATGATCCATTCCTGGGCGTGTACTATCACAAGAACAATAAGT
CTTGGATGGAGAGCGAGTTTCGCGTGTATTCCTCTGCCAACAATTGCACATTTGAGT
ACGTGTCCCAGCCCTTCCTGATGGACCTGGAGGGCAAGCAGGGCAATTTCAAGAAC
CTGAGGGAGTTCGTGTTTAAGAATATCGATGGCTACTTCAAGATCTACTCCAAGCAC
ACCCCAATCAACCTGGTGCGCGACCTGCCACAGGGCTTCTCTGCCCTGGAGCCACTG
GTGGATCTGCCCATCGGCATCAACATCACCCGGTTTCAGACACTGCTGGCCCTGCAC
AGAAGCTACCTGACACCAGGCGACAGCTCCTCTGGATGGACCGCAGGAGCTGCCGC
CTACTATGTGGGCTATCTGCAGCCCAGGACCTTCCTGCTGAAGTACAACGAGAATGG
CACCATCACAGACGCCGTGGATTGCGCCCTGGATCCCCTGTCTGAGACCAAGTGTAC
ACTGAAGAGCTTTACCGTGGAGAAGGGCATCTATCAGACAAGCAATTTCAGGGTGC
AGCCTACCGAGTCCATCGTGCGCTTTCCCAATATCACAAACCTGTGCCCTTTTGGCG
AGGTGTTCAACGCAACCCGCTTCGCCAGCGTGTACGCCTGGAATAGGAAGCGCATC
TCCAACTGCGTGGCCGACTATTCTGTGCTGTACAACAGCGCCTCCTTCTCTACCTTTA
AGTGCTATGGCGTGAGCCCCACAAAGCTGAATGACCTGTGCTTTACCAACGTGTACG
CCGATTCCTTCGTGATCAGGGGCGACGAGGTGCGCCAGATCGCCCCTGGCCAGACA
GGCAAGATCGCCGACTACAATTATAAGCTGCCTGACGATTTCACCGGCTGCGTGATC
GCCTGGAACTCTAACAATCTGGATAGCAAAGTGGGCGGCAACTACAATTATCTGTAC
CGGCTGTTTAGAAAGTCTAATCTGAAGCCATTCGAGAGGGACATCTCCACAGAGATC
TACCAGGCCGGCTCTACCCCCTGCAATGGCGTGGAGGGCTTTAACTGTTATTTCCCT
CTGCAGAGCTACGGCTTCCAGCCAACAAACGGCGTGGGCTATCAGCCCTACCGCGT
GGTGGTGCTGTCTTTTGAGCTGCTGCACGCACCTGCAACAGTGTGCGGACCAAAGAA
GAGCACCAATCTGGTGAAGAACAAGTGCGTGAACTTCAACTTCAACGGACTGACCG
GCACAGGCGTGCTGACCGAGTCCAACAAGAAGTTCCTGCCTTTTCAGCAGTTCGGCA
GGGACATCGCAGATACCACAGACGCCGTGCGCGACCCTCAGACCCTGGAGATCCTG
GATATCACACCATGCTCCTTCGGCGGCGTGTCTGTGATCACACCAGGCACCAATACA
AGCAACCAGGTGGCCGTGCTGTATCAGGACGTGAATTGTACCGAGGTGCCCGTGGC
AATCCACGCAGATCAGCTGACCCCTACATGGCGGGTGTACTCTACCGGCAGCAACG
TGTTCCAGACAAGAGCCGGATGCCTGATCGGAGCCGAGCACGTGAACAATAGCTAT
GAGTGCGACATCCCTATCGGCGCCGGCATCTGTGCCTCCTACCAGACCCAGACAAAC
TCCCCAGGGTCTGCCTCCTCTGTGGCCAGCCAGTCCATCATCGCCTATACCATGAGC
CTGGGCGCCGAGAATTCCGTGGCCTACTCCAACAATTCTATCGCCATCCCTACCAAC
TTCACAATCTCCGTGACCACAGAGATCCTGCCAGTGAGCATGACCAAGACATCCGTG
GACTGCACAATGTATATCTGTGGCGATTCCACCGAGTGCTCTAACCTGCTGCTGCAG
TACGGCTCTTTTTGTACCCAGCTGAATAGAGCCCTGACAGGCATCGCCGTGGAGCAG
GACAAGAACACACAGGAGGTGTTCGCCCAGGTGAAGCAGATCTACAAGACCCCACC
CATCAAGGACTTTGGCGGCTTCAACTTCAGCCAGATCCTGCCCGATCCTAGCAAGCC
ATCCAAGCGGTCTTTTATCGAGGACCTGCTGTTCAACAAGGTGACCCTGGCCGATGC
CGGCTTCATCAAGCAGTATGGCGATTGCCTGGGCGACATCGCCGCCAGAGACCTGA
TCTGTGCCCAGAAGTTTAATGGCCTGACCGTGCTGCCTCCACTGCTGACAGATGAGA
TGATCGCCCAGTACACATCTGCCCTGCTGGCCGGCACCATCACAAGCGGATGGACCT
TCGGCGCAGGAGCCGCCCTGCAGATCCCCTTTGCCATGCAGATGGCCTATCGGTTCA
ACGGCATCGGCGTGACCCAGAATGTGCTGTACGAGAACCAGAAGCTGATCGCCAAT
CAGTTTAACTCCGCCATCGGCAAGATCCAGGACTCTCTGAGCTCCACAGCCAGCGCC
CTGGGCAAGCTGCAGGATGTGGTGAATCAGAACGCCCAGGCCCTGAATACCCTGGT
GAAGCAGCTGTCTAGCAACTTCGGCGCCATCTCCTCTGTGCTGAATGATATCCTGAG
CAGGCTGGACCCTCCAGAGGCAGAGGTGCAGATCGACCGGCTGATCACAGGCAGAC
TGCAGTCCCTGCAGACCTACGTGACACAGCAGCTGATCAGGGCAGCAGAGATCAGG
GCCTCTGCCAATCTGGCCGCCACCAAGATGAGCGAGTGCGTGCTGGGCCAGTCCAA
GAGAGTGGACTTTTGTGGCAAGGGCTATCACCTGATGAGCTTCCCACAGTCCGCCCC
TCACGGAGTGGTGTTTCTGCACGTGACCTACGTGCCAGCCCAGGAGAAGAACTTCAC
CACAGCACCAGCAATCTGCCACGATGGCAAGGCACACTTTCCTAGGGAGGGCGTGT
TCGTGAGCAACGGCACCCACTGGTTTGTGACACAGCGCAATTTCTACGAGCCACAG
ATCATCACCACAGACAATACATTCGTGTCCGGCAACTGTGACGTGGTCATCGGCATC
GTGAACAATACCGTGTATGATCCTCTGCAGCCAGAGCTGGACTCTTTTAAGGAGGAG
CTGGATAAGTACTTCAAGAATCACACCAGCCCCGACGTGGATCTGGGCGACATCTCT
GGCATCAATGCCAGCGTGGTGAACATCCAGAAGGAGATCGACAGGCTGAACGAGGT
GGCCAAGAATCTGAACGAGTCCCTGATCGATCTGCAGGAGCTGGGCAAGTATGAGC
AGTACATCAAGTGGCCCTGGTATATCTGGCTGGGCTTCATCGCCGGCCTGATCGCCA
TCGTGATGGTGACCATCATGCTGTGCTGTATGACAAGCTGCTGTTCCTGCCTGAAGG
GCTGCTGTTCTTGTGGCAGCTGCTGTAAGTTTGATGAGGACGATAGCGAGCCTGTGC
TGAAGGGCGTGAAGCTGCACTACACCTGATAGTTTATAAAAAACATTGTGAGAATG
CAATTTAAGGACGAAAGTAGGAGCGGAAGCCGCGGCCCAGGCTCCATTCAAAAACC
ATGGCCGCTGCTCTCGGGAACATGCACCCGTCATCGTCGATTACTCTTATGCATGAT
GACCCGTCCATCCAGACGCAGTTGCTAGCCTTCCCTCTGATAAGTGAGAAGACAGA
GACAGGGACGACAAAGCTGCAGCCTCAAGTGAGAATGCAATCTTTCCTATCAACCG
ATAGCCAGAAGTATCATCTGGTCTTTATCAACACATATGGTTTCATTGCGGAGGATT
TTAATTGTAGTCCAGCAAACGGATTTGTGCCTGCACTATTTCAACCCAAGTCTAAAG
TCTTATCGTCAGCGATGGTTACACTTGGGGCTGTCCCTGCTGACACTGTTCTCCAGGA
CCTACAACGGGACTTAATCGCAATGAGATTTAAAGTCCGAAAGAGTGCGTCAGCGA
AGGAGCTAATATTATTCTCCACAGACAATGTCCCTGCAACATTAACAGGGTCATCAG
TCTGGAAGAATAAAGGAGTAATAGCTGATACAGCAACTGCAGTTAAAGCGCCCGGT
CGCATCTCATGCGATGCGGTATGCAGTTATTGCATTACATTCATCTCTTTTTGCTTCT
TCCACTCATCTGCTCTTTTTAAGGTGCCCAAGCCTCTGCTGAATTTCGAGACGGCCAT
AGCATATTCCCTTGTACTCCAGGTCGAGCTTGAGTTCCCAAATATCAAAGATACCTT
ACACGAGAAGTATCTCAAAGTTAAAGACTCGAAGTGGTATTGTACAATTGACATCC
ATATTGGGAATCTTTTAAAGAGAACCGCCAAGCAACGAAGGAGGACACCTTCAGAG
ATAATGCAGAAAGTCCGTAAGATGGGTTTCAGGATTGGGTTATACGATTTGTGGGGT
CCAACAATTGTGGTTGAATTAACTGGGTCCTCAAGTAAGTCCCTACACGGATTCTTC
TCGAGTGAGCGGCTGGCCTGCCACCCGGTATCACAGTATAATCCTCATGTTGGACAG
CTTATCTGGGCCCACGATGTATCCATCACGGGGTGCCACATGATTATATCTGAACTT
GAGAAAAAGAAGGCACTTGCAATGGCTGACCTGACCGTGAGTGATGCAGTAGCTGT
CAACACGAGCATCAAGAGCTTATCGCCTTTCAGACTGTTCAAAAAATATTAGGGCTA
GACAACTCCTAGGGTGTGTGCTTATAATTTGCATATTCTAGATTATAATCATTAACCG
CGTCCAAATGATCAGACCGCATAATTAACAGCTCAGCCAGGACTCGCAAATCCTGC
ATAGGCTCAAATAACAAGACGACATATATACTCACACAACAAGGTGTAATATCTAG
AAGGAATCAGAATTGAGACAATATCTAGGTTATCAGCGCTACTGCCCTGCAACTCAC
TATGTATGCTTGGCATTAATAAAAAACATTGTGAGAATGCAATTTAAGGACGAAAGT
AGGAGCGGAACACGCTCAACTGAACCCAAGCGCACACCAGCCCCCCGGCACCGCAC
CGGAAGGACGCACAACAGCCGACCGCCCCCACCAGGGCAGAGGCCATGCAACCAG
GCTCGGCACTCCACCTCCCGCACCTGTACATAATAATTGCCTTAGTCAGCGATGGCA
CTTTAGGCCAAACAGCTAAAATTGATAGGCTGATACAGGCAGGGATTGTCTTGGGTA
GTGGAAAGGAGCTCCATATCTCACAAGATTCAGGGACACTGGATTTATTCGTGAGGT
TGTTACCAGTACTCCCTTCGAATTTGTCCCATTGCCAGCTTGAAGCAATAACACAGT
ATAATAAGACGGTGACTAGGTTATTGGCCCCAATAGGGAAGAACCTAGAACAGGTA
CTACAAGCGAGACCGCGTGGGAGGCTGTTCGGAGCAATAATAGGGTCCATCGCGCT
TGGTGTTGCCACATCAGCACAGATTACCGCTGCAATTGCACTCGTCCGTGCACAGCA
AAATGCTAATGATATATTAGCACTTAAAAACGCTCTCCAATCCAGCAATGAGGCAAT
CCGTCAACTCACGTATGGCCAGGACAAACAGTTACTTGCAATTTCTAAAATACAAAA
AGCTGTGAATGAGCAGATCCTCCCTGCACTCGATCAGCTGGACTGTGCTGTCCTTGG
AACCAAACTAGCTGTGCAGCTTAATCTGTATCTCATTGAGATGACTACCATTTTCGG
CGAGCAAATCAATAATCCAGTCTTGGCGACCATCCCATTAAGTTACATTTTACGCCT
GACCGGTGCGGAGTTGAACAACGTCCTCATGAAGCAAGCCCGATCGTCCTTAAGCC
TTGTACAGTTAGTTTCAAAAGGCTTACTTAGTGGACAAGTAATTGGCTATGACCCAT
CTGTTCAAGGTCTGATAATAAGGGTGAATCTGATGCGAACGCAGAAGATCGATAGA
GCACTAGTTTATCAGCCGTATGTATTACCAATCACTCTTAACTCTAACATAGTAACA
CCAATCGCCCCAGAATGCGTAATTCAGAAAGGGACGATCATAGAGGGTATGTCACG
GAAGGATTGTACAGAGTTGGAGCAAGACATAATTTGTAGGACAGTTACGACATACA
CGCTTGCCAGAGACACCAGATTGTGTTTACAAGGCAATATCTCTTCTTGTAGGTATC
AACAGTCAGGCACCCAACTACACACCCCATTTATTACGTACAACGGGGCAGTTATCG
CAAACTGTGATTTAGTCTCTTGTCGGTGTCTCCGTCCTCCTATGATTATCACCCAAGT
AAAAGGGTACCCTCTTACCATAATCACAAGGTCCGTATGTCAAGAATTATCCGTTGA
CAACTTAGTCCTGAATATTGAGACACACCATAATTTCTCCCTTAATCCAACAATTATC
GACCCTCTAACAAGAGTAATAGCCACTACACCGTTAGAGATAGATTCCTTAATCCAA
GAGGCACAAGATCATGCGAATGCAGCGCTAGCTAAAGTGGAAGAGAGTGATAAATA
TCTCAGGGCAGTTACTGGCGGTAATTATTCGAATTGGTATATCGTTCTTGTCATCGTC
TTACTATTTGGGAATTTGGGCTGGTCGCTGTTGTTAACAGTACTTCTATGTAGGTCAC
GGAAACAACAGCGACGTTATCAACAAGATGATTCCGTCGGGTCAGAAAGAGGTGTT
GGGGTTGGGACCATCCAGTATATGTCATAAGTGGTGTCAGACATCACAATCCAAGTC
ACTCAGTTATCTACTCCGCTGGCTCGGAGCCATCGACAACGGCGTGAGCCCTCATCC
GGGGACAATCCACAACCTGATACTACCAGACAGTGGCTCCGCACGATGGCAGCAGC
CCACAACGTCCTGCATGTAAATATCATTGCGTTACTTTAATTAAAAAACCTCAAGGT
GACCCATCCTGGCCCATCCGATTGGAGGAGCGGAAGGTTTTAAGGTAATCAGGCAA
ATTGTTTCCAACATGGAATCCCCTCCTTCTGGCAAGGATGCGCCAGCCTTCCGCGAG
CCTAAGCGAACTTGCAGACTCTGTTACAGGGCGACGACTCTCTCCCTTAATCTCACC
ATCGTTGTATTATCAATTATAAGCATTTATGTATCAACTCAAACTGGGGCAAACAAC
TCTTGTGTCAATCCCACAATCGTAACTCCTGATTATTTAACTGGCAGCACGACAGGC
TCAGTTGAAGATCTTGCTGACTTAGAGAGTCAACTCCGGGAGATACGTAGAGACAC
AGGGATTAATCTCCCAGTTCAGATCGATAATACAGAAAATCTAATACTGACAACTCT
GGCGAGTATCAACTCGAATTTACGCTTTCTGCAAAATGCAACAACTGAGAGCCAGA
CCTGCCTATCTCCTGTCAATGACCCTCGCTTTGTAGCTGGCATAAATAGGATCCCAG
CAGGGTCGATGGCATATAATGACTTCAGCAACCTCATTGAGCACGTAAATTTCATAC
CGTCGCCGACAACGTTGTCAGGCTGCACCAGGATACCATCATTTTCACTCTCTAAGA
CGCACTGGTGTTATACCCACAATGTTATATCAAACGGGTGCCTTGACCATGCTGCAA
GTTCACAATACATCTCAATAGGGATCGTTGATACCGGCCTAAATAATGAGCCCTATT
TTAGAACAATGTCTTCAAAGTCACTGAATGACGGATTAAATAGAAAGAGTTGCTCTG
TGACCGCAGCCGCTAATGCATGTTGGTTACTCTGTAGCGTAGTAACAGAATACGAAG
CTGCGGACTATCGATCACGAACTCCTACTGCTATGGTTCTGGGGCGATTCGATTTTA
ATGGTGAATACACAGAAATAGCTGTCCCCTCCTCACTATTCGATGGCCGGTTTGCAT
CTAACTATCCAGGTGTGGGATCGGGAACTCAAGTCAATGGAACATTATATTTCCCTT
TATATGGAGGGGTCCTTAATGGGTCAGATATAGAAACGGCAAACAAGGGGAAATCC
TTCAGGCCTCAGAATCCTAAGAATAGGTGTCCAGACTCGGAGGCGATCCAAAGCTTT
AGAGCGCAAGATAGCTATTACCCGACGAGATTTGGGAAGGTGCTGATCCAACAGGC
AATCATTGCATGTAGGATCAGTAACAAAAGTTGTACTGATTTCTATCTCCTGTACTTC
GACAACAACAGAGTGATGATGGGTGCAGAGGCACGGCTATATTATCTTAATAACCA
ATTATATCTATATCAACGCTCGTCGAGCTGGTGGCCGCATCCTTTGTTCTATAGCATC
TCATTACCGTCATGTCAAGCCCTTGCTGTCTGTCAAATTACAGAGGCCCATCTGACA
CTGACTTACGCCACGTCGAGGCCAGGAATGTCTATCTGTACAGGAGCATCAAGGTGT
CCAAATAATTGCGTAGACGGTGTATATACTGATGTATGGCCATTGACAAAGAACGAT
GCTCAGGACCCCAATCTCTTCTACACTGTATACTTAAATAATTCAACTCGGCGGATT
AGCCCAACCATAAGTCTATACACTTATGACCGCAGGATCAAATCTAAGCTGGCAGTG
GGTAGTGATATAGGAGCAGCTTATACCACGAGTACCTGTTTCGGCCGATCAGATACA
GGGGCGGTTTATTGCTTAACTATAATGGAGACCGTTAACACAATATTTGGTCAGTAC
CGGATCGTGCCAATATTACTTAGAGTGACAAGCCGGTAATGGATTGTTCCTAGTGAA
GTGGGGCCTATGGTTACTCCGTAACGGAGATGACCTGATTTATGCTATGTAACTAAT
TTTGCTCAATCGGGAGGGTGATCGGCCGGGTATGTCCACAATGACAATGACTCCTAT
TCCCTTGCTAGAGCTAGCTTCCTCTTTAATCCAATACAATCCCTACCTGTAGTTTAAT
AAAAAAATCCTCTATATAAGATCCAGACGCGTGCTATATAAGCTAATAGCTTAGGAT
GGAAACGACAGGAGCGGAACATATCCCTCCTACCATGTCTTCTCATAATATTATCCT
CCCTGACCACCACTTAAATTCCCCTATTGTACTAAATAAATTGATGTACTATTGTAAA
CTCCTTAATATATTGCCTGACCCTAAGTCGCCATGGTACGAGAAGATTAAGTCTTGG
ACTAATTGCTGCATCCGTGTGTCAGACAGCAATCGCATGACTTTGTCACGCGCCTCC
AAGCTACGAGAACTATTGGCAACGTACGGCGTATATTCGAAGAACCACCAGCAATG
TTATACCACCATAATCCATCCCCAATCCCTATCACCCATCATGCAGACTGTCTCTCAA
CTGGGACGCTCAATCCCGACCTGGGCCAGGATCCGAAAGGAGATCACACACAGTAT
CATAGCCCAAACGAATAAATTTGAATCCTTATTCCACAACATCTCAAAGGACCTGAC
AGGGAAAACCAACTTGTTTGGCGGATTCTGTGATTTACATGGGAGCATAAGCACTGC
TGCAAAGCGCTTAATGAATCAACCGGGTCTCTACTTGGATTCACCTGATGCCCATGC
ATGTCAATTTTTGTTCCAGCTTAAAACCTGTCAACGGGAATTGATTCTACTTATGAGG
CAGAATGCCACAACAGAGCTGATTAGGGTTTTTGACTATCCTGAGCTACGGGTTCTT
GTGACACCTGAATATTCGGTCTGGGTTTTTACACGGACCAAACAAGTTACATTACTA
ACATTCGATTGCTTGTTAATGTACTGCGATCTTTCAGACGGCCGGCACAACATCCTA
TTCACTTGTAAGCTTTTGTCTCACTTAAACCACCTCGGGTGTCGGATAAGGGACCTG
CTGGTGCTAATTGATTCACTTGCCGAGAAACACCCCTTAATAGTTTATGATGTAGTT
GCAAGTCTGGAGTCGTTATCATATGGTGCTATACAGCTACATGACAAGGTAGCTGGT
TATGCTGGAACATTTTTCTCATTTATCCTTGCAGAAATACAGGATTCCTTAGAAACA
GTGTTGGATCAAGGTAATAGTGAATCTATCATATCCCAAATAAGGAACATATACTCA
GGTCTCACAGTAAACGAGGCTGCAGAATTGCTATGTGTTATGAGGCTTTGGGGCCAC
CCTGCCCTAAACAGTGTAGACGCTGCAAGCAAGGTGAGACAGAGTATGTGCGCTGG
CAAGCTGCTTAAATTTGATACGATCCAGTTAGTCCTTGCATTCTTCAATACACTAATA
ATTAATGGATATAGGAGAAAGCACCATGGGCGATGGCCGAATGTTTGTAGTGACTC
TATAATTGGGAATGAACTCAAGCGGATGTATTTTGATCAATGTGAGATCCCCCACGA
CTTCACATTAAAGAACTACCGGGAATTAAGTCTTCTTGAATTCGAGTGCACATTTCC
CATAGAATTATCAGATAAACTGAACATATTTTTAAAAGATAAGGCAATAGCATTCCC
GAAGTCCAGGTGGACATCTCCTTTCAAAGCAGATATCACACCGCGCCATTTGCTGCA
AGCGCCTGAGTTTAAAACCCGTGCCAACAGACTACTGCTATCATTCTTACAACTCGA
GGAATTCTCCATCGAATCAGAACTAGAATATGTGACTACCCTGGCTTACCTGGATGA
TGATGAGTTCAACGTATCTTACTCCCTCAAAGAGAAGGAGGTAAAGACAGATGGTC
GAATCTTCGCTAAGCTCACAAGGAAAATGCGGAGCTGTCAGGTGATATTCGAAGAA
TTGCTAGCAGAGCATGTCTCTCCATTATTTAAAGACAACGGCGTTACTATGGCCGAA
TTGTCACTTACTAAGAGCCTACTCGCTATTAGCAACCTAAGCTCCACCTTATTTGAGA
CGCAAACAAGGCAAGGCGATCGGAATGCAAGATTCACGCATGCGCACTTCATAACA
ACTGACCTGCAAAAGTACTGTTTAAACTGGAGGTACCAGAGTGTAAAACTGTTCGCA
CGTCAATTGAACCGGTTATTTGGTCTGCAGCATGGGTTTGAATGGATCCATTGCATA
CTAATGAAGTCCACTATGTATGTCGCAGACCCCTTTAACCCTCCACATAGCAATGCA
CGTGAGGCACTCGATGACAACTTAAATAGCGACATATTCATTGTATCACCTAGGGGG
GCCATCGAGGGACTATGCCAAAAAATGTGGACAATCATCTCAATCTCGGCGATCCAT
GCATCCGCTGCTGTGGCTGGGCTACGGGTGGCATCTATGGTCCAAGGTGATAACCAA
GTTATTGGGGTAACACGCGAGTTCCTAGCTGGGCATGATCAAACTTTTGTAGACGAT
CAATTGACGGTTTCACTTGAGAACTTCACTCAGATATTCAAGGAAGTCAACTACGGG
CTAGGTCATAATCTCAAGCTGAGGGAAACAATCAAGTCGAGCCACATGTTCATCTAC
TCAAAGAGGATCTTCTACGACGGCCGAGTGCTTCCACAACTTCTAAAGAACATAAGT
AAGTTAACCCTATCAGCTACAACTACAGGGGAGAACTGTTTAACGAGTTGCGGGGA
CCTATCATCATGCATCACACGATGTATAGAGAATGGGTTCCCTAAAGATGCAGCATT
CGTTTTGAATCAATTAGTGATTAGGATCCAGATACTTGCCGATCACTTTTATTCCATC
CTTGGAGGATGTTTCTCTGGGTTGAACCAGGCGGATATCCGTCTCCTTCTATATGAG
GGTGCAATTTTACCGGCACAATTAGGAGGGTTCAACAACCTCAACATGTCACGGCTA
TTCTGTCGGAATATTGGTGACCCATTAGTCGCGTCCATCGCAGACACCAAGAGATTT
GTGAAGTGCCAACTGCTCGTTCCTCACATTTTAGACTCTGTCGTTGCTATTACCGATA
GGAAGGGGTCTTTTACGACACTGATGATGGATCCATATTCAATAAACTTGGATTATA
TCCAGCAACCAGAGACACGATTGAAGCGACATGTGCAAAAGGTTTTACTACAAGAG
TCAGTGAATCCGCTACTCCAAGGGGTCTTCCTAGAGTCTCAACAAGAGGAAGAGGA
ACAACTTGCAGCGTTCTTGCTGGACCGAGAAGTCGTAATGCCAAGGGTTGCGCACGC
TATATTTGAGTGTACCAGCTTAGGTAGGCGCCGACATATCCAGGGCCTCATCGACAC
GACAAAGACCATTATTGCACTTGCGTTGGATACACAGCAGTTAAGTTATACTAAACG
TGAACAGATTGTGACGTACAATGCAACTTATATGAGGTCCTTAGCAAGCATGCTCAG
TTCAAGCCATAATCAGACACGTCGGTCTGTGATTGGTCACGCATCCTTCAATATCAC
CGACTGCTCGGTCATCCTGGCTCAACAAGTGAGACGTGCAAGCTGGGCGCCCTTGCT
GAATTGGCGGTCATTAGAAGGCTTAGAAGTACCCGACCCTATTGAGTCAGTAGCCG
GCTACCTGGGTTTGGATTCTAACAACTGTTTCTTATGCTGCCACGAGCAGAACAGCT
ATTCCTGGTTTTTCCTTCCCCGAATGTGCCATTTTGATGATTCGAGACAATCTAACTC
CATACAAAGGGTACCTTACATTGGTTCTAAGACTGACGAGCGCCAGATGTCTACGAT
AAATCTACTTGAGAAAACTACCTGTCATGCTAGAGCAGCTACGAGGCTTGCATCATT
ATACATTTGGGCCTTTGGGGACTCAGACGATAGTTGGGATGCAGTCGAGACTCTATC
TAACAGTCGATGCCAGATCACCCGAGAGCACTTGCAAGCTCTGTGCCCTATGCCCTC
TTCAGTGAATCTCCACCATCGATTGAACGACGGAATAACACAAGTGAAGTTTATGCC
ATCGACCAATAGCAGGGTCTCTCGATTTGTTCATATTTCCAATGACCGGCAGAATTA
CGTATTGGATGACTCCGTTACTGACAGTAATCTGATTTACCAACAAGTCATGTTGCT
AGGACTTAGTATACTTGAAACATATTTCAGAGAACCTACAGCGACGAACTTATCAAG
CTTGGTGCTGCACCTTCACACTGATGTATCTTGTTGCTTACGAGAATGCCCGATGAC
ACAATATGCGCCCCCCCTGCGAGACATCCCAGAGTTAACTATAACTGCGTCAAATCC
TTTCTTATTTGATCAAGAACCTATTAGCGAAGCAGACCTGTGTAGACTCTCAAAGAT
AGCATTCCGTAGAGCAGGAGACAACTACGATGCATACGACCAGTTTCAACTAAGAG
CAACACTGGCCGCAACTACAGGTAAGGATGTGGCAGCAACAATCTTTGGTCCTCTAG
CTGCAGTATCAGCAAAAAATGATGCAATAGTCACAAATGACTATAGCGGGAATTGG
ATTTCCGAGTTTAGGTATAGTGATCTCTATCTTTTGAGTGTTAGCCTGGGCTACGAGA
TCCTTTTGATTTTCGCGTATCAACTTTACTACCTACGGATACGGTACCGGCAAAATAT
TGTATGCTATATGGAATCTGTATTCCGACGTTGTCATTCTTTATGCTTGGGCGACTTA
ATCCAGACTATTTCGCACTCTGAAATACTGGTTGGGTTAAATGCAGCGGGCTTTAAT
CTTACCTTGGATCGAAGTGATTTGAAGGAGAATCAACTCTCGCGGCTTGCTGTGAAA
TACCTCACATTGTGTGTCCAGACTGCAATAAGTAATTTGGAGGTAGGATCAGAACCC
CTTTGTATTATTGGAGGTCAATTAGATGATGACATATCATTCCAAGTTGCCCACTTCC
TCTGTCGACGACTGTGCATCATCAGCTTGGTCCACTCAAATGTACAGAACTTACCCC
CGATTAGGGATAATGAAGTTGATGTAAAATCCAAGTTGATCTACGACCATCTTAAGC
TAGTTGCTACCACACTCAATAATAGGGACCAGTCGTATCTGCTAACACTACTGAATA
AGCCTAACATCGAGCTTCATACTCCACAGGTTTACTTCATTATGAGGAAGTGTCTGG
GATTATTAAAAGTCTATGGACCTGTACCTCAAAAGCAACCACTTCCAACCTCCCCAG
TAGTATCACTACCAAATAAGTGCAAATCGAAATGGAAGCTTGAGGAGGTAATTGAC
AGCATTGAGTCACCTAAGTCTTTTAACTGGGTTCCTGATACGACGATTCCATTGGAT
GCTGAGCAAAATCCCCCAAATCCTAACAGGGTCATCGATAAGATAAACATTCTAAG
ATCTCTGAGCCCGCGTCACTCAGTCTGGTACCGTAACCGGCAATACCGGTACGTGTT
GAGCCAGTTAGGGCATGACCAGCTAGGTGGTGCAACCCTATATCTTGGTGAAGGGA
GCGGCTCCACAATCTTGAACATAGAGCCAAAAGTCAGGAGTGATAAGATATATTAT
CATACCTATTTCTCTGCAGACAAAAGCCCTGCACAGCGCAATTTCATCCCCCAGCCA
ACTACTTTTCTGCGATCTAACTTTTATCACTACGAATTAGAGCCGTCTGGATGTGAGT
TCATCAATTGTTGGTCCGAGGATGTAAATGCAACGAATCTTACGGAACTGCGTTGCA
TAAATCACATATTAACTGTGATTCCAGTCGGATCTCTAACACGTATTATTTGTGACGT
GGAATTGGCAAATGACACGTCAATACAATCAGTGGCCACCGCATACATGAACTTAA
GCATTCTTGCACATGCTCTCCTCAACCAGGGAGGCGTYTGCATCTGTAGGTGCCACC
TGTTAGACACGTCCCATCTTGCGATTGTTTCGTTTGTACTTAAAACATTATCAAGCCA
GCTGGCGATTTCGTTTTCAGGATATTGTGGAACCAATGACCCATCTTGTGTCATAGG
AGTCACGAAAGAGTGCTCCATTAGCCTGGATGTCTTAAGTTCGATTGCAGCTGCATT
TATAAATGAATTGCCCTCAAATGAGCTGCCGGTCCCTCAATCATTATTAACGCTACT
AGGGTGTTATCAAGACCAGTTAGAGAGCCTTAGTGCACTCATTGAAAAAACATGGA
TCCAGGAGATCCGGAGGCCTCATTTAATGCAATGTGAGATGGAGTGGATAGGACTTT
TGGGAACAGATGCGCTGGGCGATGTCGACACATTTATCAGTTATCGCAATGATTCGT
GTAATTCCATCCCTGATCTACTGACGCCAGCTGTGAGTGCCCTCTTATTTGAATTAAT
CAGCCTGACACCTGAGCTTCCTGGTACGGAGGACCAGCCGAGTAGGCGTGTAATCA
GCATTGGCCAAGCTTTTAACCTGACTATTTCGGGAAAAGTTAATACGATGATCAGAT
CATGCTGTGAGCAGTGCATTAAGTTATTAGCTGCCAATGTCTCCATCCTTAGTGATGT
AGATTTGTCGTATTTTGTGAGGGGGATTAGAGATGGTTCTTTTGCTTTGGGGTTGTTA
ATCACCCAGCGTCAAATACTCAAGGCATCGAGAGCACCAAAGTACCTCAAAACGCA
TCAGGTCCAGAACTGGATTGCGTTGTTACTGGAGGTCAAACTAGAGGAAATCTTCTC
TCGCCACTACCGGAAAGTGCTATTGAGAACTCTACGGCTGTTATCCCTCTACGGGCT
CCTGCAGCAGAAGGAAAGTTGATCATGATAGATGTCTCTCTGATTTCGCCTCAGCCC
TATATACAGATACCTGCTACTGCTGTTGTAACTATGCAAATATAGGCTAACCTCGGT
CCGGAGTATTGAGGTAATGGGGAGACTCGTAATTGGAGAAAGAAGAAGAACGGGG
CAGATACCAGACCCCGGGATACACTACTATAAGCTACTTGGGTGACTCAGGACGTC
ACTAATAAGAGTACACTGGTTTTAACAATAACACTGATAGTGTAACACTGAATATTG
TAAACAGTCTCAGGGATTATTTAATTAAAAAGACATTGCGGATCATCATGTCAGGCC
TGAACCAATCAGAAACCCATCCTACAGGCATGAGTCGATTAATCCAGTTTAATTTAG
TCCAAGATGATTAGTACTCTTAGATGTTACTAAGTATCAGGATTTAGCGTACTAGAT
TAAACGTCTTTGAAAGTCATTTTACCCTGCAAGGTGCATAAGCATATGCAGGGGAAT
ACCCACCACTGGTTCGTTAGTCTAATTTATTCAAAGATGCCACCAACTGAGTGCACT
GGGAGTCTGATTGAGCTGAATCAACCTAAAGCTCAAGCTTTCCTGTGATGTAGCATG
CAGTGTGCTACTGCTGCATAGAGCCAATCAGGGGCGTCTTGAAGGCCTCCACCTTCC
TCAGGCAACGATAAAGCAGCTCACTGCTACACCCACCATGCAAATACAAGGATATG
TAGGTCCATCACCCTGCTTGCTAATGTGGTCGGCTCCAGGACCATGGCCATAGCGCT
ATCCGTCTAATTCAATCCGCCTCATCATATGCGTATACAATCCTCCACTGAGAAATG
CACCTGCTCAATCCAATGACAAACTACAACAACACGAGATCATTATGGGGTCGATTC
CCTTGAATCAGAAAGGTAAAAACGGCAGCGGAGATGGAGAATACCAGACCTAATCG
GCAAAGAACAAAAGGTTGAAGACTGATTTAAAAATCATTATAACTTTTTGTTTAGT
APMV3_SARS_CoV2-S-GSopt (SEQ ID NO: 18):
ACTAAACAGAAAATTAATACTTGTTAGTAGTCGCCGATTAATCCGCTAATATATTAG
GAGCGGAAGTCCTACACTCCGCCTCCGACCACGAATTGAAATCATCATGGCTGGAA
TTTTTAATACTTATGAGCTCTTTGTTAAGGATCAGACATGTATGCACAAGAGGGCTG
CAAGCCTAATATCGGGAGGTCAGCTTAAAAGCAATATACCAGTCTTCATCACCACAA
AAGATGATCCAGCTGTCCGATGGGACCTTGTATGTTTTAATCTCCGACTTGTTGTTAG
TGAATCATCTACCTCGGTGATCCGGCAGGGGGCTCTTATATCATTGCTTTCCATTACT
GCTAGCAATATGAGGGCTCTTGCAGCAATTGCTGGGCAGACTGATGAATCGATGATT
AATATTATAGAAGTTGTGGACTTTGATGGACTTGAGCCCCAGTGTGACACTCGCAGT
GGCTTAGACGCTCAGAAGCAGGAGATGTTTAAGGATATTGCCCGTGATATGCCGAG
GGCACTATCCAGCGGGACCCCGTTCCAGCACGGAAATGCGGAAGCGAATGGGCCTG
AAGACACACACATGTTCCTCCGATCCGCAATTAGCGTTCTGACACAGATCTGGATCC
TGGTTGCAAAAGCCATGACTAACATTGATGGCAGCAACGAGGCAAGCGATCGCCGC
CTTGCCAAGTATATTCAACAGAATAGAATTGACAGGCGATTCATGCTTGCTCAAGCT
ACCAGGACTGTATGTCAGCAGGTCATAAAGGATTCTTTGACCTTGAGAAGGTTCCTT
GTAACTGAGCTTCGAAAGTCGCGGGGGGCATTGCATAGCGGGTCATCTTACTATGCT
ATGATTGGAGACATGCAGGCATACATATTTAATGCAGGCCTTACACCATTCCTCACT
ACCCTCAGGTACGGGATCGGGACTAAATACCATGCCCTTGCAATTAGCTCACTTGCA
GGTGACCTGAATAAGATTAAGGGGTTACTGACTCTATACAAAGAGAAAGGGAGTGA
TGCGGGCTATATGGCACTTCTAGAGGATGCTGACTGCATGCAATTTGCGCCAGGTAA
TTACTCACTATTATATTCATATGCGATGGGAGTAGCAAGTGTACATGATGAAGGCAT
GAGGAATTACCAATACGCCCGGCGCTTCCTCCATAAGGGGATGTACCAATTTGGACG
TGATATCGCAACACAACAGCAACATGCACTTGATGAATCCCTCGCCCAGGAGATGA
GAATCACAGAGGCTGACAGGGCAAATCTCAAGGTAATGATGGCAAACATTGGGGAA
GCATCAAGCTTCAGTGATATGCCTAATCCAGGAGCTAGCGGCATTCCTGCCTTCAAT
GACCGCCCTGATGAGATCTTCTCTGAGCCAGGCTATCGCAAGCCCCCTCAAGATCAG
CCGCTGATAAAGCTCCCAGATCTCGAGGAAGAAGAGCAGGACGAATTCGACATGTA
GAGAGCCGCCCACCACCCAGAGCCAGCGACAAACCCAGGCAAAACAACCCACCATC
TCCGACCCGGAACACAACGCAACATCGAGGACCCCGGGCAGCCGACACCGCAGCCG
CAAGACCTCCGCATCATGAACGACCAGCATCCAAACATCTGTCCTCCCTATTCTATC
AGTTTAATAAAAAAGGCTTTACGTGCACAGACAGAGATCCTGACATAATTCAGCCG
GGGGGATTCAACTTAGGAGCGGAAGTATCCCTCCTAACCTGCACCTGCATTTCAATA
TGGATCTTGAGTTTAGTAGTGAGGAAGCAGTCGCAGCCCTCTTGGATGTGAGTTCCT
CAACCATAACCGAGGTTCTGAGCAAGCAAAGTATTCCTGACCCTAGCTTTCTCACGT
CCACGGAGGCATCCGGTGAGCAAGTTCCAGATTCGAAGGTCGCCAATCACAAAGCC
AGTGTCGATAAGACCCCAGATCAAGGCCAACCATCGGCAACGCCTTCGGCTCCTCCT
GAGACTGCCGAAAACATCAATACACCATCTTGCGAGGACGGTTTACCCCCTAACTTC
TTCATCCCAAGGGTAGAGAGTTACCACACTAACCTTTTTAAAGGGGGCCCCCAATTG
GACTCAACGGAGCGGCCGCCTGGTCACCAAATAGTTTGCGGAGATCAAGACGCGGA
CCTCTCCCGAGCCGGGATAGCGAGAAAGAAGAAGAAGCAGAAGCATTGCAAAGCG
CTCAACTTTTCGGAATCCCCACTCACAGACAATCAGGCCATAGAAGGGAGTATTCAA
TCTCATGGAGCCCTGGATCAGGAACCTTCCAGACAGAGACCTGGTGCAACCCAGCC
TGCTCTCCAGTCACCGCCTTCCCAAAACAATACAAGTGTGCATGCAGACAGTGCCCA
AGATTCTGCGATCTCTGTTTCAATCCCGCTGACTATGGTGGAATCATTGATCTCTCAA
GTGTCAAAGTTGTCGGACCAAGTCTCGCAGATACAGAAGTTGGTGAGTACCCTTCCT
CAAATTAAGACTGATATTGCTTCGATTCGGAACATGCAAGCAGCTTTGGAAGGACA
GCTCAGTATGATTCGTATATTGGATCCAGGAAACTGTTCGGAATCATCACTCAATGC
GCTGAGAGGGTTCAGTGGTAAGGCACCTGTTATTGTGAGTGGTCCAGGAAATCCCA
ATCAATGCATCAGCCAAGGCTCATCCACCACAATTTGCCTGGATGAGTTAGCCCGGC
CTGTGCCAAATCCTATACAGGTGAAACTTCAAGATGATCAGAAGGACCTATCTGCAC
AGCGACATGCTGTCATTGCATTACTCGAAACCAGAATCCCCCCGGGACCAAAACGT
GACAAATTACTGTCATTGGTTGCAATTGCAAAGACTGCAAGTGATTTAATCAAGATT
AAGAGAATGGCAGTCCTCGGCCAGTGATATCACCCTATGGCAACCAGGCTACAAAT
GTAAGCAGTTTCCGATATGTACCGAATCAATAACACGACCCCTTCCGCAAAAGCAA
GCGCGAGCATGACACACTCAGCTTCAGCCAATTCTAGCTTCACTAAGCAGGTGATCT
GACCATCTGGCTTTACTTCCGCTTTATAAAAAACTCCTAGATCTAACATGGAGGCAG
ACCGAGCTAATGCGGAGGGAGTACACAAGGGGAATCAAAGGAGCGGAAGGTACTA
AGTCACTCCACCGCCTGCCCTCGCCCATCCGCAGCCTCCCCCAACCCGCGGCGCCGC
CACCATGTTCGTGTTTCTGGTGCTGCTGCCTCTGGTGAGCTCCCAGTGCGTGAACCTG
ACCACAAGGACCCAGCTGCCCCCTGCCTATACCAATTCCTTCACACGGGGCGTGTAC
TATCCCGACAAGGTGTTTAGATCTAGCGTGCTGCACTCCACACAGGATCTGTTTCTG
CCTTTCTTTTCTAACGTGACCTGGTTCCACGCCATCCACGTGAGCGGCACCAATGGC
ACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAA
GACACAGTCCCTGCTGATCGTGAACAATGCCACCAACGTGGTCATCAAGGTGTGCG
AGTTCCAGTTTTGTAATGATCCATTCCTGGGCGTGTACTATCACAAGAACAATAAGT
CTTGGATGGAGAGCGAGTTTCGCGTGTATTCCTCTGCCAACAATTGCACATTTGAGT
ACGTGTCCCAGCCCTTCCTGATGGACCTGGAGGGCAAGCAGGGCAATTTCAAGAAC
CTGAGGGAGTTCGTGTTTAAGAATATCGATGGCTACTTCAAGATCTACTCCAAGCAC
ACCCCAATCAACCTGGTGCGCGACCTGCCACAGGGCTTCTCTGCCCTGGAGCCACTG
GTGGATCTGCCCATCGGCATCAACATCACCCGGTTTCAGACACTGCTGGCCCTGCAC
AGAAGCTACCTGACACCAGGCGACAGCTCCTCTGGATGGACCGCAGGAGCTGCCGC
CTACTATGTGGGCTATCTGCAGCCCAGGACCTTCCTGCTGAAGTACAACGAGAATGG
CACCATCACAGACGCCGTGGATTGCGCCCTGGATCCCCTGTCTGAGACCAAGTGTAC
ACTGAAGAGCTTTACCGTGGAGAAGGGCATCTATCAGACAAGCAATTTCAGGGTGC
AGCCTACCGAGTCCATCGTGCGCTTTCCCAATATCACAAACCTGTGCCCTTTTGGCG
AGGTGTTCAACGCAACCCGCTTCGCCAGCGTGTACGCCTGGAATAGGAAGCGCATC
TCCAACTGCGTGGCCGACTATTCTGTGCTGTACAACAGCGCCTCCTTCTCTACCTTTA
AGTGCTATGGCGTGAGCCCCACAAAGCTGAATGACCTGTGCTTTACCAACGTGTACG
CCGATTCCTTCGTGATCAGGGGCGACGAGGTGCGCCAGATCGCCCCTGGCCAGACA
GGCAAGATCGCCGACTACAATTATAAGCTGCCTGACGATTTCACCGGCTGCGTGATC
GCCTGGAACTCTAACAATCTGGATAGCAAAGTGGGCGGCAACTACAATTATCTGTAC
CGGCTGTTTAGAAAGTCTAATCTGAAGCCATTCGAGAGGGACATCTCCACAGAGATC
TACCAGGCCGGCTCTACCCCCTGCAATGGCGTGGAGGGCTTTAACTGTTATTTCCCT
CTGCAGAGCTACGGCTTCCAGCCAACAAACGGCGTGGGCTATCAGCCCTACCGCGT
GGTGGTGCTGTCTTTTGAGCTGCTGCACGCACCTGCAACAGTGTGCGGACCAAAGAA
GAGCACCAATCTGGTGAAGAACAAGTGCGTGAACTTCAACTTCAACGGACTGACCG
GCACAGGCGTGCTGACCGAGTCCAACAAGAAGTTCCTGCCTTTTCAGCAGTTCGGCA
GGGACATCGCAGATACCACAGACGCCGTGCGCGACCCTCAGACCCTGGAGATCCTG
GATATCACACCATGCTCCTTCGGCGGCGTGTCTGTGATCACACCAGGCACCAATACA
AGCAACCAGGTGGCCGTGCTGTATCAGGACGTGAATTGTACCGAGGTGCCCGTGGC
AATCCACGCAGATCAGCTGACCCCTACATGGCGGGTGTACTCTACCGGCAGCAACG
TGTTCCAGACAAGAGCCGGATGCCTGATCGGAGCCGAGCACGTGAACAATAGCTAT
GAGTGCGACATCCCTATCGGCGCCGGCATCTGTGCCTCCTACCAGACCCAGACAAAC
TCCCCACGGAGAGCCCGGTCTGTGGCCAGCCAGTCCATCATCGCCTATACCATGAGC
CTGGGCGCCGAGAATTCCGTGGCCTACTCCAACAATTCTATCGCCATCCCTACCAAC
TTCACAATCTCCGTGACCACAGAGATCCTGCCAGTGAGCATGACCAAGACATCCGTG
GACTGCACAATGTATATCTGTGGCGATTCCACCGAGTGCTCTAACCTGCTGCTGCAG
TACGGCTCTTTTTGTACCCAGCTGAATAGAGCCCTGACAGGCATCGCCGTGGAGCAG
GACAAGAACACACAGGAGGTGTTCGCCCAGGTGAAGCAGATCTACAAGACCCCACC
CATCAAGGACTTTGGCGGCTTCAACTTCAGCCAGATCCTGCCCGATCCTAGCAAGCC
ATCCAAGCGGTCTTTTATCGAGGACCTGCTGTTCAACAAGGTGACCCTGGCCGATGC
CGGCTTCATCAAGCAGTATGGCGATTGCCTGGGCGACATCGCCGCCAGAGACCTGA
TCTGTGCCCAGAAGTTTAATGGCCTGACCGTGCTGCCTCCACTGCTGACAGATGAGA
TGATCGCCCAGTACACATCTGCCCTGCTGGCCGGCACCATCACAAGCGGATGGACCT
TCGGCGCAGGAGCCGCCCTGCAGATCCCCTTTGCCATGCAGATGGCCTATCGGTTCA
ACGGCATCGGCGTGACCCAGAATGTGCTGTACGAGAACCAGAAGCTGATCGCCAAT
CAGTTTAACTCCGCCATCGGCAAGATCCAGGACTCTCTGAGCTCCACAGCCAGCGCC
CTGGGCAAGCTGCAGGATGTGGTGAATCAGAACGCCCAGGCCCTGAATACCCTGGT
GAAGCAGCTGTCTAGCAACTTCGGCGCCATCTCCTCTGTGCTGAATGATATCCTGAG
CAGGCTGGACAAGGTGGAGGCAGAGGTGCAGATCGACCGGCTGATCACAGGCAGA
CTGCAGTCCCTGCAGACCTACGTGACACAGCAGCTGATCAGGGCAGCAGAGATCAG
GGCCTCTGCCAATCTGGCCGCCACCAAGATGAGCGAGTGCGTGCTGGGCCAGTCCA
AGAGAGTGGACTTTTGTGGCAAGGGCTATCACCTGATGAGCTTCCCACAGTCCGCCC
CTCACGGAGTGGTGTTTCTGCACGTGACCTACGTGCCAGCCCAGGAGAAGAACTTCA
CCACAGCACCAGCAATCTGCCACGATGGCAAGGCACACTTTCCTAGGGAGGGCGTG
TTCGTGAGCAACGGCACCCACTGGTTTGTGACACAGCGCAATTTCTACGAGCCACAG
ATCATCACCACAGACAATACATTCGTGTCCGGCAACTGTGACGTGGTCATCGGCATC
GTGAACAATACCGTGTATGATCCTCTGCAGCCAGAGCTGGACTCTTTTAAGGAGGAG
CTGGATAAGTACTTCAAGAATCACACCAGCCCCGACGTGGATCTGGGCGACATCTCT
GGCATCAATGCCAGCGTGGTGAACATCCAGAAGGAGATCGACAGGCTGAACGAGGT
GGCCAAGAATCTGAACGAGTCCCTGATCGATCTGCAGGAGCTGGGCAAGTATGAGC
AGTACATCAAGTGGCCCTGGTATATCTGGCTGGGCTTCATCGCCGGCCTGATCGCCA
TCGTGATGGTGACCATCATGCTGTGCTGTATGACAAGCTGCTGTTCCTGCCTGAAGG
GCTGCTGTTCTTGTGGCAGCTGCTGTAAGTTTGATGAGGACGATAGCGAGCCTGTGC
TGAAGGGCGTGAAGCTGCACTACACCTGATAGTTTATAAAAAACATTGTGAGAATG
CAATTTAAGGACGAAAGTAGGAGCGGAAGCCGCGGCCCAGGCTCCATTCAAAAACC
ATGGCCGCTGCTCTCGGGAACATGCACCCGTCATCGTCGATTACTCTTATGCATGAT
GACCCGTCCATCCAGACGCAGTTGCTAGCCTTCCCTCTGATAAGTGAGAAGACAGA
GACAGGGACGACAAAGCTGCAGCCTCAAGTGAGAATGCAATCTTTCCTATCAACCG
ATAGCCAGAAGTATCATCTGGTCTTTATCAACACATATGGTTTCATTGCGGAGGATT
TTAATTGTAGTCCAGCAAACGGATTTGTGCCTGCACTATTTCAACCCAAGTCTAAAG
TCTTATCGTCAGCGATGGTTACACTTGGGGCTGTCCCTGCTGACACTGTTCTCCAGGA
CCTACAACGGGACTTAATCGCAATGAGATTTAAAGTCCGAAAGAGTGCGTCAGCGA
AGGAGCTAATATTATTCTCCACAGACAATGTCCCTGCAACATTAACAGGGTCATCAG
TCTGGAAGAATAAAGGAGTAATAGCTGATACAGCAACTGCAGTTAAAGCGCCCGGT
CGCATCTCATGCGATGCGGTATGCAGTTATTGCATTACATTCATCTCTTTTTGCTTCT
TCCACTCATCTGCTCTTTTTAAGGTGCCCAAGCCTCTGCTGAATTTCGAGACGGCCAT
AGCATATTCCCTTGTACTCCAGGTCGAGCTTGAGTTCCCAAATATCAAAGATACCTT
ACACGAGAAGTATCTCAAAGTTAAAGACTCGAAGTGGTATTGTACAATTGACATCC
ATATTGGGAATCTTTTAAAGAGAACCGCCAAGCAACGAAGGAGGACACCTTCAGAG
ATAATGCAGAAAGTCCGTAAGATGGGTTTCAGGATTGGGTTATACGATTTGTGGGGT
CCAACAATTGTGGTTGAATTAACTGGGTCCTCAAGTAAGTCCCTACACGGATTCTTC
TCGAGTGAGCGGCTGGCCTGCCACCCGGTATCACAGTATAATCCTCATGTTGGACAG
CTTATCTGGGCCCACGATGTATCCATCACGGGGTGCCACATGATTATATCTGAACTT
GAGAAAAAGAAGGCACTTGCAATGGCTGACCTGACCGTGAGTGATGCAGTAGCTGT
CAACACGAGCATCAAGAGCTTATCGCCTTTCAGACTGTTCAAAAAATATTAGGGCTA
GACAACTCCTAGGGTGTGTGCTTATAATTTGCATATTCTAGATTATAATCATTAACCG
CGTCCAAATGATCAGACCGCATAATTAACAGCTCAGCCAGGACTCGCAAATCCTGC
ATAGGCTCAAATAACAAGACGACATATATACTCACACAACAAGGTGTAATATCTAG
AAGGAATCAGAATTGAGACAATATCTAGGTTATCAGCGCTACTGCCCTGCAACTCAC
TATGTATGCTTGGCATTAATAAAAAACATTGTGAGAATGCAATTTAAGGACGAAAGT
AGGAGCGGAACACGCTCAACTGAACCCAAGCGCACACCAGCCCCCCGGCACCGCAC
CGGAAGGACGCACAACAGCCGACCGCCCCCACCAGGGCAGAGGCCATGCAACCAG
GCTCGGCACTCCACCTCCCGCACCTGTACATAATAATTGCCTTAGTCAGCGATGGCA
CTTTAGGCCAAACAGCTAAAATTGATAGGCTGATACAGGCAGGGATTGTCTTGGGTA
GTGGAAAGGAGCTCCATATCTCACAAGATTCAGGGACACTGGATTTATTCGTGAGGT
TGTTACCAGTACTCCCTTCGAATTTGTCCCATTGCCAGCTTGAAGCAATAACACAGT
ATAATAAGACGGTGACTAGGTTATTGGCCCCAATAGGGAAGAACCTAGAACAGGTA
CTACAAGCGAGACCGCGTGGGAGGCTGTTCGGAGCAATAATAGGGTCCATCGCGCT
TGGTGTTGCCACATCAGCACAGATTACCGCTGCAATTGCACTCGTCCGTGCACAGCA
AAATGCTAATGATATATTAGCACTTAAAAACGCTCTCCAATCCAGCAATGAGGCAAT
CCGTCAACTCACGTATGGCCAGGACAAACAGTTACTTGCAATTTCTAAAATACAAAA
AGCTGTGAATGAGCAGATCCTCCCTGCACTCGATCAGCTGGACTGTGCTGTCCTTGG
AACCAAACTAGCTGTGCAGCTTAATCTGTATCTCATTGAGATGACTACCATTTTCGG
CGAGCAAATCAATAATCCAGTCTTGGCGACCATCCCATTAAGTTACATTTTACGCCT
GACCGGTGCGGAGTTGAACAACGTCCTCATGAAGCAAGCCCGATCGTCCTTAAGCC
TTGTACAGTTAGTTTCAAAAGGCTTACTTAGTGGACAAGTAATTGGCTATGACCCAT
CTGTTCAAGGTCTGATAATAAGGGTGAATCTGATGCGAACGCAGAAGATCGATAGA
GCACTAGTTTATCAGCCGTATGTATTACCAATCACTCTTAACTCTAACATAGTAACA
CCAATCGCCCCAGAATGCGTAATTCAGAAAGGGACGATCATAGAGGGTATGTCACG
GAAGGATTGTACAGAGTTGGAGCAAGACATAATTTGTAGGACAGTTACGACATACA
CGCTTGCCAGAGACACCAGATTGTGTTTACAAGGCAATATCTCTTCTTGTAGGTATC
AACAGTCAGGCACCCAACTACACACCCCATTTATTACGTACAACGGGGCAGTTATCG
CAAACTGTGATTTAGTCTCTTGTCGGTGTCTCCGTCCTCCTATGATTATCACCCAAGT
AAAAGGGTACCCTCTTACCATAATCACAAGGTCCGTATGTCAAGAATTATCCGTTGA
CAACTTAGTCCTGAATATTGAGACACACCATAATTTCTCCCTTAATCCAACAATTATC
GACCCTCTAACAAGAGTAATAGCCACTACACCGTTAGAGATAGATTCCTTAATCCAA
GAGGCACAAGATCATGCGAATGCAGCGCTAGCTAAAGTGGAAGAGAGTGATAAATA
TCTCAGGGCAGTTACTGGCGGTAATTATTCGAATTGGTATATCGTTCTTGTCATCGTC
TTACTATTTGGGAATTTGGGCTGGTCGCTGTTGTTAACAGTACTTCTATGTAGGTCAC
GGAAACAACAGCGACGTTATCAACAAGATGATTCCGTCGGGTCAGAAAGAGGTGTT
GGGGTTGGGACCATCCAGTATATGTCATAAGTGGTGTCAGACATCACAATCCAAGTC
ACTCAGTTATCTACTCCGCTGGCTCGGAGCCATCGACAACGGCGTGAGCCCTCATCC
GGGGACAATCCACAACCTGATACTACCAGACAGTGGCTCCGCACGATGGCAGCAGC
CCACAACGTCCTGCATGTAAATATCATTGCGTTACTTTAATTAAAAAACCTCAAGGT
GACCCATCCTGGCCCATCCGATTGGAGGAGCGGAAGGTTTTAAGGTAATCAGGCAA
ATTGTTTCCAACATGGAATCCCCTCCTTCTGGCAAGGATGCGCCAGCCTTCCGCGAG
CCTAAGCGAACTTGCAGACTCTGTTACAGGGCGACGACTCTCTCCCTTAATCTCACC
ATCGTTGTATTATCAATTATAAGCATTTATGTATCAACTCAAACTGGGGCAAACAAC
TCTTGTGTCAATCCCACAATCGTAACTCCTGATTATTTAACTGGCAGCACGACAGGC
TCAGTTGAAGATCTTGCTGACTTAGAGAGTCAACTCCGGGAGATACGTAGAGACAC
AGGGATTAATCTCCCAGTTCAGATCGATAATACAGAAAATCTAATACTGACAACTCT
GGCGAGTATCAACTCGAATTTACGCTTTCTGCAAAATGCAACAACTGAGAGCCAGA
CCTGCCTATCTCCTGTCAATGACCCTCGCTTTGTAGCTGGCATAAATAGGATCCCAG
CAGGGTCGATGGCATATAATGACTTCAGCAACCTCATTGAGCACGTAAATTTCATAC
CGTCGCCGACAACGTTGTCAGGCTGCACCAGGATACCATCATTTTCACTCTCTAAGA
CGCACTGGTGTTATACCCACAATGTTATATCAAACGGGTGCCTTGACCATGCTGCAA
GTTCACAATACATCTCAATAGGGATCGTTGATACCGGCCTAAATAATGAGCCCTATT
TTAGAACAATGTCTTCAAAGTCACTGAATGACGGATTAAATAGAAAGAGTTGCTCTG
TGACCGCAGCCGCTAATGCATGTTGGTTACTCTGTAGCGTAGTAACAGAATACGAAG
CTGCGGACTATCGATCACGAACTCCTACTGCTATGGTTCTGGGGCGATTCGATTTTA
ATGGTGAATACACAGAAATAGCTGTCCCCTCCTCACTATTCGATGGCCGGTTTGCAT
CTAACTATCCAGGTGTGGGATCGGGAACTCAAGTCAATGGAACATTATATTTCCCTT
TATATGGAGGGGTCCTTAATGGGTCAGATATAGAAACGGCAAACAAGGGGAAATCC
TTCAGGCCTCAGAATCCTAAGAATAGGTGTCCAGACTCGGAGGCGATCCAAAGCTTT
AGAGCGCAAGATAGCTATTACCCGACGAGATTTGGGAAGGTGCTGATCCAACAGGC
AATCATTGCATGTAGGATCAGTAACAAAAGTTGTACTGATTTCTATCTCCTGTACTTC
GACAACAACAGAGTGATGATGGGTGCAGAGGCACGGCTATATTATCTTAATAACCA
ATTATATCTATATCAACGCTCGTCGAGCTGGTGGCCGCATCCTTTGTTCTATAGCATC
TCATTACCGTCATGTCAAGCCCTTGCTGTCTGTCAAATTACAGAGGCCCATCTGACA
CTGACTTACGCCACGTCGAGGCCAGGAATGTCTATCTGTACAGGAGCATCAAGGTGT
CCAAATAATTGCGTAGACGGTGTATATACTGATGTATGGCCATTGACAAAGAACGAT
GCTCAGGACCCCAATCTCTTCTACACTGTATACTTAAATAATTCAACTCGGCGGATT
AGCCCAACCATAAGTCTATACACTTATGACCGCAGGATCAAATCTAAGCTGGCAGTG
GGTAGTGATATAGGAGCAGCTTATACCACGAGTACCTGTTTCGGCCGATCAGATACA
GGGGCGGTTTATTGCTTAACTATAATGGAGACCGTTAACACAATATTTGGTCAGTAC
CGGATCGTGCCAATATTACTTAGAGTGACAAGCCGGTAATGGATTGTTCCTAGTGAA
GTGGGGCCTATGGTTACTCCGTAACGGAGATGACCTGATTTATGCTATGTAACTAAT
TTTGCTCAATCGGGAGGGTGATCGGCCGGGTATGTCCACAATGACAATGACTCCTAT
TCCCTTGCTAGAGCTAGCTTCCTCTTTAATCCAATACAATCCCTACCTGTAGTTTAAT
AAAAAAATCCTCTATATAAGATCCAGACGCGTGCTATATAAGCTAATAGCTTAGGAT
GGAAACGACAGGAGCGGAACATATCCCTCCTACCATGTCTTCTCATAATATTATCCT
CCCTGACCACCACTTAAATTCCCCTATTGTACTAAATAAATTGATGTACTATTGTAAA
CTCCTTAATATATTGCCTGACCCTAAGTCGCCATGGTACGAGAAGATTAAGTCTTGG
ACTAATTGCTGCATCCGTGTGTCAGACAGCAATCGCATGACTTTGTCACGCGCCTCC
AAGCTACGAGAACTATTGGCAACGTACGGCGTATATTCGAAGAACCACCAGCAATG
TTATACCACCATAATCCATCCCCAATCCCTATCACCCATCATGCAGACTGTCTCTCAA
CTGGGACGCTCAATCCCGACCTGGGCCAGGATCCGAAAGGAGATCACACACAGTAT
CATAGCCCAAACGAATAAATTTGAATCCTTATTCCACAACATCTCAAAGGACCTGAC
AGGGAAAACCAACTTGTTTGGCGGATTCTGTGATTTACATGGGAGCATAAGCACTGC
TGCAAAGCGCTTAATGAATCAACCGGGTCTCTACTTGGATTCACCTGATGCCCATGC
ATGTCAATTTTTGTTCCAGCTTAAAACCTGTCAACGGGAATTGATTCTACTTATGAGG
CAGAATGCCACAACAGAGCTGATTAGGGTTTTTGACTATCCTGAGCTACGGGTTCTT
GTGACACCTGAATATTCGGTCTGGGTTTTTACACGGACCAAACAAGTTACATTACTA
ACATTCGATTGCTTGTTAATGTACTGCGATCTTTCAGACGGCCGGCACAACATCCTA
TTCACTTGTAAGCTTTTGTCTCACTTAAACCACCTCGGGTGTCGGATAAGGGACCTG
CTGGTGCTAATTGATTCACTTGCCGAGAAACACCCCTTAATAGTTTATGATGTAGTT
GCAAGTCTGGAGTCGTTATCATATGGTGCTATACAGCTACATGACAAGGTAGCTGGT
TATGCTGGAACATTTTTCTCATTTATCCTTGCAGAAATACAGGATTCCTTAGAAACA
GTGTTGGATCAAGGTAATAGTGAATCTATCATATCCCAAATAAGGAACATATACTCA
GGTCTCACAGTAAACGAGGCTGCAGAATTGCTATGTGTTATGAGGCTTTGGGGCCAC
CCTGCCCTAAACAGTGTAGACGCTGCAAGCAAGGTGAGACAGAGTATGTGCGCTGG
CAAGCTGCTTAAATTTGATACGATCCAGTTAGTCCTTGCATTCTTCAATACACTAATA
ATTAATGGATATAGGAGAAAGCACCATGGGCGATGGCCGAATGTTTGTAGTGACTC
TATAATTGGGAATGAACTCAAGCGGATGTATTTTGATCAATGTGAGATCCCCCACGA
CTTCACATTAAAGAACTACCGGGAATTAAGTCTTCTTGAATTCGAGTGCACATTTCC
CATAGAATTATCAGATAAACTGAACATATTTTTAAAAGATAAGGCAATAGCATTCCC
GAAGTCCAGGTGGACATCTCCTTTCAAAGCAGATATCACACCGCGCCATTTGCTGCA
AGCGCCTGAGTTTAAAACCCGTGCCAACAGACTACTGCTATCATTCTTACAACTCGA
GGAATTCTCCATCGAATCAGAACTAGAATATGTGACTACCCTGGCTTACCTGGATGA
TGATGAGTTCAACGTATCTTACTCCCTCAAAGAGAAGGAGGTAAAGACAGATGGTC
GAATCTTCGCTAAGCTCACAAGGAAAATGCGGAGCTGTCAGGTGATATTCGAAGAA
TTGCTAGCAGAGCATGTCTCTCCATTATTTAAAGACAACGGCGTTACTATGGCCGAA
TTGTCACTTACTAAGAGCCTACTCGCTATTAGCAACCTAAGCTCCACCTTATTTGAGA
CGCAAACAAGGCAAGGCGATCGGAATGCAAGATTCACGCATGCGCACTTCATAACA
ACTGACCTGCAAAAGTACTGTTTAAACTGGAGGTACCAGAGTGTAAAACTGTTCGCA
CGTCAATTGAACCGGTTATTTGGTCTGCAGCATGGGTTTGAATGGATCCATTGCATA
CTAATGAAGTCCACTATGTATGTCGCAGACCCCTTTAACCCTCCACATAGCAATGCA
CGTGAGGCACTCGATGACAACTTAAATAGCGACATATTCATTGTATCACCTAGGGGG
GCCATCGAGGGACTATGCCAAAAAATGTGGACAATCATCTCAATCTCGGCGATCCAT
GCATCCGCTGCTGTGGCTGGGCTACGGGTGGCATCTATGGTCCAAGGTGATAACCAA
GTTATTGGGGTAACACGCGAGTTCCTAGCTGGGCATGATCAAACTTTTGTAGACGAT
CAATTGACGGTTTCACTTGAGAACTTCACTCAGATATTCAAGGAAGTCAACTACGGG
CTAGGTCATAATCTCAAGCTGAGGGAAACAATCAAGTCGAGCCACATGTTCATCTAC
TCAAAGAGGATCTTCTACGACGGCCGAGTGCTTCCACAACTTCTAAAGAACATAAGT
AAGTTAACCCTATCAGCTACAACTACAGGGGAGAACTGTTTAACGAGTTGCGGGGA
CCTATCATCATGCATCACACGATGTATAGAGAATGGGTTCCCTAAAGATGCAGCATT
CGTTTTGAATCAATTAGTGATTAGGATCCAGATACTTGCCGATCACTTTTATTCCATC
CTTGGAGGATGTTTCTCTGGGTTGAACCAGGCGGATATCCGTCTCCTTCTATATGAG
GGTGCAATTTTACCGGCACAATTAGGAGGGTTCAACAACCTCAACATGTCACGGCTA
TTCTGTCGGAATATTGGTGACCCATTAGTCGCGTCCATCGCAGACACCAAGAGATTT
GTGAAGTGCCAACTGCTCGTTCCTCACATTTTAGACTCTGTCGTTGCTATTACCGATA
GGAAGGGGTCTTTTACGACACTGATGATGGATCCATATTCAATAAACTTGGATTATA
TCCAGCAACCAGAGACACGATTGAAGCGACATGTGCAAAAGGTTTTACTACAAGAG
TCAGTGAATCCGCTACTCCAAGGGGTCTTCCTAGAGTCTCAACAAGAGGAAGAGGA
ACAACTTGCAGCGTTCTTGCTGGACCGAGAAGTCGTAATGCCAAGGGTTGCGCACGC
TATATTTGAGTGTACCAGCTTAGGTAGGCGCCGACATATCCAGGGCCTCATCGACAC
GACAAAGACCATTATTGCACTTGCGTTGGATACACAGCAGTTAAGTTATACTAAACG
TGAACAGATTGTGACGTACAATGCAACTTATATGAGGTCCTTAGCAAGCATGCTCAG
TTCAAGCCATAATCAGACACGTCGGTCTGTGATTGGTCACGCATCCTTCAATATCAC
CGACTGCTCGGTCATCCTGGCTCAACAAGTGAGACGTGCAAGCTGGGCGCCCTTGCT
GAATTGGCGGTCATTAGAAGGCTTAGAAGTACCCGACCCTATTGAGTCAGTAGCCG
GCTACCTGGGTTTGGATTCTAACAACTGTTTCTTATGCTGCCACGAGCAGAACAGCT
ATTCCTGGTTTTTCCTTCCCCGAATGTGCCATTTTGATGATTCGAGACAATCTAACTC
CATACAAAGGGTACCTTACATTGGTTCTAAGACTGACGAGCGCCAGATGTCTACGAT
AAATCTACTTGAGAAAACTACCTGTCATGCTAGAGCAGCTACGAGGCTTGCATCATT
ATACATTTGGGCCTTTGGGGACTCAGACGATAGTTGGGATGCAGTCGAGACTCTATC
TAACAGTCGATGCCAGATCACCCGAGAGCACTTGCAAGCTCTGTGCCCTATGCCCTC
TTCAGTGAATCTCCACCATCGATTGAACGACGGAATAACACAAGTGAAGTTTATGCC
ATCGACCAATAGCAGGGTCTCTCGATTTGTTCATATTTCCAATGACCGGCAGAATTA
CGTATTGGATGACTCCGTTACTGACAGTAATCTGATTTACCAACAAGTCATGTTGCT
AGGACTTAGTATACTTGAAACATATTTCAGAGAACCTACAGCGACGAACTTATCAAG
CTTGGTGCTGCACCTTCACACTGATGTATCTTGTTGCTTACGAGAATGCCCGATGAC
ACAATATGCGCCCCCCCTGCGAGACATCCCAGAGTTAACTATAACTGCGTCAAATCC
TTTCTTATTTGATCAAGAACCTATTAGCGAAGCAGACCTGTGTAGACTCTCAAAGAT
AGCATTCCGTAGAGCAGGAGACAACTACGATGCATACGACCAGTTTCAACTAAGAG
CAACACTGGCCGCAACTACAGGTAAGGATGTGGCAGCAACAATCTTTGGTCCTCTAG
CTGCAGTATCAGCAAAAAATGATGCAATAGTCACAAATGACTATAGCGGGAATTGG
ATTTCCGAGTTTAGGTATAGTGATCTCTATCTTTTGAGTGTTAGCCTGGGCTACGAGA
TCCTTTTGATTTTCGCGTATCAACTTTACTACCTACGGATACGGTACCGGCAAAATAT
TGTATGCTATATGGAATCTGTATTCCGACGTTGTCATTCTTTATGCTTGGGCGACTTA
ATCCAGACTATTTCGCACTCTGAAATACTGGTTGGGTTAAATGCAGCGGGCTTTAAT
CTTACCTTGGATCGAAGTGATTTGAAGGAGAATCAACTCTCGCGGCTTGCTGTGAAA
TACCTCACATTGTGTGTCCAGACTGCAATAAGTAATTTGGAGGTAGGATCAGAACCC
CTTTGTATTATTGGAGGTCAATTAGATGATGACATATCATTCCAAGTTGCCCACTTCC
TCTGTCGACGACTGTGCATCATCAGCTTGGTCCACTCAAATGTACAGAACTTACCCC
CGATTAGGGATAATGAAGTTGATGTAAAATCCAAGTTGATCTACGACCATCTTAAGC
TAGTTGCTACCACACTCAATAATAGGGACCAGTCGTATCTGCTAACACTACTGAATA
AGCCTAACATCGAGCTTCATACTCCACAGGTTTACTTCATTATGAGGAAGTGTCTGG
GATTATTAAAAGTCTATGGACCTGTACCTCAAAAGCAACCACTTCCAACCTCCCCAG
TAGTATCACTACCAAATAAGTGCAAATCGAAATGGAAGCTTGAGGAGGTAATTGAC
AGCATTGAGTCACCTAAGTCTTTTAACTGGGTTCCTGATACGACGATTCCATTGGAT
GCTGAGCAAAATCCCCCAAATCCTAACAGGGTCATCGATAAGATAAACATTCTAAG
ATCTCTGAGCCCGCGTCACTCAGTCTGGTACCGTAACCGGCAATACCGGTACGTGTT
GAGCCAGTTAGGGCATGACCAGCTAGGTGGTGCAACCCTATATCTTGGTGAAGGGA
GCGGCTCCACAATCTTGAACATAGAGCCAAAAGTCAGGAGTGATAAGATATATTAT
CATACCTATTTCTCTGCAGACAAAAGCCCTGCACAGCGCAATTTCATCCCCCAGCCA
ACTACTTTTCTGCGATCTAACTTTTATCACTACGAATTAGAGCCGTCTGGATGTGAGT
TCATCAATTGTTGGTCCGAGGATGTAAATGCAACGAATCTTACGGAACTGCGTTGCA
TAAATCACATATTAACTGTGATTCCAGTCGGATCTCTAACACGTATTATTTGTGACGT
GGAATTGGCAAATGACACGTCAATACAATCAGTGGCCACCGCATACATGAACTTAA
GCATTCTTGCACATGCTCTCCTCAACCAGGGAGGCGTYTGCATCTGTAGGTGCCACC
TGTTAGACACGTCCCATCTTGCGATTGTTTCGTTTGTACTTAAAACATTATCAAGCCA
GCTGGCGATTTCGTTTTCAGGATATTGTGGAACCAATGACCCATCTTGTGTCATAGG
AGTCACGAAAGAGTGCTCCATTAGCCTGGATGTCTTAAGTTCGATTGCAGCTGCATT
TATAAATGAATTGCCCTCAAATGAGCTGCCGGTCCCTCAATCATTATTAACGCTACT
AGGGTGTTATCAAGACCAGTTAGAGAGCCTTAGTGCACTCATTGAAAAAACATGGA
TCCAGGAGATCCGGAGGCCTCATTTAATGCAATGTGAGATGGAGTGGATAGGACTTT
TGGGAACAGATGCGCTGGGCGATGTCGACACATTTATCAGTTATCGCAATGATTCGT
GTAATTCCATCCCTGATCTACTGACGCCAGCTGTGAGTGCCCTCTTATTTGAATTAAT
CAGCCTGACACCTGAGCTTCCTGGTACGGAGGACCAGCCGAGTAGGCGTGTAATCA
GCATTGGCCAAGCTTTTAACCTGACTATTTCGGGAAAAGTTAATACGATGATCAGAT
CATGCTGTGAGCAGTGCATTAAGTTATTAGCTGCCAATGTCTCCATCCTTAGTGATGT
AGATTTGTCGTATTTTGTGAGGGGGATTAGAGATGGTTCTTTTGCTTTGGGGTTGTTA
ATCACCCAGCGTCAAATACTCAAGGCATCGAGAGCACCAAAGTACCTCAAAACGCA
TCAGGTCCAGAACTGGATTGCGTTGTTACTGGAGGTCAAACTAGAGGAAATCTTCTC
TCGCCACTACCGGAAAGTGCTATTGAGAACTCTACGGCTGTTATCCCTCTACGGGCT
CCTGCAGCAGAAGGAAAGTTGATCATGATAGATGTCTCTCTGATTTCGCCTCAGCCC
TATATACAGATACCTGCTACTGCTGTTGTAACTATGCAAATATAGGCTAACCTCGGT
CCGGAGTATTGAGGTAATGGGGAGACTCGTAATTGGAGAAAGAAGAAGAACGGGG
CAGATACCAGACCCCGGGATACACTACTATAAGCTACTTGGGTGACTCAGGACGTC
ACTAATAAGAGTACACTGGTTTTAACAATAACACTGATAGTGTAACACTGAATATTG
TAAACAGTCTCAGGGATTATTTAATTAAAAAGACATTGCGGATCATCATGTCAGGCC
TGAACCAATCAGAAACCCATCCTACAGGCATGAGTCGATTAATCCAGTTTAATTTAG
TCCAAGATGATTAGTACTCTTAGATGTTACTAAGTATCAGGATTTAGCGTACTAGAT
TAAACGTCTTTGAAAGTCATTTTACCCTGCAAGGTGCATAAGCATATGCAGGGGAAT
ACCCACCACTGGTTCGTTAGTCTAATTTATTCAAAGATGCCACCAACTGAGTGCACT
GGGAGTCTGATTGAGCTGAATCAACCTAAAGCTCAAGCTTTCCTGTGATGTAGCATG
CAGTGTGCTACTGCTGCATAGAGCCAATCAGGGGCGTCTTGAAGGCCTCCACCTTCC
TCAGGCAACGATAAAGCAGCTCACTGCTACACCCACCATGCAAATACAAGGATATG
TAGGTCCATCACCCTGCTTGCTAATGTGGTCGGCTCCAGGACCATGGCCATAGCGCT
ATCCGTCTAATTCAATCCGCCTCATCATATGCGTATACAATCCTCCACTGAGAAATG
CACCTGCTCAATCCAATGACAAACTACAACAACACGAGATCATTATGGGGTCGATTC
CCTTGAATCAGAAAGGTAAAAACGGCAGCGGAGATGGAGAATACCAGACCTAATCG
GCAAAGAACAAAAGGTTGAAGACTGATTTAAAAATCATTATAACTTTTTGTTTAGT

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A vector comprising a paramyxovirus comprising one or more polynucleotides encoding one or more coronavirus proteins comprising a spike (S) protein, a membrane (M) protein, an envelope (E) protein or a nucleocapsid (N) protein or fragments thereof.

2. The vector of claim 1, wherein the coronavirus is a severe acute respiratory syndrome coronavirus (SARS-CoV-2).

3. The vector of claim 2, wherein the coronavirus protein is a SARS-CoV-2 spike (S) protein or fragments thereof.

4. The vector of claim 1, wherein the paramyxovirus is an avian paramyxovirus type 3 virus (APMV3).

5. The vector of claim 2, wherein the SARS-CoV-2 S protein or fragments thereof, comprises one or more amino acid substitutions.

6. (canceled)

7. (canceled)

8. The vector of claim 5, wherein the SARS-CoV-2 S protein or fragments thereof, comprises one or more proline substitutions.

9. The vector of claim 8, wherein the SARS-CoV-2 S protein or fragments thereof, comprises one or more proline substitutions at amino acid positions 817, 892, 899, 942, 986 and 987.

10. The vector of claim 9, wherein the SARS-CoV-2 S protein or fragments thereof, comprises six proline substitutions at amino acid positions 817, 892, 899, 942, 986 and 987.

11. The vector of claim 1, wherein the S polynucleotide is codon optimized as compared to a wild-type polynucleotide encoding the one or more S protein or fragments thereof protein and comprises nucleotide adapters for insertion into the APMV3 full-length cDNA.

12. The vector of claim 11, wherein the S polynucleotide is inserted between the APMV3 P and M genes.

13. The vector of claim 12, wherein the S open reading frame (ORF) is transcribed by the APMV 3 polymerase complex as a distinct mRNA.

14. The vector of claim 10, wherein the SARS-CoV-2 S protein or fragments thereof, further comprise amino acid substitutions to a polybasic furin cleavage motif.

15. The vector of claim 14, wherein the polybasic furin cleavage motif comprising an RRAR amino acid sequence is substituted with a GSAS amino acid sequence.

16. The vector of claim 1, wherein the vector is formulated for administration intranasally, intramuscularly, intraperitoneally, orally or intravenously; and/or the vector is formulated as a spray, mist, or aerosol.

17. (canceled)

18. A method of producing a coronavirus vaccine comprising:
inserting a nucleic acid sequence encoding a severe acute respiratory syndrome coronavirus (SARS-CoV-2) spike (S) protein or fragments thereof, into an avian paramyxovirus type 3 virus (APMV3);
contacting a mammalian cell culture stably expressing T7 RNA polymerase with the isolated polynucleotide molecules that include a nucleic acid sequence encoding the genome or antigenome of APMV3 virus comprising a polynucleotide encoding the SARS-CoV-2 S protein or fragments thereof, together with polynucleotides encoding the N, P, and L proteins of APMV3,
harvesting and culturing the APMV3 virus in Vero cells;
injecting the APMV3 virus harvested from the Vero cells into an allantoic cavity of embryonated chicken eggs;
harvesting and isolating the APMV3 comprising the nucleic acid encoding the SARS-CoV-2 S protein or fragments thereof;
and producing the coronavirus vaccine.

19. The method of claim 18, wherein the SARS-CoV-2 S protein or fragments thereof, comprises one or more amino acid substitutions.

20. (canceled)

21. (canceled)

22. The method of claim 18, wherein the SARS-CoV-2 S protein or fragments thereof, comprises one or more proline substitutions.

23. The method of claim 22, wherein: the SARS-CoV-2 S protein or fragments thereof, comprises one or more proline substitutions at amino acid positions 817, 892, 899, 942, 986 and 987; and/or the SARS-CoV-2 S protein or fragments thereof, comprises six proline substitutions at amino acid positions homologous to positions 817, 892, 899, 942, 986 and 987 of the S protein of NCBI Reference sequence NC_045512.2.

24. (canceled)

25. The method of claim 18, wherein the S polynucleotide is codon optimized as compared to a wild-type polynucleotide encoding the one or more S protein or fragments thereof, and comprises nucleotide adapters for insertion into the APMV3 full-length cDNA.

26. The method of claim 25, wherein the SARS-CoV-2 S polynucleotide, is inserted between the APMV3 P and M genes.

27. The method of claim 26, wherein the S open reading frame (ORF) is transcribed by the APMV 3 polymerase complex as a distinct mRNA.

28. The method of claim 18, wherein the SARS-CoV-2 S protein or fragments thereof, further comprise substitutions to inactivate or mutate a polybasic furin cleavage motif.

29. The method of claim 28, wherein the polybasic furin cleavage motif comprising an RRAR amino acid sequence is substituted with a GSAS amino acid sequence.

30. A vaccine comprising the vector of claim 1, formulated as a spray, mist, or aerosol.

31. The vaccine of claim 30 wherein the coronavirus is a severe acute respiratory syndrome coronavirus (SARS-CoV-2).

32. The vaccine of claim 31, wherein the coronavirus peptide is a SARS-CoV-2 spike (S) protein or fragments thereof.

33. The vaccine of claim 32, wherein the SARS-CoV-2 S protein or fragments thereof, comprises six proline substitutions at amino acid positions 817, 892, 899, 942, 986 and 987.

34. The vaccine of claim 30, wherein the paramyxovirus recombinant vector is an avian paramyxovirus type 3 virus (APMV3).

35. (canceled)

36. A pharmaceutical composition comprising a paramyxovirus recombinant vector comprising one or more polynucleotides encoding a coronavirus protein comprising a spike (S) protein, a membrane (M) protein, an envelope (E) protein, a nucleocapsid (N) protein and combinations thereof, or fragments thereof.

37. The pharmaceutical composition of claim 36 wherein the coronavirus is a severe acute respiratory syndrome coronavirus (SARS-CoV-2).

38. The pharmaceutical composition of claim 37, wherein the coronavirus protein is a SARS-CoV-2 spike (S) protein or fragments thereof.

39. The pharmaceutical composition of claim 38, wherein the SARS-CoV-2 S protein or fragments thereof, comprises six proline substitutions at amino acid positions 817, 892, 899, 942, 986 and 987.

40. The pharmaceutical composition of claim 36, wherein the paramyxovirus is an avian paramyxovirus type 3 virus (APMV3).

41. The pharmaceutical composition of claim 36, further comprising an adjuvant and/or a secondary therapeutic agent.

42. (canceled)

43. The pharmaceutical composition of claim 41, wherein the secondary therapeutic agent comprises: a decongestant, an antihistamine, a pain reliever, a fever reducer, a cough suppressant, a cytokine, an antibiotic, an anti-viral agent, or a combination thereof.

44. A method of preventing and treating a subject at risk of contracting a severe acute respiratory syndrome coronavirus (SARS-CoV-2), comprising administering to the subject the vector of claim 1.

45. The method of claim 44, wherein administration of the vector comprises subcutaneous (sc) delivery, intramuscular (im) delivery, intradermal delivery, transdermal delivery, mucosal delivery, intravaginal delivery, intrarectal delivery, intraperitoneal delivery, inhalation delivery, aerosol delivery, or a combination thereof.

46. The method of claim 45, wherein the vector is administered via inhalation delivery, aerosol delivery or the combination thereof.

47. The method of claim 44, further comprising administering an adjuvant and/or a secondary therapeutic agent.

48. (canceled)

49. (canceled)

50. The vector of claim 1, wherein: the SARS-CoV-2 S protein further comprises one or more of L18F, T20N, P26S, D138Y, R190S, K417N, N439K, N440K, L452R, S477G, S477N, E484K, E484Q, N501Y, D614G, H655Y, P681H, and T1027I substitutions; the SARS-CoV-2 S protein further comprises one or more deletions of amino acid H69, V70, Y144, L242, A243, or L244; or the SARS-CoV-2 S protein further comprises one or more of T19R, V70F, T95I, G142D, E156-, F157-, R158G, A222V, W258L, K417N, L452R, T478K, D614G, P681R, D950N substitutions.

51. (canceled)

52. (canceled)


SEQUENCE LISTING (18 sequences)

SEQ ID NO: 1 - DNA, 23978 bp, synthetic construct
  source 1..23978: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtacatcaagtggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatgatttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagtgggtcggcatggcatctccacctcctcgcggtccggacctgggcatccgaaggaggacgcacgtccactcggatggctaagggagagcctgcagtagcataaccccttggggcctctaaacgggtcttgaggggttttttggtgaaaggaggaactataggccggccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcggcgcgcctaatacgactcactataggg

SEQ ID NO: 2 - DNA, 23978 bp, synthetic construct
  source 1..23978: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccacggagagcccggtctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggacaaggtggaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagtgggtcggcatggcatctccacctcctcgcggtccggacctgggcatccgaaggaggacgcacgtccactcggatggctaagggagagcctgcagtagcataaccccttggggcctctaaacgggtcttgaggggttttttggtgaaaggaggaactataggccggccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcggcgcgcctaatacgactcactataggg

SEQ ID NO: 3 - DNA, 23978 bp, synthetic construct
  source 1..23978: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagtgggtcggcatggcatctccacctcctcgcggtccggacctgggcatccgaaggaggacgcacgtccactcggatggctaagggagagcctgcagtagcataaccccttggggcctctaaacgggtcttgaggggttttttggtgaaaggaggaactataggccggccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcggcgcgcctaatacgactcactataggg

SEQ ID NO: 4 - DNA, 23978 bp, synthetic construct
  source 1..23978: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagtgggtcggcatggcatctccacctcctcgcggtccggacctgggcatccgaaggaggacgcacgtccactcggatggctaagggagagcctgcagtagcataaccccttggggcctctaaacgggtcttgaggggttttttggtgaaaggaggaactataggccggccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcggcgcgcctaatacgactcactataggg

SEQ ID NO: 5 - DNA, 23972 bp, synthetic construct
  source 1..23972: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaggacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcggcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatcggtaccggctgtttagaaagtctaatctgaagccattcgagagggacatctccacagagatctaccaggccggctctaagccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccagagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcagaacgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagtgggtcggcatggcatctccacctcctcgcggtccggacctgggcatccgaaggaggacgcacgtccactcggatggctaagggagagcctgcagtagcataaccccttggggcctctaaacgggtcttgaggggttttttggtgaaaggaggaactataggccggccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcggcgcgcctaatacgactcactataggg

SEQ ID NO: 6 - DNA, 11935 bp, synthetic construct
  source 1..11935: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 11854 bp, synthetic construct
  source 1..11854: mol_type = other DNA
atggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctgcagctcgagaggcctaattaattaagtcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattgatctcgatcccgcgaaattaatacgactcactatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataatacc

SEQ ID NO: 8 - DNA, 6709 bp, synthetic construct
  source 1..6709: mol_type = other DNA
ccatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagactagtggatccctgcagctcgagaggcctaattaattaagtcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattgatctcgatcccgcgaaattaatacgactcactatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataata

SEQ ID NO: 9 - DNA, 6478 bp, synthetic construct
  source 1..6478: mol_type = other DNA
ccatggatcttgagtttagtagtgaggaagcagtcgcagccctcttggatgtgagttcctcaaccataaccgaggttctgagcaagcaaagtattcctgaccccagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttagaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgactcgagaggcctaattaattaagtcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattgatctcgatcccgcgaaattaatacgactcactatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataata

SEQ ID NO: 10 - DNA, 6730 bp, synthetic construct
  source 1..6730: mol_type = other DNA
atggccggcatcttcaacacctacgagctgtttgtgaaggaccagacatgcatgcacaagagggcagcctccctgatctctggaggacagctgaagagcaacatccccgtgttcatcaccacaaaggacgatccagccgtgaggtgggacctggtgtgctttaatctgcgcctggtggtgtctgagagctccaccagcgtgatcaggcagggcgccctgatcagcctgctgtccatcaccgcctctaacatgagagccctggccgcaatcgcaggacagacagacgagagcatgatcaatatcatcgaggtggtggacttcgatggactggagccacagtgcgacacacggtccggcctggatgcccagaagcaggagatgtttaaggacatcgccagggatatgccacgcgccctgtctagcggcaccccattccagcacggaaacgcagaggcaaatggacctgaggatacacacatgtttctgagatctgccatcagcgtgctgacccagatctggatcctggtggccaaggccatgacaaacatcgacggctccaatgaggccagcgatcggagactggccaagtacatccagcagaataggatcgacaggcgcttcatgctggcccaggcaaccaggacagtgtgccagcaggtcatcaaggatagcctgaccctgcggagatttctggtgacagagctgcggaagtccagaggcgccctgcactctggctcctcttactatgccatgatcggcgacatgcaggcttacatcttcaacgccggcctgaccccatttctgaccacactgcggtacggcatcggcacaaagtatcacgccctggccatcagctccctggccggcgacctgaataagatcaagggcctgctgaccctgtacaaggagaagggctccgatgccggctatatggccctgctggaggacgccgattgtatgcagttcgcccccggcaactacagcctgctgtactcctatgctatgggcgtggcctctgtgcacgacgagggcatgaggaattaccagtatgccaggcgcttcctgcacaagggcatgtatcagtttggaagggacatcgcaacccagcagcagcacgccctggatgagagcctggcccaggagatgcggatcacagaggccgacagagccaacctgaaagtgatgatggccaatatcggcgaggccagcagcttcagcgatatgccaaacccaggagcctccggcatccccgccttcaatgaccggcctgatgagatcttttccgagcctggctacagaaagccccctcaggaccagcctctgatcaagctgccagatctggaggaggaggagcaggacgagtttgatatgtaatgaataaagatcttataataaaaattcccatagctgcagctcgagaggcctaattaattaagtcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattgatctcgatcccgcgaaattaatacgactcactatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataatacc

SEQ ID NO: 11 - DNA, 6517 bp, synthetic construct
  source 1..6517: mol_type = other DNA
atggatctggagttcagctccgaggaggcagtggccgccctgctggacgtgtctagctccaccatcacagaggtgctgtccaagcagtctatccctgatccatcttttctgaccagcacagaggccagcggagagcaggtgcccgactctaaggtggccaaccacaaggccagcgtggataagacccctgaccagggacagccatctgccacaccaagcgccccacctgagaccgccgagaacatcaatacacctagctgcgaggatggcctgccacccaacttctttatcccacgggtggagtcctaccacaccaatctgttcaagggcggcccccagctggactctacagagagacctccaggccaccagatcgtgtgcggcgaccaggatgcagacctgtccagggccggaatcgcacggaagaagaagaagcagaagcactgtaaggccctgaactttagcgagtcccctctgaccgataatcaggccatcgagggaagcatccagtcccacggcgccctggaccaggagcctagccggcagagaccaggagcaacccagcccgccctccagtctccccctagccagaacaatacatccgtgcacgccgattccgcccaggactctgccatctctgtgagcatccctctgacaatggtggagtccctgatctctcaggtgagcaagctgtccgatcaggtgagccagatccagaagctggtgtccaccctgccacagatcaagacagacatcgccagcatcaggaacatgcaggccgccctggagggacagctgtctatgatccgcatcctggatccaggcaactgtagcgagtctagcctgaatgcactgaggggcttctccggcaaggcacccgtgatcgtgtctggacccggcaaccctaatcagtgcatcagccagggctcctctaccacaatctgtctggatgagctggccaggcccgtgcctaatccaatccaggtgaagctccaggacgatcagaaggacctgtccgcccagaggcacgccgtgatcgccctgctggagacccggatcccaccaggaccaaagagagataagctgctgagcctggtggccatcgccaagacagcctccgacctgatcaagatcaagagaatggccgtgctgggccagtaatgaataaagatcttataataaaaattcccatagctgcagctcgagaggcctaattaattaagtcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattgatctcgatcccgcgaaattaatacgactcactatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataatacc

SEQ ID NO: 12 - DNA, 11953 bp, synthetic construct
  source 1..11953: mol_type = other DNA
atgagctcccacaacatcatcctgccagaccaccacctgaacagccccatcgtgctgaataagctgatgtactattgtaagctgctgaatatcctgccagatcccaagtccccttggtacgagaagatcaagtcttggaccaactgctgtatccgcgtgtccgactctaataggatgacactgtcccgcgcctctaagctgagggagctgctggccacctacggcgtgtatagcaagaaccaccagcagtgctataccacaatcatccacccccagagcctgtcccctatcatgcagaccgtgagccagctgggccggtccatcccaacatgggcacgcatccggaaggagatcacacactccatcatcgcccagaccaacaagttcgagagcctgtttcacaatatctccaaggatctgacaggcaagaccaacctgttcggcggcttttgcgacctgcacggctctatcagcacagccgccaagcgcctgatgaatcagcctggcctgtacctggacagcccagatgcccacgcctgtcagttcctgtttcagctgaagacctgccagagagagctgatcctgctgatgaggcagaacgccaccacagagctgatccgcgtgttcgattacccagagctgcgggtgctggtgaccccagagtattccgtgtgggtgttcacaagaaccaagcaggtgacactgctgacctttgactgtctgctgatgtattgcgacctgtccgatggcaggcacaacatcctgtttacctgtaagctgctgtctcacctgaatcacctgggctgccggatccgggatctgctggtgctgatcgactccctggccgagaagcaccccctgatcgtgtacgatgtggtggcctctctggagtccctgtcttatggcgccatccagctgcacgacaaggtggcaggatacgcaggcacattcttttccttcatcctggccgagatccaggattctctggagaccgtgctggaccagggcaacagcgagtccatcatctcccagatcagaaacatctattctggcctgaccgtgaatgaggccgccgagctgctgtgcgtgatgagactgtggggacaccccgccctgaacagcgtggatgcagccagcaaggtgaggcagtccatgtgcgccggcaagctgctgaagtttgacaccatccagctggtgctggccttctttaacacactgatcatcaatggctaccggagaaagcaccacggccgctggccaaacgtgtgcagcgatagcatcatcggcaatgagctgaagcggatgtacttcgatcagtgcgagatccctcacgactttacactgaagaactatagggagctgtccctgctggagttcgagtgtacctttccaatcgagctgtctgataagctgaatatcttcctgaaggacaaggccatcgcctttcccaagagcagatggacctcccctttcaaggccgacatcacaccaaggcacctgctccaggcccccgagtttaagacccgggccaacagactgctgctgagcttcctccagctggaggagtttagcatcgagtccgagctggagtacgtgaccacactggcctatctggacgatgacgagttcaacgtgagctactctctgaaggagaaggaggtgaagaccgatggccggatctttgccaagctgacaaggaagatgcgcagctgccaggtcatcttcgaggagctgctggccgagcacgtgtcccccctgtttaaggacaacggcgtgacaatggccgagctgtctctgaccaagagcctgctggccatcagcaacctgtctagcacactgttcgagacacagaccagacagggcgatcggaatgccagattcacccacgcccactttatcaccacagacctccagaagtactgtctgaactggaggtatcagtccgtgaagctgtttgcccggcagctgaatagactgttcggcctccagcacggctttgagtggatccactgcatcctgatgaagagcaccatgtacgtggccgatcccttcaacccacctcactccaatgcccgcgaggccctggatgacaacctgaacagcgacatctttatcgtgagccctcggggcgcaatcgagggcctgtgccagaagatgtggaccatcatcagcatctccgccatccacgcctctgccgccgtggccggcctgagagtggccagcatggtgcagggcgataaccaggtcatcggagtgaccagggagttcctggcaggacacgaccagacatttgtggatgaccagctgaccgtgtccctggagaatttcacacagatctttaaggaggtgaactacggcctgggccacaatctgaagctgcgcgagacaatcaagtcctctcacatgttcatctacagcaagagaatcttttatgacggcagggtgctgccacagctgctgaagaacatctctaagctgaccctgagcgccaccacaaccggcgagaattgtctgacctcctgcggcgatctgagctcctgtatcacaagatgcatcgagaacggcttccccaaggacgccgcctttgtgctgaatcagctggtcatcaggatccagatcctggccgatcacttctacagcatcctgggcggctgcttttccggcctgaaccaggcagacatcaggctgctgctgtatgagggagcaatcctgccagcacagctgggaggcttcaacaatctgaacatgtctaggctgttttgtcgcaatatcggcgatcccctggtggccagcatcgcagacacaaagcggttcgtgaagtgccagctgctggtgcctcacatcctggattccgtggtggccatcaccgaccggaagggctcttttacaaccctgatgatggatccttacagcatcaacctggactatatccagcagccagagaccaggctgaagcgccacgtgcagaaggtgctgctccaggagtctgtgaatccactgctccagggcgtgttcctggagagccagcaggaggaggaggagcagctggccgcctttctgctggacagggaggtggtcatgccccgggtggcacacgcaatcttcgagtgtacatccctgggcaggcgccggcacatccagggcctgatcgatacaaccaagaccatcatcgccctggccctggacacacagcagctgagctacaccaagcgcgagcagatcgtgacatacaacgccacctatatgcggtctctggccagcatgctgtctagctcccacaaccagacaagaaggtccgtgatcggccacgcctcttttaatatcaccgactgctccgtgatcctggcacagcaggtgcgcagggccagctgggcccctctgctgaattggagatccctggagggcctggaggtgcctgatccaatcgagtctgtggccggctacctgggcctggacagcaacaattgtttcctgtgctgtcacgagcagaactcttatagctggttctttctgcctcgcatgtgccactttgatgacagccgccagtccaattctatccagcgggtgccatacatcggcagcaagaccgatgagcggcagatgtccacaatcaacctgctggagaagacaacctgccacgccagagccgcaaccagactggcctccctgtatatctgggccttcggcgattccgatgactcttgggacgccgtggagaccctgagcaattccagatgtcagatcacaagggagcacctccaggccctgtgcccaatgccttctagcgtgaacctgcaccacagactgaatgacggcatcacccaggtgaagttcatgcccagcacaaactcccgggtgtctagatttgtgcacatcagcaacgataggcagaattacgtgctggatgactccgtgacagactctaacctgatctatcagcaggtcatgctgctgggcctgtctatcctggagacctacttcagagagcctacagccaccaatctgtcctctctggtgctgcacctgcacaccgatgtgagctgctgtctgagagagtgtcccatgacacagtatgccccacccctgagggacatccctgagctgacaatcaccgcctccaaccctttcctgtttgatcaggagccaatctctgaggccgacctgtgccgcctgagcaagatcgccttcagaagggccggcgacaattacgatgcctatgaccagtttcagctgagggccaccctggccgcaacaaccggcaaggatgtggcagcaacaatcttcggacccctggccgccgtgagcgccaagaacgatgccatcgtgaccaacgactactccggcaattggatctctgagttcaggtacagcgacctgtatctgctgtctgtgagcctgggctacgagatcctgctgatctttgcctatcagctgtactatctgcgcatccggtacagacagaatatcgtgtgctatatggagagcgtgttccggcggtgccacagcctgtgcctgggcgacctgatccagacaatctcccactctgagatcctggtgggcctgaacgccgccggctttaatctgaccctggatagatccgacctgaaggagaaccagctgtctaggctggccgtgaagtacctgaccctgtgcgtgcagaccgccatcagcaatctggaggtcggctccgagcctctgtgcatcatcggcggccagctggatgacgatatcagcttccaggtggcacacttcctgtgccggcggctgtgcatcatcagcctggtgcactccaacgtgcagaatctgcctccaatcagagataacgaggtggacgtgaagagcaagctgatctacgatcacctgaagctggtggccacaaccctgaacaatagggaccagtcctatctgctgaccctgctgaacaagcccaatatcgagctgcacacacctcaggtgtacttcatcatgcggaagtgtctgggcctgctgaaggtgtatggcccagtgccccagaagcagcctctgccaacctctccagtggtgagcctgcccaacaagtgcaagtccaagtggaagctggaggaagtgatcgacagcatcgagtcccccaagtcttttaattgggtgcctgatacaaccatcccactggacgccgagcagaacccccctaacccaaatcgcgtgatcgataagatcaatatcctgcgcagcctgtccccaaggcacagcgtgtggtacagaaaccgccagtacagatatgtgctgagccagctgggacacgaccagctgggaggagccaccctgtatctgggagagggctctggcagcacaatcctgaatatcgagccaaaggtgcgctccgataagatctactatcacacctacttctccgccgacaagtctcccgcccagcgcaactttatcccccagcctacaaccttcctgcggagcaacttctaccactatgagctggagccctccggctgtgagttcatcaactgctggtctgaggatgtgaacgccacaaatctgaccgagctgcgctgtatcaatcacatcctgaccgtgatccctgtgggctctctgacacggatcatctgcgatgtggagctggccaacgacaccagcatccagtccgtggccacagcctacatgaacctgagcatcctggcacacgcactgctgaatcagggaggcgtgtgcatctgtcggtgccacctgctggacacctctcacctggccatcgtgagcttcgtgctgaagacactgagctcccagctggccatctccttttctggctattgtggcaccaacgatcctagctgcgtgatcggcgtgacaaaggagtgtagcatctccctggacgtgctgtctagcatcgccgccgccttcatcaacgagctgccaagcaatgagctgccagtgccccagtccctgctgacactgctgggctgctaccaggatcagctggagtctctgagcgccctgatcgagaagacctggatccaggagatccgccggccacacctgatgcagtgtgagatggagtggatcggcctgctgggcaccgacgcactgggcgacgtggatacattcatctcctatagaaacgattcttgcaatagcatcccagacctgctgacccctgccgtgtctgccctgctgtttgagctgatcagcctgacacctgagctgccaggcaccgaggatcagccctccagaagagtgatctctatcggccaggctttcaacctgaccatcagcggcaaggtcaatacaatgatcaggtcctgctgtgagcagtgcatcaagctgctggccgccaacgtgtccatcctgtctgacgtggatctgtcctacttcgtgaggggcatccgcgacggctcttttgccctgggcctgctgatcacacagagacagatcctgaaggccagccgggcccccaagtatctgaagacccaccaggtgcagaattggatcgccctgctgctggaggtgaagctggaggagatcttcagccggcactacagaaaggtgctgctgaggaccctgcgcctgctgagcctgtatggcctgctccagcagaaggagtcctaatgaataaagatcttataataaaaattcccatagctgcagctcgagaggcctaattaattaagtcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattgatctcgatcccgcgaaattaatacgactcactatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataatacc

SEQ ID NO: 13 - DNA, 3813 bp, synthetic construct
  source 1..3813: mol_type = other DNA
atgttcgtgtttctggtgctgctgcctctggtgagctcccagtgcgtgaacctgaggacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcggcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatcggtaccggctgtttagaaagtctaatctgaagccattcgagagggacatctccacagagatctaccaggccggctctaagccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccagagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcagaacgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacc

SEQ ID NO: 14 - DNA, 20166 bp, synthetic construct
  source 1..20166: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagt

SEQ ID NO: 15 - DNA, 20160 bp, synthetic construct
  source 1..20160: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaggacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcggcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatcggtaccggctgtttagaaagtctaatctgaagccattcgagagggacatctccacagagatctaccaggccggctctaagccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccagagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcagaacgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagt

SEQ ID NO: 16 - DNA, 20166 bp, synthetic construct
  source 1..20166: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtacatcaagtggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatgatttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagt

SEQ ID NO: 17 - DNA, 20166 bp, synthetic construct
  source 1..20166: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagt

SEQ ID NO: 18 - DNA, 20166 bp, synthetic construct
  source 1..20166: mol_type = other DNA
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttacgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcgccgccaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccacggagagcccggtctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggacaaggtggaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtttataaaaaacattgtgagaatgcaatttaaggacgaaagtaggagcggaagccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcagcgaaggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcccacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatattagggctagacaactcctagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggagcaataatagggtccatcgcgcttggtgttgccacatcagcacagattaccgctgcaattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatcgccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacggcgtgagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgcgtgctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacataagtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccggaggcctcatttaatgcaatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagt